SEQUENCE LISTING (289 sequences)

SEQ ID NO: 1 - DNA, 4614 bp, Saccharomyces cerivisiae
atgacaatgcctcatcgctatatgtttttggcagtctttacacttctggcactaactagtgtggcctcaggagccacagaggcgtgcttaccagcaggccagaggaaaagtgggatgaatataaatttttaccagtattcattgaaagattcctccacatattcgaatgcagcatatatggcttatggatatgcctcaaaaaccaaactaggttctgtcggaggacaaactgatatctcgattgattataatattccctgtgttagttcatcaggcacatttccttgtcctcaagaagattcctatggaaactggggatgcaaaggaatgggtgcttgttctaatagtcaaggaattgcatactggagtactgatttatttggtttctatactaccccaacaaacgtaaccctagaaatgacaggttattttttaccaccacagacgggttcttacacattcaagtttgctacagttgacgactctgcaattctatcagtaggtggtgcaaccgcgttcaactgttgtgctcaacagcaaccgccgatcacatcaacgaactttaccattgacggtatcaagccatggggtggaagtttgccacctaatatcgaaggaaccgtctatatgtacgctggctactattatccaatgaaggttgtttactcgaacgctgtttcttggggtacacttccaattagtgtgacacttccagatggtaccactgtaagtgatgacttcgaagggtacgtctattcctttgacgatgacctaagtcaatctaactgtactgtccctgacccttcaaattatgctgtcagtaccactacaactacaacggaaccatggaccggtactttcacttctacatctactgaaatgaccaccgtcaccggtaccaacggcgttccaactgacgaaaccgtcattgtcatcagaactccaacaactgctagcaccatcataactacaactgagccatggaacagcacttttacctctacttctaccgaattgaccacagtcactggcaccaatggtgtacgaactgacgaaaccatcattgtaatcagaacaccaacaacagccactactgccataactacaactgagccatggaacagcacttttacctctacttctaccgaattgaccacagtcaccggtaccaatggtttgccaactgatgagaccatcattgtcatcagaacaccaacaacagccactactgccatgactacaactcagccatggaacgacacttttacctctacttctaccgaattgaccacagtcaccggtaccaatggtttgccaactgatgagaccatcattgtcatcagaacaccaacaacagccactactgccatgactacaactcagccatggaacgacacttttacctctacttctaccgaattgaccacagtcaccggtaccaatggtttgccaactgatgagaccatcattgtcatcagaacaccaacaacagccactactgccatgactacaactcagccatggaacgacacttttacctctacatccactgaaatcaccaccgtcaccggtaccaatggtttgccaactgatgagaccatcattgtcatcagaacaccaacaacagccactactgccatgactacacctcagccatggaacgacacttttacctctacatccactgaaatgaccaccgtcaccggtaccaacggtttgccaactgatgaaaccatcattgtcatcagaacaccaacaacagccactactgccataactacaactgagccatggaacagcacttttacctctacatccactgaaatgaccaccgtcaccggtaccaacggtttgccaactgatgaaaccatcattgtcatcagaacaccaacaacagccactactgccataactacaactcagccatggaacgacacttttacctctacatccactgaaatgaccaccgtcaccggtaccaacggtttgccaactgatgaaaccatcattgtcatcagaacaccaacaacagccactactgccatgactacaactcagccatggaacgacacttttacctctacatccactgaaatcaccaccgtcaccggtaccaccggtttgccaactgatgagaccatcattgtcatcagaacaccaacaacagccactactgccatgactacaactcagccatggaacgacacttttacctctacatccactgaaatgaccaccgtcaccggtaccaacggcgttccaactgacgaaaccgtcattgtcatcagaactccaactagtgaaggtctaatcagcaccaccactgaaccatggactggtactttcacctctacatccactgagatgaccaccgtcaccggtactaacggtcaaccaactgacgaaaccgtgattgttatcagaactccaaccagtgaaggtttggttacaaccaccactgaaccatggactggtacttttacttctacatctactgaaatgaccaccattactggaaccaacggcgttccaactgacgaaaccgtcattgtcatcagaactccaaccagtgaaggtctaatcagcaccaccactgaaccatggactggtacttttacttctacatctactgaaatgaccaccattactggaaccaatggtcaaccaactgacgaaaccgttattgttatcagaactccaactagtgaaggtctaatcagcactacaacggaaccatggaccggtactttcacttctacatctactgaaatgacgcacgtcaccggtaccaacggcgttccaactgacgaaaccgtcattgtcatcagaactccaaccagtgaaggtctaatcagcaccaccactgaaccatggactggcactttcacttcgacttccactgaggttaccaccatcactggaaccaacggtcaaccaactgacgaaactgtgattgttatcagaactccaaccagtgaaggtctaatcagcaccaccactgaaccatggactggtactttcacttctacatctactgaaatgaccaccgtcaccggtactaacggtcaaccaactgacgaaaccgtgattgttatcagaactccaaccagtgaaggtttggttacaaccaccactgaaccatggactggtacttttacttcgacttccactgaaatgtctactgtcactggaaccaatggcttgccaactgatgaaactgtcattgttgtcaaaactccaactactgccatctcatccagtttgtcatcatcatcttcaggacaaatcaccagctctatcacgtcttcgcgtccaattattaccccattctatcctagcaatggaacttctgtgatttcttcctcagtaatttcttcctcagtcacttcttctctattcacttcttctccagtcatttcttcctcagtcatttcttcttctacaacaacctccacttctatattttctgaatcatctaaatcatccgtcattccaaccagtagttccacctctggttcttctgagagcgaaacgagttcagctggttctgtctcttcttcctcttttatctcttctgaatcatcaaaatctcctacatattcttcttcatcattaccacttgttaccagtgcgacaacaagccaggaaactgcttcttcattaccacctgctaccactacaaaaacgagcgaacaaaccactttggttaccgtgacatcctgcgagtctcatgtgtgcactgaatccatctcccctgcgattgtttccacagctactgttactgttagcggcgtcacaacagagtataccacatggtgccctatttctactacagagacaacaaagcaaaccaaagggacaacagagcaaaccacagaaacaacaaaacaaaccacggtagttacaatttcttcttgtgaatctgacgtatgctctaagactgcttctccagccattgtatctacaagcactgctactattaacggcgttactacagaatacacaacatggtgtcctatttccaccacagaatcgaggcaacaaacaacgctagttactgttacttcctgcgaatctggtgtgtgttccgaaactgcttcacctgccattgtttcgacggccacggctactgtgaatgatgttgttacggtctatcctacatggaggccacagactgcgaatgaagagtctgtcagctctaaaatgaacagtgctaccggtgagacaacaaccaatactttagctgctgaaacgactaccaatactgtagctgctgagacgattaccaatactggagctgctgagacgaaaacagtagtcacctcttcgctttcaagatctaatcacgctgaaacacagacggcttccgcgaccgatgtgattggtcacagcagtagtgttgtttctgtatccgaaactggcaacaccaagagtctaacaagttccgggttgagtactatgtcgcaacagcctcgtagcacaccagcaagcagcatggtaggatatagtacagcttctttagaaatttcaacgtatgctggcagtgccaacagcttactggccggtagtggtttaagtgtcttcattgcgtccttattgctggcaattatttaa

SEQ ID NO: 2 - DNA, 3228 bp, Saccharomyces cerivisiae
atgacaattgcacaccactgcatatttttggtaatcttggcctttctggcactaattaatgtggcctcaggagccacagaggcgtgcttaccagcaggccagaggaaaagtgggatgaatataaatttttaccagtattcattgaaagattcctccacatattcgaatgcagcatatatggcttatggatatgcctcaaaaaccaaactaggttctgtcggaggacaaactgatatttcgattgattataatattccctgtgttagttcatcaggcacatttccttgtcctcaagaagattcctatggaaactggggatgcaaaggaatgggtgcttgttctaatagtcaaggaattgcatactggagtactgatttatttggtttctatactaccccaacaaacgtaaccctagaaatgacaggttattttttaccaccacagacgggttcttacacgttttcttttgcaacagtagatgattctgcaattttatcagtcggtggtagcattgcgttcgaatgttgtgcacaagaacaacctcccatcacgtcgactaacttcacaatcaatggtatcaagccatgggatggaagtctccctgacaatatcacagggactgtctacatgtatgcaggctactattatccgctgaaggttgtttactccaatgccgtttcctggggcacgcttccaattagcgtggaattgcctgatggtactactgttagtgataactttgaagggtacgtttactcttttgacgatgacctaagtcagtcaaattgtactatccctgatccttcaatacatactactagcactatcacaactaccaccgagccatggaccggtactttcacttctacatccactgagatgaccaccatcaccgatactaacggtcaattaactgatgaaactgtcattgtcatcagaactccaacaacagctagcaccatcacaactaccaccgagccatggaccggtactttcacctctacatccactgagatgactactgtcaccggtaccaacggtcaaccaactgacgaaactgttattgtcattagaactccaactagtgagggtttgattactacaactaccgaaccatggaccggtactttcacctctacatccactgagatgactactgtgaccggtaccaacggtcaaccaactgacgaaactgttattgtcattagaactccaactagtgagggtttgattactacaactaccgaaccatggaccggtactttcacctctacatccactgaggttaccaccatcactggtaccaacggtcaaccaactgacgaaaccgtgattgtcattagaactccaactagtgagggtttgattactacaactaccgaaccatggaccggtactttcacctctacatctactgagatgactactgtcaccggtaccaacggtcaaccaactgacgaaactgttattgttatcagaactccaaccagtgaaggtctaatcagcaccaccactgaaccatggactggtactttcacctctacatctactgaggttaccaccatcactggtaccaacggtcaaccaactgacgaaaccgtgattgtcattagaactccaactagtgagggtttgattactacaactaccgaaccatggaccggaactttcacctctacatccactgagatgactactgtgaccggtaccaacggtcaaccaactgacgaaactgttattgtcattagaactccaactagtgagggtttgattactagaactaccgaaccatggactggtactttcacttctacatctactgaggttaccaccatcaccggtaccaacggtcaaccaactgacgaaactgttattgtcatcagaactccaactactgccatctcatccagtttgtcatcttcttcaggacaaatcaccagctctatcacgtcttcgcgtccaattattaccccattctatcctagcaatggaacttctgtgatttcctcctcagtaatttcttcttcagtcacttcttctctagtcacctcttcttcattcatttcttcctctgtcatttcttcttctacaacaacctccacttctatattctctgaatcatctacatcatccgtcattccaaccagtagttccacctctggttcttctgagagcaaaacgagttcggctagttcttcctcttcttcctcttctatctcttctgaatcaccaaagtctcctacaaattcttcttcatcattaccacctgttaccagtgcgacaacaggccaggaaactgcttcttcattaccacctgctaccactacaaaaacgagcgaacaaaccactttggttaccgtgacatcctgcgaatctcatgtgtgtactgaatccatctcctctgctattgtttccacggccaccgttactgttagcggcgtcacaacagagtataccacgtggtgccctatttctaccacagagacaacaaagcaaaccaaggggacaacagagcaaaccaaggggacaacagagcaaaccacagaaacaacaaaacaaaccacagtagttacaatttcttcttgtgaatctgacatatgctctaagactgcttctccagccattgtgtctacaagcactgctactattaacggcgttaccacagaatacacaacatggtgtcctatttccaccacagaatcgaagcaacaaactacgctagttactgttacttcctgcgaatctggtgtgtgttccgaaactacttcacctgccattgtttcgacggccacggctactgtgaatgatgttgttacggtctatcctacatggagaccacagactacgaatgaacagtctgtcagctctaaaatgaacagtgctaccagtgagacaactaccaatactggggctgctgagacaaaaacagcagtcacctcttcactttcaagattcaatcacgctgaaacacagacagcttccgcgaccgatgtgattggtcacagcagtagtgttgtttctgtatccgaaactggcaacaccatgagtctaacaagttccgggttgagcactatgtcgcaacagcctcgtagcacaccagcaagtagcatggtaggatctagtacagcttctttagaaatttcaacgtatgctggcagtgccaacagcttactggccggtagtggtttaagtgtcttcattgcgtccttattgctggcaattatttaa

SEQ ID NO: 3 - DNA, 3969 bp, Saccharomyces cerivisiae
atgtctctggcacattattgtttactactagccatcgtcacattgctgggattaactaatgttgtctctgcgactacagcggcatgcctgccagcaaactcaaggaagaatggtatgaatgtaaacttttaccagtattcattgagagattcctccacatattcgaatgcagcatatatggcttatggatatgcctcaaaaactaaactgggttctgtcggaggacaaactgatatctcgattgattataatattccttgtgttagttcatcaggcacatttccttgtcctcaagaagatttatatggtaattggggatgcaaaggaattggtgcttgttctaataatccaataattgcatactggagtactgatttatttggtttctatactaccccaacaaacgtaaccctagaaatgacaggttattttttaccaccacagacgggttcttacacattcaagtttgctacagttgacgactctgcaattctatcagtcggtggtagcattgcgttcgaatgttgtgcacaagaacaacctcccatcacgtcgactaacttcaccatcaatggtatcaagccatggaatggaagtccccctgataatattacagggactgtctacatgtatgctggtttctattatccaatgaagattgtttactcaaatgccgttgcctggggtacacttccaattagtgtgacactaccagatggcactaccgttagtgatgactttgaagggtacgtatatacttttgacaacaatctaagccagccaaactgtaccattccagacccttcaaattatactgtcagtactaccataactacaacggaaccatggaccggtactttcacttctacatctactgaaatgaccaccgtcaccggtaccaacggcgttccaactgacgaaaccgtcattgtcatcagaactccaacaactgctagcaccatcataactacaactgagccatggaacagcacttttacctctacttctaccgaattgaccacagtcactggcaccaatggtgtacgaactgacgaaaccatcattgtaatcagaacaccaacaacagccactactgccataactacaactgagccatggaacagcacttttacctctacttctaccgaattgaccacagtcaccggtaccaatggtttgccaactgatgagaccatcattgtcatcagaacaccaacaacagccactactgccatgactacaactcagccatggaacgacacttttacctctacttctaccgaattgaccacagtcaccggtaccaatggtttgccaactgatgagaccatcattgtcatcagaacaccaacaacagccactactgccatgactacaactcagccatggaacgacacttttacctctacttctaccgaattgaccacagtcaccggtaccaatggtttgccaactgatgagaccatcattgtcatcagaacaccaacaacagccactactgccatgactacaactcagccatggaacgacacttttacctctacatccactgaaatcaccaccgtcaccggtaccaatggtttgccaactgatgagaccatcattgtcatcagaacaccaacaacagccactactgccatgactacaactcagccatggaacgacacttttacctctacatccactgaaatgaccaccgtcaccggtaccaacggtttgccaactgatgaaaccatcattgtcatcagaacaccaacaacagccactactgccataactacaactgagccatggaacagcacttttacctctacatccactgaaatgaccaccgtcaccggtaccaacggtttgccaactgatgaaaccatcattgtcatcagaacaccaacaacagccactactgccataactacaactcagccatggaacgacacttttacctctacatccactgaaatgaccaccgtcaccggtaccaacggtttgccaactgatgaaaccatcattgtcatcagaacaccaacaacagccactactgccatgactacaactcagccatggaacgacacttttacctctacatccactgaaatcaccaccgtcaccggtaccaacggtttgccaactgatgagaccatcattgtcatcagaacaccaacaacagccactactgccatgactacaactcagccatggaacgacacttttacctctacatccactgaaatgaccaccgtcaccggtaccaacggcgttccaactgacgaaaccgtcattgtcatcagaactccaactagtgaaggtctaatcagcaccaccactgaaccatggactggtactttcacctctacatccactgagatgaccaccgtcaccggtactaacggtcaaccaactgacgaaaccgtgattgttatcagaactccaaccagtgaaggtttggttacaactacaaccgagccatggaccggtactttcacctctacatctactgagatgaccaccatcactggaaccaacggtcaaccaactgatgaaactgtcattattgtcaaaactccaactactgccatctcatccagtttgtcatcttcttcaggacaaatcaccagctttatcacgtctgcgcgtccaattattaccccattctatcctagcaatggaacttctgtgatttcctcctcagtaatttcttcctcagacacttcttctctagtcatttcttcctcagtcacttcttctctagtcacttcttctccagtcatttcttcttcattcatttcttcccctgtcatttcttctacaacaacctccgcttctatactctctgaatcatctaaatcatccgtcattccaaccagtagttccacctctggttcttctgagagcgaaacgggttcagctagttctgcctcttcttcctcttctatctcttctgaatcaccaaagtctacatattcgtcttcatcattaccacctgttaccagtgcaacaacaagtcaggaaattacttcttcattaccacctgttaccactacaaaaacgagcgaacaaaccactttggttaccgtgacatcctgcgaatctcatgtgtgcactgaatctatctcctctgcgattgtttccacggccaccgttactgttagcggtgccacaacagagtataccacatggtgccctatttctaccacagagataacaaagcaaactacggagacaacaaagcaaaccaaggggacaacagagcaaaccacagaaacaacaaaacaaaccacagtagttacaatttcttcttgtgaatctgacgtatgctctaagactgcttctccagccattgtatctacaagcactgctactattaatggcgttaccacagaatacacaacatggtgtcctatttccaccacagaatcgaagcaacaaactacgctagttactgttacttcctgcggatctggtgtgtgttccgaaactacttcacctgccattgtttcgacggccacggctactgtgaatgatgttgttacggtctattctacatggaggccacagactacgaatgaacagtctgtcagctctaaaatgaacagtgctaccagtgagacaacaaccaatactggagctgctgagacaactaccagtactggagctgctgagacgaaaacagtagtcacctcttcaatttcaagattcaatcatgctgaaacacagacggcttccgcgaccgatgtgattggtcacagcagtagtgttgtttctgtatccgaaactggcaacaccaagagtctaacaagttccgggttgagtactatgtcgcaacagcctcgtagcacaccagcaagtagcatggtaggatctagtacagcttctttagaaatttcaacgtatgctggcagtgccaacagcttactggccggtagtggtttaagtgtcttcattgcgtccttattgctggcaattatttaa

SEQ ID NO: 4 - protein, 559 aa, Klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAIEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYTFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLRAAMDVDGPAVVAIPVDYRDNPLLMGQLHLSQIL

SEQ ID NO: 5 - protein, 571 aa, Bacillus subtilis
MLTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 6 - protein, 554 aa, Lactococcus lactis
MSEKQFGANLVVDSLINHKVKYVFGIPGAKIDRVFDLLENEEGPQMVVTRHEQGAAFMAQAVGRLTGEPGVVVVTSGPGVSNLATPLLTATSEGDAILAIGGQVKRSDRLKRAHQSMDNAGMMQSATKYSAEVLDPNTLSESIANAYRIAKSGHPGATFLSIPQDVTDAEVSIKAIQPLSDPKMGNASIDDINYLAQAIKNAVLPVILVGAGASDAKVASSLRNLLTHVNIPVVETFQGAGVISHDLEHTFYGRIGLFRNQPGDMLLKRSDLVIAVGYDPIEYEARNWNAEIDSRIIVIDNAIAEIDTYYQPERELIGDIAATLDNLLPAVRGYKIPKGTKDYLDGLHEVAEQHEFDTENTEEGRMHPLDLVSTFQEIVKDDETVTVDVGSLYIWMARHFKSYEPRHLLFSNGMQTLGVALPWAITAALLRPGKKVYSHSGDGGFLFTGQELETAVRLNLPIVQIIWNDGHYDMVKFQEEMKYGRSAAVDFGYVDYVKYAEAMRAKGYRAHSKEELAEILKSIPDTTGPVVIDVPLDYSDNIKLAEKLLPEEFY

SEQ ID NO: 7 - DNA, 1680 bp, Klebsiella pneumoniae
atggacaaacagtatccggtacgccagtgggcgcacggcgccgatctcgtcgtcagtcagctggaagctcagggagtacgccaggtgttcggcatccccggcgccaaaatcgacaaggtctttgattcactgctggattcctccattcgcattattccggtacgccacgaagccaacgccgcatttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccatcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctcaaccagtttgccctgcatcccctgcgcatcgttcgcgccatgcaggatatcgtcaacagcgacgtcacgttgaccgtggacatgggcagcttccatatctggattgcccgctacctgtacacgttccgcgcccgtcaggtgatgatctccaacggccagcagaccatgggcgtcgccctgccctgggctatcggcgcctggctggtcaatcctgagcgcaaagtggtctccgtctccggcgacggcggcttcctgcagtcgagcatggagctggagaccgccgtccgcctgaaagccaacgtgctgcatcttatctgggtcgataacggctacaacatggtcgctatccaggaagagaaaaaatatcagcgcctgtccggcgtcgagtttgggccgatggattttaaagcctatgccgaatccttcggcgcgaaagggtttgccgtggaaagcgccgaggcgctggagccgaccctgcgcgcggcgatggacgtcgacggcccggcggtagtggccatcccggtggattatcgcgataacccgctgctgatgggccagctgcatctgagtcagattctgtaa

SEQ ID NO: 8 - DNA, 1716 bp, Bacillus subtilis
atgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 9 - DNA, 1665 bp, Lactococcus lactis
atgtctgagaaacaatttggggcgaacttggttgtcgatagtttgattaaccataaagtgaagtatgtatttgggattccaggagcaaaaattgaccgggtttttgatttattagaaaatgaagaaggccctcaaatggtcgtgactcgtcatgagcaaggagctgctttcatggctcaagctgtcggtcgtttaactggcgaacctggtgtagtagttgttacgagtgggcctggtgtatcaaaccttgcgactccgcttttgaccgcgacatcagaaggtgatgctattttggctatcggtggacaagttaaacgaagtgaccgtcttaaacgtgcgcaccaatcaatggataatgctggaatgatgcaatcagcaacaaaatattcagcagaagttcttgaccctaatacactttctgaatcaattgccaacgcttatcgtattgcaaaatcaggacatccaggtgcaactttcttatcaatcccccaagatgtaacggatgccgaagtatcaatcaaagccattcaaccactttcagaccctaaaatggggaatgcctctattgatgacattaattatttagcacaagcaattaaaaatgctgtattgccagtaattttggttggagctggtgcttcagatgctaaagtcgcttcatccttgcgtaatctattgactcatgttaatattcctgtcgttgaaacattccaaggtgcaggggttatttcacatgatttagaacatactttttatggacgtatcggtcttttccgcaatcaaccaggcgatatgcttctgaaacgttctgaccttgttattgctgttggttatgacccaattgaatatgaagctcgtaactggaatgcagaaattgatagtcgaattatcgttattgataatgccattgctgaaattgatacttactaccaaccagagcgtgaattaattggtgatatcgcagcaacattggataatcttttaccagctgttcgtggctacaaaattccaaaaggaacaaaagattatctcgatggccttcatgaagttgctgagcaacacgaatttgatactgaaaatactgaagaaggtagaatgcaccctcttgatttggtcagcactttccaagaaatcgtcaaggatgatgaaacagtaaccgttgacgtaggttcactctacatttggatggcacgtcatttcaaatcatacgaaccacgtcatctcctcttctcaaacggaatgcaaacactcggagttgcacttccttgggcaattacagccgcattgttgcgcccaggtaaaaaagtttattcacactctggtgatggaggcttccttttcacagggcaagaattggaaacagctgtacgtttgaatcttccaatcgttcaaattatctggaatgacggccattatgatatggttaaattccaagaagaaatgaaatatggtcgttcagcagccgttgattttggctatgttgattacgtaaaatatgctgaagcaatgagagcaaaaggttaccgtgcacacagcaaagaagaacttgctgaaattctcaaatcaatcccagatactactggaccggtggtaattgacgttcctttggactattctgataacattaaattagcagaaaaattattgcctgaagagttttattga

SEQ ID NO: 10 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 11 - protein, 330 aa, Methanococcus maripaludis
MKVFYDSDFKLDALKEKTIAVIGYGSQGRAQSLNMKDSGLNVVVGLRKNGASWNNAKADGHNVMTIEEAAEKADIIHILIPDELQAEVYESQIKPYLKEGKTLSFSHGFNIHYGFIVPPKGVNVVLVAPKSPGKMVRRTYEEGFGVPGLICIEIDATNNAFDIVSAMAKGIGLSRAGVIQTTFKEETETDLFGEQAVLCGGVTELIKAGFETLVEAGYAPEMAYFETCHELKLIVDLIYQKGFKNMWNDVSNTAEYGGLTRRSRIVTADSKAAMKEILREIQDGRFTKEFLLEKQVSYAHLKSMRRLEGDLQIEEVGAKLRKMCGLEKEE

SEQ ID NO: 12 - protein, 342 aa, Bacillus subtilis
MVKVYYNGDIKENVLAGKTVAVIGYGSQGHAHALNLKESGVDVIVGVRQGKSFTQAQEDGHKVFSVKEAAAQAEIIMVLLPDEQQQKVYEAEIKDELTAGKSLVFAHGFNVHFHQIVPPADVDVFLVAPKGPGHLVRRTYEQGAGVPALFAIYQDVTGEARDKALAYAKGIGGARAGVLETTFKEETETDLFGEQAVLCGGLSALVKAGFETLTEAGYQPELAYFECLHELKLIVDLMYEEGLAGMRYSISDTAQWGDFVSGPRVVDAKVKESMKEVLKDIQNGTFAKEWIVENQVNRPRFNAINASENEHQIEVVGRKLREMMPFVKQGKKKEAVVSVAQN

SEQ ID NO: 13 - DNA, 1476 bp, Escherichia coli
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 14 - DNA, 1188 bp, Saccharomyces cerevisiae
atgttgagaactcaagccgccagattgatctgcaactcccgtgtcatcactgctaagagaacctttgctttggccacccgtgctgctgcttacagcagaccagctgcccgtttcgttaagccaatgatcactacccgtggtttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaataa

SEQ ID NO: 15 - DNA, 993 bp, Methanococcus maripaludis
atgaaggtattctatgactcagattttaaattagatgctttaaaagaaaaaacaattgcagtaatcggttatggaagtcaaggtagggcacagtccttaaacatgaaagacagcggattaaacgttgttgttggtttaagaaaaaacggtgcttcatggaacaacgctaaagcagacggtcacaatgtaatgaccattgaagaagctgctgaaaaagcggacatcatccacatcttaatacctgatgaattacaggcagaagtttatgaaagccagataaaaccatacctaaaagaaggaaaaacactaagcttttcacatggttttaacatccactatggattcattgttccaccaaaaggagttaacgtggttttagttgctccaaaatcacctggaaaaatggttagaagaacatacgaagaaggtttcggtgttccaggtttaatctgtattgaaattgatgcaacaaacaacgcatttgatattgtttcagcaatggcaaaaggaatcggtttatcaagagctggagttatccagacaactttcaaagaagaaacagaaactgaccttttcggtgaacaagctgttttatgcggtggagttaccgaattaatcaaggcaggatttgaaacactcgttgaagcaggatacgcaccagaaatggcatactttgaaacctgccacgaattgaaattaatcgttgacttaatctaccaaaaaggattcaaaaacatgtggaacgatgtaagtaacactgcagaatacggcggacttacaagaagaagcagaatcgttacagctgattcaaaagctgcaatgaaagaaatcttaagagaaatccaagatggaagattcacaaaagaattccttctcgaaaaacaggtaagctatgctcatttaaaatcaatgagaagactcgaaggagacttacaaatcgaagaagtcggcgcaaaattaagaaaaatgtgcggtcttgaaaaagaagaataa

SEQ ID NO: 16 - DNA, 1476 bp, Bacillus subtilis; identical to SEQ ID NO: 13

SEQ ID NO: 17 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 18 - protein, 585 aa, Saccharomyces cerevisiae
MGLLTKVATSRQFSTTRCVAKKLNKYSYIITEPKGQGASQAMLYATGFKKEDFKKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKEKLAECDNIGEYIKKTMELGILPRDILTKEAFENAITYVVATGGSTNAVLHLVAVAHSAGVKLSPDDFQRISDTTPLIGDFKPSGKYVMADLINVGGTQSVIKYLYENNMLHGNTMTVTGDTLAERAKKAPSLPEGQEIIKPLSHPIKANGHLQILYGSLAPGGAVGKITGKEGTYFKGRARVFEEEGAFIEALERGEIKKGEKTVVVIRYEGPRGAPGMPEMLKPSSALMGYGLGKDVALLTDGRFSGGSHGFLIGHIVPEAAEGGPIGLVRDGDEIIIDADNNKIDLLVSDKEMAQRKQSWVAPPPRYTRGTLSKYAKLVSNASNGCVLDA

SEQ ID NO: 19 - protein, 550 aa, Methanococcus maripaludis
MISDNVKKGVIRTPNRALLKACGYTDEDMEKPFIGIVNSFTEVVPGHIHLRTLSEAAKHGVYANGGTPFEFNTIGICDGIAMGHEGMKYSLPSREIIADAVESMARAHGFDGLVLIPTCDKIVPGMIMGALRLNIPFIVVTGGPMLPGEFQGKKYELISLFEGVGEYQVGKITEEELKCIEDCACSGAGSCAGLYTANSMACLTEALGLSLPMCATTHAVDAQKVRLAKKSGSKIVDMVKEDLKPTDILTKEAFENAILVDLALGGSTNTTLHIPAIANEIENKFITLDDFDRLSDEVPHIASIKPGGEHYMIDLHNAGGIPAVLNVLKEKIRDTKTVDGRSILEIAESVKYINYDVIRKVEAPVHETAGLRVLKGNLAPNGCVVKIGAVHPKMYKHDGPAKVYNSEDEAISAILGGKIVEGDVIVIRYEGPSGGPGMREMLSPTSAICGMGLDDSVALITDGRFSGGSRGPCIGHVSPEAAAGGVIAAIENGDIIKIDMIEKEINVDLDESVIKERLSKLGEFEPKIKKGYLSRYSKLVSSADEGAVLK

SEQ ID NO: 20 - protein, 558 aa, Bacillus subtilis
MAELRSNMITQGIDRAPHRSLLRAAGVKEEDFGKPFIAVCNSYIDIVPGHVHLQEFGKIVKEAIREAGGVPFEFNTIGVDDGIAMGHIGMRYSLPSREIIADSVETVVSAHWFDGMVCIPNCDKITPGMLMAAMRINIPTIFVSGGPMAAGRTSYGRKISLSSVFEGVGAYQAGKINENELQELEQFGCPTCGSCSGMFTANSMNCLSEALGLALPGNGTILATSPERKEFVRKSAAQLMETIRKDIKPRDIVTVKAIDNAFALDMALGGSTNTVLHTLALANEAGVEYSLERINEVAERVPHLAKLAPASDVFIEDLHEAGGVSAALNELSKKEGALHLDALTVTGKTLGETIAGHEVKDYDVIHPLDQPFTEKGGLAVLFGNLAPDGAIIKTGGVQNGITRHEGPAVVFDSQDEALDGIINRKVKEGDVVIIRYEGPKGGPGMPEMLAPTSQIVGMGLGPKVALITDGRFSGASRGLSIGHVSPEAAEGGPLAFVENGDHIIVDIEKRILDVQVPEEEWEKRKANWKGFEPKVKTGYLARYSKLVTSANTGGIMKI

SEQ ID NO: 21 - DNA, 1851 bp, Escherichia coli
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 22 - DNA, 1758 bp, Saccharomyces cerevisiae
atgggcttgttaacgaaagttgctacatctagacaattctctacaacgagatgcgttgcaaagaagctcaacaagtactcgtatatcatcactgaacctaagggccaaggtgcgtcccaggccatgctttatgccaccggtttcaagaaggaagatttcaagaagcctcaagtcggggttggttcctgttggtggtccggtaacccatgtaacatgcatctattggacttgaataacagatgttctcaatccattgaaaaagcgggtttgaaagctatgcagttcaacaccatcggtgtttcagacggtatctctatgggtactaaaggtatgagatactcgttacaaagtagagaaatcattgcagactcctttgaaaccatcatgatggcacaacactacgatgctaacatcgccatcccatcatgtgacaaaaacatgcccggtgtcatgatggccatgggtagacataacagaccttccatcatggtatatggtggtactatcttgcccggtcatccaacatgtggttcttcgaagatctctaaaaacatcgatatcgtctctgcgttccaatcctacggtgaatatatttccaagcaattcactgaagaagaaagagaagatgttgtggaacatgcatgcccaggtcctggttcttgtggtggtatgtatactgccaacacaatggcttctgccgctgaagtgctaggtttgaccattccaaactcctcttccttcccagccgtttccaaggagaagttagctgagtgtgacaacattggtgaatacatcaagaagacaatggaattgggtattttacctcgtgatatcctcacaaaagaggcttttgaaaacgccattacttatgtcgttgcaaccggtgggtccactaatgctgttttgcatttggtggctgttgctcactctgcgggtgtcaagttgtcaccagatgatttccaaagaatcagtgatactacaccattgatcggtgacttcaaaccttctggtaaatacgtcatggccgatttgattaacgttggtggtacccaatctgtgattaagtatctatatgaaaacaacatgttgcacggtaacacaatgactgttaccggtgacactttggcagaacgtgcaaagaaagcaccaagcctacctgaaggacaagagattattaagccactctcccacccaatcaaggccaacggtcacttgcaaattctgtacggttcattggcaccaggtggagctgtgggtaaaattaccggtaaggaaggtacttacttcaagggtagagcacgtgtgttcgaagaggaaggtgcctttattgaagccttggaaagaggtgaaatcaagaagggtgaaaaaaccgttgttgttatcagatatgaaggtccaagaggtgcaccaggtatgcctgaaatgctaaagccttcctctgctctgatgggttacggtttgggtaaagatgttgcattgttgactgatggtagattctctggtggttctcacgggttcttaatcggccacattgttcccgaagccgctgaaggtggtcctatcgggttggtcagagacggcgatgagattatcattgatgctgataataacaagattgacctattagtctctgataaggaaatggctcaacgtaaacaaagttgggttgcacctccacctcgttacacaagaggtactctatccaagtatgctaagttggtttccaacgcttccaacggttgtgttttagatgcttga

SEQ ID NO: 23 - DNA, 1653 bp, Methanococcus maripaludis
atgataagtgataacgtcaaaaagggagttataagaactccaaaccgagctcttttaaaggcttgcggatatacagacgaagacatggaaaaaccatttattggaattgtaaacagctttacagaagttgttcccggccacattcacttaagaacattatcagaagcggctaaacatggtgtttatgcaaacggtggaacaccatttgaatttaataccattggaatttgcgacggtattgcaatgggccacgaaggtatgaaatactctttaccttcaagagaaattattgcagacgctgttgaatcaatggcaagagcacatggatttgatggtcttgttttaattcctacgtgtgataaaatcgttcctggaatgataatgggtgctttaagactaaacattccatttattgtagttactggaggaccaatgcttcccggagaattccaaggtaaaaaatacgaacttatcagcctttttgaaggtgtcggagaataccaagttggaaaaattactgaagaagagttaaagtgcattgaagactgtgcatgttcaggtgctggaagttgtgcagggctttacactgcaaacagtatggcctgccttacagaagctttgggactctctcttccaatgtgtgcaacaacgcatgcagttgatgcccaaaaagttaggcttgctaaaaaaagtggctcaaaaattgttgatatggtaaaagaagacctaaaaccaacagacatattaacaaaagaagcttttgaaaatgctattttagttgaccttgcacttggtggatcaacaaacacaacattacacattcctgcaattgcaaatgaaattgaaaataaattcataactctcgatgactttgacaggttaagcgatgaagttccacacattgcatcaatcaaaccaggtggagaacactacatgattgatttacacaatgctggaggtattcctgcggtattgaacgttttaaaagaaaaaattagagatacaaaaacagttgatggaagaagcattttggaaatcgcagaatctgttaaatacataaattacgacgttataagaaaagtggaagctccggttcacgaaactgctggtttaagggttttaaagggaaatcttgctccaaacggttgcgttgtaaaaatcggtgcagtacatccgaaaatgtacaaacacgatggacctgcaaaagtttacaattccgaagatgaagcaatttctgcgatacttggcggaaaaattgtagaaggggacgttatagtaatcagatacgaaggaccatcaggaggccctggaatgagagaaatgctctccccaacttcagcaatctgtggaatgggtcttgatgacagcgttgcattgattactgatggaagattcagtggtggaagtaggggcccatgtatcggacacgtttctccagaagctgcagctggcggagtaattgctgcaattgaaaacggggatatcatcaaaatcgacatgattgaaaaagaaataaatgttgatttagatgaatcagtcattaaagaaagactctcaaaactgggagaatttgagcctaaaatcaaaaaaggctatttatcaagatactcaaaacttgtctcatctgctgacgaaggggcagttttaaaataa

SEQ ID NO: 24 - DNA, 1677 bp, Bacillus subtilis
atggcagaattacgcagtaatatgatcacacaaggaatcgatagagctccgcaccgcagtttgcttcgtgcagcaggggtaaaagaagaggatttcggcaagccgtttattgcggtgtgtaattcatacattgatatcgttcccggtcatgttcacttgcaggagtttgggaaaatcgtaaaagaagcaatcagagaagcagggggcgttccgtttgaatttaataccattggggtagatgatggcatcgcaatggggcatatcggtatgagatattcgctgccaagccgtgaaattatcgcagactctgtggaaacggttgtatccgcacactggtttgacggaatggtctgtattccgaactgcgacaaaatcacaccgggaatgcttatggcggcaatgcgcatcaacattccgacgatttttgtcagcggcggaccgatggcggcaggaagaacaagttacgggcgaaaaatctccctttcctcagtattcgaaggggtaggcgcctaccaagcagggaaaatcaacgaaaacgagcttcaagaactagagcagttcggatgcccaacgtgcgggtcttgctcaggcatgtttacggcgaactcaatgaactgtctgtcagaagcacttggtcttgctttgccgggtaatggaaccattctggcaacatctccggaacgcaaagagtttgtgagaaaatcggctgcgcaattaatggaaacgattcgcaaagatatcaaaccgcgtgatattgttacagtaaaagcgattgataacgcgtttgcactcgatatggcgctcggaggttctacaaataccgttcttcatacccttgcccttgcaaacgaagccggcgttgaatactctttagaacgcattaacgaagtcgctgagcgcgtgccgcacttggctaagctggcgcctgcatcggatgtgtttattgaagatcttcacgaagcgggcggcgtttcagcggctctgaatgagctttcgaagaaagaaggagcgcttcatttagatgcgctgactgttacaggaaaaactcttggagaaaccattgccggacatgaagtaaaggattatgacgtcattcacccgctggatcaaccattcactgaaaagggaggccttgctgttttattcggtaatctagctccggacggcgctatcattaaaacaggcggcgtacagaatgggattacaagacacgaagggccggctgtcgtattcgattctcaggacgaggcgcttgacggcattatcaaccgaaaagtaaaagaaggcgacgttgtcatcatcagatacgaagggccaaaaggcggacctggcatgccggaaatgctggcgccaacatcccaaatcgttggaatgggactcgggccaaaagtggcattgattacggacggacgtttttccggagcctcccgtggcctctcaatcggccacgtatcacctgaggccgctgagggcgggccgcttgcctttgttgaaaacggagaccatattatcgttgatattgaaaaacgcatcttggatgtacaagtgccagaagaagagtgggaaaaacgaaaagcgaactggaaaggttttgaaccgaaagtgaaaaccggctacctggcacgttattctaaacttgtgacaagtgccaacaccggcggtattatgaaaatctag

SEQ ID NO: 25 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 26 - protein, 330 aa, Methanococcus maripaludis; identical to SEQ ID NO: 11

SEQ ID NO: 27 - DNA, 1662 bp, Lactococcus lactis
tctagacatatgtatactgtgggggattacctgctggatcgcctgcacgaactggggattgaagaaattttcggtgtgccaggcgattataacctgcagttcctggaccagattatctcgcacaaagatatgaagtgggtcggtaacgccaacgaactgaacgcgagctatatggcagatggttatgcccgtaccaaaaaagctgctgcgtttctgacgacctttggcgttggcgaactgagcgccgtcaacggactggcaggaagctacgccgagaacctgccagttgtcgaaattgttgggtcgcctacttctaaggttcagaatgaaggcaaatttgtgcaccatactctggctgatggggattttaaacattttatgaaaatgcatgaaccggttactgcggcccgcacgctgctgacagcagagaatgctacggttgagatcgaccgcgtcctgtctgcgctgctgaaagagcgcaagccggtatatatcaatctgcctgtcgatgttgccgcagcgaaagccgaaaagccgtcgctgccactgaaaaaagaaaacagcacctccaatacatcggaccaggaaattctgaataaaatccaggaatcactgaagaatgcgaagaaaccgatcgtcatcaccggacatgagatcatctcttttggcctggaaaaaacggtcacgcagttcatttctaagaccaaactgcctatcaccaccctgaacttcggcaaatctagcgtcgatgaagcgctgccgagttttctgggtatctataatggtaccctgtccgaaccgaacctgaaagaattcgtcgaaagcgcggactttatcctgatgctgggcgtgaaactgacggatagctccacaggcgcatttacccaccatctgaacgagaataaaatgatttccctgaatatcgacgaaggcaaaatctttaacgagcgcatccagaacttcgattttgaatctctgattagttcgctgctggatctgtccgaaattgagtataaaggtaaatatattgataaaaaacaggaggattttgtgccgtctaatgcgctgctgagtcaggatcgtctgtggcaagccgtagaaaacctgacacagtctaatgaaacgattgttgcggaacagggaacttcatttttcggcgcctcatccatttttctgaaatccaaaagccatttcattggccaaccgctgtgggggagtattggttatacctttccggcggcgctgggttcacagattgcagataaggaatcacgccatctgctgtttattggtgacggcagcctgcagctgactgtccaggaactggggctggcgatccgtgaaaaaatcaatccgatttgctttatcatcaataacgacggctacaccgtcgaacgcgaaattcatggaccgaatcaaagttacaatgacatcccgatgtggaactatagcaaactgccggaatcctttggcgcgacagaggatcgcgtggtgagtaaaattgtgcgtacggaaaacgaatttgtgtcggttatgaaagaagcgcaggctgacccgaatcgcatgtattggattgaactgatcctggcaaaagaaggcgcaccgaaagttctgaaaaagatggggaaactgtttgcggagcaaaataaaagctaaggatcc

SEQ ID NO: 28 - DNA, 1647 bp, Lactococcus lactis
atgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataa

SEQ ID NO: 29 - DNA, 1644 bp, Lactococcus lactis
atgtatacagtaggagattacctgttagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggtaaattatttgctgagcaaaataaatag

SEQ ID NO: 30 - protein, 1537 aa, Saccharomyces cerivisiae
MTMPHRYMFLAVFTLLALTSVASGATEACLPAGQRKSGMNINFYQYSLKDSSTYSNAAYMAYGYASKTKLGSVGGQTDISIDYNIPCVSSSGTFPCPQEDSYGNWGCKGMGACSNSQGIAYWSTDLFGFYTTPTNVTLEMTGYFLPPQTGSYTFKFATVDDSAILSVGGATAFNCCAQQQPPITSTNFTIDGIKPWGGSLPPNIEGTVYMYAGYYYPMKVVYSNAVSWGTLPISVTLPDGTTVSDDFEGYVYSFDDDLSQSNCTVPDPSNYAVSTTTTTTEPWTGTFTSTSTEMTTVTGTNGVPTDETVIVIRTPTTASTIITTTEPWNSTFTSTSTELTTVTGTNGVRTDETIIVIRTPTTATTAITTTEPWNSTFTSTSTELTTVTGTNGLPTDETIIVIRTPTTATTAMTTTQPWNDTFTSTSTELTTVTGTNGLPTDETIIVIRTPTTATTAMTTTQPWNDTFTSTSTELTTVTGTNGLPTDETIIVIRTPTTATTAMTTTQPWNDTFTSTSTEITTVTGTNGLPTDETIIVIRTPTTATTAMTTPQPWNDTFTSTSTEMTTVTGTNGLPTDETIIVIRTPTTATTAITTTEPWNSTFTSTSTEMTTVTGTNGLPTDETIIVIRTPTTATTAITTTQPWNDTFTSTSTEMTTVTGTNGLPTDETIIVIRTPTTATTAMTTTQPWNDTFTSTSTEITTVTGTTGLPTDETIIVIRTPTTATTAMTTTQPWNDTFTSTSTEMTTVTGTNGVPTDETVIVIRTPTSEGLISTTTEPWTGTFTSTSTEMTTVTGTNGQPTDETVIVIRTPTSEGLVTTTTEPWTGTFTSTSTEMTTITGTNGVPTDETVIVIRTPTSEGLISTTTEPWTGTFTSTSTEMTTITGTNGQPTDETVIVIRTPTSEGLISTTTEPWTGTFTSTSTEMTHVTGTNGVPTDETVIVIRTPTSEGLISTTTEPWTGTFTSTSTEVTTITGTNGQPTDETVIVIRTPTSEGLISTTTEPWTGTFTSTSTEMTTVTGTNGQPTDETVIVIRTPTSEGLVTTTTEPWTGTFTSTSTEMSTVTGTNGLPTDETVIVVKTPTTAISSSLSSSSSGQITSSITSSRPIITPFYPSNGTSVISSSVISSSVTSSLFTSSPVISSSVISSSTTTSTSIFSESSKSSVIPTSSSTSGSSESETSSAGSVSSSSFISSESSKSPTYSSSSLPLVTSATTSQETASSLPPATTTKTSEQTTLVTVTSCESHVCTESISPAIVSTATVTVSGVTTEYTTWCPISTTETTKQTKGTTEQTTETTKQTTVVTISSCESDVCSKTASPAIVSTSTATINGVTTEYTTWCPISTTESRQQTTLVTVTSCESGVCSETASPAIVSTATATVNDVVTVYPTWRPQTANEESVSSKMNSATGETTTNTLAAETTTNTVAAETITNTGAAETKTVVTSSLSRSNHAETQTASATDVIGHSSSVVSVSETGNTKSLTSSGLSTMSQQPRSTPASSMVGYSTASLEISTYAGSANSLLAGSGLSVFIASLLLAII

SEQ ID NO: 31 - protein, 1075 aa, Saccharomyces cerivisiae
MTIAHHCIFLVILAFLALINVASGATEACLPAGQRKSGMNINFYQYSLKDSSTYSNAAYMAYGYASKTKLGSVGGQTDISIDYNIPCVSSSGTFPCPQEDSYGNWGCKGMGACSNSQGIAYWSTDLFGFYTTPTNVTLEMTGYFLPPQTGSYTFSFATVDDSAILSVGGSIAFECCAQEQPPITSTNFTINGIKPWDGSLPDNITGTVYMYAGYYYPLKVVYSNAVSWGTLPISVELPDGTTVSDNFEGYVYSFDDDLSQSNCTIPDPSIHTTSTITTTTEPWTGTFTSTSTEMTTITDTNGQLTDETVIVIRTPTTASTITTTTEPWTGTFTSTSTEMTTVTGTNGQPTDETVIVIRTPTSEGLITTTTEPWTGTFTSTSTEMTTVTGTNGQPTDETVIVIRTPTSEGLITTTTEPWTGTFTSTSTEVTTITGTNGQPTDETVIVIRTPTSEGLITTTTEPWTGTFTSTSTEMTTVTGTNGQPTDETVIVIRTPTSEGLISTTTEPWTGTFTSTSTEVTTITGTNGQPTDETVIVIRTPTSEGLITTTTEPWTGTFTSTSTEMTTVTGTNGQPTDETVIVIRTPTSEGLITRTTEPWTGTFTSTSTEVTTITGTNGQPTDETVIVIRTPTTAISSSLSSSSGQITSSITSSRPIITPFYPSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHSSSVVSVSETGNTMSLTSSGLSTMSQQPRSTPASSMVGSSTASLEISTYAGSANSLLAGSGLSVFIASLLLAII

SEQ ID NO: 32 - protein, 1322 aa, Saccharomyces cerivisiae
MSLAHYCLLLAIVTLLGLTNVVSATTAACLPANSRKNGMNVNFYQYSLRDSSTYSNAAYMAYGYASKTKLGSVGGQTDISIDYNIPCVSSSGTFPCPQEDLYGNWGCKGIGACSNNPIIAYWSTDLFGFYTTPTNVTLEMTGYFLPPQTGSYTFKFATVDDSAILSVGGSIAFECCAQEQPPITSTNFTINGIKPWNGSPPDNITGTVYMYAGFYYPMKIVYSNAVAWGTLPISVTLPDGTTVSDDFEGYVYTFDNNLSQPNCTIPDPSNYTVSTTITTTEPWTGTFTSTSTEMTTVTGTNGVPTDETVIVIRTPTTASTIITTTEPWNSTFTSTSTELTTVTGTNGVRTDETIIVIRTPTTATTAITTTEPWNSTFTSTSTELTTVTGTNGLPTDETIIVIRTPTTATTAMTTTQPWNDTFTSTSTELTTVTGTNGLPTDETIIVIRTPTTATTAMTTTQPWNDTFTSTSTELTTVTGTNGLPTDETIIVIRTPTTATTAMTTTQPWNDTFTSTSTEITTVTGTNGLPTDETIIVIRTPTTATTAMTTTQPWNDTFTSTSTEMTTVTGTNGLPTDETIIVIRTPTTATTAITTTEPWNSTFTSTSTEMTTVTGTNGLPTDETIIVIRTPTTATTAITTTQPWNDTFTSTSTEMTTVTGTNGLPTDETIIVIRTPTTATTAMTTTQPWNDTFTSTSTEITTVTGTNGLPTDETIIVIRTPTTATTAMTTTQPWNDTFTSTSTEMTTVTGTNGVPTDETVIVIRTPTSEGLISTTTEPWTGTFTSTSTEMTTVTGTNGQPTDETVIVIRTPTSEGLVTTTTEPWTGTFTSTSTEMTTITGTNGQPTDETVIIVKTPTTAISSSLSSSSGQITSFITSARPIITPFYPSNGTSVISSSVISSSDTSSLVISSSVTSSLVTSSPVISSSFISSPVISSTTTSASILSESSKSSVIPTSSSTSGSSESETGSASSASSSSSISSESPKSTYSSSSLPPVTSATTSQEITSSLPPVTTTKTSEQTTLVTVTSCESHVCTESISSAIVSTATVTVSGATTEYTTWCPISTTEITKQTTETTKQTKGTTEQTTETTKQTTVVTISSCESDVCSKTASPAIVSTSTATINGVTTEYTTWCPISTTESKQQTTLVTVTSCGSGVCSETTSPAIVSTATATVNDVVTVYSTWRPQTTNEQSVSSKMNSATSETTTNTGAAETTTSTGAAETKTVVTSSISRFNHAETQTASATDVIGHSSSVVSVSETGNTKSLTSSGLSTMSQQPRSTPASSMVGSSTASLEISTYAGSANSLLAGSGLSVFIASLLLAII

SEQ ID NO: 33 - DNA, 8247 bp, Saccharomyces cerivisiae
atgtcccacaacaacaggcataaaaagaataacgataaagacagctcagcagggcagtatgcaaatagcattgacaattcattaagccaggaaagcgtctcaacgaacggcgtaacaaggatggctaacttaaaggctgatgaatgcggcagtggtgatgaaggagataaaacaaagcggttttcgatttcaagtattttgagtaaaagagagacaaaagacgtgcttccggaatttgcaggcagtagttcccacaatggagtactcacggcgaattcatcaaaggatatgaactttactttggaactaagcgagaatttgttggttgagtgtaggaaattgcaatcctctaatgaagctaaaaatgagcaaatcaagtctctcaagcaaattaaagagtcattaagtgacaagattgaggagctcactaaccaaaaaaagtccttcatgaaagagttggattcaactaaagatttaaactgggatttagaatctaaattaacaaacttgagcatggaatgtaggcaattaaaagaattgaagaaaaagactgaaaaatcttggaatgatgaaaaagaaagcctgaaacttctgaaaacagatttggaaattttaacattaacaaaaaatggcatggaaaatgatcttagctctcaaaaacttcattacgataaagagattagtgaattaaaggaaaggattttagacttaaataatgaaaacgacagattacttattagtgtttctgatctaacaagtgaaattaattccttacagagcaatagaactgaaagaataaaaattcaaaagcaacttgatgacgccaaagcatctatttcttcgttaaaaagaaaagtacaaaagaagtattatcaaaaacagcatacttccgatactacagtaacatctgatcctgattctgaggggaccactagtgaagaagacatttttgatatagtgatcgaaattgaccacatgattgaaacaggcccctctgtcgaggacatttctgaagatcttgtcaagaaatactcagaaaaaaacaatatgatattgttatcgaatgattcatataaaaacttactacaaaaaagtgaaagtgcatccaaaccaaaagacgatgaattaatgaccaaagaggtggctgaaaacctgaatatgatcgcgttaccaaatgatgacaattacagcaaaaaagagttttcgttagaatctcatattaaatatttagaagcttctggctataaagttcttcctctagaggagtttgagaacctaaacgaatccctatcaaatccatcatataactatctcaaggaaaaacttcaggctttgaaaaagatacccatcgatcaaagtacgtttaacttgttaaaagagcctactattgattttttactgcctttaacatccaaaattgattgcctgataatacctaccaaagattataatgacctttttgagagtgtcaagaatccatcaattgaacaaatgaaaaaatgcctggaagcaaagaacgacttacaatcgaatatttgtaaatggctggaggagagaaacggctgtaaatggctaagtaatgatctgtatttttcaatggttaataagatagaaacaccttcgaaacaatacctgtcagataaggcaaaagaatacgaccaagtgctgattgatactaaagccttagaaggtttaaagaacccaacgatagactttctaagagaaaaagcttctgcatcagattatttattactcaaaaaagaagactacgtgagcccatcactggaatacctagttgaacatgccaaggccaccaatcaccatttactatcggatagtgcatacgaagacctagtcaagtgcaaggagaatcctgatatggaattcttgaaggagaagtctgccaaactaggccacactgtggtatccaacgaggcatattctgaattggaaaagaaactagaacaaccatcactggaatacctagttgaacatgccaaggcgaccaatcaccatttactatcggatagtgcatacgaagacctagtcaagtgcaaggagaatcctgatatggaattcttgaaggagaagtctgccaaactaggccatactgtggtatccaacgaggcatattctgaattgcaacgcaaatactcagaattggagaaggaagtagaacaaccatctctagcatacttagttgaacacgccaaggctaccgatcaccatttactatcggatagtgcatacgaagacctagtcaagtgcaaggagaatcctgatgtggaattcttgaaggagaagtctgctaaactaggccatactgtggtatctagcgaggaatattctgaattgcaacgcaaatactcagaattggagaaggaagtagaacaaccatcactagcatacctagtcgaacacgccaaggctaccgatcaccatttactatcggatagtgcatacgaagaactagtcaagtgcaaggagaatcctgatatggaattcttgaaggagaagtctgccaaactaggccacactgtggtatccaacgaggcatattctgaattggaaaagaaactagaacaaccatcactagcatacctagtcgaacatgccaaggctaccgatcaccatctgctatcggatagtgcatacgaagacctagtcaagtgcaaggaaaattctgatgtagaattcttgaaggagaagtctgctaaactaggccatactgtggtatccaacgaagcatattctgaattggaaaagaaactagaacaaccatcactagcatacctagtcgaacatgccaaggctaccgatcaccatctgctatcggatagtgcatacgaagacctagtcaagtgcaaggagaatcctgatatggaattcttgaaggagaagtctgccaaactaggccacactgtggtatccaacgaggcatattctgaattggaaaagaaactagaacaaccatcactggaatacctagttgaacatgccaaggccaccaatcaccatttactatcggatagtgcatacgaagacctagtcaagtgcaaggagaatcctgatatggaattcttgaaggagaagtctgccaaactaggccacactgtggtatccaacgaggcatattctgaattggaaaagaaactagaacaaccatcactggaatacctagttgaacatgccaaggccaccaatcaccatctgctatcggatagtgcatacgaagaactagtcaagtgcaaggaaaatcctgatgtagaattcttgaaggagaagtctgctaaactaggccatactgtggtatccaacgaagcatattctgaattggaaaagaaactagaacaaccatcactggaatacctagttgaacatgccaaggccaccaatcaccatctgctatcggatagtgcatacgaagaactagtcaagtgcaaggaaaatcctgatgtagaattcttgaaggagaagtctgctaaactaggccatactgtggtatccaacgaagcatattctgaattggaaaagaaactagaacaaccatcactagcatacctagtcgaacatgccaaggctaccgatcaccatctgctatcggatagtgcatacgaagacctagtcaagtgcaaggaaaatcctgatgtagaattcttgaaggagaagtctgctaaactaggccatactgtggtatccaacgaagcatattctgaattggaaaagaaactagaacaaccatcactagcatacctagtcgaacatgccaaggctaccgatcaccatctgctatcggatagtgcatacgaagacctagtcaagtgcaaggagaatcctgatatggaattcttgaaggagaagtctgccaaactaggccacactgtggtatccaacgaggcatattctgaattggaaaagaaactagaacaaccatcactggaatacctagttgaacatgccaaggccaccaatcaccatctgctatcggatagtgcatacgaagacctagtcaagtgcaaggagaatcctgatatggaattcttgaaggagaagtctgctaaactgggccatactgtggtatccaacaaggaatattctgaattggaaaagaaactagaacaaccatcactggaatacttagtcaaacatgccgaacaaatacaatcaaaaattatatcgatctcggacttcaacaccttagctaatccatctatggaagatatggcttcaaaattgcaaaagttagaataccagattgtttcgaacgatgagtacattgcattgaaaaatacgatggaaaagccggacgttgagttactaagatccaagttgaaaggttaccatataattgatacaacaacgtacaatgagctagtcagcaatttcaattctcctacgttgaagtttattgaagagaaagccaaaagcaaaggttatagattaatagaacctaatgaataccttgacttgaataggatagccactacaccttctaaagaagagattgataacttctgcaaacaaattgggtgttacgctttggactctaaagaatatgaaagactaaaaaattctctggagaatccctccaagaaatttatagaagaaaatgccgcattacttgatcttgtgctagtggacaaaacggagtaccaagcaatgaaagataatgcaagcaacaagaaatcacttattccttcaaccaaggcacttgatttcgttacaatgcctgccccacagcttgcttctgcagagaagtcatcactacaaaaaagaactttatctgatattgaaaatgagttaaaggccttaggctacgtcgcaattcgtaaagaaaacctgccaaacctagagaaaccaattgttgacaatgcctccaaaaatgatgtcttgaacctatgttcgaaattcagtttagtaccattgtctactgaagaatatgataatatgagaaaggaacacactaaaatcttaaatattctcggtgatccatctattgatttcctgaaggaaaaatgtgaaaaatatcaaatgctcataattagtaaacatgattacgaagaaaagcaagaagccattgaaaatccaggctacgaatttattttagaaaaagcatcagcactgggatatgaattagttagcgaggttgagctggatcgcatgaaacaaatgattgattcaccagatattgactacatgcaagaaaaggctgcccgcaatgaaatggtgttgttgaggaacgaggagaaggaagcattgcaaaagaaaatagaatatccctctttaacatttttaatcgaaaaggctgctggaatgaacaaaatacttgttgaccaaatcgagtatgatgaaactataagaaaatgcaatcatcccactcggatggagctagaggaatcctgtcatcacttgaacttggttttgctcgaccaaaacgagtactcaactctaagagaacctttggaaaatcgaaatgttgaagacttaattaacaccttgagcaaactaaactacattgcaattcctaatactatctaccaagatttaattggaaagtatgagaatccaaactttgattatctaaaggattctttgaacaaaatggattacgtcgcaatctctagacaagattatgaattgatggttgctaaatacgaaaagccacaactggattatttgaaaatttcttcagagaaaatcgaccacattgtagtgcctctgtctgagtacaatttaatggttacaaattatagaaatcccagcttgagctacttaaaagagaaagccgttttgaataatcatattttaataaaagaagatgactataaaaacattttagcagtatcagaacatccgacagtgatccacctctccgaaaaggcatctttattaaataaagtcttggtagacaaggatgattttgcgaccatgtcacgctcgattgagaaaccaactatcgatttcttatccactaaggcgctatcaatggggaaaatactagttaatgaatctacgcataaaagaaacgagaaactattatctgaaccagattctgaatttttgacaatgaaagccaaggagcaagggctaattatcatttcagaaaaggaatattctgaactgcgggatcaaatagatcgtcctagcctagatgttttgaaagaaaaggccgccatttttgatagcatcatagtagaaaacatagaataccaacaactggtaaacactacaagtccctgccctcccattacttatgaagatttgaaagtatatgcccaccaattcggtatggaattatgcctccaaaaacccaacaaactttctggagctgagcgtgcagagcgcattgatgaacaatcaataaatacgaccagcagtaactcgaccacaacatcgagcatgtttacagatgcactagatgataatatcgaagagcttaatcgtgtcgaattgcagaataatgaagattatactgacataatctcgaaatcatccacagtgaaagatgctaccattttcattcccgcctatgaaaacatcaagaattctgctgaaaaattaggctacaaattagttccgttcgaaaaatcaaatatcaatctgaaaaacattgaagctccattattttcgaaggacaacgatgacactagcgttgccagtagcatagatcttgatcacttatctagaaaagcagaaaaatatggtatgaccctcatttctgatcaggaatttgaagaatatcatatactaaaagataacgcggttaatctgaatggtggcatggaagaaatgaataatcccttgtcagaaaatcaaaacttagcagcaaaaaccacaaacacagcgcaagaaggtgccttccaaaacaccgttccccacaatgatatggacaacgaagaagtcgaatatgggccggatgatccaacattcacagtaaggcaactcaagaaacccgctggcgatcgtaatttgattttgactagtagggagaaaacactgttatcaagagatgataatataatgagtcaaaatgaggcggtttatggtgacgatatatctgatagctttgtagatgaaagccaagaaatcaaaaatgatgtagacattattaaaactcaagctatgaaatatggtatgttgtgtattcctgaaagtaattttgtgggtgcatcatatgcaagtgctcaagatatgagcgatatagttgtgctttccgcgtcctattaccataatctaatgtcacctgaagacatgaaatggaactgtgttagtaatgaagaattacaagcggaagttaaaaagcgtgggctccagattgcactaacaacaaaggaagataagaaaggtcaagccacggcatccaaacatgagtatgtgtcgcataagctaaacaataaaacatctactgtgtccacaaagtctggagcaaaaaagggacttgcagaagcagcagcaacaactgcttatgaagattccgaaagtcatccacaaatagaagagcagtctcatcgtactaatcatcataagcaccataaacgtcaacagagtctgaattctaattcaacctcaaaaaccacacattcatcgaggaatacgccagcatctagacgagatatagtagcatcatttatgtcacgtgcaggatctgccagtaggacggcatctttacaaactttagcatcattgaacgaaccaagcataatacccgcgttaacccaaaccgtcattggggaatatttgtttaagtattatccacgcttgggaccttttggattcgaatcacgtcatgaaagattcttctgggttcatccatataccttaactttgtactggtccgcttctaatcccatcctagagaatcctgccaataccaaaacaaaaggtgttgccattctaggagtagaaagtgtcacagacccaaacccatatccaacaggattgtatcacaaaagtattgttgttaccacagaaactaggactattaagtttacttgtcctacaaggcaaagacacaatatttggtataattcattacgttatttacttcaaaggaacatgcaagggataagtttagaggacatcgctgatgatccaacagataatatgtattcaggaaagattttcccattgcccggcgaaaatacaaagagctccagtaaaagacttagcgcatcgagaaggtccgtatctacaaggtctctaagacatagagtaccacaaagccgatcatttggcaatttacgatag

SEQ ID NO: 34 - DNA, 363 bp, Saccharomyces cerivisiae
atggtcaaattaacttcaatcgccgctggtgtcgctgccatcgctgctactgcttccgcaaccaccactctagctcaatctgacgaaagagtcaacttggttgaattgggtgtctacgtctctgatatcagagctcacttggcccaatactacatgttccaagccgcccacccaacggaaacctacccagttgaagttgctgaagccgttttcaactacggtgacttcaccaccatgttgactggtattgccccagaccaagtgaccagaatgatcaccggtgttccatggtactccagcagattaaagccagccatctccagtgctctatccaaggtcggtatctacactatcgcaaactag

SEQ ID NO: 35 - DNA, 4645 bp, Saccharomyces cerivisiae
atgagctttatggatcaaatcccaggaggaggaaattatccaaaactcccagtagaatgccttcctaacttcccgatccaaccatctttgaccttcagaggtagaaatgactcgcataaactgaaaaactttatctccgaaataatgttaaacatgtctatgatatcttggccgaatgatgccagtcgtattgtgtactgcagaagacatttattaaaccccgctgctcagtgggctaatgactttgtacaagaacaaggtatacttgaaataacattcgacacattcatacaaggattatatcagcatttctataagccaccagatatcaataaaatctttaatgcaatcacgcaactttccgaagctaaacttggtattgagcgtctcaaccaacgattcagaaagatttgggacagaatgccaccagacttcatgaccgaaaaagctgccataatgacatatactaggctattgacaaaggaaacctataatattgtcagaatgcacaaaccagagacattaaaagacgccatggaagaggcttaccagacaactgcactaactgaaagattcttcccaggattcgaacttgatgctgatggagacactatcatcggtgccacaacccacttacaagaagaatacgactctgactatgattcagaagataatctgacccagaatggatacgtccataccgtaaggacaagaagatcttacaataaaccaatgtcaaatcatcgaaacaggagaaataacaacccatctagagaagaatgtataaaaaatcggctatgcttctattgtaagaaagagggacatcgcctgaacgaatgtagagcacgtaaggcgagttctaaccgatcttgaactcgaatcaaaagaccaacaaactccttttatcaaaaccttaccaattgtacactatatcgccatccccgagatggacaataccgccgaaaaaaccataaaaatacaaaacacgaaagtaaaaaccctgtttgacagtggatcacccacgtcatttatccgaagagatattgtagaacttctcaaatacgaaatctacgagacccctccactccgttttagaggattcgtagccaccaaatccgccgttacatccgaagcagtcaccattgacctcaaaatcaatgacctgcatataactttagccgcgtacatactggataacatggactaccaattgttaattggaaatccaatcttacgccgctacccgaaaatcctgcacacagtactgaataccagagagagccccgactccttaaagcccaagacttatcgctccgaaaccgttaataacgttagaacctactccgctggtaatcgtggtaaccccagaaacataaaactgtcttttgcccccaccattctcgaagcaactgacccgaaatccgctggtaatcgtggtgactccagaaccaaaaccctgtctcttgcaaccactactcctgcagcaattgacccgcttacgacccttgataacccaggtagtactcaaagtacatttgcgcaattcccgatacctgaagaagcgagcatcctagaagaggatggaaaatactccaacgttgtctcaaccattcagagtgtagaacctaatgctactgatcacagcaataaggacaccttttgcactttgccagtttggttacaacagaagtatagagagatcatacgtaatgatctcccaccaagacctgccgacattaataacatccccgtaaaacatgatattgaaattaaacctggcgcaagactacctcgactacagccataccatgttacagaaaagaacgaacaagaaatcaacaaaatagttcaaaaactgctcgataacaagttcattgttccctcaaagtcgccttgcagctcccctgtagtcctcgtcccgaagaaagacggtaccttccgactctgcgtcgattaccgcaccctgaacaaagctaccatctccgacccattcccattacccagaatcgacaacctattgagccgtattggaaatgcccagatatttaccacgctagatttgcatagtggttaccaccagatcccgatggaacccaaagaccgctacaaaaccgcctttgtcacaccatccggtaagtatgaatataccgtcatgccatttggcttagtcaatgcacctagtacattcgcaagatacatggctgatacatttagagacctgagattcgtcaatgtttaccttgatgatatattaatattctccgaatctccagaagaacattggaaacatttagacacggtactagaaagattaaagaacgagaacctcattgttaagaagaaaaaatgtaaatttgcatctgaagaaactgagtttttaggctatagtattggaatccagaaaatagctccactacagcacaaatgtgcagcaatccgagactttccgacgcctaaaacagtaaaacaagcacagagatttttaggaatgattaattactacagacgattcattccaaattgctccaagattgcacagccaatccaactgtttatttgtgacaaaagtcaatggacagaaaaacaagacaaggcaattgataaactaaaagacgccttgtgtaactcccccgtcctagtaccattcaacaacaaagcaaactaccgacttacaacagacgcctcaaaagacggcattggtgctgttctagaagaagtcgacaacaagaacaaacttgttggtgtcgtcggttacttctctaaatccttagagagtgcccagaaaaactatcctgctggcgaattagaactacttggaattatcaaagcactccaccacttccgatatatgcttcacggaaagcatttcacgttaagaacagaccacattagtttgttatcattacaaaacaagaacgaacccgcacgacgcgtgcaacgctggttagatgacctagccacatatgacttcaccttagaatacctagctggacccaagaacgttgtcgcagatgccatatcccgtgccgtatatactataacccccgaaacatcccgacctatcgacacagaaagctggaaatcttactacaaatcagacccattatgtagtgctgtcttaattcatatgaaagaattgacacaacacaacgtcacacctgaagatatgtcagccttccgtagttaccagaagaaactcgaactatcagagaccttccgaaagaattattccctagaagacgaaatgatctattaccaagaccgactagtagtaccaataaaacaacagaacgcagttatgagactatatcatgaccataccttatttggaggacattttggtgtaacagtgacccttgcgaaaatcagcccaatttactattggccaaaattacaacattcgatcatacaatacatcaggacctgcgtacaatgtcaactaataaaatcacaccgaccacgcttacatggactattacaaccactccctatagcagaaggaagatggcttgatatatcaatggattttgtgacaggattacccccgacatcaaataacttgaatatgatcctcgtcgtagttgatcgtttttcgaaacgcgctcacttcatagctacaaggaaaaccttagacgcaacacaactaatagatctactctttcgatacattttttcatatcatggttttcccaggacaataaccagtgatagagatgtccgtatgaccgccgacaaatatcaagaactcacgaaaagactaggaataaaatcgacaatgtcttccgcgaaccacccccaaacagatggacaatccgaacgaacgatacagacattaaacaggttactaagagcctatgcttcaaccaatattcagaattggcatgtatatttaccacaaatcgaatttgtttacaattctacacctactagaacacttggaaaatcaccatttgaaattgatttaggatatttaccgaatacccctgctattaagtcagatgacgaagtcaacgcaagaagttttactgccgtagaacttgccaaacacctcaaagcccttaccatccaaacgaaggaacagctagaacacgctcaaatcgaaatggaaactaataacaatcaaagacgtaaacccttattgttaaacataggagatcacgtattagtgcatagagatgcatacttcaagaaaggtgcttatatgaaagtacaacaaatatacgtcggaccatttcgagttgtcaagaaaataaacgataacgcctacgaactagatttaaactctcacaagaaaaagcacagagttattaatgtacaattcctgaaaaagtttgtataccgtccagacgcgtacccaaagaataaaccaatcagctccactgaaagaattaagagagcacacgaagttactgcactcataggaatagatactacacacaaaacttacttatgtcacatgcaagatgtagacccaacactttcagtagaatactcagaagctgaattttgccaaattcccgaaagaacacgaagatcaatattagccaactttagacaactctacgaaacacaagacaaccctgagagagaggaagatgttgtatctcaaaatgagatatgtcagtatgacaatacgtcaccctga

SEQ ID NO: 36 - DNA, 714 bp, Saccharomyces cerivisiae
atgactccaaaaagagcgctaatatctcttacttcataccacggtcccttctataaagatggtgcgaaaacaggcgtttttgtagttgagattttgcggtcgttcgatactttcgaaaagcatggtttcgaagtggacttcgtttctgagactggtggatttggctgggatgaacattacttgccaaagagctttattggtggcgaagataagatgaactttgaaacgaaaaattccgccttcaataaggcgttagcgaggatcaagaccgcaaatgaagtcaacgccagcgactataaaatattctttgcatctgctggacatggtgctctatttgactatcccaaagctaaaaatctgcaagatattgcatccaagatatatgccaatgggggtgtgatcgctgccatctgtcatggaccgctccttttcgatggattaatagatatcaaaacaacaagaccattaatcgaaggcaaagctataacaggtttcccactcgagggtgaaatcgccctgggagttgacgacatcttgaggagcagaaaattgacaacggttgaacgcgttgcaaacaagaatggagccaagtacttggcgccaatccatccctgggatgactactctattacagatggaaagctagttacgggtgttaacgcaaattcttcctattcgaccacaattagagctataaacgcattatatagctga

SEQ ID NO: 37 - DNA, 2217 bp, Saccharomyces cerivisiae
atggttgccgaagaggacatcgagaagcaagtccttcaattgatagacagcttttttctgaagactacactactaatatgctccaccgaatcaagtcgataccagtcttctacagaaaatatattcctatttgacgacacatggtttgaagatcactcagaattagtgagtgagctacccgagataatatcaaaatggtctcactacgatggtcgaaaagagttgccacccttagtggtagagacatatttggatttaagacagttaaactcgtctcatttagttagattaaaggaccacgaaggccatttgtggaacgtttgcaaaggaactaagaagcaggaaatcgtgatggaacgttggcttatcgaattagataattcatccccaactttcaaatcatacagtgaagatgagactgatgttaatgaactttctaaacagctagtccttctcttccgttatttgttgactttaatacagttactacccacaacagaattataccaattattaataaagtcttataacggcccgcaaaatgaaggaagttccaatccaataacttccacgggcccactagtaagtatccggacgtgtgtccttgacggatctaaaccaattttatcgaaggggagaatagggttgagcaaaccgattattaatacatattccaatgcgcttaacgaatcaaacctgccagcccatttagatcaaaagaagatcacacctgtatggacaaagtttggactcttaagagtctcggtatcatacagacgtgattggaagtttgaaattaacaatacaaacgacgaattattttcagctcgacatgcatctgtctcacataactcacaaggaccccagaatcagccagaacaagaaggacaaagtgatcaagacatagggaaacgccaaccacaatttcaacagcagcagcagccccaacagcagcagcagcagcagcaacagcaacagagacaacaccaggtccagacacaacaacaaagacagatacctgataggagatctctttcactttctccttgtacaagagccaattcttttgaaccacaatcttggcagaagaaagtctatccaatatcgagacctgttcaaccatttaaagttggttcaattggaagtcaaagtgcgagcagaaatccctctaattcatcgtttttcaaccaaccacctgttcataggccaagtatgagctccaactacgggccacaaatgaatattgaaggtaccagtgttggaagcacctcaaagtattcctcctcctttgggaacattcgtcgtcactcaagtgtaaagacgacagagaatgctgaaaaagtatcaaaagctgtaaagagcccactacaacctcaagaatcacaagaagatttaatggattttgttaaattactcgaagaaaaacccgatctaactataaagaagacaagtggaaataatccacccaatatcaatatttctgattctctaatcagatatcagaatttgaagccaagtaatgacttattaagtgaagatttatccgtaagtttatccatggatccaaatcatacatatcacagaggcagatcagattcccactcaccattgccttcaatatccccttcgatgcattatggatcgttgaactcgagaatgtctcaaggcgccaatgcaagccatttgattgcaagaggcggtgggaattcatctactagtgccttgaatagtagaaggaattctttagataagagctcaaacaagcagggtatgtcaggcttacctcctatttttggtggagagagtacttcatatcaccacgacaacaaaatacaaaagtacaaccaattaggagtagaagaagatgatgatgacgagaatgaccgtttgctcaaccaaatgggaaacagtgctacaaaattcaaaagttcaatatctccaagatcaattgatagcatttcaagttctttcataaaaagtaggatacctatcagacaaccataccattactctcaaccaactactgcgccctttcaagctcaggcgaaatttcataaacctgcaaataagttaatcgataatggtaataggagtaatagtaacaataacaatcataatgggaatgatgcagttggtgtgatgcataatgacgaggatgatcaagatgatgatctagtatttttcatgagtgatatgaacctttctaaagaaggttaa

SEQ ID NO: 38 - protein, 254 aa, Saccharomyces cerevisiae
MAYTKIALFAAIAALASAQTQDQINELNVILNDVKSHLQEYISLASDSSSGFSLSSMPAGVLDIGMALASATDDSYTTLYSEVDFAGVSKMLTMVPWYSSRLEPALKSLNGDASSSAAPSSSAAPTSSAAPSSSAAPTSSAASSSSEAKSSSAAPSSSEAKSSSAAPSSSEAKSSSAAPSSSEAKSSSAAPSSTEAKITSAAPSSTGAKTSAISQITDGQIQATKAVSEQTENGAAKAFVGMGAGVVAAAAMLL

SEQ ID NO: 39 - protein, 251 aa, Saccharomyces cerevisiae
MAYIKIALLAAIAALASAQTQEEIDELNVILNDVKSNLQEYISLAEDSSSGFSLSSLPSGVLDIGLALASATDDSYTTLYSEVDFAAVSKMLTMVPWYSSRLLPELESLLGTSTTAASSTEASSAATSSAVASSSETTSSAVASSSEATSSAVASSSEASSSAATSSAVASSSEATSSTVASSTKAASSTKASSSAVSSAVASSTKASAISQISDGQVQATSTVSEQTENGAAKAVIGMGAGVMAAAAMLL

SEQ ID NO: 40 - protein, 269 aa, Saccharomyces cerevisiae
MSFTKIAALLAVAAASTQLVSAEVGQYEIVEFDAILADVKANLEQYMSLAMNNPDFTLPSGVLDVYQHMTTATDDSYTSYFTEMDFAQITTAMVQVPWYSSRLEPEIIAALQSAGISITSLGQTVSESGSESATASSDASSASESSSAASSSASESSSAASSSASESSSAASSSASESSSAASSSASEAAKSSSSAKSSGSSAASSAASSASSKASSAASSSAKASSSAEKSTNSSSSATSKNAGAAMDMGFFSAGVGAAIAGAAAMLL

SEQ ID NO: 41 - protein, 487 aa, Saccharomyces cerevisiae
MAYSKITLLAALAAIAYAQTQAQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVAPSSSEVVSSSVAPSSSEVVSSSVASSSSEVASSSVAPSSSEVVSSSVASSSSEVASSSVAPSSSEVVSSSVAPSSSEVVSSSVASSSSEVASSSVAPSSSEVVSSSVASSTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 42 - protein, 298 aa, Saccharomyces cerevisiae
MSRISILAVAAALVASATAASVTTTLSPYDERVNLIELAVYVSDIGAHLSEYYAFQALHKTETYPPEIAKAVFAGGDFTTMLTGISGDEVTRMITGVPWYSTRLMGAISEALANEGIATAVPASTTEASSTSTSEASSAATESSSSSESSAETSSNAASTQATVSSESSSAASTIASSAESSVASSVASSVASSASFANTTAPVSSTSSISVTPVVQNGTDSTVTKTQASTVETTITSCSNNVCSTVTKPVSSKAQSTATSVTSSASRVIDVTTNGANKFNNGVFGAAAIAGAAALLL

SEQ ID NO: 43 - protein, 1367 aa, Saccharomyces cerevisiae
MQRPFLLAYLVLSLLFNSALGFPTALVPRGSSEGTSCNSIVNGCPNLDFNWHMDQQNIMQYTLDVTSVSWVQDNTYQITIHVKGKENIDLKYLWSLKIIGVTGPKGTVQLYGYNENTYLIDNPTDFTATFEVYATQDVNSCQVWMPNFQIQFEYLQGSAAQYASSWQWGTTSFDLSTGCNNYDNQGHSQTDFPGFYWNIDCDNNCGGTKSSTTTSSTSESSTTTSSTSESSTTTSSTSESSTTTSSTSESSTSSSTTAPATPTTTSCTKEKPTPPTTTSCTKEKPTPPHHDTTPCTKKKTTTSKTCTKKTTTPVPTPSSSTTESSSAPVPTPSSSTTESSSAPVTSSTTESSSAPVPTPSSSTTESSSAPVTSSTTESSSAPVTSSTTESSSAPVPTPSSSTTESSSAPVTSSTTESSSAPVTSSTTESSSAPVTSSTTESSSAPVTSSTTESSSAPVPTPSSSTTESSSAPVTSSTTESSSAPVPTPSSSTTESSSAPVTSSTTESSSAPVPTPSSSTTESSSAPAPTPSSSTTESSSAPVTSSTTESSSAPVPTPSSSTTESSSTPVTSSTTESSSAPVPTPSSSTTESSSAPVPTPSSSTTESSSAPAPTPSSSTTESSSAPVTSSTTESSSAPVPTPSSSTTESSSAPVPTPSSSTTESSSAPVPTPSSSTTESSSAPVTSSTTESSSAPVTSSTTESSSAPVPTPSSSTTESSSAPVPTPSSSTTESSSAPVPTPSSSTTESSSAPVTSSTTESSSAPVPTPSSSTTESSSAPVPTPSSSTTESSSAPVPTPSSSTTESSVAPVPTPSSSSNITSSAPSSTPFSSSTESSSVPVPTPSSSTTESSSAPVSSSTTESSVAPVPTPSSSSNITSSAPSSIPFSSTTESFSTGTTVTPSSSKYPGSQTETSVSSTTETTIVPTKTTTSVTTPSTTTITTTVCSTGTNSAGETTSGCSPKTVTTTVPTTTTTSVTTSSTTTITTTVCSTGTNSAGETTSGCSPKTITTTVPCSTSPSETASESTTTSPTTPVTTVVSTTVVTTEYSTSTKPGGEITTTFVTKNIPTTYLTTIAPTPSVTTVTNFTPTTITTTVCSTGTNSAGETTSGCSPKTVTTTVPCSTGTGEYTTEATTLVTTAVTTTVVTTESSTGTNSAGKTTTGYTTKSVPTTYVTTLAPSAPVTPATNAVPTTITTTECSAATNAAGETTSVCSAKTIVSSASAGENTAPSATTPVTTAIPTTVITTESSVGTNSAGETTTGYTTKSIPTTYITTLIPGSNGAKNYETVATATNPISIKTTSQLATTASASSVAPVVTSPSLTGPLQSASGSAVATYSVPSISSTYQGAANIKVLGNFMWLLLALPVVF

SEQ ID NO: 44 - protein, 798 aa, Saccharomyces cerevisiae
MSYKVNSSYPDSIPPTEQPYMASQYKQDLQSNIAMATNSEQQRQQQQQQQQQQQQWINQPTAENSDLKEKMNCKNTLNEYIFDFLTKSSLKNTAAAFAQDAHLDRDKGQNPVDGPKSKENNGNQNTFSKVVDTPQGFLYEWQIFWDIFNTSSSRGGSEFAQQYYQLVLQEQRQEQIYRSLAVHAARLQHDAERRGEYSNEDIDPMHLAAMMLGNPMAPAVQMRNVNMNPIPIPMVGNPIVNNFSIPPYNNANPTTGATAVAPTAPPSGDFTNVGPTQNRSQNVTGWPVYNYPMQPTTENPVGNPCNNNTTNNTTNNKSPVNQPKSLKTMHSTDKPNNVPTSKSTRSRSATSKAKGKVKAGLVAKRRRKNNTATVSAGSTNACSPNITTPGSTTSEPAMVGSRVNKTPRSDIATNFRNQAIIFGEEDIYSNSKSSPSLDGASPSALASKQPTKVRKNTKKASTSAFPVESTNKLGGNSVVTGKKRSPPNTRVSRRKSTPSVILNADATKDENNMLRTFSNTIAPNIHSAPPTKTANSLPFPGINLGSFNKPAVSSPLSSVTESCFDPESGKIAGKNGPKRAVNSKVSASSPLSIATPRSGDAQKQRSSKVPGNVVIKPPHGFSTTNLNITLKNSKIITSQNNTVSQELPNGGNILEAQVGNDSRSSKGNRNTLSTPEEKKPSSNNQGYDFDALKNSSSLLFPNQAYASNNRTPNENSNVADETSASTNSGDNDNTLIQPSSNVGTTLGPQQTSTNENQNVHSQNLKFGNIGMVEDQGPDYDLNLLDTNENDFNFINWEG

SEQ ID NO: 45 - protein, 1169 aa, Saccharomyces cerevisiae
MPVAARYIFLTGLFLLSVANVALGTTEACLPAGEKKNGMTINFYQYSLKDSSTYSNPSYMAYGYADAEKLGSVSGQTKLSIDYSIPCNGASDTCACSDDDATEYSASQVVPVKRGVKLCSDNTTLSSKTEKRENDDCDQGAAYWSSDLFGFYTTPTNVTVEMTGYFLPPKTGTYTFGFATVDDSAILSVGGNVAFECCKQEQPPITSTDFTINGIKPWNADAPTDIKGSTYMYAGYYYPIKIVYSNAVSWGTLPVSVVLPDGTEVNDDFEGYVFSFDDNATQAHCSVPNPAEHARTCVSSATSSWSSSEVCTECTETESTSYVTPYVTSSSWSSSEVCTECTETESTSTSTPYVTSSSSSSSEVCTECTETESTSYVTPYVSSSTAAANYTSSFSSSSEVCTECTETESTSTSTPYVTSSSWSSSEVCTECTETESTSYVTPYVSSSTAAANYTSSFSSSSEVCTECTETESTSTSTPYVTSSSSSSSEVCTECTETESTSYVTPYVSSSTAAANYTSSFSSSSEVCTECTETESTSTSTPYVTSSSWSSSEVCTECTETESTSYVTPYVSSSTAAANYTSSFSSSSEVCTECTETESTSTSTPYATSSTGTATSFTASTSNTMTSLVQTDTTVSFSLSSTVSEHTNAPTSSVESNASTFISSNKGSVKSYVTSSIHSITPMYPSNQTVTSSSVVSTPITSESSESSASVTILPSTITSEFKPSTMKTKVVSISSSPTNLITSYDTTSKDSTVGSSTSSVSLISSISLPSSYSASSEQIFHSSIVSSNGQALTSFSSTKVSSSESSESHRTSPTTSSESGIKSSGVEIESTSTSSFSFHETSTASTSVQISSQFVTPSSPISTVAPRSTGLNSQTESTNSSKETMSSENSASVMPSSSATSPKTGKVTSDETSSGFSRDRTTVYRMTSETPSTNEQTTLITVSSCESNSCSNTVSSAVVSTATTTINGITTEYTTWCPLSATELTTVSKLESEEKTTLITVTSCESGVCSETASPAIVSTATATVNDVVTVYSTWSPQATNKLAVSSDIENSASKASFVSEAAETKSISRNNNFVPTSGTTSIETHTTTTSNASENSDNVSASEAVSSKSVTNPVLISVSQQPRGTPASSMIGSSTASLEMSSYLGIANHLLTNSGISIFIASLLLAIV

SEQ ID NO: 46 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 47 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 48 - protein, 533 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVI

SEQ ID NO: 49 - DNA, 1692 bp, Saccharomyces cerivisiae
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 50 - DNA, 1692 bp, Saccharomyces cerivisiae
atgtctgaaataaccttaggtaaatatttatttgaaagattgagccaagtcaactgtaacaccgtcttcggtttgccaggtgactttaacttgtctcttttggataagctttatgaagtcaaaggtatgagatgggctggtaacgctaacgaattgaacgctgcctatgctgctgatggttacgctcgtatcaagggtatgtcctgtattattaccaccttcggtgttggtgaattgtctgctttgaatggtattgccggttcttacgctgaacatgtcggtgttttgcacgttgttggtgttccatccatctcttctcaagctaagcaattgttgttgcatcataccttgggtaacggtgacttcactgttttccacagaatgtctgccaacatttctgaaaccactgccatgatcactgatattgctaacgctccagctgaaattgacagatgtatcagaaccacctacactacccaaagaccagtctacttgggtttgccagctaacttggttgacttgaacgtcccagccaagttattggaaactccaattgacttgtctttgaagccaaacgacgctgaagctgaagctgaagttgttagaactgttgttgaattgatcaaggatgctaagaacccagttatcttggctgatgcttgtgcttctagacatgatgtcaaggctgaaactaagaagttgatggacttgactcaattcccagtttacgtcaccccaatgggtaagggtgctattgacgaacaacacccaagatacggtggtgtttacgttggtaccttgtctagaccagaagttaagaaggctgtagaatctgctgatttgatattgtctatcggtgctttgttgtctgatttcaataccggttctttctcttactcctacaagaccaaaaatatcgttgaattccactctgaccacatcaagatcagaaacgccaccttcccaggtgttcaaatgaaatttgccttgcaaaaattgttggatgctattccagaagtcgtcaaggactacaaacctgttgctgtcccagctagagttccaattaccaagtctactccagctaacactccaatgaagcaagaatggatgtggaaccatttgggtaacttcttgagagaaggtgatattgttattgctgaaaccggtacttccgccttcggtattaaccaaactactttcccaacagatgtatacgctatcgtccaagtcttgtggggttccattggtttcacagtcggcgctctattgggtgctactatggccgctgaagaacttgatccaaagaagagagttattttattcattggtgacggttctctacaattgactgttcaagaaatctctaccatgattagatggggtttgaagccatacatttttgtcttgaataacaacggttacaccattgaaaaattgattcacggtcctcatgccgaatataatgaaattcaaggttgggaccacttggccttattgccaacttttggtgctagaaactacgaaacccacagagttgctaccactggtgaatgggaaaagttgactcaagacaaggacttccaagacaactctaagattagaatgattgaagttatgttgccagtctttgatgctccacaaaacttggttaaacaagctcaattgactgccgctactaacgctaaacaataa

SEQ ID NO: 51 - DNA, 1692 bp, Saccharomyces cerivisiae
atgtctgaaattactcttggaaaatacttatttgaaagattgaagcaagttaatgttaacaccatttttgggctaccaggcgacttcaacttgtccctattggacaagatttacgaggtagatggattgagatgggctggtaatgcaaatgagctgaacgccgcctatgccgccgatggttacgcacgcatcaagggtttatctgtgctggtaactacttttggcgtaggtgaattatccgccttgaatggtattgcaggatcgtatgcagaacacgtcggtgtactgcatgttgttggtgtcccctctatctccgctcaggctaagcaattgttgttgcatcataccttgggtaacggtgattttaccgtttttcacagaatgtccgccaatatctcagaaactacatcaatgattacagacattgctacagccccttcagaaatcgataggttgatcaggacaacatttataacacaaaggcctagctacttggggttgccagcgaatttggtagatctaaaggttcctggttctcttttggaaaaaccgattgatctatcattaaaacctaacgatcccgaagctgaaaaggaagttattgataccgtactagaattgatccagaattcgaaaaaccctgttatactatcggatgcctgtgcttctaggcacaacgttaaaaaagaaacccagaagttaattgatttgacgcaattcccagcttttgtgacacctctaggtaaagggtcaatagatgaacagcatcccagatatggcggtgtttatgtgggaacgctgtccaaacaagacgtgaaacaggccgttgagtcggctgatttgatcctttcggtcggtgctttgctctctgattttaacacaggttcgttttcctactcctacaagactaaaaatgtagtggagtttcattccgattacgtaaaggtgaagaacgctacgttcctcggtgtacaaatgaaatttgcactacaaaacttactgaaggttattcccgatgttgttaagggctacaagagcgttcccgtaccaaccaaaactcccgcaaacaaaggtgtacctgctagcacgcccttgaaacaagagtggttgtggaacgaattgtccaaattcttgcaagaaggtgatgttatcatttccgagaccggcacgtctgccttcggtatcaatcaaactatctttcctaaggacgcctacggtatctcgcaggtgttgtgggggtccatcggttttacaacaggagcaactttaggtgctgcctttgccgctgaggagattgaccccaacaagagagtcatcttattcataggtgacgggtctttgcagttaaccgtccaagaaatctccaccatgatcagatgggggttaaagccgtatctttttgtccttaacaacgacggctacactatcgaaaagctgattcatgggcctcacgcagagtacaacgaaatccagacctgggatcacctcgccctgttgcccgcatttggtgcgaaaaagtacgaaaatcacaagatcgccactacgggtgagtgggatgccttaaccactgattcagagttccagaaaaactcggtgatcagactaattgaactgaaactgcccgtctttgatgctccggaaagtttgatcaaacaagcgcaattgactgccgctacaaatgccaaacaataa

SEQ ID NO: 52 - DNA, 1692 bp, candida glabrata
atgtctgagattactttgggtagatacttgttcgagagattgaaccaagtcgacgttaagaccatcttcggtttgccaggtgacttcaacttgtccctattggacaagatctacgaagttgaaggtatgagatgggctggtaacgctaacgaattgaacgctgcttacgctgctgacggttacgctagaatcaagggtatgtcctgtatcatcaccaccttcggtgtcggtgaattgtctgccttgaacggtattgccggttcttacgctgaacacgtcggtgtcttgcacgtcgtcggtgtcccatccatctcctctcaagctaagcaattgttgttgcaccacaccttgggtaacggtgacttcactgtcttccacagaatgtccgctaacatctctgagaccaccgctatggtcactgacatcgctaccgctccagctgagatcgacagatgtatcagaaccacctacatcacccaaagaccagtctacttgggtctaccagctaacttggtcgacctaaaggtcccagccaagcttttggaaaccccaattgacttgtccttgaagccaaacgacccagaagccgaaactgaagtcgttgacaccgtcttggaattgatcaaggctgctaagaacccagttatcttggctgatgcttgtgcttccagacacgacgtcaaggctgaaaccaagaagttgattgacgccactcaattcccatccttcgttaccccaatgggtaagggttccatcgacgaacaacacccaagattcggtggtgtctacgtcggtaccttgtccagaccagaagttaaggaagctgttgaatccgctgacttgatcttgtctgtcggtgctttgttgtccgatttcaacactggttctttctcttactcttacaagaccaagaacatcgtcgaattccactctgactacatcaagatcagaaacgctaccttcccaggtgtccaaatgaagttcgctttgcaaaagttgttgaacgccgtcccagaagctatcaagggttacaagccagtccctgtcccagctagagtcccagaaaacaagtcctgtgacccagctaccccattgaagcaagaatggatgtggaaccaagtttccaagttcttgcaagaaggtgatgttgttatcactgaaaccggtacctccgcttttggtatcaaccaaaccccattcccaaacaacgcttacggtatctcccaagttctatggggttccatcggtttcaccaccggtgcttgtttgggtgccgctttcgctgctgaagaaatcgacccaaagaagagagttatcttgttcattggtgacggttctttgcaattgactgtccaagaaatctccaccatgatcagatggggcttgaagccatacttgttcgtcttgaacaacgacggttacaccatcgaaagattgattcacggtgaaaaggctggttacaacgacatccaaaactgggaccacttggctctattgccaaccttcggtgctaaggactacgaaaaccacagagtcgccaccaccggtgaatgggacaagttgacccaagacaaggaattcaacaagaactccaagatcagaatgatcgaagttatgttgccagttatggacgctccaacttccttgattgaacaagctaagttgaccgcttccatcaacgctaagcaagaa

SEQ ID NO: 53 - protein, 564 aa, Candida glabrata
MSEITLGRYLFERLNQVDVKTIFGLPGDFNLSLLDKIYEVEGMRWAGNANELNAAYAADGYARIKGMSCIITTFGVGELSALNGIAGSYAEHVGVLHVVGVPSISSQAKQLLLHHTLGNGDFTVFHRMSANISETTAMVTDIATAPAEIDRCIRTTYITQRPVYLGLPANLVDLKVPAKLLETPIDLSLKPNDPEAETEVVDTVLELIKAAKNPVILADACASRHDVKAETKKLIDATQFPSFVTPMGKGSIDEQHPRFGGVYVGTLSRPEVKEAVESADLILSVGALLSDFNTGSFSYSYKTKNIVEFHSDYIKIRNATFPGVQMKFALQKLLNAVPEAIKGYKPVPVPARVPENKSCDPATPLKQEWMWNQVSKFLQEGDVVITETGTSAFGINQTPFPNNAYGISQVLWGSIGFTTGACLGAAFAAEEIDPKKRVILFIGDGSLQLTVQEISTMIRWGLKPYLFVLNNDGYTIERLIHGEKAGYNDIQNWDHLALLPTFGAKDYENHRVATTGEWDKLTQDKEFNKNSKIRMIEVMLPVMDAPTSLIEQAKLTASINAKQE

SEQ ID NO: 54 - DNA, 1788 bp, Pichia stipites
atggctgaagtctcattaggaagatatctcttcgagagattgtaccaattgcaagtgcagaccatcttcggtgtccctggtgatttcaacttgtcgcttttggacaagatctacgaagtggaagatgcccatggcaagaattcgtttagatgggctggtaatgccaacgaattgaatgcatcgtacgctgctgacggttactcgagagtcaagcgtttagggtgtttggtcactacctttggtgtcggtgaattgtctgctttgaatggtattgccggttcttatgccgaacatgttggtttgcttcatgtcgtaggtgttccatcgatttcctcgcaagctaagcaattgttacttcaccacactttgggtaatggtgatttcactgttttccatagaatgtccaacaacatttctcagaccacagcctttatctccgatatcaactcggctccagctgaaattgatagatgtatcagagaggcctacgtcaaacaaagaccagtttatatcgggttaccagctaacttagttgatttgaatgttccggcctctttgcttgagtctccaatcaacttgtcgttggaaaagaacgacccagaggctcaagatgaagtcattgactctgtcttagacttgatcaaaaagtcgctgaacccaatcatcttggtcgatgcctgtgcctcgagacatgactgtaaggctgaagttactcagttgattgaacaaacccaattcccagtatttgtcactccaatgggtaaaggtaccgttgatgagggtggtgtagacggagaattgttagaagatgatcctcatttgattgccaaggtcgctgctaggttgtctgctggcaagaacgctgcctctagattcggaggtgtttatgtcggaaccttgtcgaagcccgaagtcaaggacgctgtagagagtgcagatttgattttgtctgtcggtgcccttttgtctgatttcaacactggttcattttcctactcctacagaaccaagaacatcgtcgaattccattctgattacactaagattagacaagccactttcccaggtgtgcagatgaaggaagccttgcaagaattgaacaagaaagtttcatctgctgctagtcactatgaagtcaagcctgtgcccaagatcaagttggccaatacaccagccaccagagaagtcaagttaactcaggaatggttgtggaccagagtgtcttcgtggttcagagaaggtgatattattatcaccgaaaccggtacatcctccttcggtatagttcaatccagattcccaaacaacaccatcggtatctcccaagtattgtggggttctattggtttctctgttggtgccactttgggtgctgccatggctgcccaagaactcgaccctaacaagagaaccatcttgtttgttggagatggttctttgcaattgaccgttcaggaaatctccaccataatcagatggggtaccacaccttaccttttcgtgttgaacaatgacggttacaccatcgagcgtttgatccacggtgtaaatgcctcatataatgacatccaaccatggcaaaacttggaaatcttgcctactttctcggccaagaactacgacgctgtgagaatctccaacatcggagaagcagaagatatcttgaaagacaaggaattcggaaagaactccaagattagattgatagaagtcatgttaccaagattggatgcaccatctaaccttgccaaacaagctgccattacagctgccaccaacgccgaagct

SEQ ID NO: 55 - protein, 596 aa, Pichia stipites
MAEVSLGRYLFERLYQLQVQTIFGVPGDFNLSLLDKIYEVEDAHGKNSFRWAGNANELNASYAADGYSRVKRLGCLVTTFGVGELSALNGIAGSYAEHVGLLHVVGVPSISSQAKQLLLHHTLGNGDFTVFHRMSNNISQTTAFISDINSAPAEIDRCIREAYVKQRPVYIGLPANLVDLNVPASLLESPINLSLEKNDPEAQDEVIDSVLDLIKKSSNPIILVDACASRHDCKAEVTQLIEQTQFPVFVTPMGKGTVDEGGVDGELLEDDPHLIAKVAARLSAGKNAASRFGGVYVGTLSKPEVKDAVESADLILSVGALLSDFNTGSFSYSYRTKNIVEFHSDYTKIRQATFPGVQMKEALQELNKKVSSAASHYEVKPVPKIKLANTPATREVKLTQEWLWTRVSSWFREGDIIITETGTSSFGIVQSRFPNNTIGISQVLWGSIGFSVGATLGAAMAAQELDPNKRTILFVGDGSLQLTVQEISTIIRWGTTPYLFVLNNDGYTIERLIHGVNASYNDIQPWQNLEILPTFSAKNYDAVRISNIGEAEDILKDKEFGKNSKIRLIEVMLPRLDAPSNLAKQAAITAATNAEA

SEQ ID NO: 56 - DNA, 1707 bp, Pichia stipites
atggtatcaacctacccagaatcagaggttactctaggaaggtacctctttgagcgactccaccaattgaaagtggacaccattttcggcttgccgggtgacttcaacctttccttattggacaaagtgtatgaagttccggatatgaggtgggctggaaatgccaacgaattgaatgctgcctatgctgccgatggttactccagaataaagggattgtcttgcttggtcacaacttttggtgttggtgaattgtctgctttaaacggagttggtggtgcctatgctgaacacgtaggacttctacatgtcgttggagttccatccatatcgtcacaggctaaacagttgttgctccaccataccttgggtaatggtgacttcactgtttttcacagaatgtccaatagcatttctcaaactacagcatttctctcagatatctctattgcaccaggtcaaatagatagatgcatcagagaagcatatgttcatcagagaccagtttatgttggtttaccggcaaatatggttgatctcaaggttccttctagtctcttagaaactccaattgatttgaaattgaaacaaaatgatcctgaagctcaagaagttgttgaaacagtcctgaagttggtgtcccaagctacaaaccccattatcttggtagacgcttgtgccctcagacacaattgcaaagaggaagtcaaacaattggttgatgccactaattttcaagtctttacaactccaatgggtaaatctggtatctccgaatctcatccaagattgggcggtgtctatgtcgggacaatgtcgagtcctcaagtcaaaaaagccgttgaaaatgccgatcttatactatctgttggttcgttgttatcggacttcaatacaggttcattttcatactcctacaagacgaagaatgttgttgaattccactctgactatatgaaaatcagacaggccaccttcccaggagttcaaatgaaagaagccttgcaacagttgataaaaagggtctcttcttacatcaatccaagctacattcctactcgagttcctaaaaggaaacagccattgaaagctccatcagaagctcctttgacccaagaatatttgtggtctaaagtatccggctggtttagagagggtgatattatcgtaaccgaaactggtacatctgctttcggaattattcaatcccattttcccagcaacactatcggtatatcccaagtcttgtggggctcaattggtttcacagtaggtgcaacagttggtgctgccatggcagcccaggaaatcgaccctagcaggagagtaattttgttcgtcggtgatggttcattgcagttgacggttcaggaaatctctacgttgtgtaaatgggattgtaacaatacttatctttacgtgttgaacaatgatggttacactatagaaaggttgatccacggcaaaagtgccagctacaacgatatacagccttggaaccatttatccttgcttcgcttattcaatgctaagaaataccaaaatgtcagagtatcgactgctggagaattggactctttgttctctgataagaaatttgcttctccagataggataagaatgattgaggtgatgttatcgagattggatgcaccagcaaatcttgttgctcaagcaaagttgtctgaacgggtaaaccttgaaaat

SEQ ID NO: 57 - protein, 569 aa, Pichia stipites
MVSTYPESEVTLGRYLFERLHQLKVDTIFGLPGDFNLSLLDKVYEVPDMRWAGNANELNAAYAADGYSRIKGLSCLVTTFGVGELSALNGVGGAYAEHVGLLHVVGVPSISSQAKQLLLHHTLGNGDFTVFHRMSNSISQTTAFLSDISIAPGQIDRCIREAYVHQRPVYVGLPANMVDLKVPSSLLETPIDLKLKQNDPEAQEVVETVLKLVSQATNPIILVDACALRHNCKEEVKQLVDATNFQVFTTPMGKSGISESHPRLGGVYVGTMSSPQVKKAVENADLILSVGSLLSDFNTGSFSYSYKTKNVVEFHSDYMKIRQATFPGVQMKEALQQLIKRVSSYINPSYIPTRVPKRKQPLKAPSEAPLTQEYLWSKVSGWFREGDIIVTETGTSAFGIIQSHFPSNTIGISQVLWGSIGFTVGATVGAAMAAQEIDPSRRVILFVGDGSLQLTVQEISTLCKWDCNNTYLYVLNNDGYTIERLIHGKSASYNDIQPWNHLSLLRLFNAKKYQNVRVSTAGELDSLFSDKKFASPDRIRMIEVMLSRLDAPANLVAQAKLSERVNLEN

SEQ ID NO: 58 - DNA, 1692 bp, Kluyveromyces lactis
atgtctgaaattacattaggtcgttacttgttcgaaagattaaagcaagtcgaagttcaaaccatctttggtctaccaggtgatttcaacttgtccctattggacaatatctacgaagtcccaggtatgagatgggctggtaatgccaacgaattgaacgctgcttacgctgctgatggttacgccagattaaagggtatgtcctgtatcatcaccaccttcggtgtcggtgaattgtctgctttgaacggtattgccggttcttacgctgaacacgttggtgtcttgcacgttgtcggtgttccatccgtctcttctcaagctaagcaattgttgttgcaccacaccttgggtaacggtgacttcactgttttccacagaatgtgctccaacatttctgaaaccactgctatgatcaccgatatcaacactgccccagctgaaatcgacagatgtatcagaaccacttacgtttcccaaagaccagtctacttgggtttgccagctaacttggtcgacttgactgtcccagcttctttgttggacactccaattgatttgagcttgaagccaaatgacccagaagccgaagaagaagtcatcgaaaacgtcttgcaactgatcaaggaagctaagaacccagttatcttggctgatgcttgttgttccagacacgatgccaaggctgagaccaagaagttgatcgacttgactcaattcccagccttcgttaccccaatgggtaagggttccattgacgaaaagcacccaagattcggtggtgtctacgtcggtaccctatcttctccagctgtcaaggaagccgttgaatctgctcacttggttctatcggtcggtgctctattgtccgatttcaacactggttctttctcttactcttacaagaccaagaacattgtcgaattccactctgactacaccaagatcagaaggcctaccttcccaggtgtccaaatgaagttcgctttacaaaaattgttgactaaggttgccgatgctgctaagggttacaagccagttccagttccatctgaaccagaacacaacgaagatgtcgctgactccactccattgaagcaagaatgggtctggactcaagtcggtgaattcttgagagaaggtgatgttgttatcactgaaaccggtacctctgccttcggtatcaaccaaactcatttcccaaacaacacatacggtatctctcaagttttatggggttccattggtttcaccactggtgctaccttgggtgctgccttcgctgccgaagaaattgatccaaagaagagagttatcttattcattggtgacggttctttgcaattgactgttcaagaaatctccaccatgatcagatggggcttgaagccatacttgttcgtattgaacaacgacggttacaccattgaaagattgattcacggtgaaaccgctcaatacaactgtatccaaaactggcaacacttggaattattgccaactttcggtgccaaggactacgaagctgtcagagtttccaccactggtgaatggaacaagttgaccactgacgaaaagttccaagacaacaccagaatcagattgatcgaagttatgttgccaactatggatgctccatctaacttggttaagcaagctcaattgactgctgcatccaacgctaagaactaa

SEQ ID NO: 59 - protein, 563 aa, Kluyveromyces lactis
MSEITLGRYLFERLKQVEVQTIFGLPGDFNLSLLDNIYEVPGMRWAGNANELNAAYAADGYARLKGMSCIITTFGVGELSALNGIAGSYAEHVGVLHVVGVPSVSSQAKQLLLHHTLGNGDFTVFHRMSSNISETTAMITDINTAPAEIDRCIRTTYVSQRPVYLGLPANLVDLTVPASLLDTPIDLSLKPNDPEAEEEVIENVLQLIKEAKNPVILADACCSRHDAKAETKKLIDLTQFPAFVTPMGKGSIDEKHPRFGGVYVGTLSSPAVKEAVESADLVLSVGALLSDFNTGSFSYSYKTKNIVEFHSDYTKIRSATFPGVQMKFALQKLLTKVADAAKGYKPVPVPSEPEHNEAVADSTPLKQEWVWTQVGEFLREGDVVITETGTSAFGINQTHFPNNTYGISQVLWGSIGFTTGATLGAAFAAEEIDPKKRVILFIGDGSLQLTVQEISTMIRWGLKPYLFVLNNDGYTIERLIHGETAQYNCIQNWQHLELLPTFGAKDYEAVRVSTTGEWNKLTTDEKFQDNTRIRLIEVMLPTMDAPSNLVKQAQLTAATNAKN

SEQ ID NO: 60 - DNA, 1716 bp, Yarrowia lipolytica
atgagcgactccgaaccccaaatggtcgacctgggcgactatctctttgcccgattcaagcagctaggcgtggactccgtctttggagtgcccggcgacttcaacctcaccctgttggaccacgtgtacaatgtcgacatgcggtgggttgggaacacaaacgagctgaatgccggctactcggccgacggctactcccgggtcaagcggctggcatgtcttgtcaccacctttggcgtgggagagctgtctgccgtggctgctgtggcaggctcgtacgccgagcatgtgggcgtggtgcatgttgtgggcgttcccagcacctctgctgagaacaagcatctgctgctgcaccacacactcggtaacggcgacttccgggtctttgcccagatgtccaaactcatctccgagtacacccaccatattgaggaccccagcgaggctgccgacgtaatcgacaccgccatccgaatcgcctacacccaccagcggcccgtttacattgctgtgccctccaacttctccgaggtcgatattgccgaccaggctagactggatacccccctggacctttcgctgcagcccaacgaccccgagagccagtacgaggtgattgaggagatttgctcgcgtatcaaggccgccaagaagcccgtgattctcgtcgacgcctgcgcttcgcgatacagatgtgtggacgagaccaaggagctggccaagatcaccaactttgcctactttgtcactcccatgggtaagggttctgtggacgaggatactgaccggtacggaggaacatacgtcggatcgctgactgctcctgctactgccgaggtggttgagacagctgatctcatcatctccgtaggagctcttctgtcggacttcaacaccggttccttctcgtactcctactccaccaaaaacgtggtggaattgcattcggaccacgtcaaaatcaagtccgccacctacaacaacgtcggcatgaaaatgctgttcccgcccctgctcgaagccgtcaagaaactggttgccgagacccctgactttgcatccaaggctctggctgttcccgacaccactcccaagatccccgaggtacccgatgatcacattacgacccaggcatggctgtggcagcgtctcagttactttctgaggcccaccgacatcgtggtcaccgagaccggaacctcgtcctttggaatcatccagaccaagttcccccacaacgtccgaggtatctcgcaggtgctgtggggctctattggatactcggtgggagcagcctgtggagcctccattgctgcacaggagattgacccccagcagcgagtgattctgtttgtgggcgacggctctcttcagctgacggtgaccgagatctcgtgcatgatccgcaacaacgtcaagccgtacatttttgtgctcaacaacgacggctacaccatcgagaggctcattcacggcgaaaacgcctcgtacaacgatgtgcacatgtggaagtactccaagattctcgacacgttcaacgccaaggcccacgagtcgattgtggtcaacaccaagggcgagatggacgctctgttcgacaacgaagagtttgccaagcccgacaagatccggctcattgaggtcatgtgcgacaagatggacgcgcctgcctcgttgatcaagcaggctgagctctctgccaagaccaacgtttag

SEQ ID NO: 61 - protein, 571 aa, Yarrowia lipolytica
MSDSEPQMVDLGDYLFARFKQLGVDSVFGVPGDFNLTLLDHVYNVDMRWVGNTNELNAGYSADGYSRVKRLACLVTTFGVGELSAVAAVAGSYAEHVGVVHVVGVPSTSAENKHLLLHHTLGNGDFRVFAQMSKLISEYTHHIEDPSEAADVIDTAIRIAYTHQRPVYIAVPSNFSEVDIADQARLDTPLDLSLQPNDPESQYEVIEEICSRIKAAKKPVILVDACASRYRCVDETKELAKITNFAYFVTPMGKGSVDEDTDRYGGTYVGSLTAPATAEVVETADLIISVGALLSDFNTGSFSYSYSTKNVVELHSDHVKIKSATYNNVGMKMLFPPLLEAVKKLVAETPDFASKALAVPDTTPKIPEVPDDHITTQAWLWQRLSYFLRPTDIVVTETGTSSFGIIQTKFPHNVRGISQVLWGSIGYSVGAACGASIAAQEIDPQQRVILFVGDGSLQLTVTEISCMIRNNVKPYIFVLNNDGYTIERLIHGENASYNDVHMWKYSKILDTFNAKAHESIVVNTKGEMDALFDNEEFAKPDKIRLIEVMCDKMDAPASLIKQAELSAKTNV

SEQ ID NO: 62 - DNA, 1716 bp, Schizosaccharomyces pombe
atgagtggggatattttagtcggtgaatatctattcaaaaggcttgaacaattaggggtcaagtccattcttggtgttccaggagatttcaatttagctctacttgacttaattgagaaagttggagatgagaaatttcgttgggttggcaataccaatgagttgaatggtgcttatgccgctgatggttatgctcgtgttaatggtctttcagccattgttacaacgttcggcgtgggagagctttccgctattaatggagtggcaggttcttatgcggagcatgtcccagtagttcatattgttggaatgccttccacaaaggtgcaagatactggagctttgcttcatcatactttaggagatggagactttcgcactttcatggatatgtttaagaaagtttctgcctacagtataatgatcgataacggaaacgatgcagctgaaaagatcgatgaagccttgtcgatttgttataaaaaggctaggcctgtttacattggtattccttctgatgctggctacttcaaagcatcttcatcaaatcttgggaaaagactaaagctcgaggaggatactaacgatccagcagttgagcaagaagtcatcaatcatatctcggaaatggttgtcaatgcaaagaaaccagtgattttaattgacgcttgtgctgtaagacatcgtgtcgttccagaagtacatgagctgattaaattgacccatttccctacatatgtaactcccatgggtaaatctgcaattgacgaaacttcgcaattttttgacggcgtttatgttggttcaatttcagatcctgaagttaaagacagaattgaatccactgatctgttgctatccatcggtgctctcaaatcagactttaacacgggttccttctcttaccacctcagccaaaagaatgccgttgagtttcattcagaccacatgcgcattcgatatgctctttatccaaatgtagccatgaagtatattcttcgcaaactgttgaaagtacttgatgcttctatgtgtcattccaaggctgctcctaccattggctacaacatcaagcctaagcatgcggaaggatattcttccaacgagattactcattgctggttttggcctaaatttagtgaatttttgaagccccgagatgttttgatcaccgagactggaactgcaaactttggtgtccttgattgcaggtttccaaaggatgtaacagccatttcccaggtattatggggatctattggatactccgttggtgcaatgtttggtgctgttttggccgtccacgattctaaagagcccgatcgtcgtaccattcttgtagtaggtgatggatccttacaactgacgattacagagatttcaacctgcattcgccataacctcaaaccaattattttcataattaacaacgacggttacaccattgagcgtttaattcatggtttgcatgctagctataacgaaattaacactaaatggggctaccaacagattcccaagtttttcggagctgctgaaaaccacttccgcacttactgtgttaaaactcctactgacgttgaaaagttgtttagcgacaaggagtttgcaaatgcagatgtcattcaagtagttgagcttgtaatgcctatgttggatgcacctcgtgtcctagttgagcaagccaagttgacgtctaagatcaataagcaatga

SEQ ID NO: 63 - protein, 571 aa, Schizosaccharomyces pombe
MSGDILVGEYLFKRLEQLGVKSILGVPGDFNLALLDLIEKVGDEKFRWVGNTNELNGAYAADGYARVNGLSAIVTTFGVGELSAINGVAGSYAEHVPVVHIVGMPSTKVQDTGALLHHTLGDGDFRTFMDMFKKVSAYSIMIDNGNDAAEKIDEALSICYKKARPVYIGIPSDAGYFKASSSNLGKRLKLEEDTNDPAVEQEVINHISEMVVNAKKPVILIDACAVRHRVVPEVHELIKLTHFPTYVTPMGKSAIDETSQFFDGVYVGSISDPEVKDRIESTDLLLSIGALKSDFNTGSFSYHLSQKNAVEFHSDHMRIRYALYPNVAMKYILRKLLKVLDASMCHSKAAPTIGYNIKPKHAEGYSSNEITHCWFWPKFSEFLKPRDVLITETGTANFGVLDCRFPKDVTAISQVLWGSIGYSVGAMFGAVLAVHDSKEPDRRTILVVGDGSLQLTITEISTCIRHNLKPIIFIINNDGYTIERLIHGLHASYNEINTKWGYQQIPKFFGAAENHFRTYCVKTPTDVEKLFSDKEFANADVIQVVELVMPMLDAPRVLVEQAKLTSKINKQ

SEQ ID NO: 64 - DNA, 1689 bp, Zygosaccharomyces rouxii
atgtctgaaattactctaggtcgttacttgttcgaaagattaaagcaagttgacactaacaccatcttcggtgttccaggtgacttcaacttgtccttgttggacaaggtctacgaagtgcaaggtctaagatgggctggtaacgctaacgaattgaacgctgcctacgctgctgacggttacgccagagttaagggtttggctgctttgatcaccaccttcggtgtcggtgaattgtctgctttgaacggtattgcaggttcttacgctgaacacgttggtgttttgcacattgttggtgttccatctgtctcttctcaagctaagcaattgttgttgcaccacaccttgggtaacggtgacttcactgttttccacagaatgtccgccaacatctctgaaaccaccgctatgttgaccgacatcactgctgctccagctgaaattgaccgttgcatcagagttgcttacgtcaaccaaagaccagtctacttgggtctaccagctaacttggttgaccaaaaggtcccagcttctttgttgaacactccaattgatctatctctaaaggagaacgacccagaagctgaaaccgaagttgttgacaccgttttggaattgatcaaggaagctaagaacccagttatcttggctgatgcttgctgctccagacacgacgtcaaggctgaaaccaagaagttgatcgacttgactcaattcccatctttcgttactcctatgggtaagggttccatcgacgaacaaaacccaagattcggtggtgtctacgtcggtactctatccagcccagaagttaaggaagctgttgaatctgctgacttggttctatctgtcggtgctctattgtccgatttcaacactggttctttctcttactcttacaagaccaagaacgttgttgaattccactctgaccacatcaagatcagaaacgctaccttcccaggtgttcaaatgaaattcgttttgaagaaactattgcaagctgtcccagaagctgtcaagaactacaagccaggtccagtcccagctccgccatctccaaacgctgaagttgctgactctaccaccttgaagcaagaatggttatggagacaagtcggtagcttcttgagagaaggtgatgttgttattaccgaaactggtacctctgctttcggtatcaaccaaactcacttccctaaccaaacttacggtatctctcaagtcttgtggggttctattggttacaccactggttccactttgggtgctgccttcgctgctgaagaaattgaccctaagaagagagttatcttgttcattggtgacggttctctacaattgaccgttcaagaaatctccaccatgatcagatggggtctaaagccatacttgttcgttttgaacaacgatggttacaccattgaaagattgattcacggtgaaaccgctgaatacaactgtatccaaccatggaagcacttggaattgttgaacaccttcggtgccaaggactacgaaaaccacagagtctccactgtcggtgaatggaacaagttgactcaagatccaaaattcaacgaaaactctagaattagaatgatcgaagttatgcttgaagtcatggacgctccatcttctttggtcgctcaagctcaattgaccgctgctactaacgctaagcaa

SEQ ID NO: 65 - protein, 563 aa, Zygosaccharomyces rouxii
MSEITLGRYLFERLKQVDTNTIFGVPGDFNLSLLDKVYEVQGLRWAGNANELNAAYAADGYARVKGLAALITTFGVGELSALNGIAGSYAEHVGVLHIVGVPSVSSQAKQLLLHHTLGNGDFTVFHRMSANISETTAMLTDITAAPAEIDRCIRVAYVNQRPVYLGLPANLVDQKVPASLLNTPIDLSLKENDPEAETEVVDTVLELIKEAKNPVILADACCSRHDVKAETKKLIDLTQFPSFVTPMGKGSIDEQNPRFGGVYVGTLSSPEVKEAVESADLVLSVGALLSDFNTGSFSYSYKTKNVVEFHSDHIKIRNATFPGVQMKFVLKKLLQAVPEAVKNYKPGPVPAPPSPNAEVADSTTLKQEWLWRQVGSFLREGDVVITETGTSAFGINQTHFPNQTYGISQVLWGSIGYTTGSTLGAAFAAEEIDPKKRVILFIGDGSLQLTVQEISTMIRWGLKPYLFVLNNDGYTIERLIHGETAEYNCIQPWKHLELLNTFGAKDYENHRVSTVGEWNKLTQDPKFNENSRIRMIEVMLEVMDAPSSLVAQAQLTAATNAKQ

SEQ ID NO: 66 - protein, 570 aa, Bacillus subtilis
MTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 67 - protein, 343 aa, Anaerostipes caccae
MEECKMAKIYYQEDCNLSLLDGKTIAVIGYGSQGHAHALNAKESGCNVIIGLYEGAKEWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 68 - protein, 343 aa, Anaerostipes caccae
MEECKMAKIYYQEDCNLSLLDGKTIAVIGYGSQGHAHALNAKESGCNVIIGLYEGAKDWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 69 - protein, 338 aa, Pseudomonas fluorescens
MKVFYDKDCDLSIIQGKKVAIIGYGSQGHAQACNLKDSGVDVTVGLRKGSATVAKAEAHGLKVTDVAAAVAGADLVMILTPDEFQSQLYKNEIEPNIKKGATLAFSHGFAIHYNQVVPRADLDVIMIAPKAPGHTVRSEFVKGGGIPDLIAIYQDASGNAKNVALSYAAGVGGGRTGIIETTFKDETETDLFGEQAVLCGGTVELVKAGFETLVEAGYAPEMAYFECLHELKLIVDLMYEGGIANMNYSISNNAEYGEYVTGPEVINAESRQAMRNALKRIQDGEYAKMFISEGATGYPSMTAKRRNNAAHGIEIIGEQLRSMMPWIGANKIVDKAKN

SEQ ID NO: 70 - protein, 571 aa, Streptococcus mutans DHAD
MTDKKTLKDLRNRSSVYDSMVKSPNRAMLRATGMQDEDFEKPIVGVISTWAENTPCNIHLHDFGKLAKVGVKEAGAWPVQFGTITVSDGIAMGTQGMRFSLTSRDIIADSIEAAMGGHNADAFVAIGGCDKNMPGSVIAMANMDIPAIFAYGGTIAPGNLDGKDIDLVSVFEGVGHWNHGDMTKEEVKALECNACPGPGGCGGMYTANTMATAIEVLGLSLPGSSSHPAESAEKKADIEEAGRAVVKMLEMGLKPSDILTREAFEDAITVTMALGGSTNSTLHLLAIAHAANVELTLDDFNTFQEKVPHLADLKPSGQYVFQDLYKVGGVPAVMKYLLKNGFLHGDRITCTGKTVAENLKAFDDLTPGQKVIMPLENPKREDGPLIILHGNLAPDGAVAKVSGVKVRRHVGPAKVFNSEEEAIEAVLNDDIVDGDVVVVRFVGPKGGPGMPEMLSLSSMIVGKGQGEKVALLTDGRFSGGTYGLVVGHIAPEAQDGGPIAYLQTGDIVTIDQDTKELHFDISDEELKHRQETIELPPLYSRGILGKYAHIVSSASRGAVTDFWKPEETGKK

SEQ ID NO: 71 - protein, 546 aa, Macrococcus caseolyticus
MKQRIGQYLIDALHVNGVDKIFGVPGDFTLAFLDDIIRHDNVEWVGNTNELNAAYAADGYARVNGLAAVSTTFGVGELSAVNGIAGSYAERVPVIKISGGPSSVAQQEGRYVHHSLGEGIFDSYSKMYAHITATTTILSVDNAVDEIDRVIHCALKEKRPVHIHLPIDVALTEIEIPHAPKVYTHESQNVDAYIQAVEKKLMSAKQPVIIAGHEINSFKLHEQLEQFVNQTNIPVAQLSLGKSAFNEENEHYLGIYDGKIAKENVREYVDNADVILNIGAKLTDSATAGFSYKFDTNNIIYINHNDFKAEDVISDNVSLIDLVNGLNSIDYRNETHYPSYQRSDMKYELNDAPLTQSNYFKMMNAFLEKDDILLAEQGTSFFGAYDLSLYKGNQFIGQPLWGSIGYTFPSLLGSQLADMHRRNILLIGDGSLQLTVQALSTMIRKDIKPIIFVINNDGYTVERLIHGMEEPYNDIQMWNYKQLPEVFGGKDTVKVHDAKTSNELKTVMDSVKADKDHMHFIEVHMAVEDAPKKLIDIAKAFSDANK

SEQ ID NO: 72 - protein, 548 aa, Listeria grayi
MYTVGQYLVDRLEEIGIDKVFGVPGDYNLTFLDYIQNHEGLSWQGNTNELNAAYAADGYARERGVSALVTTFGVGELSAINGTAGSFAEQVPVIHIVGSPTMNVQSNKKLVHHSLGMGNFHNFSEMAKEVTAATTMLTEENAASEIDRVLETALLEKRPVYINLPIDIAHKAIVKPAKALQTEKSSGEREAQLAEIILSHLEKAAQPIVIAGHEIARFQIRERFENWINQTKLPVTNLAYGKGSFNEENEHFIGTYYPAFSDKNVLDYVDNSDFVLHFGGKIIDNSTSSFSQGFKTENTLTAANDIIMLPDGSTYSGISLNGLLAELEKLNFTFADTAAKQAELAVFEPQAETPLKQDRFHQAVMNFLQADDVLVTEQGTSSFGLMLAPLKKGMNLISQTLWGSIGYTLPAMIGSQIAAPERRHILSIGDGSFQLTAQEMSTIFREKLTPVIFIINNDGYTVERAIHGEDESYNDIPTWNLQLVAETFGGDAETVDTHNVFTETDFANTLAAIDATPQKAHVVEVHMEQMDMPESLRQIGLALSKQNS

SEQ ID NO: 73 - protein, 348 aa, Achromobacter xylosoxidans
MKALVYHGDHKISLEDKPKPTLQKPTDVVVRVLKTTICGTDLGIYKGKNPEVADGRILGHEGVGVIEEVGESVTQFKKGDKVLISCVTSCGSCDYCKKQLYSHCRDGGWILGYMIDGVQAEYVRIPHADNSLYKIPQTIDDEIAVLLSDILPTGHEIGVQYGNVQPGDAVAIVGAGPVGMSVLLTAQFYSPSTIIVIDMDENRLQLAKELGATHTINSGTENVVEAVHRIAAEGVDVAIEAVGIPATWDICQEIVKPGAHIANVGVHGVKVDFEIQKLWIKNLTITTGLVNTNTTPMLMKVASTDKLPLKKMITHRFELAEIEHAYQVFLNGAKEKAMKIILSNAGAA

SEQ ID NO: 74 - protein, 347 aa, Beijerickia indica
MKALVYRGPGQKLVEERQKPELKEPGDAIVKVTKTTICGTDLHILKGDVATCKPGRVLGHEGVGVIESVGSGVTAFQPGDRVLISCISSCGKCSFCRRGMFSHCTTGGWILGNEIDGTQAEYVRVPHADTSLYRIPAGADEEALVMLSDILPTGFECGVLNGKVAPGSSVAIVGAGPVGLAALLTAQFYSPAEIIMIDLDDNRLGLAKQFGATRTVNSTGGNAAAEVKALTEGLGVDTAIEAVGIPATFELCQNIVAPGGTIANVGVHGSKVDLHLESLWSHNVTITTRLVDTATTPMLLKTVQSHKLDPSRLITHRFSLDQILDAYETFGQAASTQALKVIISMEA

SEQ ID NO: 75 - protein, 267 aa, Saccharomyces cerevisiae
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 76 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 77 - DNA, 32 bp, Artificial Sequence
  Primer oBP622
aattggtaccccaaaaggaatattgggtcaga

SEQ ID NO: 78 - DNA, 49 bp, Artificial sequence
  Primer oBP623
ccattgtttaaacggcgcgccggatcctttgcgaaaccctatgctctgt

SEQ ID NO: 79 - DNA, 49 bp, Primer oBP624
gcaaaggatccggcgcgccgtttaaacaatggaaggtcgggatgagcat

SEQ ID NO: 80 - DNA, 34 bp, Artificial sequence
  Primer oBP625
aattggccggcctacgtaacattctgtcaaccaa

SEQ ID NO: 81 - DNA, 34 bp, Artificial sequence
  Primer oBP626
aattgcggccgcttcatatatgacgtaataaaat

SEQ ID NO: 82 - DNA, 34 bp, Artificial sequence
  Primer oBP627
aattttaattaattttttttcttggaatcagtac

SEQ ID NO: 83 - DNA, 40 bp, Artificial sequence
  Primer HY21
ttaaggcgcgcctatttgtaatacgtatacgaattccttc

SEQ ID NO: 84 - DNA, 56 bp, Artificial sequence
  Primer HY24
acttaataactttaccggctgttgacattttgttcttcttgttattgtattgtgtt

SEQ ID NO: 85 - DNA, 56 bp, Artificial sequence
  Primer HY25
aacacaatacaataacaagaagaacaaaatgtcaacagccggtaaagttattaagt

SEQ ID NO: 86 - DNA, 40 bp, Artificial sequence
  Primer HY4
ggaagtttaaacaccacaggtgttgtcctctgaggacata

SEQ ID NO: 87 - DNA, 30 bp, Artificial sequence
  Primer URA3-end F
gcatatttgagaagatgcggccagcaaaac

SEQ ID NO: 88 - DNA, 22 bp, Artificial sequence
  Primer oBP636
catttttttccctctaagaagc

SEQ ID NO: 89 - DNA, 22 bp, Artificial sequence
  Primer oBP637
tttttgcacagttaaactaccc

SEQ ID NO: 90 - DNA, 40 bp, Artificial sequence
  Primer oBP691
aattggatccgcgatcgcgacgttctctccgttgttcaaa

SEQ ID NO: 91 - DNA, 41 bp, Artificial sequence
  Primer oBP692
aattggcgcgccatttaaatatatatgtatatatataacac

SEQ ID NO: 92 - DNA, 34 bp, Artificial sequence
  Primer oBP693
aattgtttaaacaaaggatgatattgttctatta

SEQ ID NO: 93 - DNA, 33 bp, Artificial sequence
  Primer oBP694
aattggccggccgcaacgacgacaatgccaaac

SEQ ID NO: 94 - DNA, 34 bp, Artificial sequence
  Primer oBP695
aattgcggccgcatgacaggtgaaagaattgaaa

SEQ ID NO: 95 - DNA, 34 bp, Artificial sequence
  Primer oBP696
aattttaattaaacgggcatcttatagtgtcgtt

SEQ ID NO: 96 - DNA, 40 bp, Artificial sequence
  Primer HY16
ttaaggcgcgccccgcacgccgaaatgcatgcaagtaacc

SEQ ID NO: 97 - DNA, 56 bp, Artificial sequence
  Primer HY19
acttaataactttaccggctgttgacattttgattgatttgactgtgttattttgc

SEQ ID NO: 98 - DNA, 56 bp, Artificial sequence
  Primer HY20
gcaaaataacacagtcaaatcaatcaaaatgtcaacagccggtaaagttattaagt

SEQ ID NO: 99 - DNA, 22 bp, Artificial sequence
  Primer oBP730
ttgctccaaagagatgtcttta

SEQ ID NO: 100 - DNA, 22 bp, Artificial sequence
  Primer oBP731
tgttcccacaatctattaccta

SEQ ID NO: 101 - DNA, 80 bp, Artificial sequence
  Primer BK505
ttccggtttctttgaaatttttttgattcggtaatctccgagcagaaggagcattgcggattacgtattctaatgttcag

SEQ ID NO: 102 - DNA, 81 bp, Artificial Sequence
  Primer BK506
gggtaataactgatataattaaattgaagctctaatttgtgagtttagtacaccttggctaactcgttgtatcatcactgg

SEQ ID NO: 103 - DNA, 38 bp, Artificial Sequence
  Primer LA468
gcctcgagttttaatgttacttctcttgcagttaggga

SEQ ID NO: 104 - DNA, 31 bp, Artificial Sequence
  Primer LA492
gctaaattcgagtgaaacacaggaagaccag

SEQ ID NO: 105 - DNA, 23 bp, Artificial Sequence
  Primer AK109-1
agtcacatcaagatcgtttatgg

SEQ ID NO: 106 - DNA, 23 bp, Artificial Sequence
  Primer AK109-2
gcacggaatatgggactacttcg

SEQ ID NO: 107 - DNA, 23 bp, Artificial Sequence
  Primer AK109-3
actccacttcaagtaagagtttg

SEQ ID NO: 108 - DNA, 24 bp, Artificial Sequence
  Primer oBP452
ttctcgacgtgggcctttttcttg

SEQ ID NO: 109 - DNA, 49 bp, Artificial Sequence
  Primer oBP453
tgcagctttaaataatcggtgtcactactttgccttcgtttatcttgcc

SEQ ID NO: 110 - DNA, 49 bp, Artificial Sequence
  Primer oBP454
gagcaggcaagataaacgaaggcaaagtagtgacaccgattatttaaag

SEQ ID NO: 111 - DNA, 49 bp, Artificial Sequence
  Primer oBP455
tatggaccctgaaaccacagccacattgtaaccaccacgacggttgttg

SEQ ID NO: 112 - DNA, 49 bp, Artificial Sequence
  Primer oBP456
tttagcaacaaccgtcgtggtggttacaatgtggctgtggtttcagggt

SEQ ID NO: 113 - DNA, 49 bp, Artificial Sequence
  Primer oBP457
ccagaaaccctatacctgtgtggacgtaaggccatgaagctttttcttt

SEQ ID NO: 114 - DNA, 49 bp, Artificial Sequence
  Primer oBP458
attggaaagaaaaagcttcatggccttacgtccacacaggtatagggtt

SEQ ID NO: 115 - DNA, 22 bp, Artificial Sequence
  Primer oBP459
cataagaacacctttggtggag

SEQ ID NO: 116 - DNA, 22 bp, Artificial Sequence
  Primer BP460
aggattatcattcataagtttc

SEQ ID NO: 117 - DNA, 20 bp, Artificial Sequence
  Primer LA135
cttggcagcaacaggactag

SEQ ID NO: 118 - DNA, 23 bp, Artificial Sequence
  Primer BP461
ttcttggagctgggacatgtttg

SEQ ID NO: 119 - DNA, 22 bp, Artificial Sequence
  Primer LA92
gagaagatgcggccagcaaaac

SEQ ID NO: 120 - DNA, 80 bp, Artificial Sequence
  Primer LA678
caacgttaacaccgttttcggtttgccaggtgacttcaacttgtccttgtgcattgcggattacgtattctaatgttcag

SEQ ID NO: 121 - DNA, 81 bp, Artificial Sequence
  Primer LA679
gtggagcatcgaagactggcaacatgatttcaatcattctgatcttagagcaccttggctaactcgttgtatcatcactgg

SEQ ID NO: 122 - DNA, 23 bp, Artificial Sequence
  Primer LA337
ctcatttgaatcagcttatggtg

SEQ ID NO: 123 - DNA, 24 bp, Artificial Sequence
  Primer LA692
ggaagtcattgacaccatcttggc

SEQ ID NO: 124 - DNA, 24 bp, Artificial Sequence
  Primer LA693
agaagctgggacagcagcgttagc

SEQ ID NO: 125 - DNA, 96 bp, Artificial Sequence
  Primer LA722
tgccaattatttacctaaacatctataaccttcaaaagtaaaaaaatacacaaacgttgaatcatcaccttggctaactcgttgtatcatcactgg

SEQ ID NO: 126 - DNA, 80 bp, Artificial Sequence
  Primer LA733
cataatcaatctcaaagagaacaacacaatacaataacaagaagaacaaagcattgcggattacgtattctaatgttcag

SEQ ID NO: 127 - DNA, 30 bp, Artificial Sequence
  Primer LA453
caccgaagaagaatgcaaaaatttcagctc

SEQ ID NO: 128 - DNA, 25 bp, Artificial Sequence
  Primer LA694
gctgaagttgttagaactgttgttg

SEQ ID NO: 129 - DNA, 21 bp, Artificial Sequence
  Primer LA695
tgttagctggagtagacttgg

SEQ ID NO: 130 - DNA, 22 bp, Artificial sequence
  Primer oBP594
agctgtctcgtgttgtgggttt

SEQ ID NO: 131 - DNA, 49 bp, Artificial sequence
  Primer oBP595
cttaataatagaacaatatcatcctttacgggcatcttatagtgtcgtt

SEQ ID NO: 132 - DNA, 49 bp, Artificial sequence
  Primer oBP596
gcgccaacgacactataagatgcccgtaaaggatgatattgttctatta

SEQ ID NO: 133 - DNA, 49 bp, Artificial sequence
  Primer oBP597
tatggaccctgaaaccacagccacattgcaacgacgacaatgccaaacc

SEQ ID NO: 134 - DNA, 49 bp, Artificial sequence
  Primer oBP598
tccttggtttggcattgtcgtcgttgcaatgtggctgtggtttcagggt

SEQ ID NO: 135 - DNA, 49 bp, Artificial sequence
  Primer oBP599
atcctctcgcggagtccctgttcagtaaaggccatgaagctttttcttt

SEQ ID NO: 136 - DNA, 49 bp, Artificial sequence
  Primer oBP600
attggaaagaaaaagcttcatggcctttactgaacagggactccgcgag

SEQ ID NO: 137 - DNA, 22 bp, Artificial sequence
  Primer oBP601
tcataccacaatcttagaccat

SEQ ID NO: 138 - DNA, 21 bp, Artificial sequence
  Primer oBP602
tgttcaaacccctaaccaacc

SEQ ID NO: 139 - DNA, 22 bp, Artificial sequence
  Primer oBP603
tgttcccacaatctattaccta

SEQ ID NO: 140 - DNA, 90 bp, Artificial sequence
  Primer LA512
gtattttggtagattcaattctctttccctttccttttccttcgctccccttccttatcagcattgcggattacgtattctaatgttcag

SEQ ID NO: 141 - DNA, 90 bp, Artificial sequence
  Primer LA513
ttggttgggggaaaaagaggcaacaggaaagatcagagggggagggggggggagagtgtcaccttggctaactcgttgtatcatcactgg

SEQ ID NO: 142 - DNA, 29 bp, Artificial sequence
  Primer LA516
ctcgaaacaataagacgacgatggctctg

SEQ ID NO: 143 - DNA, 30 bp, Artificial sequence
  Primer LA514
cactatctggtgcaaacttggcaccggaag

SEQ ID NO: 144 - DNA, 29 bp, Artificial sequence
  Primer LA515
tgtttgtagccactcgtgaacttctctgc

SEQ ID NO: 145 - DNA, 96 bp, Artificial sequence
  Primer LA829
ccaaatttacaatatctcctgaattcttggcttggaatatgggcagtacagcttgtgtgatattgcaccttggctaactcgttgtatcatcactgg

SEQ ID NO: 146 - DNA, 90 bp, Artificial sequence
  Primer LA834
atgtcccaaggtagaaaagctgcagaaagattggctaagaagactgtcctcattacaggtgatctgaaatgaataacaatactgacagta

SEQ ID NO: 147 - DNA, 29 bp, Artificial sequence
  Primer N1257
gatgatgctatttggtgcagagggtgatg

SEQ ID NO: 148 - DNA, 22 bp, Artificial sequence
  Primer LA740
cgataatcctgctgtcattatc

SEQ ID NO: 149 - DNA, 29 bp, Artificial sequence
  Primer LA830
cacggcaaacttagaggcacaatagatag

SEQ ID NO: 150 - DNA, 92 bp, Artificial sequence
  Primer LA850
atgactaagctacactttgacactgctgaaccagtcaagatcacacttccaaatggtttgacataaattaccgtcgctcgtgatttgtttgc

SEQ ID NO: 151 - DNA, 94 bp, Artificial sequence
  Primer LA851
ttacaacttaattctgacagcttttacttcagtgtatgcatggtagacttcttcacccatttccaccttggctaactcgttgtatcatcactgg

SEQ ID NO: 152 - DNA, 24 bp, Artificial sequence
  Primer N1262
cacgtaagggcatgatagaattgg

SEQ ID NO: 153 - DNA, 26 bp, Artificial sequence
  Primer N1263
ggatatagcagttgttgtacactagc

SEQ ID NO: 154 - DNA, 80 bp, Artificial sequence
  Primer LA855
gcacaatatttcaagctataccaagcatacaatcaactatctcatatacaacctggtaaaacctctagtggagtagtaga

SEQ ID NO: 155 - DNA, 83 bp, Artificial sequence
  Primer LA856
gcttatttagaagtgtcaacaacgtatctaccaacgatttgacccttttccacaccttggctaactcgttgtatcatcactgg

SEQ ID NO: 156 - DNA, 25 bp, Artificial sequence
  Primer LA414
ccagagctgatgaggggtatctcga

SEQ ID NO: 157 - DNA, 25 bp, Artificial sequence
  Primer LA749
caagtcttttgtgccttcccgtcgg

SEQ ID NO: 158 - DNA, 25 bp, Artificial sequence
  Primer LA413
ggacataaaatacacaccgagattc

SEQ ID NO: 159 - DNA, 90 bp, Artificial sequence
  Primer LA860
tctcaattattattttctactcataacctcacgcaaaataacacagtcaaatcaatcaaaatgaaagcattagtgtataggggcccaggc

SEQ ID NO: 160 - DNA, 81 bp, Artificial sequence; identical to SEQ ID NO: 121
  Primer LA679

SEQ ID NO: 161 - DNA, 23 bp, Artificial sequence
  Primer LA337
ctcatttgaatcagcttatggtg

SEQ ID NO: 162 - DNA, 26 bp, Artificial sequence
  Primer N1093
tttcaagatgcaaatcaactttgcta

SEQ ID NO: 163 - DNA, 20 bp, Artificial sequence
  Primer LA681
ttattgcttagcgttggtag

SEQ ID NO: 164 - DNA, 3930 bp, Artificial sequence
  pUC19-URA3MCS
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatccggcgcgccgtttaaacggccggccaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggccttgcggccgcttaattaatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 165 - DNA, 12896 bp, Artificial Sequence
  pBP915
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggcgcgccactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccacccgggccccccctcgaggtcgacggtatcgataagcttgatatcgaattcctgcagcccgggggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaaaaagttaaaattgtacgaatagattcaccacttcttaacaaatcaaacccttcattgattttctcgaatggcaatacatgtgtaattaaaggatcaagagcaaacttcttcgccataaagtcggcaacaagttttggaacactatccttgctcttaaaaccgccaaatatagctcccttccatgtacgaccgcttagcaacagcataggattcatcgacaaattttgtgaatcaggaggaacacctacgatcacactgactccatatgcctcttgacagcaggacaacgcagttaccatagtatcaagacggcctataacttcaaaagagaaatcaactccaccgtttgacatttcagtaaggacttcttgtattggtttcttataatcttgagggttaacacattcagtagccccgacctccttagcttttgcaaatttgtccttattgatgtctacacctataatcctcgctgcgcctgcagctttacaccccataataacgcttagtcctactcctcctaaaccgaatactgcacaagtcgaaccctgtgtaacctttgcaactttaactgcggaaccgtaaccggtggaaaatccgcaccctatcaagcaaactttttccagtggtgaagctgcatcgattttagcgacagatatctcgtccaccactgtgtattgggaaaatgtagaagtaccaaggaaatggtgtataggtttccctctgcatgtaaatctgcttgtaccatcctgcatagtacctctaggcatagacaaatcatttttaaggcagaaattaccctcaggatgtttgcagactctacacttaccacattgaggagtgaacagtgggatcactttatcaccaggacgaacagtggtaacaccttcacctatggattcaacgattccggcagcctcgtgtcccgcgattactggcaaaggagtaactagagtgccactcaccacatggtcgtcggatctacagattccggtggcaaccatcttgattctaacctcgtgtgcttttggtggcgctacttctacttcttctatgctaaacggctttttctcttcccacaaaactgccgctttacacttaataactttaccggctgttgacatcctcagctagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgctgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcatgcggatcccccgggctgcaggaattcgatatcaagcttatcgataccgtcgactggccattaatctttcccatattagatttcgccaagccatgaaagttcaagaaaggtctttagacgaattacccttcatttctcaaactggcgtcaagggatcctggtatggttttatcgttttatttctggttcttatagcatcgttttggacttctctgttcccattaggcggttcaggagccagcgcagaatcattctttgaaggatacttatcctttccaattttgattgtctgttacgttggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatcttggctctcttgggctcaaggtgacaaggtcctcgaaaatagggcgcgccccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 166 - DNA, 12497 bp, Artificial Sequence
  pYZ107F-OLE1p
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcagtaaggccctgcaggcctatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctcgcgaactccaaaatgagctatcaaaaacgatagatcgattaggatgactttgaaatgactccgcagtggactggccgttaatttcaagcgtgagtaaaatagtgcatgacaaaagatgagctaggcttttgtaaaaatatcttacgttgtaaaattttagaaatcattatttccttcatatcattttgtcattgaccttcagaagaaaagagccgaccaataatataaataaataaataaaaataatattccattatttctaaacagattcaatactcattaaaaaactatatcaattaatttgaattaacgcggccgcttaaccacagcaaccaggacaacattttttgccagtttcttcaggcttccaaaagtctgttacggctcccctagaagcagacgaaacgatgtgagcatatttaccaaggataccgcgtgaatagagcggtggcaattcaatggtctcttgacgatgttttaactcttcatcggagatatcaaagtgtaattccttagtgtcttggtcaatagtgactatgtctcctgtttgcaggtaggcgattggaccgccatcttgtgcttcaggagcgatatgacccacgacaagaccataagtaccacctgagaagcggccatctgtcagaagggcaactttttcaccttgccctttaccaacaatcattgatgaaagggaaagcatttcaggcataccaggaccgccctttggtcctacaaaacgtacgacaacaacatcaccatcaacaatatcatcattcaagacagcttcaatggcttcttcttcagaattaaagaccttagcaggaccgacatgacgacgcacttttacaccagaaactttggcaacggcaccgtctggagccaagttaccatggagaataatgagcggaccatcttcacgtttaggattttcaagcggcataataaccttttgaccaggtgttaaatcatcaaaagccttcaaattttcagcgactgttttgccagtacaagtgatacggtcaccatgaaggaagccatttttaaggagatatttcataactgctggtacccctccgaccttgtaaaggtcttggaatacatattgaccagaaggtttcaaatcagccaaatgaggaactttttcttggaaagtattgaaatcatcaagtgtcaattccacattagcagcatgggcaatagctaagaggtgaagggttgagttggttgaacctcccagagccatagttacagtaatagcatcttcaaaagcttcacgcgttaaaatgtcagaaggttttaagcccatttcgagcattttgacaacagcgcgaccagcttcttcaatatctgctttcttttctgcggattcagccgggtgagaagatgaacccggaaggctaagtcccaaaacttcaatagctgtcgccattgtgttagcagtatacataccaccgcagcctccaggaccgggacaagcattacattccaaagctttaacttcttctttggtcatatcgccgtggttccaatggccgacaccttcaaagacagagactaaatcgatatctttgccgtctaaattaccaggtgcaattgttccgccgtaagcaaaaatggctgggatatccatgttagccatagcgataacagaaccgggcatgtttttatcacaaccgccaatggctacaaaagcatccgcattatgacctcccatggctgcttcaatagaatctgcaataatatcacgagatgtcaaggagaaacgcattccttgggttcccatggcgattccatcagaaaccgtgattgttccgaactgaactggccaagcaccagcttccttaacaccgactttggctagtttaccaaagtcatgtaagtggatattacaaggtgtgttttcagcccaagttgaaatgacaccgacgataggtttttcaaagtcttcatcttgcataccagttgcacgcaacatagcacgattaggtgatttaaccattgaatcgtaaacagaactacgatttcttaagtctttaagagtttttttgtcagtcatactcacgtgctttgttgtaatgttttagtgctgtttataatatgatcaccacaactatctattactatgatgttctattctacgtaatacaaaatataaacggaaacagaagtaggaaagatggaaatagaacaataaatgaatcaagatctgcccccatatatatatgtatatgctgatttgcaagactcgatgagccaggagccgatgatttgctgcatatattgttaactactattatttccacctttgtgtgccatccccatagccgtaacaatagggataggtgtgtctgagtgagcaagactcgtagaagcacacctggttgggcactagataaggtttgttgagtgttcaacgtccgaaagaaagctgccgactatgcgaagagaaccttaagccgttattacctctgcctgtcacaggcgatgtgatgctaacgaacagcaccagagccaagccaactggggcggtctgcagagaaggctgggatacccgaaatagctcgctcaacagctttttttcttctacggaagcccaccagataagcgcctttgttgggcccgctaaccccgggacatgcccgggctcggagttagtttttgcacggccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 167 - DNA, 4519 bp, Artificial sequence
  pLA54
caccttggctaactcgttgtatcatcactggataacttcgtataatgtatgctatacgaagttatcgaacagagaaactaaatccacattaattgagagttctatctattagaaaatgcaaactccaactaaatgggaaaacagataacctcttttatttttttttaatgtttgatattcgagtctttttcttttgttaggtttatattcatcatttcaatgaataaaagaagcttcttattttggttgcaaagaatgaaaaaaaaggattttttcatacttctaaagcttcaattataaccaaaaattttataaatgaagagaaaaaatctagtagtatcaagttaaacttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatctcgagttttaatgttacttctcttgcagttagggaactataatgtaactcaaaataagattaaacaaactaaaataaaaagaagttatacagaaaaacccatataaaccagtactaatccataataataatacacaaaaaaactatcaaataaaaccagaaaacagattgaatagaaaaattttttcgatctccttttatattcaaaattcgatatatgaaaaagggaactctcagaaaatcaccaaatcaatttaattagatttttcttttccttctagcgttggaaagaaaaatttttctttttttttttagaaatgaaaaatttttgccgtaggaatcaccgtataaaccctgtataaacgctactctgttcacctgtgtaggctatgattgacccagtgttcattgttattgcgagagagcgggagaaaagaaccgatacaagagatccatgctggtatagttgtctgtccaacactttgatgaacttgtaggacgatgatgtgtatttagacgagtacgtgtgtgactattaagtagttatgatagagaggtttgtacggtgtgttctgtgtaattcgattgagaaaatggttatgaatccctagataacttcgtataatgtatgctatacgaagttatctgaacattagaatacgtaatccgcaatgcggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtac

SEQ ID NO: 168 - DNA, 4242 bp, Artificial sequence
  pLA59
aaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccgcaatgcggatccgcattgcggattacgtattctaatgttcagtaccgttcgtataatgtatgctatacgaagttatgcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 169 - DNA, 7523 bp, Artificial sequence
  pLA34
ccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaatgtccaatttactgcccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtccgccaccgcggtggagct

SEQ ID NO: 170 - DNA, 31 bp, Artificial sequence
  Primer LA811
aacgaagcatctgtgcttcattttgtagaac

SEQ ID NO: 171 - DNA, 59 bp, Artificial sequence
  Primer LA817
cgatccacttgtatatttggatgaatttttgaggaattctgaaccagtcctaaaacgag

SEQ ID NO: 172 - DNA, 31 bp, Artificial sequence
  Primer LA812
aacaaagatatgctattgaagtgcaagatgg

SEQ ID NO: 173 - DNA, 33 bp, Artificial sequence
  Primer LA818
ctcaaaaattcatccaaatatacaagtggatcg

SEQ ID NO: 174 - DNA, 6903 bp, Artificial sequence
  pLA71
aaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcgatctgaaatgaataacaatactgacagtagatctgaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaatctagagctgaggatgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctagttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttaggttttgggacgctcgaaggctttaatttggatccgcattgcggattacgtattctaatgttcagtaccgttcgtataatgtatgctatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 175 - DNA, 6924 bp, Artificial sequence
  pLA78
gatccgcattgcggattacgtattctaatgttcagtaccgttcgtataatgtatgctatacgaagttatgcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttccaattaccgtcgctcgtgatttgtttgcaaaaagaacaaaactgaaaaaacccagacacgctcgacttcctgtcttcctattgattgcagcttccaatttcgtcacacaacaaggtcctgtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaagtttaaacatgtataccgtaggacagtacttggtagatagactagaagagattggtatcgataaggttttcggtgtgccaggggattacaatttgacttttctagattacattcaaaatcacgaaggactttcctggcaagggaatactaatgaactaaacgcagcatatgcagcagatggctacgcccgtgaaagaggcgtatcagctcttgttactacattcggagtgggtgaactgtcagccattaacggaacagctggtagttttgcagaacaagtccctgtcatccacatcgtgggttctccaactatgaatgtgcaatccaacaaaaagctggttcatcattccttaggaatgggtaactttcataactttagtgaaatggctaaggaagtcactgccgctacaaccatgcttactgaagagaatgcagcttcagagatcgacagagtattagaaacagccttgttggaaaagaggccagtatacatcaatcttccaattgatatagctcataaagcaatagttaaacctgcaaaagcactacaaacagagaaatcatctggtgagagagaggcacaacttgcagaaatcatactatcacacttagaaaaggccgctcaacctatcgtaatcgccggtcatgagatcgcccgtttccagataagagaaagatttgaaaactggataaaccaaacaaagttgccagtaaccaatttggcatatggcaaaggctctttcaatgaagagaacgaacatttcattggtacctattacccagctttttctgacaaaaacgttctggattacgttgacaatagtgacttcgttttacattttggtgggaaaatcattgacaattctacctcctcattttctcaaggctttaagactgaaaacactttaaccgctgcaaatgacatcattatgctgccagatgggtctacttactctgggatttctcttaacggtcttttggcagagctggaaaaactaaactttacttttgctgatactgctgctaaacaagctgaattagctgttttcgaaccacaggccgaaacaccactaaagcaagacagatttcaccaagctgttatgaactttttgcaagctgatgatgtgttggtcactgagcaggggacatcatctttcggtttgatgttggcacctctgaaaaagggtatgaatttgatcagtcaaacattatggggctccataggatacacattacctgctatgattggttcacaaattgctgccccagaaaggagacacattctatccatcggtgatggatcttttcaactgacagcacaggaaatgtccaccatcttcagagagaaattgacaccagtgatattcattatcaataacgatggctatacagtcgaaagagccatccatggagaggatgagagttacaatgatataccaacttggaacttgcaattagttgctgaaacatttggtggtgatgccgaaactgtcgacactcacaacgttttcacagaaacagacttcgctaatactttagctgctatcgatgctactcctcaaaaagcacatgtcgttgaagttcatatggaacaaatggatatgccagaatcattgagacagattggcttagccttatctaagcaaaactcttaagtttaaactaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacg

SEQ ID NO: 176 - DNA, 22 bp, Artificial sequence
  Primer LA92
gagaagatgcggccagcaaaac

SEQ ID NO: 177 - DNA, 6761 bp, Artificial sequence
  pLA65
gatccgcattgcggattacgtattctaatgttcagtaccgttcgtataatgtatgctatacgaagttatgcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttatgaaagcattagtgtataggggcccaggccagaagttggtggaagagagacagaagccagagcttaaggaacctggtgacgctatagtgaaggtaacaaagactacaatttgcggaaccgatctacacattcttaaaggtgacgttgcgacttgtaaacccggtcgtgtattagggcatgaaggagtgggggttattgaatcagtcggatctggggttactgctttccaaccaggcgatagagttttgatatcatgtatatcgagttgcggaaagtgctcattttgtagaagaggaatgttcagtcactgtacgaccgggggttggattctgggcaacgaaattgatggtacccaagcagagtacgtaagagtaccacatgctgacacatccctttatcgtattccggcaggtgcggatgaagaggccttagtcatgttatcagatattctaccaacgggttttgagtgcggagtcctaaacggcaaagtcgcacctggttcttcggtggctatagtaggtgctggtcccgttggtttggccgccttactgacagcacaattctactccccagctgaaatcataatgatcgatcttgatgataacaggctgggattagccaaacaatttggtgccaccagaacagtaaactccacgggtggtaacgccgcagccgaagtgaaagctcttactgaaggcttaggtgttgatactgcgattgaagcagttgggatacctgctacatttgaattgtgtcagaatatcgtagctcccggtggaactatcgctaatgtcggcgttcacggtagcaaagttgatttgcatcttgaaagtttatggtcccataatgtcacgattactacaaggttggttgacacggctaccaccccgatgttactgaaaactgttcaaagtcacaagctagatccatctagattgataacacatagattcagcctggaccagatcttggacgcatatgaaacttttggccaagctgcgtctactcaagcactaaaagtcatcatttcgatggaggcttgattaattaagagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtg

SEQ ID NO: 178 - DNA, 9612 bp, Artificial sequence
  pLH702
aaacagtatggaagaatgtaagatggctaagatttactaccaagaagactgtaacttgtccttgttggatggtaagactatcgccgttatcggttacggttctcaaggtcacgctcatgccctgaatgctaaggaatccggttgtaacgttatcattggtttatacgaaggtgctaaggattggaaaagagctgaagaacaaggtttcgaagtctacaccgctgctgaagctgctaagaaggctgacatcattatgatcttgatcaacgatgaaaagcaggctaccatgtacaaaaacgacatcgaaccaaacttggaagccggtaacatgttgatgttcgctcacggtttcaacatccatttcggttgtattgttccaccaaaggacgttgatgtcactatgatcgctccaaagggtccaggtcacaccgttagatccgaatacgaagaaggtaaaggtgtcccatgcttggttgctgtcgaacaagacgctactggcaaggctttggatatggctttggcctacgctttagccatcggtggtgctagagccggtgtcttggaaactaccttcagaaccgaaactgaaaccgacttgttcggtgaacaagctgttttatgtggtggtgtctgcgctttgatgcaggccggttttgaaaccttggttgaagccggttacgacccaagaaacgcttacttcgaatgtatccacgaaatgaagttgatcgttgacttgatctaccaatctggtttctccggtatgcgttactctatctccaacactgctgaatacggtgactacattaccggtccaaagatcattactgaagataccaagaaggctatgaagaagattttgtctgacattcaagatggtacctttgccaaggacttcttggttgacatgtctgatgctggttcccaggtccacttcaaggctatgagaaagttggcctccgaacacccagctgaagttgtcggtgaagaaattagatccttgtactcctggtccgacgaagacaagttgattaacaactgatattttcctctggccctgcaggcctatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatctcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagtcaccggtggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgttt

SEQ ID NO: 179 - DNA, 7938 bp, Artificial sequence
  pYZ067DkivDDhADH
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtggccggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaacacgtgagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgaggatcccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 180 - DNA, 500 bp, Saccharomyces cerevisiae
cacttctacatctactgaaatgaccaccgtcaccggtaccaacggcgttccaactgacgaaaccgtcattgtcatcagaactccaacaactgctagcaccatcataactacaactgagccatggaacagcacttttacctctacttctaccgaattgaccacagtcactggcaccaatggtgtacgaactgacgaaaccatcattgtaatcagaacaccaacaacagccactactgccataactacaactgagccatggaacagcacttttacctctacttctaccgaattgaccacagtcaccggtaccaatggtttgccaactgatgagaccatcattgtcatcagaacaccaacaacagccactactgccatgactacaactcagccatggaacgacacttttacctctacttctaccgaattgaccacagtcaccggtaccaatggtttgccaactgatgagaccatcattgtcatcagaacaccaacaacagcca

SEQ ID NO: 181 - DNA, 500 bp, Saccharomyces cerevisiae
atactggagtactgatttatttggtttctatactaccccaacaaacgtaaccctagaaatgacaggttattttttaccaccacagacgggttcttacacattcaagtttgctacagttgacgactctgcaattctatcagtcggtggtagcattgcgttcgaatgttgtgcacaagaacaacctcccatcacgtcgactaacttcaccatcaatggtatcaagccatggaatggaagtccccctgataatattacagggactgtctacatgtatgctggtttctattatccaatgaagattgtttactcaaatgccgttgcctggggtacacttccaattagtgtgacactaccagatggcactaccgttagtgatgactttgaagggtacgtatatacttttgacaacaatctaagccagccaaactgtaccattccagacccttcaaattatactgtcagtactaccataactacaacggaaccatggaccggtactt

SEQ ID NO: 182 - DNA, 500 bp, Saccharomyces cerevisiae
ctactgccatgactacaactcagccatggaacgacacttttacctctacttctaccgaattgaccacagtcaccggtaccaatggtttgccaactgatgagaccatcattgtcatcagaacaccaacaacagccactactgccatgactacaactcagccatggaacgacacttttacctctacatccactgaaatcaccaccgtcaccggtaccaatggtttgccaactgatgagaccatcattgtcatcagaacaccaacaacagccactactgccatgactacaactcagccatggaacgacacttttacctctacatccactgaaatgaccaccgtcaccggtaccaacggtttgccaactgatgaaaccatcattgtcatcagaacaccaacaacagccactactgccataactacaactgagccatggaacagcacttttacctctacatccactgaaatgaccaccgtcaccggtaccaacggtttgccaact

SEQ ID NO: 183 - DNA, 23 bp, Artificial sequence
  Primer AK09-1_MAT
agtcacatcaagatcgtttatgg

SEQ ID NO: 184 - DNA, 23 bp, Artificial sequence
  Primer AK09-2_HML
gcacggaatatgggactacttcg

SEQ ID NO: 185 - DNA, 23 bp, Artificial sequence
  Primer AK09-3_HMR
actccacttcaagtaagagtttg

SEQ ID NO: 186 - DNA, 80 bp, Artificial sequence
  Primer 315
cttcgaagaatatactaaaaaatgagcaggcaagataaacgaaggcaaaggcattgcggattacgtattctaatgttcag

SEQ ID NO: 187 - DNA, 81 bp, Artificial sequence
  Primer 316
tatacacatgtatatatatcgtatgctgcagctttaaataatcggtgtcacaccttggctaactcgttgtatcatcactgg

SEQ ID NO: 188 - DNA, 22 bp, Artificial sequence
  Primer 92
gagaagatgcggccagcaaaac

SEQ ID NO: 189 - DNA, 25 bp, Artificial sequence
  Primer 346
ggaataccacttgccacctatcacc

SEQ ID NO: 190 - DNA, 22 bp, Artificial sequence
  Primer oBP440
tacgtacggaccaatcgaagtg

SEQ ID NO: 191 - DNA, 49 bp, Artificial sequence
  Primer oBP441
aattcgtttgagtacactactaatggctttgttggcaatatgtttttgc

SEQ ID NO: 192 - DNA, 49 bp, Artificial sequence
  Primer oBP442
atatagcaaaaacatattgccaacaaagccattagtagtgtactcaaac

SEQ ID NO: 193 - DNA, 49 bp, Artificial sequence
  Primer oBP443
tatggaccctgaaaccacagccacattcttgttatttataaaaagacac

SEQ ID NO: 194 - DNA, 49 bp, Artificial sequence
  Primer oBP444
ctcccgtgtctttttataaataacaagaatgtggctgtggtttcagggt

SEQ ID NO: 195 - DNA, 49 bp, Artificial sequence
  Primer oBP445
taccgtaggcgtccttaggaaagatagaaggccatgaagctttttcttt

SEQ ID NO: 196 - DNA, 49 bp, Artificial sequence
  Primer oBP446
attggaaagaaaaagcttcatggccttctatctttcctaaggacgccta

SEQ ID NO: 197 - DNA, 21 bp, Artificial sequence
  Primer oBP447
ttattgtttggcatttgtagc

SEQ ID NO: 198 - DNA, 22 bp, Artificial sequence
  Primer oBP448
ccaagcatctcataaacctatg

SEQ ID NO: 199 - DNA, 22 bp, Artificial sequence
  Primer oBP449
tgtgcagatgcagatgtgagac

SEQ ID NO: 200 - DNA, 17 bp, Artificial sequence
  Primer oBP554
agttattgataccgtac

SEQ ID NO: 201 - DNA, 19 bp, Artificial sequence
  Primer oBP555
cgagataccgtaggcgtcc

SEQ ID NO: 202 - DNA, 24 bp, Artificial sequence
  Primer oBP513
ttatgtatgctcttctgacttttc

SEQ ID NO: 203 - DNA, 49 bp, Artificial sequence
  Primer oBP515
aataattagagattaaatcgctcattttttgccagtttcttcaggcttc

SEQ ID NO: 204 - DNA, 49 bp, Artificial sequence
  Primer oBP516
agcctgaagaaactggcaaaaaatgagcgatttaatctctaattattag

SEQ ID NO: 205 - DNA, 49 bp, Artificial sequence
  Primer oBP517
tatggaccctgaaaccacagccacatttttcaatcattggagcaatcat

SEQ ID NO: 206 - DNA, 49 bp, Artificial sequence
  Primer oBP518
taaaatgattgctccaatgattgaaaaatgtggctgtggtttcagggtc

SEQ ID NO: 207 - DNA, 49 bp, Artificial sequence
  Primer oBP519
accgtaggtgttgtttgggaaagtggaaggccatgaagctttttctttc

SEQ ID NO: 208 - DNA, 49 bp, Artificial sequence
  Primer oBP520
ttggaaagaaaaagcttcatggccttccactttcccaaacaacacctac

SEQ ID NO: 209 - DNA, 23 bp, Artificial sequence
  Primer oBP521
ttattgcttagcgttggtagcag

SEQ ID NO: 210 - DNA, 16 bp, Artificial sequence
  Primer oBP550
gtcattgacaccatct

SEQ ID NO: 211 - DNA, 19 bp, Artificial sequence
  Primer oBP551
agagataccgtaggtgttg

SEQ ID NO: 212 - DNA, 28 bp, Artificial sequence
  Primer ilvDSm(1354F)
ggaccaaagggcggtcctggtatgcctg

SEQ ID NO: 213 - DNA, 22 bp, Artificial sequence
  Primer oBP512
aaagttggcatagcggaaactt

SEQ ID NO: 214 - DNA, 26 bp, Artificial sequence
  Primer ilvDSm(788R)
gcttcacgcgttaaaatgtcagaagg

SEQ ID NO: 215 - DNA, 23 bp, Artificial sequence
  Primer MAT1
agtcacatcaagatcgtttatgg

SEQ ID NO: 216 - DNA, 23 bp, Artificial sequence
  Primer MAT2
gcacggaatatgggcatacttcg

SEQ ID NO: 217 - DNA, 23 bp, Artificial sequence
  Primer MAT3
actccacttcaagtaagagtttg

SEQ ID NO: 218 - DNA, 22 bp, Artificial sequence
  Primer oBP448
ccaagcatctcataaacctatg

SEQ ID NO: 219 - DNA, 22 bp, Artificial sequence
  Primer oBP449
tgtgcagatgcagatgtgagac

SEQ ID NO: 220 - DNA, 29 bp, Artificial sequence
  Primer T-A(PDC5)
ctgtcgctaacacctgtatggttgcaacc

SEQ ID NO: 221 - DNA, 48 bp, Artificial sequence
  Primer B-A(kivD)
gatagtcacctactgtatacattttgttcttcttgttattgtattgtg

SEQ ID NO: 222 - DNA, 57 bp, Artificial sequence
  Primer T-kivD(A)
acacaatacaataacaagaagaacaaaatgtatacagtaggtgactatctgttggac

SEQ ID NO: 223 - DNA, 56 bp, Artificial sequence
  Primer B-kivD(B)
tcaggcagcgcctgcgttcgagtcagctcttgttttgttctgcaaataacttaccc

SEQ ID NO: 224 - DNA, 47 bp, Artificial sequence
  Primer T-B(kivD)
atttgcagaacaaaacaagagctgactcgaacgcaggcgctgcctga

SEQ ID NO: 225 - DNA, 49 bp, Artificial sequence
  Primer oBP546
agcgtatacatctgttgggaaagtagaaggccatgaagctttttctttc

SEQ ID NO: 226 - DNA, 49 bp, Artificial sequence
  Primer oBP547
ttggaaagaaaaagcttcatggccttctactttcccaacagatgtatac

SEQ ID NO: 227 - DNA, 22 bp, Artificial sequence
  Primer oBP539
ttattgtttagcgttagtagcg

SEQ ID NO: 228 - DNA, 21 bp, Artificial sequence
  Primer oBP540
taggcataatcaccgaagaag

SEQ ID NO: 229 - DNA, 29 bp, Artificial sequence
  Primer kivD(652R)
ctgagtaacagtcttctctaggccgaacg

SEQ ID NO: 230 - DNA, 17 bp, Artificial sequence
  Primer oBP552
agttgttagaactgttg

SEQ ID NO: 231 - DNA, 19 bp, Artificial sequence
  Primer oBP553
gacgatagcgtatacatct

SEQ ID NO: 232 - DNA, 29 bp, Artificial sequence
  Primer kivD(602F)
caagagattctgaacaaaatacaggaaag

SEQ ID NO: 233 - DNA, 27 bp, Artificial sequence
  Primer kivD(1250F)
ccccgcagctctaggcagccaaattgc

SEQ ID NO: 234 - DNA, 32 bp, Artificial sequence
  Primer JZ067
cgtcgtgaaggcagtttagttctcggacttgc

SEQ ID NO: 235 - DNA, 61 bp, Artificial sequence
  Primer JZ088
ctttttgcaaacaaatcacgagcgacggtaattttttggccaaatgccacagccgatctgc

SEQ ID NO: 236 - DNA, 61 bp, Artificial sequence
  Primer JZ087
gcagatcggctgtggcatttggccaaaaaattaccgtcgctcgtgatttgtttgcaaaaag

SEQ ID NO: 237 - DNA, 55 bp, Artificial sequence
  Primer JZ068
aataattcgtttgagtacactactaatggcaccacaggtgttgtcctctgaggac

SEQ ID NO: 238 - DNA, 55 bp, Artificial sequence
  Primer JZ069
gtcctcagaggacaacacctgtggtgccattagtagtgtactcaaacgaattatt

SEQ ID NO: 239 - DNA, 54 bp, Artificial sequence
  Primer JZ070
ggaccctgaaaccacagccacattaacttgttatttataaaaagacacgggagg

SEQ ID NO: 240 - DNA, 54 bp, Artificial sequence
  Primer JZ071
cctcccgtgtctttttataaataacaagttaatgtggctgtggtttcagggtcc

SEQ ID NO: 241 - DNA, 54 bp, Artificial sequence
  Primer JZ072
gtgaataaggtgtgaactctataacaaaggccatgaagctttttctttccaatt

SEQ ID NO: 242 - DNA, 54 bp, Artificial sequence
  Primer JZ073
aattggaaagaaaaagcttcatggcctttgttatagagttcacaccttattcac

SEQ ID NO: 243 - DNA, 31 bp, Artificial sequence
  Primer JZ074
tttgttggcaatatgtttttgctatattacg

SEQ ID NO: 244 - DNA, 32 bp, Artificial sequence
  Primer JZ061
gagagctgctcaacgcggaatggagataacgg

SEQ ID NO: 245 - DNA, 26 bp, Artificial sequence
  Primer JZ060
ccttcactatagcgtcaccaggttcc

SEQ ID NO: 246 - DNA, 32 bp, Artificial sequence
  Primer JZ062
ggtaaataaatgtgcagatgcagatgtgagac

SEQ ID NO: 247 - DNA, 26 bp, Artificial sequence
  Primer 643R
cggctgcggcgttaccacccgtggag

SEQ ID NO: 248 - DNA, 28 bp, Artificial sequence
  Primer T-HIS3(up300)
ttggtgagcgctaggagtcactgccagg

SEQ ID NO: 249 - DNA, 28 bp, Artificial sequence
  Primer B-HIS3(down273)
cggaataccacttgccacctatcaccac

SEQ ID NO: 250 - DNA, 32 bp, Artificial sequence
  Primer JZ151
aagattctgtccagaaacaacatcaacatcgc

SEQ ID NO: 251 - DNA, 62 bp, Artificial sequence
  Primer JZ317
gttgaaggaattcgtatacgtattacaaatatatcaaaatacgttctcaatgttctatttcc

SEQ ID NO: 252 - DNA, 62 bp, Artificial sequence
  Primer JZ316
ggaaatagaacattgagaacgtattttgatatatttgtaatacgtatacgaattccttcaac

SEQ ID NO: 253 - DNA, 61 bp, Artificial sequence
  Primer JZ313
gtatacagatttacttagtttagctaggtccgcaaattaaagccttcgagcgtcccaaaac

SEQ ID NO: 254 - DNA, 61 bp, Artificial sequence
  Primer JZ312
gttttgggacgctcgaaggctttaatttgcggacctagctaaactaagtaaatctgtatac

SEQ ID NO: 255 - DNA, 58 bp, Artificial sequence
  Primer JZ157
ttatggaccctgaaaccacagccacattaaagaggcttgactttattgtaatctgaga

SEQ ID NO: 256 - DNA, 58 bp, Artificial sequence
  Primer JZ156
tctcagattacaataaagtcaagcctctttaatgtggctgtggtttcagggtccataa

SEQ ID NO: 257 - DNA, 54 bp, Artificial sequence
  Primer JZ159
gtcactgccaagagcctttccggcataaggccatgaagctttttctttccaatt

SEQ ID NO: 258 - DNA, 54 bp, Artificial sequence
  Primer JZ158
aattggaaagaaaaagcttcatggccttatgccggaaaggctcttggcagtgac

SEQ ID NO: 259 - DNA, 33 bp, Artificial sequence
  Primer JZ160
ttatccacggaagatatgatgaggtgacgcttg

SEQ ID NO: 260 - DNA, 30 bp, Artificial sequence; identical to SEQ ID NO: 87
  Primer URA3F

SEQ ID NO: 261 - DNA, 35 bp, Artificial sequence
  Primer JZ161
aacatatgtttgagatccagctgtttcgagtgacg

SEQ ID NO: 262 - DNA, 36 bp, Artificial sequence
  Primer URA3R
ctgtgctccttccttcgttcttccttctgctcggag

SEQ ID NO: 263 - DNA, 30 bp, Artificial sequence
  Primer JZ320
cgtaaacctgcattaaggtaagattatatc

SEQ ID NO: 264 - DNA, 34 bp, Artificial sequence
  Primer JZ150
gaacgaactagagaccaccctggcccataccaag

SEQ ID NO: 265 - DNA, 32 bp, Artificial sequence
  266
cgatatcggttcgcacgccatttggatgtcac

SEQ ID NO: 266 - DNA, 44 bp, Artificial sequence
  Primer B-A(kivDLg)
ctgtcctacggtatacattttgttcttcttgttattgtattgtg

SEQ ID NO: 267 - DNA, 52 bp, Artificial sequence
  Primer T-kivDLg(A)
acacaatacaataacaagaagaacaaaatgtataccgtaggacagtacttgg

SEQ ID NO: 268 - DNA, 52 bp, Artificial sequence
  Primer B-kivDLg(B)
tcaggcagcgcctgcgttcgagttaagagttttgcttagataaggctaagcc

SEQ ID NO: 269 - DNA, 43 bp, Artificial sequence
  Primer T-B(kivDLg)
ttatctaagcaaaactcttaactcgaacgcaggcgctgcctga

SEQ ID NO: 270 - DNA, 49 bp, Artificial sequence; identical to SEQ ID NO: 225
  Primer oBP546

SEQ ID NO: 271 - DNA, 49 bp, Artificial sequence; identical to SEQ ID NO: 226
  Primer oBP547

SEQ ID NO: 272 - DNA, 22 bp, Artificial sequence
  Primer oBP539
ttattgtttagcgttagtagcg

SEQ ID NO: 273 - DNA, 31 bp, Artificial sequence
  Primer kivDLg(569R)
gtgtgatagtatgatttctgcaagttgtgcc

SEQ ID NO: 274 - DNA, 26 bp, Artificial sequence
  Primer kivDLg(530F)
gctcataaagcaatagttaaacctgc

SEQ ID NO: 275 - DNA, 29 bp, Artificial sequence
  Primer kivDLg(1162F)
ggggacatcatctttcggtttgatgttgg

SEQ ID NO: 276 - DNA, 7821 bp, Artificial sequence
  pWZ009
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgccgaaatgcatgcaagtaacctattcaaagtaatatctcatacatgtttcatgagggtaacaacatgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaagtttaaacagtatggaagaatgtaagatggctaagatttactaccaagaagactgtaacttgtccttgttggatggtaagactatcgccgttatcggttacggttctcaaggtcacgctcatgccctgaatgctaaggaatccggttgtaacgttatcattggtttatacgaaggtgctaaggattggaaaagagctgaagaacaaggtttcgaagtctacaccgctgctgaagctgctaagaaggctgacatcattatgatcttgatcaacgatgaaaagcaggctaccatgtacaaaaacgacatcgaaccaaacttggaagccggtaacatgttgatgttcgctcacggtttcaacatccatttcggttgtattgttccaccaaaggacgttgatgtcactatgatcgctccaaagggtccaggtcacaccgttagatccgaatacgaagaaggtaaaggtgtcccatgcttggttgctgtcgaacaagacgctactggcaaggctttggatatggctttggcctacgctttagccatcggtggtgctagagccggtgtcttggaaactaccttcagaaccgaaactgaaaccgacttgttcggtgaacaagctgttttatgtggtggtgtctgcgctttgatgcaggccggttttgaaaccttggttgaagccggttacgacccaagaaacgcttacttcgaatgtatccacgaaatgaagttgatcgttgacttgatctaccaatctggtttctccggtatgcgttactctatctccaacactgctgaatacggtgactacattaccggtccaaagatcattactgaagataccaagaaggctatgaagaagattttgtctgacattcaagatggtacctttgccaaggacttcttggttgacatgtctgatgctggttcccaggtccacttcaaggctatgagaaagttggcctccgaacacccagctgaagttgtcggtgaagaaattagatccttgtactcctggtccgacgaagacaagttgattaacaacggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatgtgtgcgtgtatgtgtacacctgtatttaatttccttactcgcgggtttttcttttttctcaattcttggcttcctctttctcgagcggaccggatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgccgaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 277 - DNA, 8148 bp, Artificial sequence
  pWZ001
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtggccggcatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaacacgtgagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgaggatcccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 278 - DNA, 4236 bp, Artificial sequence
  pLA33
aaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccgcattgcggattacgtattctaatgttcagataacttcgtatagcatacattatacgaagttatgcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtatagcatacattatacgaagttatccagtgatgatacaacgagttagccaaggtgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 279 - DNA, 5231 bp, Artificial sequence
  pUC19-URA3-sadB-PDC5fragmentB
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatccggcgcgccatgaaagctctggtttatcacggtgaccacaagatctcgcttgaagacaagcccaagcccacccttcaaaagcccacggatgtagtagtacgggttttgaagaccacgatctgcggcacggatctcggcatctacaaaggcaagaatccagaggtcgccgacgggcgcatcctgggccatgaaggggtaggcgtcatcgaggaagtgggcgagagtgtcacgcagttcaagaaaggcgacaaggtcctgatttcctgcgtcacttcttgcggctcgtgcgactactgcaagaagcagctttactcccattgccgcgacggcgggtggatcctgggttacatgatcgatggcgtgcaggccgaatacgtccgcatcccgcatgccgacaacagcctctacaagatcccccagacaattgacgacgaaatcgccgtcctgctgagcgacatcctgcccaccggccacgaaatcggcgtccagtatgggaatgtccagccgggcgatgcggtggctattgtcggcgcgggccccgtcggcatgtccgtactgttgaccgcccagttctactccccctcgaccatcatcgtgatcgacatggacgagaatcgcctccagctcgccaaggagctcggggcaacgcacaccatcaactccggcacggagaacgttgtcgaagccgtgcataggattgcggcagagggagtcgatgttgcgatcgaggcggtgggcataccggcgacttgggacatctgccaggagatcgtcaagcccggcgcgcacatcgccaacgtcggcgtgcatggcgtcaaggttgacttcgagattcagaagctctggatcaagaacctgacgatcaccacgggactggtgaacacgaacacgacgcccatgctgatgaaggtcgcctcgaccgacaagcttccgttgaagaagatgattacccatcgcttcgagctggccgagatcgagcacgcctatcaggtattcctcaatggcgccaaggagaaggcgatgaagatcatcctctcgaacgcaggcgctgcctgagctaattaacataaaactcatgattcaacgtttgtgtatttttttacttttgaaggttatagatgtttaggtaaataattggcatagatatagttttagtataataaatttctgatttggtttaaaatatcaactattttttttcacatatgttcttgtaattacttttctgtcctgtcttccaggttaaagattagcttctaatattttaggtggtttattatttaattttatgctgattaatttatttacttgtttaaacggccggccaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggccttgcggccgcttaattaatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 280 - DNA, 12812 bp, Artificial sequence
  pWS360
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggcgcgccactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccacccgggccccccctcgaggtcgacggtatcgataagcttgatatcgaattcctgcagcccgggggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaagcctccatcgaaatgatgacttttagtgcttgagtagacgcagcttggccaaaagtttcatatgcgtccaagatctggtccaggctgaatctatgtgttatcaatctagatggatctagcttgtgactttgaacagttttcagtaacatcggggtggtagccgtgtcaaccaaccttgtagtaatcgtgacattatgggaccataaactttcaagatgcaaatcaactttgctaccgtgaacgccgacattagcgatagttccaccgggagctacgatattctgacacaattcaaatgtagcaggtatcccaactgcttcaatcgcagtatcaacacctaagccttcagtaagagctttcacttcggctgcggcgttaccacccgtggagtttactgttctggtggcaccaaattgtttggctaatcccagcctgttatcatcaagatcgatcattatgatttcagctggggagtagaattgtgctgtcagtaaggcggccaaaccaacgggaccagcacctactatagccaccgaagaaccaggtgcgactttgccgtttaggactccgcactcaaaacccgttggtagaatatctgataacatgactaaggcctcttcatccgcacctgccggaatacgataaagggatgtgtcagcatgtggtactcttacgtactctgcttgggtaccatcaatttcgttgcccagaatccaacccccggtcgtacagtgactgaacattcctcttctacaaaatgagcactttccgcaactcgatatacatgatatcaaaactctatcgcctggttggaaagcagtaaccccagatccgactgattcaataacccccactccttcatgccctaatacacgaccgggtttacaagtcgcaacgtcacctttaagaatgtgtagatcggttccgcaaattgtagtctttgttaccttcactatagcgtcaccaggttccttaagctctggcttctgtctctcttccaccaacttctggcctgggcccctatacactaatgctttcatcctcagctagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgctgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcatgcggatcccccgggctgcaggaattcgatatcaagcttatcgataccgtcgactggccattaatctttcccatattagatttcgccaagccatgaaagttcaagaaaggtctttagacgaattacccttcatttctcaaactggcgtcaagggatcctggtatggttttatcgttttatttctggttcttatagcatcgttttggacttctctgttcccattaggcggttcaggagccagcgcagaatcattctttgaaggatacttatcctttccaattttgattgtctgttacgttggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatcttggctctcttgggctcaaggtgacaaggtcctcgaaaatagggcgcgccccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 281 - DNA, 12359 bp, Artificial sequence
  pYZ152
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcagtaaggccctgcaggcctatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctcgcgaccgcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgattaattaactagagagctttcgttttcatgagttccccgaattctttcggaagcttgtcacttgctaaattaatgttatcactgtagtcaaccgggacatcgatgatgacaggaccttcagcgttcatgccttgacgcagaacatctgccagctggtctggtgattctacgcgcaagccagttgctccgaagctttccgcatatttcacgatatcgatatttccgaaatcgaccgcagatgtacggttatattttttcaattgctggaatgcaaccatgtcatatgtgctgtcgttccatacaatgtgtacaattggtgcttttagtcgaactgctgtctctaattccattgctgagaataagaaaccgccgtcaccagagacagaaaccactttttctcccggtttcaccaatgaagcgccgattgcccaaggaagcgcaacgccgagtgtttgcataccgttactgatcattaatgttaacggctcgtagctgcggaaataacgtgacatccaaatggcgtgcgaaccgatatcgcaagttactgtaacatgatcatcgactgcattacgcaactctttaacgatttcaagagggtgcgctctgtctgatttccaatctgcaggcacctgctcaccttcatgcatatattgttttaaatcagaaaggattttctgctcacgctctgcaaattccactttcacagcatcgtgttcgatatgattgatcgtggacggaatgtcaccgatcaattcaagatcaggctggtaagcatgatcaatgtcagcgataatctcgtctaaatggataattgtccggtctccattgatattccagaatttcggatcatattcaatcgggtcatagccgatcgtcagaacaacatctgcctgctctagcagtaaatcgccaggctggttgcggaacaaaccgatacggccaaaatattgatcctctaaatctctagaaagggtaccggcagcttgatatgtttcaacaaatggaagctgaacctttttcaaaagcttgcgaaccgctttaattgcttccggtcttccgcctttcatgccgaccaaaacgacaggaagttttgctgtttggatttttgctatggccgcactgattgcatcatctgctgcaggaccgagttttggcgctgcaacagcacgcacgtttttcgtatttgtgacttcattcacaacatcttgcggaaagctcacaaaagcggccccagcctgccctgctgacgctatcctaaatgcatttgtaacagcttccggtatattttttacatcttgaacttctacactgtattttgtaatcggctggaatagcgccgcattatccaaagattgatgtgtccgttttaaacgatctgcacggatcacgtttccagcaagcgcaacgacagggtctccttcagtgttcgctgtcagcaggcctgttgccaagttagaggcacccggtcctgatgtgactaacacgactcccggttttccagttaaacggccgactgcttgggccatgaatgctgcgttttgttcgtgccgggcaacgataatttcaggtcctttatcttgtaaagcgtcaaataccgcatcaatttttgcacctggaatgccaaatacatgtgtgacaccttgctccactaagcaatcaacaacaagctccgcccctctgtttttcacaagggatttttgttcttttgttgcttttgtcaacatcctcacgtgtttgttcttcttgttattgtattgtgttgttctctttgagattgattatgtgaaataagtgtaataagaaagagaggaaaggacttactacagtatattgatcgagaatggcagctcttatatacaagttcttttagcaagcgccgctgcattattcaagtctcatcatatgaaatttctttcgagagattgtcataatcaaaaaattgcataatgcatttcttgcaacacattttctgatataatcttaccttaatgcaggtttacgtattagtttttctaaaagaaacgcgacctttggatatggaggcttttcccataaacgcatgtagtatgcatttacgatgagaatcaatttttttccaaggggcgcaaaacgcataaacgcataaagtatgcatcagaaggattctcacctggttgcaaccatacaggtgttagcgacagtaatagaaaaaaaattaaaataatggtgttattgttatttgctttatttccttggcctttgttgaaggaattcgtatacgtattacaaatagccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 282 - DNA, 8289 bp, Artificial sequence
  pBP1719
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaggcgcgccaattaccgtcgctcgtgatttgtttgcaaaaagaacaaaactgaaaaaacccagacacgctcgacttcctgtcttcctattgattgcagcttccaatttcgtcacacaacaaggtcctgtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaagtttaaacatgtataccgtaggacagtacttggtagatagactagaagagattggtatcgataaggttttcggtgtgccaggggattacaatttgacttttctagattacattcaaaatcacgaaggactttcctggcaagggaatactaatgaactaaacgcagcatatgcagcagatggctacgcccgtgaaagaggcgtatcagctcttgttactacattcggagtgggtgaactgtcagccattaacggaacagctggtagttttgcagaacaagtccctgtcatccacatcgtgggttctccaactatgaatgtgcaatccaacaaaaagctggttcatcattccttaggaatgggtaactttcataactttagtgaaatggctaaggaagtcactgccgctacaaccatgcttactgaagagaatgcagcttcagagatcgacagagtattagaaacagccttgttggaaaagaggccagtatacatcaatcttccaattgatatagctcataaagcaatagttaaacctgcaaaagcactacaaacagagaaatcatctggtgagagagaggcacaacttgcagaaatcatactatcacacttagaaaaggccgctcaacctatcgtaatcgccggtcatgagatcgcccgtttccagataagagaaagatttgaaaactggataaaccaaacaaagttgccagtaaccaatttggcatatggcaaaggctctttcaatgaagagaacgaacatttcattggtacctattacccagctttttctgacaaaaacgttctggattacgttgacaatagtgacttcgttttacattttggtgggaaaatcattgacaattctacctcctcattttctcaaggctttaagactgaaaacactttaaccgctgcaaatgacatcattatgctgccagatgggtctacttactctgggatttctcttaacggtcttttggcagagctggaaaaactaaactttacttttgctgatactgctgctaaacaagctgaattagctgttttcgaaccacaggccgaaacaccactaaagcaagacagatttcaccaagctgttatgaactttttgcaagctgatgatgtgttggtcactgagcaggggacatcatctttcggtttgatgttggcacctctgaaaaagggtatgaatttgatcagtcaaacattatggggctccataggatacacattacctgctatgattggttcacaaattgctgccccagaaaggagacacattctatccatcggtgatggatcttttcaactgacagcacaggaaatgtccaccatcttcagagagaaattgacaccagtgatattcattatcaataacgatggctatacagtcgaaagagccatccatggagaggatgagagttacaatgatataccaacttggaacttgcaattagttgctgaaacatttggtggtgatgccgaaactgtcgacactcacaacgttttcacagaaacagacttcgctaatactttagctgctatcgatgctactcctcaaaaagcacatgtcgttgaagttcatatggaacaaatggatatgccagaatcattgagacagattggcttagccttatctaagcaaaactcttaagtttaaactaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacggatccacagcctagccttcagttgggctctatcttcatcgtcattcattgcatctactagccccttacctgagcttcaagacgttatatcgcttttatgtatcatgatcttatcttgagatatgaatacataaatatatttactcaagtgtatacgtgcatgctttttttggccggccaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggccttgcggccgcgtgcctcatctatatttctgaaatcgaaatcacattttattggtcaacccttgtggggatctataggatacactttccccgcagctctaggcagccaaattgcagataaagaatctagacatttattgtttatcggagatggatcattgcaactgactgtccaagaattaggactagccattagagagaagataaacccaatctgctttatcattaataacgatggttacacggttgagagggaaattcatggtccgaaccagagttataatgacattcctatgtggaattactcaaaactgccagaaagtttcggggcaacggaagacagagttgtgtccaaaattgtgagaacagaaaatgaattcgtatccgtgatgaaagaagctcaagcagatccaaataggatgtattggatagaacttattctagcaaaggagggtgcacctaaagttttgaaaaagatgggtaagttatttgcagaacaaaacaagagctgattaattaagtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 283 - DNA, 5231 bp, Artificial sequence; identical to SEQ ID NO: 279
  pBP904

SEQ ID NO: 284 - DNA, 10528 bp, Artificial sequence
  pNZ001
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgccgaaatgcatgcaagtaacctattcaaagtaatatctcatacatgtttcatgagggtaacaacatgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaagtttaaacagtatggaagaatgtaagatggctaagatttactaccaagaagactgtaacttgtccttgttggatggtaagactatcgccgttatcggttacggttctcaaggtcacgctcatgccctgaatgctaaggaatccggttgtaacgttatcattggtttatacgaaggtgctaaggattggaaaagagctgaagaacaaggtttcgaagtctacaccgctgctgaagctgctaagaaggctgacatcattatgatcttgatcaacgatgaaaagcaggctaccatgtacaaaaacgacatcgaaccaaacttggaagccggtaacatgttgatgttcgctcacggtttcaacatccatttcggttgtattgttccaccaaaggacgttgatgtcactatgatcgctccaaagggtccaggtcacaccgttagatccgaatacgaagaaggtaaaggtgtcccatgcttggttgctgtcgaacaagacgctactggcaaggctttggatatggctttggcctacgctttagccatcggtggtgctagagccggtgtcttggaaactaccttcagaaccgaaactgaaaccgacttgttcggtgaacaagctgttttatgtggtggtgtctgcgctttgatgcaggccggttttgaaaccttggttgaagccggttacgacccaagaaacgcttacttcgaatgtatccacgaaatgaagttgatcgttgacttgatctaccaatctggtttctccggtatgcgttactctatctccaacactgctgaatacggtgactacattaccggtccaaagatcattactgaagataccaagaaggctatgaagaagattttgtctgacattcaagatggtacctttgccaaggacttcttggttgacatgtctgatgctggttcccaggtccacttcaaggctatgagaaagttggcctccgaacacccagctgaagttgtcggtgaagaaattagatccttgtactcctggtccgacgaagacaagttgattaacaactgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatgtgtgcgtgtatgtgtacacctgtatttaatttccttactcgcgggtttttcttttttctcaattcttggcttcctctttctcgagcggaccggaattaccgtcgctcgtgatttgtttgcaaaaagaacaaaactgaaaaaacccagacacgctcgacttcctgtcttcctattgattgcagcttccaatttcgtcacacaacaaggtcctgtcgacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataatacacgtgagtagtgagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgaggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgccgaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 285 - DNA, 15539 bp, Artificial sequence
  pLH468
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggcgcgccactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccacccgggccccccctcgaggtcgacggtatcgataagcttgatatcgaattcctgcagcccgggggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaaaaagttaaaattgtacgaatagattcaccacttcttaacaaatcaaacccttcattgattttctcgaatggcaatacatgtgtaattaaaggatcaagagcaaacttcttcgccataaagtcggcaacaagttttggaacactatccttgctcttaaaaccgccaaatatagctcccttccatgtacgaccgcttagcaacagcataggattcatcgacaaattttgtgaatcaggaggaacacctacgatcacactgactccatatgcctcttgacagcaggacaacgcagttaccatagtatcaagacggcctataacttcaaaagagaaatcaactccaccgtttgacatttcagtaaggacttcttgtattggtttcttataatcttgagggttaacacattcagtagccccgacctccttagcttttgcaaatttgtccttattgatgtctacacctataatcctcgctgcgcctgcagctttacaccccataataacgcttagtcctactcctcctaaaccgaatactgcacaagtcgaaccctgtgtaacctttgcaactttaactgcggaaccgtaaccggtggaaaatccgcaccctatcaagcaaactttttccagtggtgaagctgcatcgattttagcgacagatatctcgtccaccactgtgtattgggaaaatgtagaagtaccaaggaaatggtgtataggtttccctctgcatgtaaatctgcttgtaccatcctgcatagtacctctaggcatagacaaatcatttttaaggcagaaattaccctcaggatgtttgcagactctacacttaccacattgaggagtgaacagtgggatcactttatcaccaggacgaacagtggtaacaccttcacctatggattcaacgattccggcagcctcgtgtcccgcgattactggcaaaggagtaactagagtgccactcaccacatggtcgtcggatctacagattccggtggcaaccatcttgattctaacctcgtgtgcttttggtggcgctacttctacttcttctatgctaaacggctttttctcttcccacaaaactgccgctttacacttaataactttaccggctgttgacatcctcagctagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgctgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcatgcggatcccccgggctgcaggaattcgatatcaagcttatcgataccgtcgactggccattaatctttcccatattagatttcgccaagccatgaaagttcaagaaaggtctttagacgaattacccttcatttctcaaactggcgtcaagggatcctggtatggttttatcgttttatttctggttcttatagcatcgttttggacttctctgttcccattaggcggttcaggagccagcgcagaatcattctttgaaggatacttatcctttccaattttgattgtctgttacgttggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcacggccctgcaggcctcagctcttgttttgttctgcaaataacttacccatctttttcaaaactttaggtgcaccctcctttgctagaataagttctatccaatacatcctatttggatctgcttgagcttctttcatcacggatacgaattcattttctgttctcacaattttggacacaactctgtcttccgttgccccgaaactttctggcagttttgagtaattccacataggaatgtcattataactctggttcggaccatgaatttccctctcaaccgtgtaaccatcgttattaatgataaagcagattgggtttatcttctctctaatggctagtcctaattcttggacagtcagttgcaatgatccatctccgataaacaataaatgtctagattctttatctgcaatttggctgcctagagctgcggggaaagtgtatcctatagatccccacaagggttgaccaataaaatgtgatttcgatttcagaaatatagatgaggcaccgaagaaagaagtgccttgttcagccacgatcgtctcattactttgggtcaaattttcgacagcttgccacagtctatcttgtgacaacagcgcgttagaaggtacaaaatcttcttgctttttatctatgtacttgcctttatattcaatttcggacaagtcaagaagagatgatatcagggattcgaagtcgaaattttggattctttcgttgaaaattttaccttcatcgatattcaaggaaatcattttattttcattaagatggtgagtaaatgcacccgtactagaatcggtaagctttacacccaacataagaataaaatcagcagattccacaaattccttcaagtttggctctgacagagtaccgttgtaaatccccaaaaatgagggcaatgcttcatcaacagatgatttaccaaagttcaaagtagtaataggtaacttagtctttgaaataaactgagtaacagtcttctctaggccgaacgatataatttcatggcctgtgattacaattggtttcttggcattcttcagactttcctgtattttgttcagaatctcttgatcagatgtattcgacgtggaattttccttcttaagaggcaaggatggtttttcagccttagcggcagctacatctacaggtaaattgatgtaaaccggctttctttcctttagtaaggcagacaacactctatcaatttcaacagttgcattctcggctgtcaataaagtcctggcagcagtaaccggttcgtgcatcttcataaagtgcttgaaatcaccatcagccaacgtatggtgaacaaacttaccttcgttctgcactttcgaggtaggagatcccacgatctcaacaacaggcaggttctcagcataggagcccgctaagccattaactgcggataattcgccaacaccaaatgtagtcaagaatgccgcagcctttttcgttcttgcgtacccgtcggccatataggaggcatttaactcattagcatttcccacccatttcatatctttgtgtgaaataatttgatctagaaattgcaaattgtagtcacctggtactccgaatatttcttctatacctaattcgtgtaatctgtccaacagatagtcacctactgtatacattttgtttactagtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgaaaaagcgtgttttttattcaaaatgattctaactcccttacgtaatcaaggaatctttttgccttggcctccgcgtcattaaacttcttgttgttgacgctaacattcaacgctagtatatattcgtttttttcaggtaagttcttttcaacgggtcttactgatgaggcagtcgcgtctgaacctgttaagaggtcaaatatgtcttcttgaccgtacgtgtcttgcatgttattagctttgggaatttgcatcaagtcataggaaaatttaaatcttggctctcttgggctcaaggtgacaaggtcctcgaaaatagggcgcgccccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 286 - DNA, 34 bp, Artificial sequence
  Primer HY31
gccgactttatggcgaagaagtttgctcttgatc

SEQ ID NO: 287 - DNA, 21 bp, Artificial sequence
  Primer oBP511
tttttggtggttccggcttcc

SEQ ID NO: 288 - DNA, 8289 bp, Artificial Sequence; identical to SEQ ID NO: 282
  pBP1719 (= pUC19-ura3MCS-U(PGK1)Pfbai-kivD Lg(y)-ADH1 BAC-kivD.LI fragment C plasmid

SEQ ID NO: 289 - DNA, 6081 bp, Saccharomyces cerevisiae
atgtcatcaaaacctgatactggttcggaaatttctggccctcagcgacaggaagaacaagaacaacagatagagcagagctcacctacggaagcaaacgatagaagcattcatgatgaggtaccaaaagtcaagaagcgtcacgaacaaaatagtggtcacaaatcaagaaggaatagcgcatatagttattacagcccacggtcgctttctatgaccaaaagcagggagagtatcactccaaatggtatggatgatgtaagtatttcgaacgtggaacatccaaggccgacagaaccgaaaatcaaaaggggtccatatttactgaagaaaacattgagcagtctttcaatgacgagcgcgaatagtactcatgatgataataaagaccacggttacgctttgaattcatccaagacgcacaactacacatctactcataaccatcatgacggtcatcatgatcatcatcatgttcagttttttcccaataggaagccatcattagcggaaaccctattcaaaaggttttcagggtcaaacagtcacgatggcaataagtcaggaaaggaaagtaaagttgctaacctttccctttcaacggtaaatcctgcacctgctaataggaaaccttctaaagactccactttatctaatcacttggctgataacgtgccaagcactttacgaaggaaagtgtcctcattggtacgtggttcttccgtccatgatataaataatggtattgcagataaacagattagaccaaaggctgttgcgcaatcagaaaatacattacattcatccgatgttcccaatagcaaacgctcgcacagaaaaagctttctgctaggctccacatcttcttcaagcagtagaagaggttcaaatgtcagttcaatgactaacagtgacagtgcaagtatggcgacgtcgggtagtcatgttctccaacataacgtatctaatgtttctccaactactaaaagtaaggacagcgttaacagcgaatccgccgatcacactaataataaatccgagaaagtgactccagaatataatgagaacattccggaaaattctaactctgacaacaaacgcgaagccacaacgcctactatagaaacacccatttcatgtaaaccatcccttttcaggctagatacaaaccttgaggatgttactgatattacaaagacggtgccacccaccgctgtcaattctacactaaattctacacacgggactgagactgcctcacccaaaacggtgatcatgcctgaaggtcctaggaagtcggtgtcaatggctgatctctccgtcgctgccgcagcacctaatggtgaattcacatcaacttccaatgatagatcacaatgggtagcacctcaaagctgggatgtggaaaccaaaaggaaaaaaacaaaacctaaagggagatcgaaatcaagaaggtcaagtatagatgctgatgaacttgatcccatgtcaccggggccaccttcaaaaaaagactctcgtcatcatcacgatcgaaaggataacgaatcaatggtcactgcgggtgacagtaactcaagttttgttgatatatgtaaagaaaacgttccgaatgatagcaagaccgcactcgatactaaatctgtgaaccgcttaaaaagtaatttggctatgagtcccccaagtatacgatatgctccatcaaatttagatggggactacgacacgtcttccacttcctcatctttaccgtcctcatctattagttcagaagatacatcttcctgcagcgattcctcttcgtacactaacgcgtatatggaggccaaccgagagcaggataataaaacaccgatcctgaataaaacgaaatcgtataccaagaaatttacatcctcttcggtaaatatgaattcaccagatggtgcccagagttctggattattactacaagatgagaaggacgatgaggtcgagtgccaactggaacattactataaagatttcagtgatttagatccaaagaggcactatgctattcgtatattcaatactgatgacacttttacgactctctcatgtactccagcgactaccgtcgaagagataatacctgcacttaaaagaaaatttaacattacagcgcaagggaattttcaaatttccctgaaggtgggaaagttgtcaaaaattttgagaccaacttcgaaacctattttaattgaaagaaaacttttacttttgaatggttatcgaaagtcagacccacttcatattatgggtatagaggatttaagttttgtttttaagtttcttttccatcctgtcacaccttctcactttactcctgaacaagaacaaagaataatgagaagcgaatttgttcacgtagatttaaggaatatggatctgactacacctcccatcattttttaccagcatacgtcagaaatagaaagtttagacgtttctaataacgcaaatatattcctacctctggagttcattgaaagctcgattaaattattaagtttgagaatggttaatattagagcatctaaatttccttccaatatcactaaggcgtataaactagtatctttggaattacagagaaacttcataagaaaagtaccgaactcaatcatgaaactgagtaatttaacgatattaaaccttcaatgtaatgagcttgaaagcctaccggctggatttgttgaactgaaaaatctgcaattgctagacttgtcttcaaacaagttcatgcactacccagaagttattaactactgcaccaatcttttacaaatagacctatcatataataaaatccaaagcttaccacagtccactaagtacctagtaaagcttgcgaagatgaacctttctcataacaaactaaattttataggcgacttatcggaaatgacagatttgaggacgctgaacctaagatataacagaatatcatcaattaagacaaatgcgtctaacttgcagaacctttttttaacagataatagaatttcgaactttgaagacactttgccgaaactaagagcccttgaaattcaagagaatccaatcacttctatatccttcaaagatttttatccaaaaaacatgacaagtttgacgttgaacaaggcacagttatcgagtattcctggagaattactcaccaaactatctttcctcgagaaacttgaacttaatcagaataatttgactagactgccacaggagatatccaagttgactaaattagttttcctttcagtggcgagaaacaaactagagtatattccacccgagctatctcaactgaaaagtttgaggacattagatctacattctaacaacataagggactttgttgacggtatggaaaaccttgaactaacatcgctaaatatttcatcgaatgcattcggtaactctagcttagaaaattctttttaccataacatgtcatatgggtcaaagttatctaaaagcctgatgttttttattgctgcagacaatcaatttgatgatgctatgtggcctcttttcaattgctttgtcaatctgaaagtgctaaatctttcttacaacaatttttcagatgtatcgcacatgaaacttgagagcattaccgaattgtacctctccggtaataagctcacgacattgtcgggtgatacagttttgaaatggagctctttaaagactttaatgttgaatagtaaccaaatgttatctctgcctgcagaattatcaaatctctcacagctaagtgtatttgatgttggagcaaatcaattaaagtataatatatcaaactatcattacgattggaactggaggaataataaagaactaaaatatttgaatttttcaggaaatcgaaggtttgaaataaagtcatttataagtcacgatattgatgctgatttgtcagatctgacagtattacctcagttaaaggtactaggtttaatggacgtaactttaaatactaccaaagtaccggatgaaaatgtcaatttccgtttaaggacaactgcatcaataataaatgggatgcgctacggtgttgctgatacattaggtcaaagagactatgtgtcatctcgtgatgttacctttgaaagattccgcggaaatgacgacgaatgcttactatgtcttcatgatagtaaaaaccaaaatgcagattatggccacaatatatcaagaattgttagagatatttacgataaaatactgatcagacaactggaaaggtatggagacgaaacagatgataatataaaaactgcacttcgtttcagttttttgcaactgaataaggagattaacggaatgctaaattctgttgataatggtgccgatgttgccaatctttcatatgcagacttgctaagtggcgcttgctctactgtgatatatatcagagggaagaaactcttcgctgcaaatttaggtgactgtatggctattttatccaaaaacaatggtgactaccaaacgctaaccaaacaacatctcccaacaaagcgggaagaatacgagaggatcagaatatctggcgggtatgtcaacaatggaaaattagatggtgttgtagatgtgtctagagcagtgggtttttttgatttgcttccccacattcatgcttctcccgacatatctgtcgtgacattaacaaaagcagacgagatgcttattgtagcaacgcataagttatgggaatacatggacgtggatacagtttgtgatatcgcgcgtgagaatagtactgatccactccgtgccgcagctgagttgaaggatcatgccatggcttacggctgtacagagaatattacaattttgtgccttgctctttacgagaacattcagcaacaaaatcggttcactttaaataaaaactctttaatgactagaagaagtactttcgaggatactacattaagaagacttcaacctgagatttctccgccaacaggtaacctagcaatggtcttcactgatatcaaaagctcaaccttcttatgggagctattccctaacgcaatgaggaccgcaataaaaactcacaatgacattatgcgtcgtcaactacgaatttacggtggttacgaagtaaagacagaaggagacgcctttatggtggcatttcctacgccaactagtggtctgacatggtgcttaagtgttcaattaaaactcttggatgcacaatggccggaggaaattacctcagttcaagacggctgccaagttacggatagaaatggtaacattatctatcaaggcctatcagttagaatgggtattcattggggctgcccagttccagagcttgatttagtgactcaaagaatggactatttggggccgatggtcaataaggcagcaagggtccagggcgtcgctgacggtggtcagattgcaatgagtagtgatttttactctgaattcaacaagataatgaagtatcatgagcgagtagtgaagggcaaggaatctctcaaggaagtttatggtgaagaaattatcggagaggttcttgaaagagaaattgccatgctggaaagtattggttgggcattttttgactttggcgagcataagctaaagggactcgaaaccaaagaactcgttactattgcgtatcctaagattcttgcttccagacacgaatttgcatctgaagatgagcagtcaaaattaatcaatgaaacgatgttgtttcgtttaagagtcatttcaaacagactggaatctataatgtcagctttaagcggcggatttattgaactagactctcggacggagggaagttatattaaatttaaccctaaagttgaaaatggtattatgcaatcgatttctgagaaggatgcgttgttattttttgatcatgtaattactagaatcgaatccagtgtggcattattacatttacgacaacagaggtgttcaggactggaaatttgcagaaacgataaaacatctgctcgaagcaatattttcaatgttgttgacgaacttttacaaatggttaagaacgcaaaggatttatcaacttga

(19) United States
(12) Patent Application Publication
(10) Pub. No.: US 2016/0138050 A1
(43) Pub. Date: May 19, 2016

(54) YEAST WITH INCREASED BUTANOL TOLERANCE INVOLVING CELL WALL PROTEINS

(71) Applicant: BUTAMAX ADVANCED BIOFUELS LLC, Wilmington, DE (US)

(72) Inventor: Michael G. BRAMUCCI, Oxfrod, PA (US)

(73) Assignee: Butamax Advanced Biofuels LLC, Wilmington, DE (US)

(21) Appl. No.: 14/902,891

(22) PCT Filed: Jul. 14, 2014
(86) PCT No.: PCT/US14/46474

§ 371 Date: Jan. 5, 2016

Related U.S. Application Data
(60) Provisional application No. 61/846,771, filed on Jul. 16, 2013.

(51) Int. Cl. C12P 7/16; C12P 7/04; C12N 15/81

(52) U.S. Cl. CPC C12P 7/16; C12N 15/81; C12N 15/815; C12P 7/04

(57) ABSTRACT

Provided herein are recombinant yeast host cells and methods for their use for production of fermentation products from a pyruvate utilizing pathway. The yeast host cells provided herein comprise at least one genetic modification in a pyruvate decarboxylase gene and at least one genetic modification in an endogenous cell wall protein, which confers resistance to butanol and increased glucose utilization.

### CROSS REFERENCE TO RELATED APPLICATION

This application claims benefit of priority from U.S. Provisional Application No. 61/846,771, filed Jul. 16, 2013, which is hereby incorporated by reference in its entirety.

### GOVERNMENT LICENSE RIGHTS

This invention was made with Government support under Agreement DE-AR0000006 awarded by the United States Department of Energy. The Government has certain rights in this invention.

### REFERENCE TO SEQUENCE LISTING SUBMITTED ELECTRONICALLY

The content of the electronically submitted sequence listing in ASCII text file (Name: 20140714_CL5880WOPCT_SequenceListing_ascii.txt, Size: 597,855 bytes, and Date of Creation: Jul. 8, 2014) filed with the application is incorporated herein by reference in its entirety.

### FIELD OF THE INVENTION

The invention relates to the field of microbiology and genetic engineering. More specifically, yeast genes that are involved in the cell response to butanol were identified. These genes may be engineered to improve growth yield in the presence of butanol.

### BACKGROUND OF THE INVENTION

Butanol is an important industrial chemical, useful as a fuel additive, as a feedstock chemical in the plastics industry, and as a foodgrade extractant in the food and flavor industry. Each year 10 to 12 billion pounds of butanol are produced by petrochemical means and the need for this commodity chemical will likely increase.

Butanol may be made through chemical synthesis or by fermentation. Isobutanol is a component of “fusel oil”, which can form under certain conditions as a result of incomplete metabolism of amino acids by yeast. Under some circumstances, isobutanol, may be produced from catabolism of L-valine. (See, e.g., Dickinson et al., J. Biol. Chem. 273(40):25752-25756 (1998)). Additionally, recombinant microbial production hosts, expressing an isobutanol biosynthetic pathway have been described. (Donaldson et al., commonly owned U.S. Pat. Nos. 7,851,188 and 7,993,889).

Efficient biological production of butanols may be limited by butanol toxicity to the host microorganism used in fermentation for butanol production. Accordingly, there is a need for genetic modifications which may confer tolerance to butanol.

### SUMMARY OF THE INVENTION

Provided herein are recombinant yeast cells comprising a pyruvate utilizing biosynthetic pathway and further comprising at least one genetic modification in an endogenous cell wall protein gene and at least one genetic modification in an endogenous pyruvate decarboxylase gene. In some embodiments the recombinant yeast cell has an increased tolerance to butanol as compared to a recombinant yeast cell that lacks the at least one genetic modification in an endogenous cell wall protein.

In some embodiments the pyruvate decarboxylase gene is PDC1, PDC5, PDC6, or combinations thereof. In some embodiments there is at least one genetic modification in the endogenous cell wall protein that causes a defect in flocculation and/or filamentous growth as compared to a yeast cell without said genetic modification. In some embodiments the endogenous cell wall protein is FLO1, FLO5, FLO9, FLO10, FLO11, or combinations thereof. In further embodiments the endogenous cell wall protein is FLO1, FLO5, FLO9, or combinations thereof.

In some embodiments the genetic modification in the endogenous cell wall protein gene results in a decrease in flocculation and/or filamentous growth as compared to a microorganism that lacks the at least one genetic modification. In some embodiments the endogenous cell wall protein gene encodes a polypeptide having at least 80% sequence identity to SEQ ID NO: 30, SEQ ID NO: 31, or SEQ ID NO: 32. In some embodiments the endogenous cell wall protein gene encodes a polypeptide having at least 90% sequence identity to SEQ ID NO: 30, SEQ ID NO: 31, or SEQ ID NO: 32. In some embodiments the endogenous cell wall protein gene encodes a polypeptide having at least 95% sequence identity to SEQ ID NO: 30, SEQ ID NO: 31, or SEQ ID NO: 32. In some embodiments the at least one genetic modification in an endogenous cell wall protein gene is in a regulatory sequence of the endogenous cell wall protein gene.

In some embodiments the yeast further comprise a mutation in a gene selected from the group consisting of CYR1, NUM1, PAU10, YGR109W-B, HSP32, ATG13, and combinations thereof. In some embodiments the yeast further comprise a mutation in a gene that regulates the endogenous cell wall protein. In further embodiments the gene that regulates the endogenous cell wall protein is FLOG.

In some embodiments the pyruvate utilizing biosynthetic pathway is an engineered C3-C6 alcohol production pathway. In some embodiments the C3-C6 alcohol is selected from the group consisting of propanol, butanol, pentanol, and hexanol. In some embodiments the C3-C6 alcohol is butanol. In some embodiments the butanol is isobutanol. In some embodiments the engineered pathway comprises the following substrate to product conversions: a) pyruvate to acetolactate; b) acetolactate to 2,3-dihydroxyisovalerate; c) 2,3-dihydroxyisovalerate to α-ketoisovalerate; d) α-ketoisovalerate to isobutyraldehyde; and e) isobutyraldehyde to isobutanol; and wherein (i) the substrate to product conversion of step (a) is performed by a recombinantly expressed acetolactate synthase enzyme; (ii) the substrate to product conversion of step (b) is performed by a recombinantly expressed acetohydroxy acid isomeroreductase enzyme; (iii) the substrate to product conversion of step (c) is performed by a recombinantly expressed acetohydroxy acid dehydratase enzyme; (iv) the substrate to product conversion of step (d) is performed by a recombinantly expressed decarboxylase enzyme; and (v) the substrate to product conversion of step (e) is performed by an alcohol dehydrogenase enzyme; whereby isobutanol is produced from pyruvate via the substrate to product conversions of steps (a)-(e).

In some embodiments the microorganism comprises a recombinantly expressed acetolactate synthase enzyme selected from the group consisting of: (a) an acetolactate synthase having the EC number 2.2.1.6; (b) a polypeptide that has at least 90% identity to any one or more of SEQ ID NO:4, SEQ ID NO:5, or SEQ ID NO:6; (c) a polypeptide encoded by a nucleic acid sequence that has at least 90% identity to any one or more of SEQ ID NOs: 7, 8, or 9; (d) a polypeptide encoded by a nucleic acid sequence that is complementary to any one or more of SEQ ID NOs: 7, 8 or 9; (e) a polypeptide encoded by a nucleic acid sequence that hybridizes under stringent conditions any one or more of SEQ ID NOs: 7, 8, or 9; and (f) any two or more of (a), (b), (c), (d) or (e).

In some embodiments the microorganism comprises a recombinantly expressed acetohydroxy acid isomeroreductase enzyme selected from the group consisting of: (a) an acetohydroxy acid isomeroreductase having the EC number 1.1.1.86; (b) an acetohydroxy acid isomeroreductase that matches the KARI Profile HMI with an E value of <10⁻³ using hmmsearch; (c) a polypeptide that has at least 90% identity to any one or more of SEQ ID NOs: 10; 11 or 12; (d) a polypeptide encoded by a nucleic acid sequence that has at least 90% identity to any one or more of SEQ ID NOs: 13, 14, 15 or 16; (e) a polypeptide encoded by a nucleic acid sequence that is complementary to any one or more of SEQ ID NOs: 13, 14, 15 or 16; (f) is a polypeptide encoded by a nucleic acid sequence that hybridizes under stringent conditions any one or more of SEQ ID NOs: 13, 14, 15 or 16; and (g) any two or more of (a), (b), (c), (d), (e) or (f).

In some embodiments the microorganism comprises a recombinantly expressed acetohydroxy acid dehydratase enzyme selected from the group consisting of: (a) an acetohydroxy acid dehydratase having the EC number 4.2.1.9; (b) a polypeptide that has at least 90% identity to any one or more of SEQ ID NO: 17; SEQ ID NO: 18, SEQ ID NO: 19 or SEQ ID NO: 20; (c) a polypeptide encoded by a nucleic acid sequence that has at least 90% identity to any one or more of SEQ ID NOs: 21, 22, 23, or 24; (d) a polypeptide encoded by a nucleic acid sequence that is complementary to any one or more of SEQ ID NOs: 21, 22, 23 or 24; (e) a polypeptide encoded by a nucleic acid sequence that hybridizes under stringent conditions any one or more of SEQ ID NOs: 21, 22, 23, or 24; and (f) any two or more of (a), (b), (c), (d) or (e).

In some embodiments the microorganism comprises a decarboxylase enzyme selected from the group consisting of: (a) an α-keto acid decarboxylase having the EC number 4.1.1.72; (b) a pyruvate decarboxylase having the EC number 4.1.1.1; (c) a polypeptide that has at least 90% identity to SEQ ID NO: 25; SEQ ID NO: 26, or both; (d) a polypeptide encoded by a nucleic acid sequence that has at least 90% identity to any one or more of SEQ ID NOs: 27, 28, or 29; (e) is a polypeptide encoded by a nucleic acid sequence that is complementary to any one or more of SEQ ID NOs: 27, 28, or 29; (f) is a polypeptide encoded by a nucleic acid sequence that hybridizes under stringent conditions any one or more of SEQ ID: 27, 28, or 29; and (g) any two or more of (a), (b), (c), (d), (e) or (f).

In some embodiments the yeast is a member of the genus selected from the group consisting of Saccharomyces, Schizosaccharomyces, Hansenula, Candida, Kluyveromyces, Yarrowia, Issatchenkia, or Pichia. In some embodiments the yeast is selected from the group consisting of Saccharomyces cerevisiae, Schizosaccharomyces pombe, Kluyveromyces lactis, Kluyveromyces thermotolerans, Kluyveromyces marxianus, Candida glabrata, Candida albicans, Pichia stipitis, or Yarrowia lipolytica. In some embodiments the yeast is Saccharomyces cerevisiae.

In some embodiments the yeast has an increased glucose utilization rate as compared to a corresponding microorganism that does not have at least one genetic modification in an endogenous cell wall protein gene.

Also provided herein is a method of producing a fermentation product from a pyruvate biosynthetic pathway comprising providing the recombinant yeast described herein and growing the yeast under conditions whereby the fermentation product is produced from pyruvate. In some embodiments the fermentation product is a C3-C6 alcohol. In some embodiments the C3-C6 alcohol is selected from the group consisting of propanol, butanol, pentanol, and hexanol. In some embodiments the C3-C6 alcohol is butanol. In some embodiments the butanol is isobutanol.

In some embodiments the method comprises providing a yeast comprising an engineered isobutanol production pathway. In some embodiments the method comprises providing a yeast comprising a recombinantly expressed acetolactate synthase enzyme as described herein. In some embodiments the method comprises providing a yeast comprising a recombinantly expressed acetohydroxy acid isomeroreductase enzyme as described herein. In some embodiments the method comprises providing a yeast comprising a recombinantly expressed acetohydroxy acid dehydratase enzyme as described herein. In some embodiments the method comprises providing a yeast comprising a decarboxylase enzyme as described herein.

In some embodiments the butanol is recovered from the fermentation medium. In some embodiments the butanol is recovered by distillation, liquid-liquid extraction, extraction, adsorption, decantation, pervaporation, or combinations thereof. In some embodiments solids are removed from the fermentation medium. In some embodiments the solids are removed by centrifugation, filtration, or decantation. In some embodiments the solids are removed before recovering the butanol.

In some embodiments the fermentation product is produced by batch, fed-batch, or continuous fermentation.

Also provided herein is a method of using a C3-C6 alcohol, produced by the methods provided herein, as a component of a bio-based fuel. In some embodiments the C3-C6 alcohol is selected from the group consisting of propanol, butanol, pentanol, and hexanol. In some embodiments the C3-C6 alcohol is butanol. In some embodiments the butanol is isobutanol.

Also provided herein is a bio-based fuel comprising a C3-C6 alcohol produced by the methods provided herein. In some embodiments the C3-C6 alcohol is selected from the group consisting of propanol, butanol, pentanol, and hexanol. In some embodiments the C3-C6 alcohol is butanol. In some embodiments the butanol is isobutanol.

Also provided herein is a method for improving production of a butanol comprising: a) providing a recombinant yeast microorganism comprising an engineered butanol biosynthetic pathway selected from the group consisting of: (i) a 1-butanol pathway; (ii) a 2-butanol pathway; (iii) an isobutanol biosynthetic pathway; and wherein the yeast microorganism of (a) also comprises at least one genetic modification which decreases flocculation and/or filamentous growth; and b) contacting the yeast microorganism with fermentable sugar whereby the microorganism produces butanol and wherein the microorganism has improved tolerance to the butanol as compared to a yeast microorganism without at least one genetic modification decreasing flocculation and/or filamentous growth.

Also provided herein is a method for improving glucose utilization during fermentative production of a butanol comprising: a) providing a recombinant yeast microorganism comprising an engineered butanol biosynthetic pathway selected from the group consisting of: (i) a 1-butanol pathway; (ii) a 2-butanol pathway; (iii) an isobutanol biosynthetic pathway; and wherein the yeast microorganism of (a) also comprises at least one genetic modification which decreases flocculation and/or filamentous growth; and b) contacting the yeast microorganism with fermentable sugar whereby the microorganism produces butanol and wherein the microorganism has an improved glucose utilization rate as compared to a yeast microorganism without at least one genetic modification decreasing flocculation and/or filamentous growth.

Also provided herein is a method for producing a recombinant yeast microorganism having increased tolerance to a butanol comprising: a) providing a recombinant yeast microorganism comprising an engineered butanol biosynthetic pathway selected from the group consisting of: (i) a 1-butanol pathway; (ii) a 2-butanol pathway; (iii) an isobutanol biosynthetic pathway; and b) engineering the yeast microorganism of (a) to comprise at least one genetic modification which decreases flocculation and/or filamentous growth as compared to a microorganism lacking the at least one genetic modification.

Also provided herein are a variant polypeptides. In some embodiments the variant polypeptide comprises at least 90%, at least 95%, or at least 99% identity to SEQ ID NO: 32 and a substitution at an amino acid that corresponds to at least one, at least two, at least three, or at least four of positions F287, S600, T966, and T1221 of SEQ ID NO: 32. In a further embodiment the substitution at F287 is S, the substitution at S600 is G, the substitution at T966 is A, and the substitution at T1221 is A. In a further embodiment the variant polypeptide has the sequence of SEQ ID NO: 32.

In some embodiments the variant polypeptide comprises at least 90%, at least 95%, or at least 99% identity to SEQ ID NO: 30 and a substitution at an amino acid that corresponds to at least one or at least two of positions R349 and G1407 of SEQ ID NO: 30. In a further embodiment the substitution at R349 is P and the substitution at G1407 is S. In a further embodiment the variant polypeptide has the sequence of SEQ ID NO: 30.

In some embodiments the variant polypeptide comprises at least 90%, at least 95%, or at least 99% identity to SEQ ID NO: 31 and a substitution at an amino acid that corresponds to position T848 of SEQ ID NO: 31. In a further embodiment the substitution at T848 is I. In a further embodiment the variant polypeptide has the sequence of SEQ ID NO: 31.

Also provided herein are polynucleotides encoding the variant polypeptides. In some embodiments the polynucleotide encodes a variant polypeptide comprising at least 90%, at least 95%, or at least 99% identity to SEQ ID NO: 32 and a substitution at an amino acid the corresponds to at least one, at least two, at least three, or at least four of positions F287, S600, T966, and T1221 of SEQ ID NO: 32. In a further embodiment the polynucleotide encodes a variant polypeptide wherein the substitution at F287 is S, the substitution at S600 is G, the substitution at T966 is A, and the substitution at T1221 is A. In a further embodiment the polynucleotide encodes a variant polypeptide having the sequence of SEQ ID NO: 32.

In some embodiments the polynucleotide encodes a variant polypeptide comprising at least 90%, at least 95%, or at least 99% identity to SEQ ID NO: 30 and a substitution at an amino acid that corresponds to at least one or at least two of positions R349 and G1407 of SEQ ID NO: 30. In a further embodiment the polynucleotide encodes a variant polypeptide wherein the substitution at R349 is P and the substitution at G1407 is S. In a further embodiment the polynucleotide encodes a variant polypeptide having the sequence of SEQ ID NO: 30.

In some embodiments the polynucleotide encodes a variant polypeptide comprising at least 90%, at least 95%, or at least 99% identity to SEQ ID NO: 31 and a substitution at an amino acid that corresponds to position T848 of SEQ ID NO: 31. In a further embodiment the polynucleotide encodes a variant polypeptide wherein the substitution at T848 is I. In a further embodiment the polynucleotide encodes a variant polypeptide having the sequence of SEQ ID NO: 31.

In some embodiments the polynucleotides encoding the variant polypeptides are codon-optimized for a host cell.

### BRIEF DESCRIPTION OF THE DRAWINGS

The various embodiments of the invention can be more fully understood from the following detailed description and the accompanying sequence descriptions, which form a part of this application.

FIG. 1 depicts different isobutanol biosynthetic pathways. The steps labeled “a”, “b”, “c”, “d”, “e”, “f”, “g”, “h”, “i”, “j”, and “k” represent substrate to product conversions described below. “a” may be catalyzed, for example, by acetolactate synthase. “b” may be catalyzed, for example, by acetohydroxyacid reductoisomerase. “c” may be catalyzed, for example, by acetohydroxy acid dehydratase. “d” may be catalyzed, for example, by branched-chain keto acid decarboxylase. “e” may be catalyzed, for example, by branched chain alcohol dehydrogenase. “f” may be catalyzed, for example, by branched chain keto acid dehydrogenase. “g” may be catalyzed, for example, by acetylating aldehyde dehydrogenase. “h” may be catalyzed, for example, by transaminase or valine dehydrogenase. “i” may be catalyzed, for example, by valine decarboxylase. “j” may be catalyzed, for example, by omega transaminase. “k” may be catalyzed, for example by isobutyryl-CoA mutase.

FIG. 2 depicts growth curves of evolved isobutanol tolerant strains compared to their non-evolved parental strain.

FIG. 3 depicts a graph of O₂ uptake by evolved isobutanol tolerant strains compared to their non-evolved parental strain.

FIG. 4 depicts a graph of specific O₂ uptake by evolved isobutanol tolerant strains compared to their non-evolved parental strain.

FIG. 5 depicts a graph of glucose consumption by evolved isobutanol tolerant strains compared to their non-evolved parental strain.

FIG. 6 depicts a graph of isobutanol production in evolved isobutanol tolerant strains compared to their non-evolved parental strain.

FIG. 7 depicts a graph of isobutanol yields of evolved isobutanol tolerant strains compared to their non-evolved parental strain.

FIG. 8 depicts a graph of isobutryic acid production in evolved isobutanol tolerant strains compared to their non-evolved parental strain.

FIG. 9 depicts a graph of engineered isobutanol biosynthetic pathway yields of evolved isobutanol tolerant strains compared to their non-evolved parental strain.

### DETAILED DESCRIPTION

As described herein, Applicants employed environmental evolution to isolate strains of yeast tolerant to higher levels of butanol. From this environmental evolution, strains were isolated that were tolerant to butanol in the fermentation medium. Furthermore, the isolated strains had an increased ability to utilize glucose and produce a fermentation product from a pyruvate utilizing pathway in the presence of butanol in the fermentation medium. Analysis of the isolated butanol tolerant strains revealed that the evolved strains had acquired mutations in nine genes (FLO1, FLO5, FLO9, NUM1, PAU10, YGR109W-B, HSP32, ATG13, and CYR1). In another embodiment, yeast cells comprising mutations in one or more of FLO1, FLO5, and FLO9, and further comprising reduced pyruvate decarboxylase activity had increased glucose utilization, as compared to yeast cells not expressing a mutant FLO gene, suggesting that the environmental evolution methods disclosed herein provide the ability to identify genes that have a role in conferring tolerance to alcohols and increasing production of fermentation products.

The present invention relates to recombinant yeast cells that are engineered for the production of a fermentation product that is synthesized from pyruvate and that additionally comprise reduced pyruvate decarboxylase activity and a genetic alteration in an endogenous cell wall protein. These yeast cells have increased tolerance to butanol and an increased rate of glucose utilization in the presence of butanol, and they can be used for the production of C3-C6 alcohols, such as butanol, which are valuable as fuel additives to reduce demand for fossil fuels.

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

In order to further define this invention, the following terms and definitions are herein provided.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “consists of” or variations such as “consist of” or “consisting of,” as used throughout the specification and claims, indicate the inclusion of any recited integer or group of integers, but that no additional integer or group of integers may be added to the specified method, structure, or composition.

As used herein, the term “consists essentially of” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. §2111.03.

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the application.

As used herein, the term “about” modifying the quantity of an ingredient or reactant of the invention employed refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

In some instances, “biomass” as used herein refers to the cell biomass of the fermentation product-producing microorganism.

The term “bio-based fuel” as used herein refers to a fuel in which the carbon contained within the fuel is derived from recently living biomass. “Recently living biomass” are defined as organic materials having a ¹⁴C/¹²C isotope ratio in the range of from 1:0 to greater than 0:1 in contrast to a fossil-based material which has a ¹⁴C/¹²C isotope ratio of 0.1. The ¹⁴C/¹²C isotope ratio can be measured using methods known in the art such as the ASTM test method D 6866-05 (Determining the Biobased Content of Natural Range Materials Using Radiocarbon and Isotope Ratio Mass Spectrometry Analysis). A bio-based fuel is a fuel in its own right, but may be blended with petroleum-derived fuels to generate a fuel. A bio-based fuel may be used as a replacement for petrochemically-derived gasoline, diesel fuel, or jet fuel.

The term “fermentation product” includes any desired product of interest, including, but not limited to lactic acid, 3-hydroxy-propionic acid, acrylic acid, acetic acid, succinic acid, citric acid, fumaric acid, malic acid, itaconic acid, 1,3-propane-diol, ethylene, glycerol, isobutyrate, butanol and other lower alkyl alcohols, etc.

The term “lower alkyl alcohol” refers to any straight-chain or branched, saturated or unsaturated, alcohol molecule with 1-10 carbon atoms.

The term “C3-C6 alcohol” refers to any alcohol with 3-6 carbon atoms.

The term “pyruvate utilizing biosynthetic pathway” refers to any enzyme pathway that utilizes pyruvate as its starting substrate.

The term “C3-C6 alcohol pathway” as used herein refers to an enzyme pathway to produce C3-C6 alcohols. For example, engineered isopropanol biosynthetic pathways are disclosed in U.S. Patent Appl. Pub. No. 2008/0293125, which is incorporated herein by reference. From time to time “C3-C6 alcohol pathway” is used synonymously with “C3-C6 alcohol production pathway”.

The term “butanol” refers to 1-butanol, 2-butanol, 2-butanone, isobutanol, or mixtures thereof. Isobutanol is also known as 2-methyl-1-propanol.

The term “engineered” as used herein refers to an enzyme pathway that is not present endogenously in a microorganism and is deliberately constructed to produce a fermentation product from a starting substrate through a series of specific substrate to product conversions.

The term “butanol biosynthetic pathway” as used herein refers to an enzyme pathway to produce 1-butanol, 2-butanol, 2-butanone or isobutanol. For example, engineered isobutanol biosynthetic pathways are disclosed in U.S. Pat. Nos. 7,851,188 and 7,993,889, which are incorporated by reference herein. Additionally, an example of an engineered 1-butanol pathway is disclosed in U.S. Patent Appl. Pub. No. 2008/0182308, which is incorporated by reference herein. Examples of engineered 2-butanol and 2-butanone biosynthetic pathways are disclosed in U.S. Pat. No. 8,206,970 and U.S. Patent Pub. No. 2009/0155870, which are incorporated by reference herein. From time to time “butanol biosynthetic pathway” is used synonymously with “butanol production pathway”.

The term “isobutanol biosynthetic pathway” refers to the enzymatic pathway to produce isobutanol. From time to time “isobutanol biosynthetic pathway” is used synonymously with “isobutanol production pathway”.

The term “2-butanone biosynthetic pathway” as used herein refers to an enzyme pathway to produce 2-butanone.

A “recombinant microbial host cell” is defined as a host cell that has been genetically manipulated to express a biosynthetic production pathway, wherein the host cell either produces a biosynthetic product in greater quantities relative to an unmodified host cell or produces a biosynthetic product that is not ordinarily produced by an unmodified host cell.

The term “fermentable carbon substrate” refers to a carbon source capable of being metabolized by the microorganisms such as those disclosed herein. Suitable fermentable carbon substrates include, but are not limited to, monosaccharides, such as glucose or fructose; disaccharides, such as lactose or sucrose; oligosaccharides; polysaccharides, such as starch, cellulose, or lignocellulose, hemicellulose; one-carbon substrates, fatty acids; and a combination of these.

“Fermentation medium” as used herein means the mixture of water, sugars (fermentable carbon substrates), dissolved solids, microorganisms producing fermentation products, fermentation product and all other constituents of the material in which the fermentation product is being made by the reaction of fermentable carbon substrates to fermentation products, water and carbon dioxide (CO₂) by the microorganisms present. From time to time, as used herein the term “fermentation broth” and “fermentation mixture” can be used synonymously with “fermentation medium.”

The term “aerobic conditions” as used herein means growth conditions in the presence of oxygen.

The term “microaerobic conditions” as used herein means growth conditions with low levels of dissolved oxygen. For example, the oxygen level may be less than about 1% of air-saturation.

The term “anaerobic conditions” as used herein means growth conditions in the absence of oxygen.

“Butanol tolerance” or “tolerance to butanol” as used herein refers to the degree of effect butanol has on one or more of the following characteristics of a host cell in the presence of fermentation medium containing aqueous butanol: aerobic growth rate or anaerobic growth rate (typically a change in grams dry cell weight per liter fermentation medium per unit time, which may be expressed as “mu”), change in biomass (which may be expressed, for example, as a change in grams dry cell weight per liter fermentation medium, or as a change in optical density (O.D.)) over the course of a fermentation, volumetric productivity (which may be expressed in grams butanol produced per liter of fermentation medium per unit time), specific sugar consumption rate (“qS” typically expressed in grams sugar consumed per gram of dry cell weight of cells per hour), specific isobutanol production rate (“qP” typically expressed in grams butanol produced per gram of dry cell weight of cells per hour), or yield of butanol (grams of butanol produced per grams sugar consumed). It will be appreciated that increased butanol concentrations may impact one or more of the listed characteristics. Accordingly, an improvement in butanol tolerance can be demonstrated by a reduction or elimination of such impact on one or more of the listed characteristics.

The term “carbon substrate” refers to a carbon source capable of being metabolized by the recombinant host cells disclosed herein. Non-limiting examples of carbon substrates are provided herein and include, but are not limited to, monosaccharides, oligosaccharides, polysaccharides, ethanol, lactate, succinate, glycerol, carbon dioxide, methanol, glucose, fructose, sucrose, xylose, arabinose, dextrose, and mixtures thereof.

As used herein, the term “yield” refers to the amount of product per amount of carbon source in g/g. The yield may be exemplified for glucose as the carbon source. It is understood unless otherwise noted that yield is expressed as a percentage of the theoretical yield. In reference to a microorganism or metabolic pathway, “theoretical yield” is defined as the maximum amount of product that can be generated per total amount of substrate as dictated by the stoichiometry of the metabolic pathway used to make the product. For example, the theoretical yield for one typical conversion of glucose to isopropanol is 0.33 g/g. As such, a yield of isopropanol from glucose of 29.7 g/g would be expressed as 90% of theoretical or 90% theoretical yield. It is understood that while in the present disclosure the yield is exemplified for glucose as a carbon source, the invention can be applied to other carbon sources and the yield may vary depending on the carbon source used. One skilled in the art can calculate yields on various carbon sources.

The term “effective titer” as used herein, refers to the total amount of C3-C6 alcohol produced by fermentation per liter of fermentation medium. The total amount of C3-C6 alcohol includes: (i) the amount of C3-C6 alcohol in the fermentation medium; (ii) the amount of C3-C6 alcohol recovered from the organic extractant; and (iii) the amount of C3-C6 alcohol recovered from the gas phase, if gas stripping is used.

The term “effective rate” as used herein, refers to the total amount of C3-C6 alcohol produced by fermentation per liter of fermentation medium per hour of fermentation.

The term “effective yield” as used herein, refers to the amount of C3-C6 alcohol produced per unit of fermentable carbon substrate consumed by the biocatalyst.

The term “specific productivity” as used herein, refers to the g of C3-C6 alcohol produced per g of dry cell weight of cells per unit time.

As used herein the term “coding sequence” refers to a DNA sequence that encodes for a specific amino acid sequence. “regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

The terms “derivative” and “analog” refer to a polypeptide differing from the enzymes of the invention, but retaining essential properties thereof. The term “derivative” may also refer to a host cells differing from the host cells of the invention, but retaining essential properties thereof. Generally, derivatives and analogs are overall closely similar, and, in many regions, identical to the enzymes of the invention. The terms “derived-from”, “derivative” and “analog” when referring to enzymes of the invention include any polypeptides which retain at least some of the activity of the corresponding native polypeptide or the activity of its catalytic domain.

Derivatives of enzymes disclosed herein are polypeptides which may have been altered so as to exhibit features not found on the native polypeptide. Derivatives can be covalently modified by substitution (e.g. amino acid substitution), chemical, enzymatic, or other appropriate means with a moiety other than a naturally occurring amino acid (e.g., a detectable moiety such as an enzyme or radioisotope). Examples of derivatives include fusion proteins, or proteins which are based on a naturally occurring protein sequence, but which have been altered. For example, proteins can be designed by knowledge of a particular amino acid sequence, and/or a particular secondary, tertiary, and/or quaternary structure. Derivatives include proteins that are modified based on the knowledge of a previous sequence, natural or synthetic, which is then optionally modified, often, but not necessarily to confer some improved function. These sequences, or proteins, are then said to be derived from a particular protein or amino acid sequence. In some embodiments of the invention, a derivative must retain at least 50% identity, at least 60% identity, at least 70% identity, at least 80% identity, at least 85% identity, at least 87% identity, at least 90% identity, at least 91% identity, at least 92% identity, at least 93% identity, at least 94% identity, at least 95% identity, at least 96% identity, at least 97% identity, at least 98% identity, or at least 99% identity to the sequence the derivative is “derived-from.” In some embodiments of the invention, an enzyme is said to be derived-from an enzyme naturally found in a particular species if, using molecular genetic techniques, the DNA sequence for part or all of the enzyme is amplified and placed into a new host cell.

### Screening for C3-C6 Alcohol Tolerance

The invention relates to the discovery that modifying endogenous cell wall proteins while reducing pyruvate decarboxylase activity has the effect of increasing tolerance of yeast cells to isobutanol. Furthermore, the invention relates to the discovery that yeast comprising modified cell wall proteins and reduced pyruvate decarboxylase activity have increased glucose utilization. These discoveries came from the selection for isobutanol tolerance in high density yeast cultures.

Tolerance to C3-C6 alcohols can be selected for by growing high density cultures of yeast comprising an engineered C3-C6 alcohol production pathway and further comprising reduced pyruvate decarboxylase activity in media comprising a C3-C6 alcohol present at initially at low percentage. Because yeast comprising reduced pyruvate decarboxylase activity have a low tolerance to glucose, media comprising ethanol as the carbon source is utilized. After each round of growth, the surviving cells can be inoculated into fresh media comprising a higher percentage of C3-C6 than the previous culture and grown again to select for cells that can tolerate the higher percentage of C3-C6 alcohol in the media. Following several rounds of selection, involving increasing amounts of C3-C6 alcohol being present in the media, cultures of yeast are obtained that have evolved to survive in higher concentrations of C3-C6 alcohol.

Alternatively, yeast comprising an engineered C3-C6 alcohol production pathway and further comprising reduced pyruvate decarboxylase activity can be cultured in a chemostat in growth medium comprising ethanol and a C3-C6 alcohol present at initially at low percentage. The chemostat can be operated in a continuous feed mode in which the amount of C3-C6 alcohol and glucose entering the chemostat is increased overtime. The addition of either increased concentrations of glucose or a C3-C6 alcohol results in a gradual increase in C3-C6 alcohol concentration in the chemostat. After extensive culturing of the yeast in the presence of increased C3-C6 alcohol concentrations, the cultures can be plated onto solid media to select for evolved strains that tolerated the increased alcohol concentration in the chemostat.

Because the goal of evolving yeast to tolerate higher levels of C3-C6 alcohol is the ability to use them in the fermentative production of alcohol, it is important to select for strains that can ultimately utilize glucose to produce C3-C6 alcohol through an engineered C3-C6 alcohol production pathway. To accomplish this, the evolved cultures obtained by the method described above can then be sub-cultured to obtain isolated colonies of yeast. The isolated colonies can then be cultured in media comprising glucose and a C3-C6 alcohol. Monitoring of the growth rates of the cultures then allows for the identification of glucose utilizing strains that are also tolerant to C3-C6 alcohol.

From the methods described above, evolved isolates can then be tested for glucose utilization in the presence of C3-C6 alcohol by monitoring glucose consumption of the identified strains. Evolved strains can be grown in the presence of a set amount of glucose in medium which further comprises a C3-C6 alcohol. Samples can be removed at different time points and the amount of glucose remaining in the medium can be measured. Strains with increased rates of glucose consumption compared to their non-evolved parental strain can then be selected for further analysis by the methods describe herein.

The evolved strains selected for further analysis can then be subjected to whole genome sequencing using methods that are well known in the art. For example, one such method involves sequencing-by-synthesis (E. R. Mardis. 2008. Next-Generation DNA Sequencing Methods. Annu. Rev. Genom. Human Genet. 9:387-402.). Genomic DNA is randomly sheared and specific adapters are ligated to both ends of the fragments which are then denatured. The ligated fragments are arrayed in a flow cell. Primers, fluorescently labeled, 3′-OH blocked nucleotides and DNA polymerase are added to the flow cell. The primed DNA fragments are extended by one nucleotide during the incorporation step. The unused nucleotides and DNA polymerase molecules are then washed away and the optics system scans the flow cell to image the arrayed fragments. After imaging, the fluorescent labels and the 3′-OH blocking groups are cleaved and washed away, preparing the fragments for another round of fluorescent nucleotide incorporation. Assembled genomic sequences of the evolved strains can be compared to the non-evolved parental strain to identify mutations that are present in the evolved strains but not in the non-evolved parental strain.

### Identification of Mutations in Isobutanol Tolerant Strains

Employing the method described above, mutations in nine genes were identified in seven separate strains that were evolved to have increased tolerance to isobutanol. Genomic sequencing of the evolved strains identified mutations in FLO1 (SEQ ID NO: 1); FLO5 (SEQ ID NO:2); FLO9 (SEQ ID NO: 3), NUM1 (SEQ ID NO: 33), PAU10 (SEQ ID NO: 34), YGR109W-B (SEQ ID NO: 35), CYR1 (SEQ ID NO: 289), HSP32 (SEQ ID NO: 36), and ATG13 (SEQ ID NO: 37).

FLO1 encodes a lectin-like protein that is involved in flocculation. (Journal of Applied Microbiology (2011) 110:1-18). FLO1 is a cell wall protein that binds mannose chains on the surface of other cells and promotes flocculation. (Eukaryotic Cell (2011) 10:110-117). Mutations in FLO1 result in a decrease in flocculation. (Id.)

FLO5 encodes a lectin-like protein that is involved in flocculation. (Journal of Applied Microbiology (2011) 110:1-18). FLO5 is a paralog of FLO1 and is a cell wall protein that binds mannose chains on the surface of other cells to promote flocculation. (Yeast (1995) 11:735-45; Proc. Natl. Acad. Sci. U.S.A. (2010) 107:22511-22516).

FLO9 encodes a lectin-like protein that is involved in flocculation (Journal of Applied Microbiology (2011) 110:1-18). Null mutations in FLO9 result in reduced filamentous and invasive growth (Genetics (1996) 144:967-978). Exposure to fusel alcohols such as isobutanol results in invasive and filamentous growth (Folia Microbiologica (2008) 53:3-14). Since invasive/filamentous growth may be an adaptation to solid media, mutations in FLO9 may enable cells to grow better in suspension in liquid media.

NUM1 encodes a protein required for nuclear migration during cell division. (Molecular and General Genetics (1991) 230:277-287). Mutations in NUM1 result defective mitotic spindle movement and nuclear segregation due to defects in dynein-dependent microtubule sliding in the yeast bud during cell division. (Journal of Cell Biology (2000) 151:1337-1344).

PAU10 encodes a protein of unknown function and is a member of the seripauperin multigene family. Seripauperins are serine-poor proteins that are homologous to a serine-rich protein, Srp1p. (Gene (1994) 148:149-153).

YGR109W-B is a Ty3 transposable element located on chromosome VII. Ty3 transposable elements prefer to integrate within the region of RNA polymerase III transcription initiation. (Genes and Development (1992) 6:117-128).

HSP32 encodes a possible chaperone and cysteine protease that is similar to yeast Hsp31p and Escherichia coli Hsp31. The function of Hsp31 like proteins is unknown.

ATG13 encodes a protein involved in autophagy. (Gene (1997) 192:207-213). Atg13p is important for cell viability during starvation conditions, and it is part of a protein kinase complex that is required for vesicle expansion during autophagy. (FEBS Letters (2007) 581:2156-2161).

CYR1 (also known as YJL005W in Saccharomyces cerevisiae) encodes an adenylate cyclase. Adenylate cyclase synthesizes cyclic-AMP (“cAMP”) from ATP. (Cell (1985) 43:493-505). In yeast, CYR1 is an essential gene and has roles in nutrient signaling, cell cycle progression, sporulation, cell growth, response to stress, and longevity. (Microbiology and Molecular Biology Reviews (2003) 67:376-399; Microbiology and Molecular Biology Reviews (2006) 70:253-282). Null mutations in CYR1 block cell division. (Proc. Natl. Acad. Sci. USA (1982) 79:2355-2359). However, viable mutations of CYR1 have been isolated. For example, an E1682K mutation located in the catalytic domain of CYR1 was identified in a screen for genes that confer increased stress resistance during fermentation. (U.S. Patent Appl. Pub. No. 2004/0175831).

### Endogenous Cell Wall Proteins

The identification that variants of FLO1, FLO5, and FLO9 confer tolerance to butanol indicates that genetic modifications in cell wall proteins may result in C3-C6 alcohol tolerance. The yeast cell wall comprises interlinked β-glucan polysaccharides and chitin and acts as the supporting scaffold for highly glycosylated mannoproteins. (G3: Genes|Genomes|Genetics (2012) 2:131-141). Other screens for tolerance to butanol have also identified genes that when overexpressed are presumed to affect the expression of cell wall proteins. (See U.S. Patent Appl. Pub. Nos. 2010/0167363, 2010/0167364, and 2010/0167365, all herein incorporated by reference). One such gene, MSS11 has been implicated in regulating FLO1 expression. (G3: Genes|Genomes|Genetics (2012) 2:131-141). Overexpression of MSS11 results in an increase in FLO1 expression, as well as an increase in expression of FLO5 and FLO9. (Id.)

Given the connection between MSS11 and FLO gene expression, other endogenous cell wall protein genes regulated by MSS11 are good targets for genetic modifications to increase tolerance to butanol. Similar to its effect on FLO1, FLO5, and FLO9, overexpression of MSS11 results in an increase in expression of other cell wall proteins, such as, TIR1 (SEQ ID NO: 38), TIR2 (SEQ ID NO: 39), TIR3 (SEQ ID NO: 40), TIR4 (SEQ ID NO: 41), DAN1 (SEQ ID NO: 42), and FLO11 (SEQ ID NO: 43). (Id.) Other cell wall proteins not specifically enumerated above can also be targeted for genetic modification.

The term “cell wall protein” refers to any protein that comprises a component of or is localized to the yeast cell wall.

### FLO Gene Family

The FLO family of genes (FLO1, FLO5, FLOG, FLO9, FLO10, and FLO11) are of particular interest because the sequencing data indicates that seven of the isolated strains developed mutations in one or more of FLO1, FLO5, and FLO9.

FLO1, FLO5, and FLO9 have been described above. FLO8 (SEQ ID NO: 44) is a transcription factor that in conjunction with MSS11 regulates FLO1 expression. (Curr. Genet. (2006) 49:375-83). FLO10 (SEQ ID NO: 45) has some sequence similarity to FLO1, with the greatest similarity in its N-terminal region. (Yeast (1995) 11:1001-13). FLO11 (SEQ ID NO: 43) encodes a GPI-anchored cell wall protein that is also regulated by MSS11 and FLOG. (Journal of Bacteriology (1996) 178:7144-7151; G3: Genes|Genomes|Genetics (2012) 2:131-141). Genetic modifications in the members of the FLO family of genes results in decreased flocculation and/or decreased filamentous growth. (Journal of Applied Microbiology (2011) 110:1-18).

Additionally, the sequences of the FLO gene coding regions provided herein may be used to identify other homologs in nature. For example each of the FLO gene nucleic acid fragments described herein may be used to isolate genes encoding homologous proteins. Isolation of homologous genes using sequence-dependent protocols is well known in the art. Examples of sequence-dependent protocols include, but are not limited to: 1) methods of nucleic acid hybridization; 2) methods of DNA and RNA amplification, as exemplified by various uses of nucleic acid amplification technologies [e.g., polymerase chain reaction (PCR), Mullis et al., U.S. Pat. No. 4,683,202; ligase chain reaction (LCR), Tabor, S. et al., Proc. Natl. Acad. Sci. U.S.A. 82:1074 (1985); or strand displacement amplification (SDA), Walker, et al., Proc. Natl. Acad. Sci. U.S.A., 89:392 (1992)]; and 3) methods of library construction and screening by complementation.

For example, genes encoding similar proteins or polypeptides to the FLO family genes provided herein could be isolated directly by using all or a portion of the instant nucleic acid fragments as DNA hybridization probes to screen libraries from any desired organism using methodology well known to those skilled in the art. Specific oligonucleotide probes based upon the disclosed nucleic acid sequences can be designed and synthesized by methods known in the art (Maniatis, supra). Moreover, the entire sequences can be used directly to synthesize DNA probes by methods known to the skilled artisan (e.g., random primers DNA labeling, nick translation or end-labeling techniques), or RNA probes using available in vitro transcription systems. In addition, specific primers can be designed and used to amplify a part of (or full-length of) the instant sequences. The resulting amplification products can be labeled directly during amplification reactions or labeled after amplification reactions, and used as probes to isolate full-length DNA fragments by hybridization under conditions of appropriate stringency. Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The use of oligonucleotides as specific hybridization probes in the Diagnosis of Genetic Disorders”, in Human Genetic Diseases: A Practical Approach, K. E. Davis Ed., (1986) pp. 33-50, IRL: Herndon, Va.; and Rychlik, W., In Methods in Molecular Biology, White, B. A. Ed., (1993) Vol. 15, pp 31-39, PCR Protocols: Current Methods and Applications. Humania: Totowa, N.J.).

Generally two short segments of the described sequences may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the described nucleic acid fragments, and the sequence of the other primer takes advantage of the presence of the polyadenylic acid tracts to the 3′ end of the mRNA precursor encoding microbial genes.

Alternatively, the second primer sequence may be based upon sequences derived from the cloning vector. For example, the skilled artisan can follow the RACE protocol (Frohman et al., Proc. Natl. Acad. Sci. U.S.A. 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (e.g., BRL, Gaithersburg, Md.), specific 3′ or 5′ cDNA fragments can be isolated (Ohara et al., Proc. Natl. Acad. Sci. USA 86:5673 (1989); Loh et al., Science 243:217 (1989)).

Alternatively, the provided FLO gene encoding sequences may be employed as hybridization reagents for the identification of homologs. The basic components of a nucleic acid hybridization test include a probe, a sample suspected of containing the gene or gene fragment of interest, and a specific hybridization method. Probes are typically single-stranded nucleic acid sequences that are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions that will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration, the shorter the hybridization incubation time needed. Optionally, a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen, Nucl. Acids Res. 19:5143-5151 (1991)). Suitable chaotropic agents include guanidinium chloride, guanidinium thiocyanate, sodium thiocyanate, lithium tetrachloroacetate, sodium perchlorate, rubidium tetrachloroacetate, potassium iodide and cesium trifluoroacetate, among others. Typically, the chaotropic agent will be present at a final concentration of about 3 M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers (e.g., sodium citrate, Tris-HCl, PIPES or HEPES (pH range about 6-9)), about 0.05 to 0.2% detergent (e.g., sodium dodecylsulfate), or between 0.5-20 mM EDTA, FICOLL (Pharmacia Inc.) (about 300-500 kdal), polyvinylpyrrolidone (about 250-500 kdal) and serum albumin. Also included in the typical hybridization solution will be unlabeled carrier nucleic acids from about 0.1 to 5 mg/mL, fragmented nucleic DNA (e.g., calf thymus or salmon sperm DNA, or yeast RNA), and optionally from about 0.5 to 2% wt/vol glycine. Other additives may also be included, such as volume exclusion agents that include a variety of polar water-soluble or swellable agents (e.g., polyethylene glycol), anionic polymers (e.g., polyacrylate or polymethylacrylate) and anionic saccharidic polymers (e.g., dextran sulfate).

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

### Pyruvate Decarboxylase

The term “pyruvate decarboxylase” refers to an enzyme that catalyzes the decarboxylation of pyruvic acid to acetaldehyde and carbon dioxide. Pyruvate decarboxylases are known by the EC number 4.1.1.1. These enzymes are found in a number of yeast, including Saccharomyces cerevisiae (GenBank No: NP_013145 (SEQ ID NO: 46), CAA97705 (SEQ ID NO: 47), CAA97091 (SEQ ID NO: 48)).

U.S. Appl. Pub. No. 2009/0305363 (incorporated by reference) discloses increased conversion of pyruvate to acetolactate by engineering yeast for expression of a cytosol-localized acetolactate synthase and substantial elimination of pyruvate decarboxylase activity. A genetic modification which has the effect of reducing glucose repression wherein the yeast production host cell is pdc− is described in U.S. Appl. Publication No. 2011/0124060, incorporated herein by reference. In some embodiments, the pyruvate decarboxylase that is deleted or downregulated is selected from the group consisting of: PDC1, PDC5, PDC6, and combinations thereof. In some embodiments, the pyruvate decarboxylase is selected from those enzymes in Table 2.

TABLE 2
SEQ ID Numbers of PDC Target Gene
Coding Regions and Proteins.

| | SEQ ID NO: | SEQ ID NO: |
|---|---|---|
| Description | Nucleic Acid | Amino Acid |
| PDC1 pyruvate decarboxylase from | 49 | 46 |
| Saccharomyces cerevisiae | | |
| PDC5 pyruvate decarboxylase | 50 | 47 |
| from Saccharomyces cerevisiae | | |
| PDC6 pyruvate decarboxylase | 51 | 48 |
| Saccharomyces cerevisiae | | |
| pyruvate decarboxylase from | 52 | 53 |
| Candida glabrata | | |
| PDC1 pyruvate decarboxylase from | 54 | 55 |
| Pichia stipites | | |
| PDC2 pyruvate decarboxylase from | 56 | 57 |
| Pichia stipites | | |
| pyruvate decarboxylase from | 58 | 59 |
| Kluyveromyces lactis | | |
| pyruvate decarboxylase from | 60 | 61 |
| Yarrowia lipolytica | | |
| pyruvate decarboxylase from | 62 | 63 |
| Schizosaccharomyces pombe | | |
| pyruvate decarboxylase from | 64 | 65 |
| Zygosaccharomyces rouxii | | |

Yeasts may have one or more genes encoding pyruvate decarboxylase. For example, there is one gene encoding pyruvate decarboxylase in Candida glabrata and Schizosaccharomyces pombe, while there are three isozymes of pyruvate decarboxylase encoded by the PDC1, PCD5, and PDC6 genes in Saccharomyces. In some embodiments, in the present yeast cells at least one PDC gene is inactivated. If the yeast cell used has more than one expressed (active) PDC gene, then each of the active PDC genes may be modified or inactivated thereby producing a pdc− cell. For example, in S. cerevisiae the PDC1, PDC5, and PDC6 genes may be modified or inactivated. If a PDC gene is not active under the fermentation conditions to be used then such a gene would not need to be modified or inactivated.

Other target genes, such as those encoding pyruvate decarboxylase proteins having at least 70-75%, at least 75-80%, at least 80-85%, at least 85%-90%, at least 90%-95%, or at least 96%, at least 97%, at least 98%, or at least 99% sequence identity to the pyruvate decarboxylases of SEQ ID NOs: 46, 47, 48, 53, 55, 57, 59, 61, 63, or 65 may be identified in the literature and in bioinformatics databases well known to the skilled person. In addition, the methods described herein for identifying FLO family gene homologs can be employed to identify pyruvate decarboxylase genes in microorganisms of interest using the pyruvate decarboxylase sequences provided herein.

### Reduction in Pyruvate Decarboxylase Activity and Genetic Modifications in Endogenous Cell Wall Proteins Results in Increased Glucose Utilization in the Presence of Butanol

Yeast strains comprising reduced pyruvate decarboxylase activity can be modified to contain a genetic modification in at least one endogenous cell wall protein. The resultant strains can then be transformed to comprise an engineered isobutanol biosynthetic pathway. The resultant engineered isobutanol biosynthetic pathway comprising strains obtained from the transformations can then be monitored over time to measure their rate of glucose utilization. In accordance with the present invention, yeast strains comprising reduced pyruvate decarboxylase activity and at least one genetic modification in an endogenous cell wall protein have an increased rate of glucose utilization in the presence of butanol compared to a strain comprising reduced pyruvate decarboxylase activity alone. See Tables 9-11.

In some embodiments the at least one genetic modification is in the coding region of the endogenous cell wall protein. In a further embodiment, the at least one genetic modification is in a regulatory region of the endogenous cell wall protein. In some embodiments the endogenous cell wall proteins is one of FLO1, FLO5, FLO9, FLO10, FLO11, or combinations thereof. In some embodiments, the yeast further comprise a genetic modification in a gene that regulates an endogenous cell wall protein. In a further embodiment, the regulator of the endogenous cell wall protein is FLOG. In accordance with the present invention, yeast strains comprising at least one genetic modification in a cell wall protein may further comprise a mutation in CYR1, NUM1, PAU10, YGR109W-B, HSP32, ATG13, or combinations thereof.

### Polypeptides and Polynucleotides for Use in the Invention

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. A polypeptide may be derived from a natural biological source or produced by recombinant technology, but is not necessarily translated from a designated nucleic acid sequence. It may be generated in any manner, including by chemical synthesis. The polypeptides used in this invention comprise full-length polypeptides and fragments thereof.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purposes of the invention, as are native or recombinant polypeptides which have been separated, fractionated, or partially or substantially purified by any suitable technique.

A polypeptide of the invention may be of a size of about 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

Also included as polypeptides of the present invention are derivatives, analogs, or variants of the foregoing polypeptides, and any combination thereof. The terms “active variant,” “active fragment,” “active derivative,” and “analog” refer to polypeptides of the present invention. Variants of polypeptides of the present invention include polypeptides with altered amino acid sequences due to amino acid substitutions, deletions, and/or insertions. Variants may occur naturally or be non-naturally occurring. Non-naturally occurring variants may be produced using art-known mutagenesis techniques. Variant polypeptides may comprise conservative or non-conservative amino acid substitutions, deletions and/or additions. Derivatives of polypeptides of the present invention, are polypeptides which have been altered so as to exhibit additional features not found on the native polypeptide. Examples include fusion proteins. Variant polypeptides may also be referred to herein as “polypeptide analogs.” As used herein a “derivative” of a polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

A “fragment” is a unique portion of a polypeptide or other enzyme used in the invention which is identical in sequence to but shorter in length than the parent full-length sequence. A fragment may comprise up to the entire length of the defined sequence, minus one amino acid residue. For example, a fragment may comprise from 5 to 1000 contiguous amino acid residues. A fragment may be at least 5, 10, 15, 20, 25, 30, 40, 50, 60, 75, 100, 150, 250 or at least 500 contiguous amino acid residues in length. Fragments may be preferentially selected from certain regions of a molecule. For example, a polypeptide fragment may comprise a certain length of contiguous amino acids selected from the first 100 or 200 amino acids of a polypeptide as shown in a certain defined sequence. Clearly these lengths are exemplary, and any length that is supported by the specification, including the Sequence Listing, tables, and figures, may be encompassed by the present embodiments.

Alternatively, recombinant variants encoding these same or similar polypeptides can be synthesized or selected by making use of the “redundancy” in the genetic code. Various codon substitutions, such as the silent changes which produce various restriction sites, may be introduced to optimize cloning into a plasmid or viral vector or expression in a host cell system.

Preferably, amino acid “substitutions” are the result of replacing one amino acid with another amino acid having similar structural and/or chemical properties, i.e., conservative amino acid replacements, or they can be result of replacing one amino acid with an amino acid having different structural and/or chemical properties, i.e., non-conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Alternatively, “non-conservative” amino acid substitutions can be made by selecting the differences in polarity, charge, solubility, hydrophobicity, hydrophilicity, or the amphipathic nature of any of these amino acids. “Insertions” or “deletions” are preferably in the range of about 1 to about 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

By a polypeptide having an amino acid or polypeptide sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the references sequence.

As a practical matter, whether any particular polypeptide is at least 60%, 65%, 70%, 75%, 80%, 85%, 90%, 95%, 96%, 97%, 98%, or 99% identical to a reference polypeptide can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. Appl. Biosci. 6:237-245 (1990). In a sequence alignment, the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty-0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case, the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to be made for the purposes of the present invention.

Polypeptides and other enzymes suitable for use in the present invention and fragments thereof are encoded by polynucleotides. The term “polynucleotide” is intended to encompass a singular nucleic acid as well as plural nucleic acids, and refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. Polynucleotides according to the present invention further include such molecules produced synthetically. Polynucleotides of the invention may be native to the host cell or heterologous. In addition, a polynucleotide or a nucleic acid may be or may include a regulatory element such as a promoter, ribosome binding site, or a transcription terminator.

In certain embodiments, the polynucleotide or nucleic acid is DNA. In the case of DNA, a polynucleotide comprising a nucleic acid, which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. Other transcription control elements, besides a promoter, for example enhancers, operators, repressors, and transcription termination signals, can be operably associated with the polynucleotide. Suitable promoters and other transcription control regions are disclosed herein.

A polynucleotide or polypeptide sequence can be referred to as “isolated,” in which it has been placed in an environment other than its native environment or is produced synthetically or is a non-naturally occurring, or engineered, sequence. For example, a heterologous polynucleotide encoding a polypeptide or polypeptide fragment having enzymatic activity (e.g., the ability to convert a substrate to xylulose) contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide fragment in the form of a polymer of DNA can be comprised of one or more segments of cDNA, genomic DNA, or synthetic DNA.

The term “gene” refers to a nucleic acid fragment that is capable of being expressed as a specific protein, optionally including regulatory sequences preceding (5′ non-coding sequences) and following (3′ non-coding sequences) the coding sequence.

As used herein, a “coding region” or “ORF” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ non-translated regions, and the like, are not part of a coding region.

A variety of translation control elements are known to those of ordinary skill in the art. These include, but are not limited to ribosome binding sites, translation initiation and termination codons, and elements derived from viral systems (particularly an internal ribosome entry site, or IRES). In other embodiments, a polynucleotide of the present invention is RNA, for example, in the form of messenger RNA (mRNA). RNA of the present invention may be single stranded or double stranded.

Polynucleotide and nucleic acid coding regions of the present invention may be associated with additional coding regions which encode secretory or signal peptides, which direct the secretion of a polypeptide encoded by a polynucleotide of the present invention.

As used herein, the term “transformation” refers to the transfer of a nucleic acid fragment into the genome of a host organism, resulting in genetically stable inheritance. Host organisms containing the transformed nucleic acid fragments are referred to as “recombinant” or “transformed” organisms.

The term “expression,” as used herein, refers to the transcription and stable accumulation of sense (mRNA) or antisense RNA derived from the nucleic acid fragment of the invention. Expression may also refer to translation of mRNA into a polypeptide.

The terms “plasmid,” “vector,” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

The term “artificial” refers to a synthetic, or non-host cell derived composition, e.g., a chemically-synthesized oligonucleotide.

As used herein, “native” refers to the form of a polynucleotide, gene, or polypeptide as found in nature with its own regulatory sequences, if present.

The term “endogenous,” when used in reference to a polynucleotide, a gene, or a polypeptide refers to a native polynucleotide or gene in its natural location in the genome of an organism, or for a native polypeptide, is transcribed and translated from this location in the genome.

The term “heterologous” when used in reference to a polynucleotide, a gene, or a polypeptide refers to a polynucleotide, gene, or polypeptide not normally found in the host organism. “Heterologous” also includes a native coding region, or portion thereof, that is reintroduced into the source organism in a form that is different from the corresponding native gene, e.g., not in its natural location in the organism's genome. The heterologous polynucleotide or gene may be introduced into the host organism by, e.g., gene transfer. A heterologous gene may include a native coding region with non-native regulatory regions that is reintroduced into the native host. A “transgene” is a gene that has been introduced into the genome by a transformation procedure.

“Deletion” or “deleted” or “disruption” or “disrupted” or “elimination” or “eliminated” used with regard to a gene or set of genes describes various activities for example, 1) deleting coding regions and/or regulatory (promoter) regions, 2) inserting exogenous nucleic acid sequences into coding regions and/regulatory (promoter) regions, and 3) altering coding regions and/or regulatory (promoter) regions (for example, by making DNA base pair changes). Such changes would either prevent expression of the protein of interest or result in the expression of a protein that is non-functional/shows no activity. Specific disruptions may be obtained by random mutation followed by screening or selection, or, in cases where the gene sequences are known, specific disruptions may be obtained by direct intervention using molecular biology methods know to those skilled in the art.

The terms “mutation” or “genetic modification” as used herein indicate any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory sequences. A genetic alteration may be a mutation of any type. For instance, the mutation may constitute a point mutation, a frame-shift mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring or spontaneous. In other embodiments, the mutations are the result of treatment with mutagenic agents such as ethyl methanesulfonate or ultraviolet light. In still other embodiments, the mutations in the microorganism genome are the result of genetic engineering.

The term “recombinant genetic expression element” refers to a nucleic acid fragment that expresses one or more specific proteins, including regulatory sequences preceding (5′ non-coding sequences) and following (3′ termination sequences) coding sequences for the proteins. A chimeric gene is a recombinant genetic expression element. The coding regions of an operon may form a recombinant genetic expression element, along with an operably linked promoter and termination region.

“Regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, enhancers, operators, repressors, transcription termination signals, translation leader sequences, introns, polyadenylation recognition sequences, RNA processing site, effector binding site and stem-loop structure.

The term “promoter” refers to a nucleic acid sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. “Inducible promoters,” on the other hand, cause a gene to be expressed when the promoter is induced or turned on by a promoter-specific signal or molecule. It is further recognized that since in most cases the exact boundaries of regulatory sequences have not been completely defined, DNA fragments of different lengths may have identical promoter activity. For example, it will be understood that “FBA1 promoter” can be used to refer to a fragment derived from the promoter region of the FBA1 gene.

The term “terminator” as used herein refers to DNA sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, RNA processing or stability, or translation of the associated coding sequence. It is recognized that since in most cases the exact boundaries of regulatory sequences have not been completely defined, DNA fragments of different lengths may have identical terminator activity. For example, it will be understood that “CYC1 terminator” can be used to refer to a fragment derived from the terminator region of the CYC1 gene.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 3. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

TABLE 3
The Standard Genetic Code

| | T | C | A | G |
|---|---|---|---|---|
| T | TTT Phe (F) | TCT Ser (S) | TAT Tyr (Y) | TGT Cys (C) |
| | TTC Phe (F) | TCC Ser (S) | TAC Tyr (Y) | TGC |
| | TTA Leu (L) | TCA Ser (S) | TAA Ter | TGA Ter |
| | TTG Leu (L) | TCG Ser (S) | TAG Ter | TGG Trp (W) |
| C | CTT Leu (L) | CCT Pro (P) | CAT His (H) | CGT Arg (R) |
| | CTC Leu (L) | CCC Pro (P) | CAC His (H) | CGC Arg (R) |
| | CTA Leu (L) | CCA Pro (P) | CAA Gln (Q) | CGA Arg (R) |
| | CTG Leu (L) | CCG Pro (P) | CAG Gln (Q) | CGG Arg (R) |
| A | ATT Ile (I) | ACT Thr (T) | AAT Asn (N) | AGT Ser (S) |
| | ATC Ile (I) | ACC Thr (T) | AAC Asn (N) | AGC Ser (S) |
| | ATA Ile (I) | ACA Thr (T) | AAA Lys (K) | AGA Arg (R) |
| | ATG Met (M) | ACG Thr (T) | AAG Lys (K) | AGG Arg (R) |
| G | GTT Val (V) | GCT Ala (A) | GAT Asp (D) | GGT Gly (G) |
| | GTC Val (V) | GCC Ala (A) | GAC Asp (D) | GGC Gly (G) |
| | GTA Val (V) | GCA Ala (A) | GAA Glu (E) | GGA Gly (G) |
| | GTG Val (V) | GCG Ala (A) | GAG Glu (E) | GGG Gly (G) |

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon-optimization.

Given the large number of gene sequences available for a wide variety of animal, plant and microbial species, it is possible to calculate the relative frequencies of codon usage. Codon usage tables are readily available, for example, at the “Codon Usage Database” available at http://www.kazusa.or.jp/codon/ (visited Jun. 26, 2012), and these tables can be adapted in a number of ways. See Nakamura, Y., et al. Nucl. Acids Res. 28:292 (2000). Codon usage tables for yeast, calculated from GenBank Release 128.0 [15 Feb. 2002], are reproduced below as Table 4. This table uses mRNA nomenclature, and so instead of thymine (T) which is found in DNA, the tables use uracil (U) which is found in RNA. The Table has been adapted so that frequencies are calculated for each amino acid, rather than for all 64 codons.

TABLE 4
Codon Usage Table for Saccharomyces cerevisiae Genes

| Amino Acid | Codon | Number | Frequency per thousand |
|---|---|---|---|
| Phe | UUU | 170666 | 26.1 |
| Phe | UUC | 120510 | 18.4 |
| Leu | UUA | 170884 | 26.2 |
| Leu | UUG | 177573 | 27.2 |
| Leu | CUU | 80076 | 12.3 |
| Leu | CUC | 35545 | 5.4 |
| Leu | CUA | 87619 | 13.4 |
| Leu | CUG | 68494 | 10.5 |
| Ile | AUU | 196893 | 30.1 |
| Ile | AUC | 112176 | 17.2 |
| Ile | AUA | 116254 | 17.8 |
| Met | AUG | 136805 | 20.9 |
| Val | GUU | 144243 | 22.1 |
| Val | GUC | 76947 | 11.8 |
| Val | GUA | 76927 | 11.8 |
| Val | GUG | 70337 | 10.8 |
| Ser | UCU | 153557 | 23.5 |
| Ser | UCC | 92923 | 14.2 |
| Ser | UCA | 122028 | 18.7 |
| Ser | UCG | 55951 | 8.6 |
| Ser | AGU | 92466 | 14.2 |
| Ser | AGC | 63726 | 9.8 |
| Pro | CCU | 88263 | 13.5 |
| Pro | CCC | 44309 | 6.8 |
| Pro | CCA | 119641 | 18.3 |
| Pro | CCG | 34597 | 5.3 |
| Thr | ACU | 132522 | 20.3 |
| Thr | ACC | 83207 | 12.7 |
| Thr | ACA | 116084 | 17.8 |
| Thr | ACG | 52045 | 8.0 |
| Ala | GCU | 138358 | 21.2 |
| Ala | GCC | 82357 | 12.6 |
| Ala | GCA | 105910 | 16.2 |
| Ala | GCG | 40358 | 6.2 |
| Tyr | UAU | 122728 | 18.8 |
| Tyr | UAC | 96596 | 14.8 |
| His | CAU | 89007 | 13.6 |
| His | CAC | 50785 | 7.8 |
| Gln | CAA | 178251 | 27.3 |
| Gln | CAG | 79121 | 12.1 |
| Asn | AAU | 233124 | 35.7 |
| Asn | AAC | 162199 | 24.8 |
| Lys | AAA | 273618 | 41.9 |
| Lys | AAG | 201361 | 30.8 |
| Asp | GAU | 245641 | 37.6 |
| Asp | GAC | 132048 | 20.2 |
| Glu | GAA | 297944 | 45.6 |
| Glu | GAG | 125717 | 19.2 |
| Cys | UGU | 52903 | 8.1 |
| Cys | UGC | 31095 | 4.8 |
| Trp | UGG | 67789 | 10.4 |
| Arg | CGU | 41791 | 6.4 |
| Arg | CGC | 16993 | 2.6 |
| Arg | CGA | 19562 | 3.0 |
| Arg | CGG | 11351 | 1.7 |
| Arg | AGA | 139081 | 21.3 |
| Arg | AGG | 60289 | 9.2 |
| Gly | GGU | 156109 | 23.9 |
| Gly | GGC | 63903 | 9.8 |
| Gly | GGA | 71216 | 10.9 |
| Gly | GGG | 39359 | 6.0 |
| Stop | UAA | 6913 | 1.1 |
| Stop | UAG | 3312 | 0.5 |
| Stop | UGA | 4447 | 0.7 |

By utilizing this or similar tables, one of ordinary skill in the art can apply the frequencies to any given polypeptide sequence, and produce a nucleic acid fragment of a codon-optimized coding region which encodes the polypeptide, but which uses codons optimal for a given species.

Randomly assigning codons at an optimized frequency to encode a given polypeptide sequence can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the VectorNTI Suite, available from InforMax, Inc., Bethesda, Md., and the “backtranslate” function in the GCG—Wisconsin Package, available from Accelrys, Inc., San Diego, Calif. In addition, various resources are publicly available to codon-optimize coding region sequences, e.g., the “JAVA Codon Adaptation Tool” at http://www.jcat.de/ (visited Jun. 25, 2012) and the “Codon optimization tool” available at http://www.entelechon.com/2008/10/backtranslation-tool/ (visited Jun. 25, 2012). Constructing a rudimentary algorithm to assign codons based on a given frequency can also easily be accomplished with basic mathematical functions by one of ordinary skill in the art.

Standard recombinant DNA and molecular cloning techniques used here are well known in the art and are described by Sambrook et al. (Sambrook, Fritsch, and Maniatis, Molecular Cloning: A Laboratory Manual, Second Edition, Cold Spring Harbor Laboratory Press, Cold Spring Harbor, N. Y., 1989) (hereinafter “Maniatis”); and by Silhavy et al. (Silhavy et al., Experiments with Gene Fusions, Cold Spring Harbor Laboratory Press Cold Spring Harbor, N. Y., 1984); and by Ausubel, F. M. et al., (Ausubel et al., Current Protocols in Molecular Biology, published by Greene Publishing Assoc. and Wiley-Interscience, 1987).

### Biosynthetic Pathways

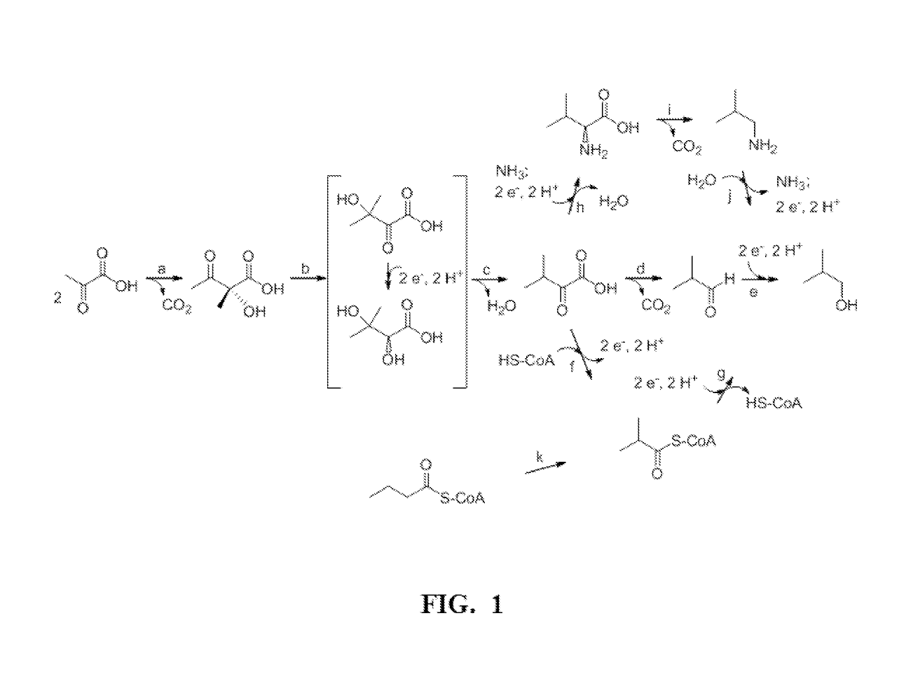

Biosynthetic pathways for the production of isobutanol that may be used include those described in U.S. Pat. Nos. 7,851,188 and 7,993,889, which are incorporated herein by reference. Isobutanol pathways are referred to with their lettering in FIG. 1. In one embodiment, the engineered isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by acetohydroxy acid reductoisomerase;
c) 2,3-dihydroxyisovalerate to α-ketoisovalerate, which may be catalyzed, for example, by acetohydroxy acid dehydratase;
d) α-ketoisovalerate to isobutyraldehyde, which may be catalyzed, for example, by a branched-chain keto acid decarboxylase; and,
e) isobutyraldehyde to isobutanol, which may be catalyzed, for example, by a branched-chain alcohol dehydrogenase.

In another embodiment, the engineered isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by ketol-acid reductoisomerase;
c) 2,3-dihydroxyisovalerate to α-ketoisovalerate, which may be catalyzed, for example, by acetohydroxy acid dehydratase;
h) α-ketoisovalerate to valine, which may be catalyzed, for example, by transaminase or valine dehydrogenase;
i) valine to isobutylamine, which may be catalyzed, for example, by valine decarboxylase;
j) isobutylamine to isobutyraldehyde, which may be catalyzed by, for example, omega transaminase; and,
e) isobutyraldehyde to isobutanol, which may be catalyzed, for example, by a branched-chain alcohol dehydrogenase.

In another embodiment, the engineered isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by acetohydroxy acid reductoisomerase;
c) 2,3-dihydroxyisovalerate to α-ketoisovalerate, which may be catalyzed, for example, by acetohydroxy acid dehydratase;
f) α-ketoisovalerate to isobutyryl-CoA, which may be catalyzed, for example, by branched-chain keto acid dehydrogenase;
g) isobutyryl-CoA to isobutyraldehyde, which may be catalyzed, for example, by acelylating aldehyde dehydrogenase; and,
e) isobutyraldehyde to isobutanol, which may be catalyzed, for example, by a branched-chain alcohol dehydrogenase.

In another embodiment, the isobutanol biosynthetic pathway comprises the substrate to product conversions shown as steps k, g, and e in FIG. 1.

Engineered biosynthetic pathways for the production of 1-butanol that may be used include those described in U.S. Patent Appl. Pub. No. 2008/0182308, which is incorporated herein by reference. In one embodiment, the 1-butanol biosynthetic pathway comprises the following substrate to product conversions:
a) acetyl-CoA to acetoacetyl-CoA, which may be catalyzed, for example, by acetyl-CoA acetyl transferase;
b) acetoacetyl-CoA to 3-hydroxybutyryl-CoA, which may be catalyzed, for example, by 3-hydroxybutyryl-CoA dehydrogenase;
c) 3-hydroxybutyryl-CoA to crotonyl-CoA, which may be catalyzed, for example, by crotonase;
d) crotonyl-CoA to butyryl-CoA, which may be catalyzed, for example, by butyryl-CoA dehydrogenase;
e) butyryl-CoA to butyraldehyde, which may be catalyzed, for example, by butyraldehyde dehydrogenase; and,
f) butyraldehyde to 1-butanol, which may be catalyzed, for example, by butanol dehydrogenase.

Engineered biosynthetic pathways for the production of 2-butanol that may be used include those described in U.S. Pat. No. 8,206,970 and U.S. Patent Appl. Pub. No. 2009/0155870, which are incorporated herein by reference. In one embodiment, the 2-butanol biosynthetic pathway comprises the following substrate to product conversions:
a) pyruvate to α-acetolactate, which may be catalyzed, for example, by acetolactate synthase;
b) α-acetolactate to acetoin, which may be catalyzed, for example, by acetolactate decarboxylase;
c) acetoin to 3-amino-2-butanol, which may be catalyzed, for example, acetonin aminase;
d) 3-amino-2-butanol to 3-amino-2-butanol phosphate, which may be catalyzed, for example, by aminobutanol kinase;
e) 3-amino-2-butanol phosphate to 2-butanone, which may be catalyzed, for example, by aminobutanol phosphate phosphorylase; and,
f) 2-butanone to 2-butanol, which may be catalyzed, for example, by butanol dehydrogenase.

In another embodiment, the engineered 2-butanol biosynthetic pathway comprises the following substrate to product conversions:
a) pyruvate to α-acetolactate, which may be catalyzed, for example, by acetolactate synthase;
b) α-acetolactate to acetoin, which may be catalyzed, for example, by acetolactate decarboxylase;
c) acetoin to 2,3-butanediol, which may be catalyzed, for example, by butanediol dehydrogenase;
d) 2,3-butanediol to 2-butanone, which may be catalyzed, for example, by dial dehydratase; and,
e) 2-butanone to 2-butanol, which may be catalyzed, for example, by butanol dehydrogenase.

Engineered biosynthetic pathways for the production of 2-butanone that may be used include those described in U.S. Pat. No. 8,206,970 and U.S. Patent Appl. Pub. No. 2009/0155870, which are incorporated herein by reference. In one embodiment, the engineered 2-butanone biosynthetic pathway comprises the following substrate to product conversions:
a) pyruvate to α-acetolactate, which may be catalyzed, for example, by acetolactate synthase;
b) α-acetolactate to acetoin, which may be catalyzed, for example, by acetolactate decarboxylase;
c) acetoin to 3-amino-2-butanol, which may be catalyzed, for example, acetonin aminase;
d) 3-amino-2-butanol to 3-amino-2-butanol phosphate, which may be catalyzed, for example, by aminobutanol kinase; and,
e) 3-amino-2-butanol phosphate to 2-butanone, which may be catalyzed, for example, by aminobutanol phosphate phosphorylase.

In another embodiment, the engineered 2-butanone biosynthetic pathway comprises the following substrate to product conversions:
a) pyruvate to α-acetolactate, which may be catalyzed, for example, by acetolactate synthase;
b) α-acetolactate to acetoin which may be catalyzed, for example, by acetolactate decarboxylase;
c) acetoin to 2,3-butanediol, which may be catalyzed, for example, by butanediol dehydrogenase;
d) 2,3-butanediol to 2-butanone, which may be catalyzed, for example, by diol dehydratase.

In one embodiment, the invention produces butanol from plant derived carbon sources, avoiding the negative environmental impact associated with standard petrochemical processes for butanol production. In one embodiment, the invention provides a method for the production of butanol using recombinant industrial host cells comprising an engineered butanol pathway.

In some embodiments, the engineered butanol biosynthetic pathway comprises at least one polynucleotide, at least two polynucleotides, at least three polynucleotides, or at least four polynucleotides that is/are heterologous to the host cell. In embodiments, each substrate to product conversion of an engineered butanol biosynthetic pathway in a recombinant host cell is catalyzed by a heterologous polypeptide. In embodiments, the polypeptide catalyzing the substrate to product conversions of acetolactate to 2,3-dihydroxyisovalerate and/or the polypeptide catalyzing the substrate to product conversion of isobutyraldehyde to isobutanol are capable of utilizing NADH as a cofactor.

The terms “acetohydroxyacid synthase,” “acetolactate synthase” and “acetolactate synthetase” (abbreviated “ALS”) are used interchangeably herein to refer to an enzyme that catalyzes the conversion of pyruvate to acetolactate and CO₂. Example acetolactate synthases are known by the EC number 2.2.1.6 (Enzyme Nomenclature 1992, Academic Press, San Diego). These unmodified enzymes are available from a number of sources, including, but not limited to, Bacillus subtilis (GenBank Nos: CAB15618 (SEQ ID NO: 66), Z99122), Klebsiella pneumoniae (GenBank Nos: AAA25079, M73842), and Lactococcus lactis (GenBank Nos: AAA25161, L16975).

The term “ketol-acid reductoisomerase” (“KARI”), and “acetohydroxy acid isomeroreductase” will be used interchangeably and refer to enzymes capable of catalyzing the reaction of (S)-acetolactate to 2,3-dihydroxyisovalerate. Example KARI enzymes may be classified as EC number EC 1.1.1.86 (Enzyme Nomenclature 1992, Academic Press, San Diego), and are available from a vast array of microorganisms, including, but not limited to, Escherichia coli (GenBank Nos: NP_418222, NC_000913), Saccharomyces cerevisiae (GenBank Nos: NP_013459, NM_001182244), Methanococcus maripaludis (GenBank Nos: CAF30210, BX957220), and Bacillus subtilis (GenBank Nos: CAB14789, Z99118). KARIs include Anaerostipes caccae KARI variants “K9G9” and “K9D3” (SEQ ID NOs: 67 and 68, respectively). Ketol-acid reductoisomerase (KARI) enzymes are described in U.S. Patent Appl. Pub. Nos. 2008/0261230 A1, 2009/0163376 A1, 2010/0197519 A1, and PCT Appl. Pub. No. WO 2011/041415, which are incorporated herein by reference. Examples of KARIs disclosed therein are those from Lactococcus lactis, Vibrio cholera, Pseudomonas aeruginosa PAO1, and Pseudomonas fluorescens PF5 variants (SEQ ID NO: 69). In some embodiments, the KARI utilizes NADH. In some embodiments, the KARI utilizes NADPH.

In addition, suitable KARI enzymes include proteins that match the KARI Profile HMM with an E value of <10⁻³ using hmmsearch program in the HMMER package. The theory behind profile HMMs is described in R. Durbin, S. Eddy, A. Krogh, and G. Mitchison, Biological sequence analysis: probabilistic models of proteins and nucleic acids, Cambridge University Press, 1998; Krogh et al., J. Mol. Biol. 235: 1501-1531, 1994. A KARI Profile HMM generated from the alignment of the twenty-five KARIs with experimentally verified function is provided in U.S. Patent Appl. Pub. No. 2011/0313206, which is incorporated herein by reference. Further, KARI enzymes that are a member of a Glade identified through molecular phylogenetic analysis called the SLSL Glade are described in U.S. Patent Appl. Pub. No. 2011/0244536, incorporated herein by reference.

The term “acetohydroxy acid dehydratase” and “dihydroxyacid dehydratase” (“DHAD”) refers to an enzyme that catalyzes the conversion of 2,3-dihydroxyisovalerate to α-ketoisovalerate. Example acetohydroxy acid dehydratases are known by the EC number 4.2.1.9. Such enzymes are available from a vast array of microorganisms, including, but not limited to, E. coli (GenBank Nos: YP_026248, NC_000913), S. cerevisiae (GenBank Nos: NP_012550, NM_001181674), M. maripaludis (GenBank Nos: CAF29874, BX957219), B. subtilis (GenBank Nos: CAB14105, Z99115), L. lactis, and N. crassa. U.S. Patent Appl. Pub. No. 2010/0081154, and U.S. Pat. No. 7,851,188, which are incorporated herein by reference, describe dihydroxyacid dehydratases (DHADs), including a DHAD from Streptococcus mutans (SEQ ID NO: 70).

The term “branched-chain α-keto acid decarboxylase” or “α-ketoacid decarboxylase” or “α-ketoisovalerate decarboxylase” or “2-ketoisovalerate decarboxylase” (“KIVD”) refers to an enzyme that catalyzes the conversion of α-ketoisovalerate to isobutyraldehyde and CO₂. Example branched-chain α-keto acid decarboxylases are known by the EC number 4.1.1.72 and are available from a number of sources, including, but not limited to, Lactococcus lactis (GenBank Nos: AAS49166, AY548760; CAG34226, AJ746364), Salmonella typhimurium (GenBank Nos: NP_461346, NC_003197), Clostridium acetobutylicum (GenBank Nos: NP_149189, NC_001988), M. caseolyticus (SEQ ID NO: 71), and L. grayi (SEQ ID NO: 72).

The term “alcohol dehydrogenase” (“ADH”) refers to an enzyme that catalyzes the conversion of isobutyraldehyde to isobutanol, 2-butanone to 2-butanol, and/or butyraldehyde to 1-butanol. Alcohol dehydrogenases may be “branched chain alcohol dehydrogenases” or may be referred to as “butanol dehydrogenases.” Example alcohol dehydrogenases suitable for embodiments disclosed herein may be known by the EC number 1.1.1.265, but may also be classified under other alcohol dehydrogenases, for example, according to published utilization of NADH (typically 1.1.1.1) or NADPH (typically 1.1.1.2) as cofactors. Such enzymes are available from a number of sources, including, but not limited to, S. cerevisiae (GenBank Nos: NP_010656; NC_001136; NP_014051; NC_001145); E. coli (GenBank Nos: NP_417484; NC_000913), C. acetobutylicum (GenBank Nos: NP_349892, NC_003030; NP_349891, NC_003030; NP_149325, NC_001988), Pyrococcus furiosus (GenBank Nos: AAC25556, AF013169), Acinetobacter sp. (GenBank Nos: AAG10026, AF282240), Rhodococcus ruber (GenBank Nos: CAD36475, AJ491307), Achromobacter xylosoxidans (SEQ ID NO: 73), and Beijerinkia indica (SEQ ID NO: 74).

The term “branched-chain keto acid dehydrogenase” refers to an enzyme that catalyzes the conversion of α-ketoisovalerate to isobutyryl-CoA (isobutyryl-coenzyme A), typically using NAD⁺ (nicotinamide adenine dinucleotide) as an electron acceptor. Example branched-chain keto acid dehydrogenases are known by the EC number 1.2.4.4. Such branched-chain keto acid dehydrogenases are comprised of four subunits and sequences from all subunits are available from a vast array of microorganisms, including, but not limited to, B. subtilis (GenBank Nos: CAB14336, Z99116; CAB14335, Z99116; CAB14334, Z99116; and CAB14337, Z99116) and Pseudomonas putida (GenBank Nos: AAA65614, M57613; AAA65615, M57613; AAA65617), M57613); and AAA65618, M57613).

The term “acylating aldehyde dehydrogenase” refers to an enzyme that catalyzes the conversion of isobutyryl-CoA to isobutyraldehyde, typically using either NADH or NADPH as an electron donor. Example acylating aldehyde dehydrogenases are known by the EC numbers 1.2.1.10 and 1.2.1.57. Such enzymes are available from multiple sources, including, but not limited to, Clostridium beijerinckii (GenBank Nos: AAD31841, AF157306), C. acetobutylicum (GenBank Nos: NP_149325, NC_001988; NP_149199, NC_001988), P. putida (GenBank Nos: AAA89106, U13232), and Thermus thermophilus (GenBank Nos: YP_145486, NC_006461).

The term “transaminase” refers to an enzyme that catalyzes the conversion of α-ketoisovalerate to L-valine, using either alanine or glutamate as an amine donor. Example transaminases are known by the EC numbers 2.6.1.42 and 2.6.1.66. Such enzymes are available from a number of sources. Examples of sources for alanine-dependent enzymes include, but are not limited to, E. coli (GenBank Nos: YP_026231, NC_000913) and Bacillus licheniformis (GenBank Nos: YP_093743, NC_006322). Examples of sources for glutamate-dependent enzymes include, but are not limited to, E. coli (GenBank Nos: YP_026247, NC_000913), S. cerevisiae (GenBank Nos: NP_012682, NC_001142) and Methanobacterium thermoautotrophicum (GenBank Nos: NP_276546, NC_000916).

The term “valine dehydrogenase” refers to an enzyme that catalyzes the conversion of α-ketoisovalerate to L-valine, typically using NAD(P)H as an electron donor and ammonia as an amine donor. Example valine dehydrogenases are known by the EC numbers 1.4.1.8 and 1.4.1.9 and such enzymes are available from a number of sources, including, but not limited to, Streptomyces coelicolor (GenBank Nos: NP_628270, NC_003888) and B. subtilis (GenBank Nos: CAB14339, Z99116).

The term “valine decarboxylase” refers to an enzyme that catalyzes the conversion of L-valine to isobutylamine and CO₂. Example valine decarboxylases are known by the EC number 4.1.1.14. Such enzymes are found in Streptomyces, such as for example, Streptomyces viridifaciens (GenBank Nos: AAN10242, AY116644).

The term “omega transaminase” refers to an enzyme that catalyzes the conversion of isobutylamine to isobutyraldehyde using a suitable amino acid as an amine donor. Example omega transaminases are known by the EC number 2.6.1.18 and are available from a number of sources, including, but not limited to, Alcaligenes denitrificans (AAP92672, AY330220), Ralstonia eutropha (GenBank Nos: YP_294474, NC_007347), Shewanella oneidensis (GenBank Nos: NP_719046, NC_004347), and P. putida (GenBank Nos: AAN66223, AE016776).

The term “acetyl-CoA acetyltransferase” refers to an enzyme that catalyzes the conversion of two molecules of acetyl-CoA to acetoacetyl-CoA and coenzyme A (CoA). Example acetyl-CoA acetyltransferases are acetyl-CoA acetyltransferases with substrate preferences (reaction in the forward direction) for a short chain acyl-CoA and acetyl-CoA and are classified as E.C. 2.3.1.9 [Enzyme Nomenclature 1992, Academic Press, San Diego]; although, enzymes with a broader substrate range (E.C. 2.3.1.16) will be functional as well. Acetyl-CoA acetyltransferases are available from a number of sources, for example, Escherichia coli (GenBank Nos: NP_416728, NC_000913; NCBI (National Center for Biotechnology Information) amino acid sequence, NCBI nucleotide sequence), Clostridium acetobutylicum (GenBank Nos: NP_349476.1, NC_003030; NP_149242, NC_001988, Bacillus subtilis (GenBank Nos: NP_390297, NC_000964), and Saccharomyces cerevisiae (GenBank Nos: NP_015297, NC_001148).

The term “3-hydroxybutyryl-CoA dehydrogenase” refers to an enzyme that catalyzes the conversion of acetoacetyl-CoA to 3-hydroxybutyryl-CoA. 3-Example hydroxybutyryl-CoA dehydrogenases may be reduced nicotinamide adenine dinucleotide (NADH)-dependent, with a substrate preference for (S)-3-hydroxybutyryl-CoA or (R)-3-hydroxybutyryl-CoA. Examples may be classified as E.C. 1.1.1.35 and E.C. 1.1.1.30, respectively. Additionally, 3-hydroxybutyryl-CoA dehydrogenases may be reduced nicotinamide adenine dinucleotide phosphate (NADPH)-dependent, with a substrate preference for (S)-3-hydroxybutyryl-CoA or (R)-3-hydroxybutyryl-CoA and are classified as E.C. 1.1.1.157 and E.C. 1.1.1.36, respectively. 3-Hydroxybutyryl-CoA dehydrogenases are available from a number of sources, for example, C. acetobutylicum (GenBank NOs: NP_349314, NC_003030), B. subtilis (GenBank NOs: AAB09614, U29084), Ralstonia eutropha (GenBank NOs: YP_294481, NC_007347), and Alcaligenes eutrophus (GenBank NOs: AAA21973, J04987).

The term “crotonase” refers to an enzyme that catalyzes the conversion of 3-hydroxybutyryl-CoA to crotonyl-CoA and H₂O. Example crotonases may have a substrate preference for (S)-3-hydroxybutyryl-CoA or (R)-3-hydroxybutyryl-CoA and may be classified as E.C. 4.2.1.17 and E.C. 4.2.1.55, respectively. Crotonases are available from a number of sources, for example, E. coli (GenBank NOs: NP_415911, NC_000913), C. acetobutylicum (GenBank NOs: NP_349318, NC_003030), B. subtilis (GenBank NOs: CAB13705, Z99113), and Aeromonas caviae (GenBank NOs: BAA21816, D88825).

The term “butyryl-CoA dehydrogenase” refers to an enzyme that catalyzes the conversion of crotonyl-CoA to butyryl-CoA. Example butyryl-CoA dehydrogenases may be NADH-dependent, NADPH-dependent, or flavin-dependent and may be classified as E.C. 1.3.1.44, E.C. 1.3.1.38, and E.C. 1.3.99.2, respectively. Butyryl-CoA dehydrogenases are available from a number of sources, for example, C. acetobutylicum (GenBank NOs: NP_347102, NC_003030), Euglena gracilis (GenBank NOs: Q5EU90), AY741582), Streptomyces collinus (GenBank NOs: AAA92890, U37135), and Streptomyces coelicolor (GenBank NOs: CAA22721, AL939127).

The term “butyraldehyde dehydrogenase” refers to an enzyme that catalyzes the conversion of butyryl-CoA to butyraldehyde, using NADH or NADPH as cofactor. Butyraldehyde dehydrogenases with a preference for NADH are known as E.C. 1.2.1.57 and are available from, for example, Clostridium beijerinckii (GenBank NOs: AAD31841, AF157306) and C. acetobutylicum (GenBank NOs: NP_149325, NC_001988).

The term “isobutyryl-CoA mutase” refers to an enzyme that catalyzes the conversion of butyryl-CoA to isobutyryl-CoA. This enzyme uses coenzyme B₁₂ as cofactor. Example isobutyryl-CoA mutases are known by the EC number 5.4.99.13. These enzymes are found in a number of Streptomyces, including, but not limited to, Streptomyces cinnamonensis (GenBank Nos: AAC08713, U67612; CAB59633, AJ246005), S. coelicolor (GenBank Nos: CAB70645, AL939123; CAB92663, AL939121), and Streptomyces avermitilis (GenBank Nos: NP_824008, NC_003155; NP_824637, NC_003155).

The term “acetolactate decarboxylase” refers to a polypeptide (or polypeptides) having an enzyme activity that catalyzes the conversion of alpha-acetolactate to acetoin. Example acetolactate decarboxylases are known as EC 4.1.1.5 and are available, for example, from Bacillus subtilis (GenBank Nos: AAA22223, L04470), Klebsiella terrigena (GenBank Nos: AAA25054, L04507) and Klebsiella pneumoniae (GenBank Nos: AAU43774, AY722056).

The term “acetoin aminase” or “acetoin transaminase” refers to a polypeptide (or polypeptides) having an enzyme activity that catalyzes the conversion of acetoin to 3-amino-2-butanol. Acetoin aminase may utilize the cofactor pyridoxal 5′-phosphate or NADH (reduced nicotinamide adenine dinucleotide) or NADPH (reduced nicotinamide adenine dinucleotide phosphate). The resulting product may have (R) or (S) stereochemistry at the 3-position. The pyridoxal phosphate-dependent enzyme may use an amino acid such as alanine or glutamate as the amino donor. The NADH- and NADPH-dependent enzymes may use ammonia as a second substrate. A suitable example of an NADH dependent acetoin aminase, also known as amino alcohol dehydrogenase, is described by Ito et al. (U.S. Pat. No. 6,432,688). An example of a pyridoxal-dependent acetoin aminase is the amine:pyruvate aminotransferase (also called amine:pyruvate transaminase) described by Shin and Kim (J. Org. Chem. 67:2848-2853 (2002)).

The term “acetoin kinase” refers to a polypeptide (or polypeptides) having an enzyme activity that catalyzes the conversion of acetoin to phosphoacetoin. Acetoin kinase may utilize ATP (adenosine triphosphate) or phosphoenolpyruvate as the phosphate donor in the reaction. Enzymes that catalyze the analogous reaction on the similar substrate dihydroxyacetone, for example, include enzymes known as EC 2.7.1.29 (Garcia-Alles et al. (2004) Biochemistry 43:13037-13046).

The term “acetoin phosphate aminase” refers to a polypeptide (or polypeptides) having an enzyme activity that catalyzes the conversion of phosphoacetoin to 3-amino-2-butanol 0-phosphate. Acetoin phosphate aminase may use the cofactor pyridoxal 5′-phosphate, NADH or NADPH. The resulting product may have (R) or (S) stereochemistry at the 3-position. The pyridoxal phosphate-dependent enzyme may use an amino acid such as alanine or glutamate. The NADH and NADPH-dependent enzymes may use ammonia as a second substrate. Although there are no reports of enzymes catalyzing this reaction on phosphoacetoin, there is a pyridoxal phosphate-dependent enzyme that is proposed to carry out the analogous reaction on the similar substrate serinol phosphate (Yasuta et al. (2001) Appl. Environ. Microbial. 67:4999-5009.

The term “aminobutanol phosphate phospholyase”, also called “amino alcohol 0-phosphate lyase”, refers to a polypeptide (or polypeptides) having an enzyme activity that catalyzes the conversion of 3-amino-2-butanol 0-phosphate to 2-butanone. Amino butanol phosphate phospho-lyase may utilize the cofactor pyridoxal 5′-phosphate. There are reports of enzymes that catalyze the analogous reaction on the similar substrate 1-amino-2-propanol phosphate (Jones et al. (1973) Biochem 1 134:167-182). U.S. Patent Appl. Pub. No. 2007/0259410 describes an aminobutanol phosphate phospho-lyase from the organism Erwinia carotovora.

The term “aminobutanol kinase” refers to a polypeptide (or polypeptides) having an enzyme activity that catalyzes the conversion of 3-amino-2-butanol to 3-amino-2butanol 0-phosphate. Amino butanol kinase may utilize ATP as the phosphate donor. Although there are no reports of enzymes catalyzing this reaction on 3-amino-2-butanol, there are reports of enzymes that catalyze the analogous reaction on the similar substrates ethanolamine and 1-amino-2-propanol (Jones et al., supra). U.S. Patent Appl. Pub. No. 2009/0155870 describes, in Example 14, an amino alcohol kinase of Envinia carotovora subsp. Atroseptica.

The term “butanediol dehydrogenase” also known as “acetoin reductase” refers to a polypeptide (or polypeptides) having an enzyme activity that catalyzes the conversion of acetoin to 2,3-butanediol. Butanedial dehydrogenases are a subset of the broad family of alcohol dehydrogenases. Butanediol dehydrogenase enzymes may have specificity for production of (R)- or (S)-stereochemistry in the alcohol product. (S)-specific butanediol dehydrogenases are known as EC 1.1.1.76 and are available, for example, from Klebsiella pneumoniae (GenBank Nos: BBA13085, D86412). (R)-specific butanediol dehydrogenases are known as EC 1.1.1.4 and are available, for example, from Bacillus cereus (GenBank Nos. NP_830481, NC_004722; AAP07682, AE017000), and Lactococcus lactis (GenBank Nos. AAK04995, AE006323).

The term “butanediol dehydratase”, also known as “diol dehydratase” or “propanediol dehydratase” refers to a polypeptide (or polypeptides) having an enzyme activity that catalyzes the conversion of 2,3-butanediol to 2-butanone. Butanediol dehydratase may utilize the cofactor adenosyl cobalamin (also known as coenzyme B12 or vitamin B12; although vitamin B12 may refer also to other forms of cobalamin that are not coenzyme B12). Adenosyl cobalamin-dependent enzymes are known as EC 4.2.1.28 and are available, for example, from Klebsiella oxytoca (GenBank Nos: AA08099 (alpha subunit), D45071; BAA08100 (beta subunit), D45071; and BBA08101 (gamma subunit), D45071 (Note all three subunits are required for activity)], and Klebsiella pneumonia (GenBank Nos: AAC98384 (alpha subunit), AF102064; GenBank Nos: AAC98385 (beta subunit), AF102064, GenBank Nos: AAC98386 (gamma subunit), AF102064). Other suitable diol dehydratases include, but are not limited to, B12-dependent diol dehydratases available from Salmonella typhimurium (GenBank Nos: AAB84102 (large subunit), AF026270; GenBank Nos: AAB84103 (medium subunit), AF026270; GenBank Nos: AAB84104 (small subunit), AF026270); and Lactobacillus collinoides (GenBank Nos: CAC82541 (large subunit), AJ297723; GenBank Nos: CAC82542 (medium subunit); AJ297723; GenBank Nos: CAD01091 (small subunit), AJ297723); and enzymes from Lactobacillus brevis (particularly strains CNRZ 734 and CNRZ 735, Speranza et al., J. Agric. Food Chem. (1997) 45:3476-3480), and nucleotide sequences that encode the corresponding enzymes. Methods of diol dehydratase gene isolation are well known in the art (e.g., U.S. Pat. No. 5,686,276).

It will be appreciated that host cells comprising an engineered butanol biosynthetic pathway as provided herein may further comprise one or more additional modifications. In some embodiments, host cells contain a deletion or downregulation of a polynucleotide encoding a polypeptide that catalyzes the conversion of glyceraldehyde-3-phosphate to glycerate 1,3, bisphosphate. In some embodiments, the enzyme that catalyzes this reaction is glyceraldehyde-3-phosphate dehydrogenase. In some embodiments, the host cells comprise modifications to reduce glycerol-3-phosphate dehydrogenase activity and/or disruption in at least one gene encoding a polypeptide having pyruvate decarboxylase activity or a disruption in at least one gene encoding a regulatory element controlling pyruvate decarboxylase gene expression as described in U.S. Patent Appl. Pub. No. 2009/0305363 (incorporated herein by reference). In some embodiments, the host cells comprise modifications that provide for increased carbon flux through an Entner-Doudoroff Pathway or reducing equivalents balance as described in U.S. Patent Appl. Pub. No. 2010/0120105 (incorporated herein by reference). Other modifications include integration of at least one polynucleotide encoding a polypeptide that catalyzes a step in a pyruvate-utilizing biosynthetic pathway. Other modifications include at least one deletion, mutation, and/or substitution in an endogenous polynucleotide encoding a polypeptide having acetolactate reductase activity as described in PCT Publication No. WO 2011/159853 (incorporated herein by reference). In embodiments, the polypeptide having acetolactate reductase activity is YMR226C (SEQ ID NOs: 75) of Saccharomyces cerevisae or a homolog thereof. Additional modifications include a deletion, mutation, and/or substitution in an endogenous polynucleotide encoding a polypeptide having aldehyde dehydrogenase and/or aldehyde oxidase activity as described in PCT Publication No. WO 2011/159853 (incorporated herein by reference). In embodiments, the polypeptide having aldehyde dehydrogenase activity is ALD6 from Saccharomyces cerevisiae (SEQ ID NO: 76) or a homolog thereof.

Recombinant host cells may further comprise (a) at least one heterologous polynucleotide encoding a polypeptide having dihydroxy-acid dehydratase activity; and (b)(i) at least one deletion, mutation, and/or substitution in an endogenous gene encoding a polypeptide affecting Fe-S cluster biosynthesis; and/or (ii) at least one heterologous polynucleotide encoding a polypeptide affecting Fe-S cluster biosynthesis. In embodiments, the polypeptide affecting Fe—S cluster biosynthesis is encoded by AFT1, AFT2, FRA2, GRX3 or CCC1. AFT1 and AFT2 are described in WO 2001/103300, which is incorporated herein by reference. In embodiments, the polypeptide affecting Fe-S cluster biosynthesis is constitutive mutant AFT1 L99A, AFT1 L102A, AFT1 C291F, or AFT1 C293F.

### Butanol Production

Disclosed herein are processes suitable for production of butanol from a carbon substrate and employing a microorganism. In some embodiments, microorganisms may comprise an engineered butanol biosynthetic pathway, such as, but not limited to engineered isobutanol biosynthetic pathways disclosed elsewhere herein. The ability to utilize carbon substrates to produce isobutanol can be confirmed using methods known in the art, including, but not limited to those described in U.S. Pat. No. 7,851,188, which is incorporated herein by reference. For example, a specific high performance liquid chromatography (HPLC) method utilized a Shodex SH-1011 column with a Shodex SH-G guard column, both purchased from Waters Corporation (Milford, Mass.), with refractive index (RI) detection. Chromatographic separation was achieved using 0.01 M H₂SO₄ as the mobile phase with a flow rate of 0.5 mL/min and a column temperature of 50° C. Isobutanol had a retention time of 46.6 min under the conditions used. Alternatively, gas chromatography (GC) methods are available. For example, a specific GC method utilized an HP-INNOWax column (30 m×0.53 mm id, 1 μm film thickness, Agilent Technologies, Wilmington, Del.), with a flame ionization detector (FID). The carrier gas was helium at a flow rate of 4.5 mL/min, measured at 150° C. with constant head pressure; injector split was 1:25 at 200° C.; oven temperature was 45° C. for 1 min, 45 to 220° C. at 10° C./min, and 220° C. for 5 min; and FID detection was employed at 240° C. with 26 mL/min helium makeup gas. The retention time of isobutanol was 4.5 min.

One embodiment of the invention is directed to a microorganism comprising a pyruvate utilizing biosynthetic pathway, wherein the microorganism further comprises reduced pyruvate decarboxylase activity and modified adenylate cyclase activity. In a further embodiment, the pyruvate utilizing biosynthetic pathway is an engineered butanol production pathway. In some embodiments, the engineered butanol production pathway is an engineered isobutanol production pathway

In some embodiments, the engineered isobutanol production pathway comprises the following substrate to product conversions: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyraldehyde, and (e) isobutyraldehyde to isobutanol.

In some embodiments, the microorganism is a member of a genus of Saccharomyces, Schizosaccharomyces, Hansenula, Candida, Kluyveromyces, Yarrowia, Issatchenkia, or Pichia. In some embodiments, the microorganism is Saccharomyces cerevisiae.

In some embodiments, the engineered microorganism contains one or more polypeptides selected from a group of enzymes having the following Enzyme Commission Numbers: EC 2.2.1.6, EC 1.1.1.86, EC 4.2.1.9, EC 4.1.1.72, EC 1.1.1.1, EC 1.1.1.265, EC 1.1.1.2, EC 1.2.4.4, EC 1.3.99.2, EC 1.2.1.57, EC 1.2.1.10, EC 2.6.1.66, EC 2.6.1.42, EC 1.4.1.9, EC 1.4.1.8, EC 4.1.1.14, EC 2.6.1.18, EC 2.3.1.9, EC 2.3.1.16, EC 1.1.130, EC 1.1.1.35, EC 1.1.1.157, EC 1.1.1.36, EC 4.2.1.17, EC 4.2.1.55, EC 1.3.1.44, EC 1.3.1.38, EC 5.4.99.13, EC 4.1.1.5, EC 2.7.1.29, EC 1.1.1.76, EC 1.2.1.57, and EC 4.2.1.28.

In some embodiments, the engineered microorganism contains one or more polypeptides selected from acetolactate synthase, acetohydroxy acid isomeroreductase, acetohydroxy acid dehydratase, branched-chain alpha-keto acid decarboxylase, branched-chain alcohol dehydrogenase, acylating aldehyde dehydrogenase, branched-chain keto acid dehydrogenase, butyryl-CoA dehydrogenase, butyraldehyde dehydrogenase, transaminase, valine dehydrogenase, valine decarboxylase, omega transaminase, acetyl-CoA acetyltransferase, 3-hydroxybutyryl-CoA dehydrogenase, crotonase, butyryl-CoA dehydrogenase, isobutyryl-CoA mutase, acetolactate decarboxylase, acetonin aminase, butanol dehydrogenase, butyraldehyde dehydrogenase, acetoin kinase, acetoin phosphate aminase, aminobutanol phosphate phospholyase, aminobutanol kinase, butanediol dehydrogenase, and butanediol dehydratase.

In some embodiments, the engineered microorganism contains a polypeptide selected using a KARI Profile HMM. A KARI Profile HMI generated from the alignment of the twenty-five KARIs with experimentally verified function is given in U.S. Patent Appl. Pub. No. 2011/0313206, incorporated herein by reference. Suitable KARI enzymes include proteins that match the KARI Profile HMM with an E value of <10⁻³ using hmmsearch program in the HMMER package. The theory behind profile HMMs is described in R. Durbin, S. Eddy, A. Krogh, and G. Mitchison, Biological sequence analysis: probabilistic models of proteins and nucleic acids, Cambridge University Press, 1998; Krogh et al., J. Mol. Biol. 235: 1501-1531, 1994. Further, KARI enzymes that are a member of a clade identified through molecular phylogenetic analysis called the SLSL clade are described in U.S. Patent Appl. Pub. No. 2011/0244536, incorporated herein by reference. Additional suitable KARI enzymes are described in U.S. Patent Appl. Pub. Nos. 2008/0261230, 2009/0163376, and 2010/0197519, each incorporated herein by reference.

In some embodiments, the carbon substrate is selected from the group consisting of: oligosaccharides, polysaccharides, monosaccharides, and mixtures thereof. In some embodiments, the carbon substrate is selected from the group consisting of: fructose, glucose, lactose, maltose, galactose, sucrose, starch, cellulose, feedstocks, ethanol, lactate, succinate, glycerol, corn mash, sugar cane, biomass, a C5 sugar, such as xylose and arabinose, and mixtures thereof.

In some embodiments, one or more of the substrate to product conversions utilizes NADH or NADPH as a cofactor.

In some embodiments, enzymes from the biosynthetic pathway are localized to the cytosol. In some embodiments, enzymes from the biosynthetic pathway that are usually localized to the mitochondria are localized to the cytosol. In some embodiments, an enzyme from the biosynthetic pathway is localized to the cytosol by removing the mitochondrial targeting sequence. In some embodiments, mitochondrial targeting is eliminated by generating new start codons as described in e.g., U.S. Pat. No. 7,851,188, which is incorporated herein by reference in its entirety. In some embodiments, the enzyme from the biosynthetic pathway that is localized to the cytosol is DHAD. In some embodiments, the enzyme from the biosynthetic pathway that is localized to the cytosol is KARI.

In some embodiments, microorganisms are contacted with carbon substrates under conditions whereby a fermentation product is produced. In some embodiments, the fermentation product is butanol. In some embodiments, the butanol is isobutanol.

In some embodiments, the butanologen produces butanol at least 90% of effective yield, at least 91% of effective yield, at least 92% of effective yield, at least 93% of effective yield, at least 94% of effective yield, at least 95% of effective yield, at least 96% of effective yield, at least 97% of effective yield, at least 98% of effective yield, or at least 99% of effective yield. In some embodiments, the butanologen produces butanol at least 55% to at least 75% of effective yield, at least 50% to at least 80% of effective yield, at least 45% to at least 85% of effective yield, at least 40% to at least 90% of effective yield, at least 35% to at least 95% of effective yield, at least 30% to at least 99% of effective yield, at least 25% to at least 99% of effective yield, at least 10% to at least 99% of effective yield or at least 10% to 100% of effective yield.

### Microorganisms

In embodiments, suitable microorganisms include any microorganism useful for genetic modification and recombinant gene expression and that is capable of producing a C3-C6 alcohol by fermentation. In other embodiments, the microorganism is a butanologen. In other embodiments, the butanologen is a yeast host cell. In other embodiments, the yeast host cell can be a member of the genera Schizosaccharomyces, Issatchenkia, Kluyveromyces, Yarrowia, Pichia, Candida, Hansenula, or Saccharomyces. In other embodiments, the host cell can be Saccharomyces cerevisiae, Schizosaccharomyces pombe, Kluyveromyces lactis, Kluyveromyces thermotolerans, Kluyveromyces marxianus, Candida glabrata, Candida albicans, Pichia stipitis, or Yarrowia lipolytica. In some embodiments, the host cell is a member of the genera Saccharomyces. In some embodiments, the host cell is Kluyveromyces lactis, Candida glabrata or Schizosaccharomyces pombe. In some embodiments, the host cell is Saccharomyces cerevisiae. S. cerevisiae yeast are known in the art and are available from a variety of sources, including, but not limited to, American Type Culture Collection (Rockville, Md.), Centraalbureau voor Schimmelcultures (CBS) Fungal Biodiversity Centre, LeSaffre, Gert Strand AB, Ferm Solutions, North American Bioproducts, Martrex, and Lallemand. S. cerevisiae include, but are not limited to, BY4741, CEN.PK 113-7D, Ethanol Red® yeast, Ferm Pro™ yeast, Bio-Ferm® XR yeast, Gert Strand Prestige Batch Turbo alcohol yeast, Gert Strand Pot Distillers yeast, Gert Strand Distillers Turbo yeast, FerMax™ Green yeast, FerMax™ Gold yeast, Thermosacc® yeast, BG-1, PE-2, CAT-1, CBS7959, CBS7960, and CBS7961.

In some embodiments the microorganism is a diploid cell. In a further embodiment the organism is a MATa/MATa diploid, a MATα/MATα diploid, or a MATα/MATa diploid. In some embodiments the organism is a haploid. In a further embodiment the organism is a MATa haploid or a MATa haploid.

In some embodiments, the microorganism expresses an engineered C3-C6 alcohol production pathway. In some embodiments the microorganism is a butanologen that expresses an engineered butanol biosynthetic pathway. In some embodiments, the butanologen is an isobutanologen expressing an engineered isobutanol biosynthetic pathway.

### Carbon Substrates

Suitable carbon substrates may include, but are not limited to, monosaccharides such as fructose or glucose, oligosaccharides such as lactose, maltose, galactose, or sucrose, polysaccharides such as starch or cellulose or mixtures thereof and unpurified mixtures from renewable feedstocks such as cheese whey permeate, cornsteep liquor, sugar beet molasses, and barley malt. Other carbon substrates may include ethanol, lactate, succinate, or glycerol.

“Sugar” includes monosaccharides such as fructose or glucose, oligosaccharides such as lactose, maltose, galactose, or sucrose, polysaccharides such as starch or cellulose, C5 sugars such as xylose and arabinose, and mixtures thereof.

Additionally the carbon substrate may also be one-carbon substrates such as carbon dioxide, or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeasts are known to utilize the carbon from methylamine to form trehalose or glycerol (Bellion et al., Microb. Growth C1 Compd., [Int. Symp.], 7th (1993), 415-32, Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, in some embodiments, the carbon substrates are glucose, fructose, and sucrose, or mixtures of these with C5 sugars such as xylose and arabinose for yeasts cells modified to use C5 sugars. Sucrose may be derived from renewable sugar sources such as sugar cane, sugar beets, cassava, sweet sorghum, and mixtures thereof. Glucose and dextrose may be derived from renewable grain sources through saccharification of starch based feedstocks including grains such as corn, wheat, rye, barley, oats, and mixtures thereof. In addition, fermentable sugars may be derived from renewable cellulosic or lignocellulosic biomass through processes of pretreatment and saccharification, as described, for example, in U.S. Patent Application Publication No. 2007/0031918 A1, which is incorporated herein by reference. Biomass includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

In some embodiments, the carbon substrate is glucose derived from corn. In some embodiments, the carbon substrate is glucose derived from wheat. In some embodiments, the carbon substrate is sucrose derived from sugar cane.

In addition to an appropriate carbon source, fermentation media must contain suitable minerals, salts, cofactors, buffers and other components, known to those skilled in the art, suitable for the growth of the cultures and promotion of an enzymatic pathway described herein.

### Fermentation Conditions

Typically cells are grown at a temperature in the range of about 20° C. to about 40° C. in an appropriate medium. Suitable growth media in the present invention include common commercially prepared media such as Sabouraud Dextrose (SD) broth, Yeast Medium (YM) broth, or broth that includes yeast nitrogen base, ammonium sulfate, and dextrose (as the carbon/energy source) or YPD Medium, a blend of peptone, yeast extract, and dextrose in optimal proportions for growing most Saccharomyces cerevisiae strains. Other defined or synthetic growth media may also be used, and the appropriate medium for growth of the particular microorganism will be known by one skilled in the art of microbiology or fermentation science. The use of agents known to modulate catabolite repression directly or indirectly, e.g., cyclic adenosine 2′: 3′-monophosphate, may also be incorporated into the fermentation medium.

Suitable pH ranges for the fermentation are between pH 3.0 to pH 7.5, where pH 4.5 to pH 6.5 is preferred as the initial condition. Fermentations may be performed under aerobic or anaerobic conditions, where anaerobic or microaerobic conditions are preferred.

The amount of butanol produced in the fermentation medium can be determined using a number of methods known in the art, for example, high performance liquid chromatography (HPLC) or gas chromatography (GC).

### Industrial Batch and Continuous Fermentations

Isobutanol, or other products, may be produced using a batch method of fermentation. A classical batch fermentation is a closed system where the composition of the medium is set at the beginning of the fermentation and not subject to artificial alterations during the fermentation. A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Batch and fed-batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992).

Isobutanol, or other products, may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology and a variety of methods are detailed by Brock, supra.

It is contemplated that the production of isobutanol, or other products, may be practiced using batch, fed-batch or continuous processes and that any known mode of fermentation would be suitable. Additionally, it is contemplated that cells may be immobilized on a substrate as whole cell catalysts and subjected to fermentation conditions for isobutanol production.

Methods for Butanol Isolation from the Fermentation Medium

Bioproduced butanol may be isolated from the fermentation medium using methods known in the art for ABE fermentations (see, e.g., Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. Then, the isobutanol may be isolated from the fermentation medium using methods such as distillation, azeotropic distillation, liquid-liquid extraction, adsorption, gas stripping, membrane evaporation, or pervaporation.

Because butanol forms a low boiling point, azeotropic mixture with water, distillation can be used to separate the mixture up to its azeotropic composition. Distillation may be used in combination with another separation method to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer (see, e.g., Doherty and Malone, Conceptual Design of Distillation Systems, McGraw Hill, New York, 2001).

The butanol-water mixture forms a heterogeneous azeotrope so that distillation may be used in combination with decantation to isolate and purify the butanol. In this method, the butanol containing fermentation broth is distilled to near the azeotropic composition. Then, the azeotropic mixture is condensed, and the butanol is separated from the fermentation medium by decantation. The decanted aqueous phase may be returned to the first distillation column as reflux. The butanol-rich decanted organic phase may be further purified by distillation in a second distillation column.

The butanol can also be isolated from the fermentation medium using liquid-liquid extraction in combination with distillation. In this method, the butanol is extracted from the fermentation broth using liquid-liquid extraction with a suitable solvent. The butanol-containing organic phase is then distilled to separate the butanol from the solvent.

Distillation in combination with adsorption can also be used to isolate butanol from the fermentation medium. In this method, the fermentation broth containing the butanol is distilled to near the azeotropic composition and then the remaining water is removed by use of an adsorbent, such as molecular sieves (Aden et al., Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the butanol from the fermentation medium. In this method, the fermentation broth containing the butanol is distilled to near the azeotropic composition, and then the remaining water is removed by pervaporation through a hydrophilic membrane (Guo et al., J. Membr. Sci. 245, 199-210 (2004)).

In situ product removal (ISPR) (also referred to as extractive fermentation) can be used to remove butanol (or other fermentative alcohol) from the fermentation vessel as it is produced, thereby allowing the microorganism to produce butanol at high yields. One method for ISPR for removing fermentative alcohol that has been described in the art is liquid-liquid extraction. In general, with regard to butanol fermentation, for example, the fermentation medium, which includes the microorganism, is contacted with an organic extractant at a time before the butanol concentration reaches a toxic level. The organic extractant and the fermentation medium form a biphasic mixture. The butanol partitions into the organic extractant phase, decreasing the concentration in the aqueous phase containing the microorganism, thereby limiting the exposure of the microorganism to the inhibitory butanol.

Liquid-liquid extraction can be performed, for example, according to the processes described in U.S. Patent Appl. Pub. No. 2009/0305370, the disclosure of which is hereby incorporated in its entirety. U.S. Patent Appl. Pub. No. 2009/0305370 describes methods for producing and recovering butanol from a fermentation broth using liquid-liquid extraction, the methods comprising the step of contacting the fermentation broth with a water immiscible extractant to form a two-phase mixture comprising an aqueous phase and an organic phase. Typically, the extractant can be an organic extractant selected from the group consisting of saturated, mono-unsaturated, poly-unsaturated (and mixtures thereof) C₁₂ to C₂₂ fatty alcohols, C₁₂ to C₂₂ fatty acids, esters of C₁₂ to C₂₂ fatty acids, C₁₂ to C₂₂ fatty aldehydes, and mixtures thereof. The extractant(s) for ISPR can be non-alcohol extractants. The ISPR extractant can be an exogenous organic extractant such as oleyl alcohol, behenyl alcohol, cetyl alcohol, lauryl alcohol, myristyl alcohol, stearyl alcohol, 1-undecanol, oleic acid, lauric acid, myristic acid, stearic acid, methyl myristate, methyl oleate, undecanal, lauric aldehyde, 20-methylundecanal, and mixtures thereof.

In some embodiments, an ester can be formed by contacting the alcohol in a fermentation medium with an organic acid (e.g., fatty acids) and a catalyst capable of esterifying the alcohol with the organic acid. In such embodiments, the organic acid can serve as an ISPR extractant into which the alcohol esters partition. The organic acid can be supplied to the fermentation vessel and/or derived from the biomass supplying fermentable carbon fed to the fermentation vessel. Lipids present in the feedstock can be catalytically hydrolyzed to organic acid, and the same catalyst (e.g., enzymes) can esterify the organic acid with the alcohol. The catalyst can be supplied to the feedstock prior to fermentation, or can be supplied to the fermentation vessel before or contemporaneously with the supplying of the feedstock. When the catalyst is supplied to the fermentation vessel, alcohol esters can be obtained by hydrolysis of the lipids into organic acid and substantially simultaneous esterification of the organic acid with butanol present in the fermentation vessel. Organic acid and/or native oil not derived from the feedstock can also be fed to the fermentation vessel, with the native oil being hydrolyzed into organic acid. Any organic acid not esterified with the alcohol can serve as part of the ISPR extractant. The extractant containing alcohol esters can be separated from the fermentation medium, and the alcohol can be recovered from the extractant. The extractant can be recycled to the fermentation vessel. Thus, in the case of butanol production, for example, the conversion of the butanol to an ester reduces the free butanol concentration in the fermentation medium, shielding the microorganism from the toxic effect of increasing butanol concentration. In addition, unfractionated grain can be used as feedstock without separation of lipids therein, since the lipids can be catalytically hydrolyzed to organic acid, thereby decreasing the rate of build-up of lipids in the ISPR extractant. Other butanol product recovery and/or ISPR methods may be employed, including those described in U.S. Pat. No. 8,101,808, incorporated herein by reference.

In situ product removal can be carried out in a batch mode or a continuous mode. In a continuous mode of in situ product removal, product is continually removed from the reactor. In a batchwise mode of in situ product removal, a volume of organic extractant is added to the fermentation vessel and the extractant is not removed during the process. For in situ product removal, the organic extractant can contact the fermentation medium at the start of the fermentation forming a biphasic fermentation medium. Alternatively, the organic extractant can contact the fermentation medium after the microorganism has achieved a desired amount of growth, which can be determined by measuring the optical density of the culture. Further, the organic extractant can contact the fermentation medium at a time at which the product alcohol level in the fermentation medium reaches a preselected level. In the case of butanol production according to some embodiments of the present invention, the organic acid extractant can contact the fermentation medium at a time before the butanol concentration reaches a toxic level, so as to esterify the butanol with the organic acid to produce butanol esters and consequently reduce the concentration of butanol in the fermentation vessel. The ester-containing organic phase can then be removed from the fermentation vessel (and separated from the fermentation broth which constitutes the aqueous phase) after a desired effective titer of the butanol esters is achieved. In some embodiments, the ester-containing organic phase is separated from the aqueous phase after fermentation of the available fermentable sugar in the fermentation vessel is substantially complete.

Butanol titer in any phase can be determined by methods known in the art, such as via high performance liquid chromatography (HPLC) or gas chromatography, as described, for example, in U.S. Patent Appl. Pub. No. 2009/0305370, which is incorporated herein by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “s” means second(s), “min” means minute(s), “h” means hour(s), “psi” means pounds per square inch, “nm” means nanometers, “d” means day(s), “μL” means microliter(s), “mL” means milliliter(s), “L” means liter(s), “mm” means millimeter(s), “nm” means nanometers, “mM” means millimolar, “μM” means micromolar, “M” means molar, “mmol” means millimole(s), “μmol” means micromole(s)”, “g” means gram(s), “μg” means microgram(s) and “ng” means nanogram(s), “PCR” means polymerase chain reaction, “OD” means optical density, “OD₆₀₀” means the optical density measured at a wavelength of 600 nm, “cfu” means colony forming units, “kDa” means kilodaltons, “g” means the gravitation constant, “bp” means base pair(s), “kb” means kilobase pair(s), “% w/v” means weight/volume percent, % v/v″ means volume/volume percent, “HPLC” means high performance liquid chromatography, and “GC” means gas chromatography

### General Methods

Materials and methods suitable for the maintenance and growth of yeast cultures are well known in the art. Techniques suitable for use in the following Examples may be found as set out in Yeast Protocols, Second Edition (Wei Xiao, ed; Humana Press, Totowa, N.J. (2006))). All reagents were obtained from Aldrich Chemicals (Milwaukee, Wis.), BD Diagnostic Systems (Sparks, Md.), Life Technologies (Rockville, Md.), Sigma Chemical Company (St. Louis, Mo.), or Teknova (Half Moon Bay, Calif.) unless otherwise specified.

YPD contains per liter: 10 g yeast extract, 20 g peptone, and 20 g dextrose. YPE contains per liter: 10 g yeast extract, 20 g peptone, and 1% ethanol.

The oligonucleotide primers to use in the following Examples are given in Table 5. All the oligonucleotide primers are synthesized by Sigma-Genosys (Woodlands, Tex.).

The strains referenced in the following Examples are given in Table 6.

TABLE 5
Primers

| SEQ | | |
|---|---|---|
| ID NO: | | |
| 77 | oBP622 | AATTGGTACCCCAAAAGGAATATTGGGTCAGA |
| 78 | oBP623 | CCATTGTTTAAACGGCGCGCCGGATCCTTTGCGAAACCCTAT |
| | | GCTCTGT |
| 79 | oBP624 | GCAAAGGATCCGGCGCGCCGTTTAAACAATGGAAGGTCGGG |
| | | ATGAGCAT |
| 80 | oBP625 | AATTGGCCGGCCTACGTAACATTCTGTCAACCAA |
| 81 | oBP626 | AATTGCGGCCGCTTCATATATGACGTAATAAAAT |
| 82 | oBP627 | AATTTTAATTAATTTTTTTTCTTGGAATCAGTAC |
| 83 | HY21 | TTAAGGCGCGCCTATTTGTAATACGTATACGAATTCCTTC |
| 84 | HY24 | ACTTAATAACTTTACCGGCTGTTGACATTTTGTTCTTCTTGTT |
| | | ATTGTATTGTGTT |
| 85 | HY25 | AACACAATACAATAACAAGAAGAACAAAATGTCAACAGCCG |
| | | GTAAAGTTATTAAGT |
| 86 | HY4 | GGAAGTTTAAACACCACAGGTGTTGTCCTCTGAGGACATA |
| 87 | URA3-end F | GCATATTTGAGAAGATGCGGCCAGCAAAAC |
| 88 | oBP636 | CATTTTTTTCCCTCTAAGAAGC |
| 89 | oBP637 | TTTTTGCACAGTTAAACTACCC |
| 90 | oBP691 | AATTGGATCCGCGATCGCGACGTTCTCTCCGTTGTTCAAA |
| 91 | oBP692 | AATTGGCGCGCCATTTAAATATATATGTATATATATAACAC |
| 92 | oBP693 | AATTGTTTAAACAAAGGATGATATTGTTCTATTA |
| 93 | oBP694 | AATTGGCCGGCCGCAACGACGACAATGCCAAAC |
| 94 | oBP695 | AATTGCGGCCGCATGACAGGTGAAAGAATTGAAA |
| 95 | oBP696 | AATTTTAATTAAACGGGCATCTTATAGTGTCGTT |
| 96 | HY16 | TTAAGGCGCGCCCCGCACGCCGAAATGCATGCAAGTAACC |
| 97 | HY19 | ACTTAATAACTTTACCGGCTGTTGACATTTTGATTGATTTGAC |
| | | TGTGTTATTTTGC |
| 98 | HY20 | GCAAAATAACACAGTCAAATCAATCAAAATGTCAACAGCCG |
| | | GTAAAGTTATTAAGT |
| 99 | oBP730 | TTGCTCCAAAGAGATGTCTTTA |
| 100 | oBP731 | TGTTCCCACAATCTATTACCTA |
| 101 | BK505 | TTCCGGTTTCTTTGAAATTTTTTTGATTCGGTAATCTCCGAGC |
| | | AGAAGGAGCATTGCGGA |
| | | TTACGTATTCTAATGTTCAG |
| 102 | BK506 | GGGTAATAACTGATATAATTAAATTGAAGCTCTAATTTGTGA |
| | | GTTTAGTACACCTTGGCT |
| | | AACTCGTTGTATCATCACTGG |
| 103 | LA468 | GCCTCGAGTTTTAATGTTACTTCTCTTGCAGTTAGGGA |
| 104 | LA492 | GCTAAATTCGAGTGAAACACAGGAAGACCAG |
| 105 | AK109-1 | AGTCACATCAAGATCGTTTATGG |
| 106 | AK109-2 | GCACGGAATATGGGACTACTTCG |
| 107 | AK109-3 | ACTCCACTTCAAGTAAGAGTTTG |
| 108 | oBP452 | TTCTCGACGTGGGCCTTTTTCTTG |
| 109 | oBP453 | TGCAGCTTTAAATAATCGGTGTCACTACTTTGCCTTCGTTTAT |
| | | CTTGCC |
| 110 | oBP454 | GAGCAGGCAAGATAAACGAAGGCAAAGTAGTGACACCGATT |
| | | ATTTAAAG |
| 111 | oBP455 | TATGGACCCTGAAACCACAGCCACATTGTAACCACCACGAC |
| | | GGTTGTTG |
| 112 | oBP456 | TTTAGCAACAACCGTCGTGGTGGTTACAATGTGGCTGTGGTT |
| | | TCAGGGT |
| 113 | oBP457 | CCAGAAACCCTATACCTGTGTGGACGTAAGGCCATGAAGCTT |
| | | TTTCTTT |
| 114 | oBP458 | ATTGGAAAGAAAAAGCTTCATGGCCTTACGTCCACACAGGT |
| | | ATAGGGTT |
| 115 | oBP459 | CATAAGAACACCTTTGGTGGAG |
| 116 | oBP460 | AGGATTATCATTCATAAGTTTC |
| 117 | LA135 | CTTGGCAGCAACAGGACTAG |
| 118 | oBP461 | TTCTTGGAGCTGGGACATGTTTG |
| 119 | LA92 | GAGAAGATGCGGCCAGCAAAAC |
| 120 | LA678 | CAACGTTAACACCGTTTTCGGTTTGCCAGGTGACTTCAACTT |
| | | GTCCTTGTGCATTGCGGA |
| | | TTACGTATTCTAATGTTCAG |
| 121 | LA679 | GTGGAGCATCGAAGACTGGCAACATGATTTCAATCATTCTGA |
| | | TCTTAGAGCACCTTGGCT |
| | | AACTCGTTGTATCATCACTGG |
| 122 | LA337 | CTCATTTGAATCAGCTTATGGTG |
| 123 | LA692 | GGAAGTCATTGACACCATCTTGGC |
| 124 | LA693 | AGAAGCTGGGACAGCAGCGTTAGC |
| 125 | LA722 | TGCCAATTATTTACCTAAACATCTATAACCTTCAAAAGTAAA |
| | | AAAATACACAAACGTTGA |
| | | ATCATCACCTTGGCTAACTCGTTGTATCATCACTGG |
| 126 | LA733 | CATAATCAATCTCAAAGAGAACAACACAATACAATAACAAG |
| | | AAGAACAAAGCATTGCGGATTACGTATTCTAATGTTCAG |
| 127 | LA453 | CACCGAAGAAGAATGCAAAAATTTCAGCTC |
| 128 | LA694 | GCTGAAGTTGTTAGAACTGTTGTTG |
| 129 | LA695 | TGTTAGCTGGAGTAGACTTGG |
| 130 | oBP594 | AGCTGTCTCGTGTTGTGGGTTT |
| 131 | oBP595 | CTTAATAATAGAACAATATCATCCTTTACGGGCATCTTATAG |
| | | TGTCGTT |
| 132 | oBP596 | GCGCCAACGACACTATAAGATGCCCGTAAAGGATGATATTG |
| | | TTCTATTA |
| 133 | oBP597 | TATGGACCCTGAAACCACAGCCACATTGCAACGACGACAAT |
| | | GCCAAACC |
| 134 | oBP598 | TCCTTGGTTTGGCATTGTCGTCGTTGCAATGTGGCTGTGGTTT |
| | | CAGGGT |
| 135 | oBP599 | ATCCTCTCGCGGAGTCCCTGTTCAGTAAAGGCCATGAAGCTT |
| | | TTTCTTT |
| 136 | oBP600 | ATTGGAAAGAAAAAGCTTCATGGCCTTTACTGAACAGGGAC |
| | | TCCGCGAG |
| 137 | oBP601 | TCATACCACAATCTTAGACCAT |
| 138 | oBP602 | TGTTCAAACCCCTAACCAACC |
| 139 | oBP603 | TGTTCCCACAATCTATTACCTA |
| 140 | LA512 | GTATTTTGGTAGATTCAATTCTCTTTCCCTTTCCTTTTCCTTCG |
| | | CTCCCCTTCCTTATCAGCATTGCGGATTACGTATTCTAATGTT |
| | | CAG |
| 141 | LA513 | TTGGTTGGGGGAAAAAGAGGCAACAGGAAAGATCAGAGGG |
| | | GGAGGGGGGGGGAGAGTGTCACCTTGGCTAACTCGTTGTAT |
| | | CATCACTGG |
| 142 | LA516 | CTCGAAACAATAAGACGACGATGGCTCTG |
| 143 | LA514 | CACTATCTGGTGCAAACTTGGCACCGGAAG |
| 144 | LA515 | TGTTTGTAGCCACTCGTGAACTTCTCTGC |
| 145 | LA829 | CCAAATTTACAATATCTCCTGAATTCTTGGCTTGGAATATGG |
| | | GCAGTACAGCTTGTGTGA |
| | | TATTGCACCTTGGCTAACTCGTTGTATCATCACTGG |
| 146 | LA834 | ATGTCCCAAGGTAGAAAAGCTGCAGAAAGATTGGCTAAGAA |
| | | GACTGTCCTCATTACAGGTGATCTGAAATGAATAACAATACT |
| | | GACAGTA |
| 147 | N1257 | GATGATGCTATTTGGTGCAGAGGGTGATG |
| 148 | LA740 | CGATAATCCTGCTGTCATTATC |
| 149 | LA830 | CACGGCAAACTTAGAGGCACAATAGATAG |
| 150 | LA850 | ATGACTAAGCTACACTTTGACACTGCTGAACCAGTCAAGATC |
| | | ACACTTCCAAATGGTTTG |
| | | ACATAAATTACCGTCGCTCGTGATTTGTTTGC |
| 151 | LA851 | TTACAACTTAATTCTGACAGCTTTTACTTCAGTGTATGCATGG |
| | | TAGACTTCTTCACCCAT |
| | | TTCCACCTTGGCTAACTCGTTGTATCATCACTGG |
| 152 | N1262 | CACGTAAGGGCATGATAGAATTGG |
| 153 | N1263 | GGATATAGCAGTTGTTGTACACTAGC |
| 154 | LA855 | GCACAATATTTCAAGCTATACCAAGCATACAATCAACTATCT |
| | | CATATACAACCTGGTAAA |
| | | ACCTCTAGTGGAGTAGTAGA |
| 155 | LA856 | GCTTATTTAGAAGTGTCAACAACGTATCTACCAACGATTTGA |
| | | CCCTTTTCCACACCTTGG |
| | | CTAACTCGTTGTATCATCACTGG |
| 156 | LA414 | CCAGAGCTGATGAGGGGTATCTCGA |
| 157 | LA749 | CAAGTCTTTTGTGCCTTCCCGTCGG |
| 158 | LA413 | GGACATAAAATACACACCGAGATTC |
| 159 | LA860 | TCTCAATTATTATTTTCTACTCATAACCTCACGCAAAATAACA |
| | | CAGTCAAATCAATCAAA |
| | | ATGAAAGCATTAGTGTATAGGGGCCCAGGC |
| 160 | LA679 | GTGGAGCATCGAAGACTGGCAACATGATTTCAATCATTCTGA |
| | | TCTTAGAGCACCTTGGCT |
| | | AACTCGTTGTATCATCACTGG |
| 161 | LA337 | CTCATTTGAATCAGCTTATGGTG |
| 162 | N1093 | TTTCAAGATGCAAATCAACTTTGCTA |
| 163 | LA681 | TTATTGCTTAGCGTTGGTAG |
| 170 | LA811 | AACGAAGCATCTGTGCTTCATTTTGTAGAAC |
| 171 | LA817 | CGATCCACTTGTATATTTGGATGAATTTTTGAGGAATTCTGA |
| | | ACCAGTCCTAAAACGAG |
| 172 | LA812 | AACAAAGATATGCTATTGAATGCAAGATGG |
| 173 | LA818 | CTCAAAAATTCATCCAAATAACAAGTGGATCG |
| 176 | LA92 | GAGAAGATGCGGCCAGCAAAAC |
| 183 | AK09-1_MAT | AGTCACATCAAGATCGTTTATGG |
| 184 | AK09-2_HML | GCACGGAATATGGGACTACTTCG |
| 185 | AK09-03_HMR | ACTCCACTTCAAGTAAGAGTTTG |
| 186 | 315 | CTTCGAAGAATATACTAAAAAATGAGCAGGCAAGATAAACG |
| | | AAGGCAAAGGCATTGCGGATTACGTATTCTAATGTTCAG |
| 187 | 316 | TATACACATGTATATATATCGTATGCTGCAGCTTTAAATAAT |
| | | CGGTGTCACACCTTGGCTAACTCGTTGTATCATCACTGG |
| 188 | 92 | GAGAAGATGCGGCCAGCAAAAC |
| 189 | 346 | GGAATACCACTTGCCACCTATCACC |
| 190 | oBP440 | TACGTACGGACCAATCGAAGTG |
| 191 | oBP441 | AATTCGTTTGAGTACACTACTAATGGCTTTGTTGGCAATATG |
| | | TTTTTGC |
| 192 | oBP442 | ATATAGCAAAAACATATTGCCAACAAAGCCATTAGTAGTGT |
| | | ACTCAAAC |
| 193 | oBP443 | TATGGACCCTGAAACCACAGCCACATTCTTGTTATTTATAAA |
| | | AAGACAC |
| 194 | oBP444 | CTCCCGTGTCTTTTTATAAATAACAAGAATGTGGCTGTGGTTT |
| | | CAGGGT |
| 195 | oBP445 | TACCGTAGGCGTCCTTAGGAAAGATAGAAGGCCATGAAGCT |
| | | TTTTCTTT |
| 196 | oBP446 | ATTGGAAAGAAAAAGCTTCATGGCCTTCTATCTTTCCTAAGG |
| | | ACGCCTA |
| 197 | oBP447 | TTATTGTTTGGCATTTGTAGC |
| 198 | oBP448 | CCAAGCATCTCATAAACCTATG |
| 199 | oBP449 | TGTGCAGATGCAGATGTGAGAC |
| 200 | oBP554 | AGTTATTGATACCGTAC |
| 201 | oBP555 | CGAGATACCGTAGGCGTCC |
| 202 | oBP513 | TTATGTATGCTCTTCTGACTTTTC |
| 203 | oBP515 | AATAATTAGAGATTAAATCGCTCATTTTTTGCCAGTTTCTTCA |
| | | GGCTTC |
| 204 | oBP516 | AGCCTGAAGAAACTGGCAAAAAATGAGCGATTTAATCTCTA |
| | | ATTATTAG |
| 205 | oBP517 | TATGGACCCTGAAACCACAGCCACATTTTTCAATCATTGGAG |
| | | CAATCAT |
| 206 | oBP518 | TAAAATGATTGCTCCAATGATTGAAAAATGTGGCTGTGGTTT |
| | | CAGGGTC |
| 207 | oBP519 | ACCGTAGGTGTTGTTTGGGAAAGTGGAAGGCCATGAAGCTTT |
| | | TTCTTTC |
| 208 | oBP520 | TTGGAAAGAAAAAGCTTCATGGCCTTCCACTTTCCCAAACAA |
| | | CACCTAC |
| 209 | oBP521 | TTATTGCTTAGCGTTGGTAGCAG |
| 210 | oBP550 | GTCATTGACACCATCT |
| 211 | oBP551 | AGAGATACCGTAGGTGTTG |
| 212 | ilvDSm(1354F) | GGACCAAAGGGCGGTCCTGGTATGCCTG |
| 213 | oBP512 | AAAGTTGGCATAGCGGAAACTT |
| 214 | ilvDsm(788R) | GCTTCACGCGTTAAAATGTCAGAAGG |
| 215 | MAT1 | AGTCACATCAAGATCGTTTATGG |
| 216 | MAT2 | GCACGGAATATGGGCATACTTCG |
| 217 | MAT3 | ACTCCACTTCAAGTAAGAGTTTG |
| 218 | BP448 | CCAAGCATCTCATAAACCTATG |
| 219 | BP449 | TGTGCAGATGCAGATGTGAGAC |
| 220 | T-A(PDC5) | CTGTCGCTAACACCTGTATGGTTGCAACC |
| 221 | B-A(kivD) | GATAGTCACCTACTGTATACATTTTGTTCTTCTTGTTATTGTA |
| | | TTGTG |
| 222 | T-kivD(A) | ACACAATACAATAACAAGAAGAACAAAATGTATACAGTAGG |
| | | TGACTATCTGTTGGAC |
| 223 | BkivD(B) | TCAGGCAGCGCCTGCGTTCGAGTCAGCTCTTGTTTTGTTCTGC |
| | | AAATAACTTACCC |
| 224 | T-B(kivD) | ATTTGCAGAACAAAACAAGAGCTGACTCGAACGCAGGCGCT |
| | | GCCTGA |
| 225 | oBP546 | AGCGTATACATCTGTTGGGAAAGTAGAAGGCCATGAAGCTTT |
| | | TTCTTTC |
| 226 | oBP547 | TTGGAAAGAAAAAGCTTCATGGCCTTCTACTTTCCCAACAGA |
| | | TGTATAC |
| 227 | pBP539 | TTATTGTTTAGCGTTAGTAGCG |
| 228 | oBP540 | TAGGCATAATCACCGAAGAAG |
| 229 | kivD(652R) | CTGAGTAACAGTCTTCTCTAGGCCGAACG |
| 230 | oBP552 | AGTTGTTAGAACTGTTG |
| 231 | oBP553 | GACGATAGCGTATACATCT |
| 232 | kivD(602F) | CAAGAGATTCTGAACAAAATACAGGAAAG |
| 233 | kivD(1250F) | CCCCGCAGCTCTAGGCAGCCAAATTGC |
| 234 | JZ067 | CGTCGTGAAGGCAGTTTAGTTCTCGGACTTGC |
| 235 | JZ088 | CTTTTTGCAAACAAATCACGAGCGACGGTAATTTTTTGGCCA |
| | | AATGCCACAGCCGATCTGC |
| 236 | JZ087 | GCAGATCGGCTGTGGCATTTGGCCAAAAAATTACCGTCGCTC |
| | | GTGATTTGTTTGCAAAAAG |
| 237 | JZ068 | AATAATTCGTTTGAGTACACTACTAATGGCACCACAGGTGTT |
| | | GTCCTCTGAGGAC |
| 238 | JZ069 | GTCCTCAGAGGACAACACCTGTGGTGCCATTAGTAGTGTACT |
| | | CAAACGAATTATT |
| 239 | JZ070 | GGACCCTGAAACCACAGCCACATTAACTTGTTATTTATAAAA |
| | | AGACACGGGAGG |
| 240 | JZ071 | CCTCCCGTGTCTTTTTATAAATAACAAGTTAATGTGGCTGTG |
| | | GTTTCAGGGTCC |
| 241 | JZ072 | GTGAATAAGGTGTGAACTCTATAACAAAGGCCATGAAGCTTT |
| | | TTCTTTCCAATT |
| 242 | JZ073 | AATTGGAAAGAAAAAGCTTCATGGCCTTTGTTATAGAGTTCA |
| | | CACCTTATTCAC |
| 243 | JZ074 | TTTGTTGGCAATATGTTTTTGCTATATTACG |
| 244 | JZ061 | GAGAGCTGCTCAACGCGGAATGGAGATAACGG |
| 245 | JZ060 | CCTTCACTATAGCGTCACCAGGTTCC |
| 246 | JZ062 | GGTAAATAAATGTGCAGATGCAGATGTGAGAC |
| 247 | 643R | CGGCTGCGGCGTTACCACCCGTGGAG |
| 248 | T-HIS3(up300) | TTGGTGAGCGCTAGGAGTCACTGCCAGG |
| 249 | B- | CGGAATACCACTTGCCACCTATCACCAC |
| | HIS3(down273) | |
| 250 | JZ151 | AAGATTCTGTCCAGAAACAACATCAACATCGC |
| 251 | JZ317 | GTTGAAGGAATTCGTATACGTATTACAAATATATCAAAATAC |
| | | GTTCTCAATGTTCTATTTCC |
| 252 | JZ316 | GGAAATAGAACATTGAGAACGTATTTTGATATATTTGTAATA |
| | | CGTATACGAATTCCTTCAAC |
| 253 | JZ313 | GTATACAGATTTACTTAGTTTAGCTAGGTCCGCAAATTAAAG |
| | | CCTTCGAGCGTCCCAAAAC |
| 254 | JZ312 | GTTTTGGGACGCTCGAAGGCTTTAATTTGCGGACCTAGCTAA |
| | | ACTAAGTAAATCTGTATAC |
| 255 | JZ157 | TTATGGACCCTGAAACCACAGCCACATTAAAGAGGCTTGACT |
| | | TTATTGTAATCTGAGA |
| 256 | JZ156 | TCTCAGATTACAATAAAGTCAAGCCTCTTTAATGTGGCTGTG |
| | | GTTTCAGGGTCCATAA |
| 257 | JZ159 | GTCACTGCCAAGAGCCTTTCCGGCATAAGGCCATGAAGCTTT |
| | | TTCTTTCCAATT |
| 258 | JZ158 | AATTGGAAAGAAAAAGCTTCATGGCCTTATGCCGGAAAGGC |
| | | TCTTGGCAGTGAC |
| 259 | JZ160 | TTATCCACGGAAGATATGATGAGGTGACGCTTG |
| 260 | URA3F | GCATATTTGAGAAGATGCGGCCAGCAAAAC |
| 261 | JZ161 | AACATATGTTTGAGATCCAGCTGTTTCGAGTGACG |
| 262 | URA3R | CTGTGCTCCTTCCTTCGTTCTTCCTTCTGCTCGGAG |
| 263 | JZ320 | CGTAAACCTGCATTAAGGTAAGATTATATC |
| 264 | JZ150 | GAACGAACTAGAGACCACCCTGGCCCATACCAAG |
| 265 | JZ319 | CGATATCGGTTCGCACGCCATTTGGATGTCAC |
| 266 | B-A(kivDLg) | CTGTCCTACGGTATACATTTTGTTCTTCTTGTTATTGTATTGT |
| | | G |
| 267 | T-kivDLg(A) | ACACAATACAATAACAAGAAGAACAAAATGTATACCGTAGG |
| | | ACAGTACTTGG |
| 268 | B-kivDLg(B) | TCAGGCAGCGCCTGCGTTCGAGTTAAGAGTTTTGCTTAGATA |
| | | AGGCTAAGCC |
| 269 | T-B(kivDLg) | TTATCTAAGCAAAACTCTTAACTCGAACGCAGGCGCTGCCTG |
| | | A |
| 270 | oBP546(new) | GTATCCTATAGATCCCCACAAAAGGCCATGAAGCTTTTTCTT |
| | | TC |
| 271 | oBP547(new) | AAGAAAAAGCTTCATGGCCTTTTGTGGGGATCTATAGGATAC |
| | | ACTTTCC |
| 272 | oBP539(new) | TCAGCTCTTGTTTTGTTCTGCAAATAAC |
| 273 | kivDLg(569R) | GTGTGATAGTATGATTTCTGCAAGTTGTGCC |
| 274 | kivDLg(530F) | GCTCATAAAGCAATAGTTAAACCTGC |
| 275 | kivDLg(1162F) | GGGGACATCATCTTTCGGTTTGATGTTGG |
| 286 | HY31 | GCCGACTTTATGGCGAAGAAGTTTGCTCTTGATC |
| 287 | oBP511 | TTTTTGGTGGTTCCGGCTTCC |

TABLE 6
Strains referenced in the Examples

| Strain Name | Genotype | Description |
|---|---|---|
| PNY2211 | MATa ura3Δ::loxP his3Δ pdc6Δ | PCT Publication No. |
| | pdc1Δ::P[PDC1]-DHAD\|ilvD_Sm-PDC1t- | WO2012033832, |
| | P[FBA1]-ALS\|alsS_Bs-CYC1t | incorporated herein by |
| | pdc5Δ::P[PDC5]-ADH\|sadB_Ax-PDC5t | reference |
| | gpd2Δ::loxP fra2Δ | |
| | adh1Δ::UAS(PGK1)P[FBA1]-kivD_Ll(y)- | |
| | ADH1t | |
| PNY1528 | MATa ura3Δ::loxP his3Δ pdc6Δ | Herein |
| | pdc1Δ::P[PDC1]-DHAD\|ilvD_Sm-PDC1t- | |
| | P[FBA1]-ALS\|alsS_Bs-CYC1t | |
| | pdc5Δ::P[PDC5]-ADH\|sadB_Ax-PDC5t | |
| | gpd2Δ::loxP fra2Δ::P[PDC1]-ADH\|adh_Hl- | |
| | ADH1t adh1Δ::UAS(PGK1)P[FBA1]- | |
| | kivD_Ll(y)-ADH1t yprcΔ15Δ::P[PDC5]- | |
| | ADH\|adh_Hl-ADH1t | |
| PNY1530 | PNY1528 with plasmid pYZ107F-OLE1p | Herein |
| | containing (P[ILV5]-KARI\|ilvC_Ll-ILV5t | |
| | P[OLE1]-DHAD\|ilvD_Sm-FBA1t) | |
| PNY2242 | MATa ura3Δ::loxP his3Δ pdc6Δ | U.S. Patent Appl. Pub. |
| | pdc1Δ::P[PDC1]-DHAD\|ilvD_Sm-PDC1t- | No. 2013/0071891, |
| | P[FBA1]-ALS\|alsS_Bs-CYC1t | incorporated herein by |
| | pdc5Δ::P[PDC5]-ADH\|sadB_Ax-PDC5t | reference |
| | gpd2Δ::loxP fra2Δ::P[PDC1]-ADH\|adh_Hl- | |
| | ADH1t adh1Δ::UAS(PGK1)P[FBA1]- | |
| | kivD_Ll(y)-ADH1t yprcΔ15Δ::P[PDC5]- | |
| | ADH\|adh_Hl-ADH1t ymr226cΔ ald6Δ::loxP; | |
| | pLH702, pYZ067DkivDDhADH | |
| PNY2068 | MATa ura3Δ::loxP-kanMX4-loxP his3Δ | Herein |
| | pdc1Δ::loxP71/66 pdc5Δ::loxP71/66 fra2Δ 2μ | |
| | plasmid (CEN.PK2) gpd2Δ | |
| | ymr226CΔ::P[FBA1]-ALS\|alsS_Bs-CYC1t- | |
| | loxP71/66 ald6Δ::UAS(PGK1)P[FBA1]- | |
| | KIVD\|Lg(y)-TDH3t-loxP71/66 | |
| | adh1Δ::P[ILV5]-ADH\|Bi(y)-ADHt-loxP71/66 | |
| | pdc1Δ::P[PDC1]-ADH\|Bi(y)-ADHt-loxP71/66 | |
| PNY2071 | MATa ura3Δ::loxP his3Δ pdc5Δ::loxP66/71 | Herein |
| | fra2Δ 2μ plasmid (CEN.PK2) | |
| | gpd2Δ::loxP71/66 ymr226CΔ::P[FBA1]- | |
| | ALS\|alsS_Bs-CYC1t-loxP71/66 | |
| | ald6Δ::UAS(PGK1)P[FBA1]-KIVD\|Lg(y)- | |
| | TDH3t-loxP71/66 adh1Δ::P[ILV5]- | |
| | ADH\|Bi(y)-ADHt-loxP71/66 | |
| | pdc1Δ::P[PDC1]-ADH\|Bi(y)-ADHt-loxP71/66 | |
| | pLH702, pYZ067DkivDDhADH | |
| PNY1716 | MATa ura3Δ::loxP his3Δ::loxP pdc6Δ | Herein |
| | pdc1Δ::ilvD pdc5Δ::kivD(y) | |
| PNY0684 | MATa ura3Δ::loxP.pdc1Δ::ilvD | Herein |
| | pdc5Δ::kivDLg | |
| | pdc6Δ::USA.ENO2p.Bi.ADH.ymr226CΔ::pdc | |
| | 5p.Als./pNZ001.PDC1.K9D3.U.ENO2p.ilvD | |

### Construction of Strains Used in the Examples

### Construction of PNY1528

A. Construction of PNY1528 (hADH Integrations in PNY2211)

PNY1528 was constructed in strain PNY2211 (described in PCT Publication No. WO 2012/033832, incorporated herein by reference). Deletions/integrations were created by homologous recombination with PCR products containing regions of homology upstream and downstream of the target region and the URA3 gene for selection of transformants. The URA3 gene was removed by homologous recombination to create a scarless deletion/integration.

The scarless deletion/integration procedure was adapted from Akada et al., Yeast, 23:399 (2006). The PCR cassette for each deletion/integration was made by combining four fragments, A-B-U-C, and the gene to be integrated by cloning the individual fragments into a plasmid prior to the entire cassette being amplified by PCR for the deletion/integration procedure. The gene to be integrated was included in the cassette between fragments A and B. The PCR cassette contained a selectable/counter-selectable marker, URA3 (Fragment U), consisting of the native CEN.PK 113-7D URA3 gene, along with the promoter (250 bp upstream of the URA3 gene) and terminator (150 bp downstream of the URA3 gene) regions. Fragments A and C (each approximately 100 to 500 bp long) corresponded to the sequence immediately upstream of the target region (Fragment A) and the 3′ sequence of the target region (Fragment C). Fragments A and C were used for integration of the cassette into the chromosome by homologous recombination. Fragment B (500 bp long) corresponded to the 500 bp immediately downstream of the target region and was used for excision of the URA3 marker and Fragment C from the chromosome by homologous recombination, as a direct repeat of the sequence corresponding to Fragment B was created upon integration of the cassette into the chromosome.

The integration cassettes were constructed in plasmid pUC19-URA3MCS (SEQ ID NO: 164). The vector is pUC19 based and contains the sequence of the URA3 gene from Saccharomyces cerevisiae CEN.PK 113-7D situated within a multiple cloning site (MCS). pUC19 contains the pMB1 replicon and a gene coding for beta-lactamase for replication and selection in Escherichia coli. In addition to the coding sequence for URA3, the sequences from upstream (250 bp) and downstream (150 bp) of this gene are present for expression of the URA3 gene in yeast.

B. YPRCΔ15 deletion and horse liver adh integration

The YPRCΔ15 locus was deleted and replaced with the horse liver adh gene, codon-optimized for expression in Saccharomyces cerevisiae, along with the PDC5 promoter region (538 bp) from Saccharomyces cerevisiae and the ADH1 terminator region (316 bp) from Saccharomyces cerevisiae. The scarless cassette for the YPRCΔ15 deletion-P[PDC5]-adh_HL(y)-ADH1t integration was first cloned into plasmid pUC19-URA3MCS.

Fragments A-B-U-C were amplified using Phusion High Fidelity PCR Master Mix (New England BioLabs; Ipswich, Mass.) and CEN.PK 113-7D genomic DNA as template, prepared with a Gentra Puregene Yeast/Bact kit (Qiagen; Valencia, Calif.). YPRCΔ15 Fragment A was amplified from genomic DNA with primer oBP622 (SEQ ID NO: 77), containing a KpnI restriction site, and primer oBP623 (SEQ ID NO: 78), containing a 5′ tail with homology to the 5′ end of YPRCΔ15 Fragment B. YPRCΔ15 Fragment B was amplified from genomic DNA with primer oBP624 (SEQ ID NO: 79), containing a 5′ tail with homology to the 3′ end of YPRCΔ15 Fragment A, and primer oBP625 (SEQ ID NO: 80), containing a FseI restriction site. PCR products were purified with a PCR Purification kit (Qiagen). YPRCΔ15 Fragment A—YPRCΔ15 Fragment B was created by overlapping PCR by mixing the YPRCΔ15 Fragment A and YPRCΔ15 Fragment B PCR products and amplifying with primers oBP622 (SEQ ID NO: 77) and oBP625 (SEQ ID NO: 80). The resulting PCR product was digested with KpnI and FseI and ligated with T4 DNA ligase into the corresponding sites of pUC19-URA3MCS after digestion with the appropriate enzymes. YPRCΔ15 Fragment C was amplified from genomic DNA with primer oBP626 (SEQ ID NO: 81), containing a NotI restriction site, and primer oBP627 (SEQ ID NO: 82), containing a PacI restriction site. The YPRCΔ15 Fragment C PCR product was digested with NotI and PacI and ligated with T4 DNA ligase into the corresponding sites of the plasmid containing YPRCΔ15 Fragments AB. The PDC5 promoter region was amplified from CEN.PK 113-7D genomic DNA with primer HY21 (SEQ ID NO: 83), containing an AscI restriction site, and primer HY24 (SEQ ID NO: 84), containing a 5′ tail with homology to the 5′ end of adh_H1(y). adh_H1(y)-ADH1t was amplified from pBP915 (SEQ ID NO: 165) with primers HY25 (SEQ ID NO: 85), containing a 5′ tail with homology to the 3′ end of P[PDC5], and HY4 (SEQ ID NO: 86), containing a PmeI restriction site. PCR products were purified with a PCR Purification kit (Qiagen). P[PDC5]-adh_HL(y)-ADH1t was created by overlapping PCR by mixing the P[PDC5] and adh_HL(y)-ADH1t PCR products and amplifying with primers HY21 (SEQ ID NO: 83) and HY4 (SEQ ID NO: 86). The resulting PCR product was digested with AscI and PmeI and ligated with T4 DNA ligase into the corresponding sites of the plasmid containing YPRCΔ15 Fragments ABC. The entire integration cassette was amplified from the resulting plasmid with primers oBP622 (SEQ ID NO: 77) and oBP627 (SEQ ID NO: 82).

Competent cells of PNY2211 were made and transformed with the YPRCΔ15 deletion-P[PDC5]-adh_HL(y)-ADH1t integration cassette PCR product using a Frozen-EZ Yeast Transformation II kit (Zymo Research; Orange, Calif.). Transformation mixtures were plated on synthetic complete media lacking uracil supplemented with 1% ethanol at 30° C. Transformants were screened for by PCR with primers URA3-end F (SEQ ID NO: 87) and oBP637 (SEQ ID NO: 89). Correct transformants were grown in YPE (1% ethanol) and plated on synthetic complete medium supplemented with 1% ethanol and containing 5-fluoro-orotic acid (0.1%) at 30° C. to select for isolates that lost the URA3 marker. The deletion of YPRCΔ15 and integration of P[PDC5]-adh_HL(y)-ADH1t were confirmed by PCR with external primers oBP636 (SEQ ID NO: 88) and oBP637 (SEQ ID NO: 89) using genomic DNA prepared with a YeaStar Genomic DNA kit (Zymo Research). A correct isolate of the following genotype was selected for further modification: CEN.PK 113-7D MATa ura3Δ::loxP his3Δ pdc6Δ pdc1Δ::P[PDC1]-DHAD|ilvD_Sm-PDC1t-P[FBA1]-ALS|alsS_Bs-CYC1t pdc5Δ::P[PDC5]-ADH|sadB_Ax-PDC5t gpd2Δ::loxP fra2Δ adh1Δ::UAS(PGK1)P[FBA1]-kivD_L1(y)-ADH1t yprcΔ15Δ::P[PDC5]-ADH|adh_H1-ADH1t.

C. Horse Liver Adh Integration at fra2Δ

The horse liver adh gene, codon-optimized for expression in Saccharomyces cerevisiae, along with the PDC1 promoter region (870 bp) from Saccharomyces cerevisiae and the ADH1 terminator region (316 bp) from Saccharomyces cerevisiae, was integrated into the site of the fra2 deletion in the PNY2211 variant with adh_H1(y) integrated at YPRCΔ15. The scarless cassette for the fra2Δ-P[PDC1]-adh_HL(y)-ADH1t integration was first cloned into plasmid pUC19-URA3MCS.

Fragments A-B-U-C were amplified using Phusion High Fidelity PCR Master Mix (New England BioLabs; Ipswich, Mass.) and CEN.PK 113-7D genomic DNA as template, prepared with a Gentra Puregene Yeast/Bact kit (Qiagen; Valencia, Calif.). fra2Δ Fragment C was amplified from genomic DNA with primer oBP695 (SEQ ID NO: 94), containing a NotI restriction site, and primer oBP696 (SEQ ID NO: 95), containing a PacI restriction site. The fra2Δ Fragment C PCR product was digested with NotI and PacI and ligated with T4 DNA ligase into the corresponding sites of pUC19-URA3MCS. fra2Δ Fragment B was amplified from genomic DNA with primer oBP693 (SEQ ID NO: 92), containing a PmeI restriction site, and primer oBP694 (SEQ ID NO: 93), containing a FseI restriction site. The resulting PCR product was digested with PmeI and FseI and ligated with T4 DNA ligase into the corresponding sites of the plasmid containing fra2Δ fragment C after digestion with the appropriate enzymes. fra2Δ Fragment A was amplified from genomic DNA with primer oBP691 (SEQ ID NO: 90), containing BamHI and AsiSI restriction sites, and primer oBP692 (SEQ ID NO: 91), containing AscI and SwaI restriction sites. The fra2Δ fragment A PCR product was digested with BamHI and AscI and ligated with T4 DNA ligase into the corresponding sites of the plasmid containing fra2Δ fragments BC after digestion with the appropriate enzymes. The PDC1 promoter region was amplified from CEN.PK 113-7D genomic DNA with primer HY16 (SEQ ID NO: 96), containing an AscI restriction site, and primer HY19 (SEQ ID NO: 97), containing a 5′ tail with homology to the 5′ end of adh_H1(y). adh_H1(y)-ADH1t was amplified from pBP915 with primers HY20 (SEQ ID NO: 98), containing a 5′ tail with homology to the 3′ end of P[PDC1], and HY4 (SEQ ID NO: 86), containing a PmeI restriction site. PCR products were purified with a PCR Purification kit (Qiagen). P[PDC1]-adh_HL(y)-ADH1t was created by overlapping PCR by mixing the P[PDC1] and adh_HL(y)-ADH1t PCR products and amplifying with primers HY16 (SEQ ID NO: 96) and HY4 (SEQ ID NO: 86). The resulting PCR product was digested with AscI and PmeI and ligated with T4 DNA ligase into the corresponding sites of the plasmid containing fra2Δ Fragments ABC. The entire integration cassette was amplified from the resulting plasmid with primers oBP691 (SEQ ID NO: 90) and oBP696 (SEQ ID NO: 95).

Competent cells of the PNY2211 variant with adh_H1(y) integrated at YPRCΔ15 were made and transformed with the fra2Δ-P[PDC1]-adh_HL(y)-ADH1t integration cassette PCR product using a Frozen-EZ Yeast Transformation II kit (Zymo Research). Transformation mixtures were plated on synthetic complete media lacking uracil supplemented with 1% ethanol at 30° C. Transformants were screened for by PCR with primers URA3-end F (SEQ ID NO: 87) and oBP731 (SEQ ID NO: 100). Correct transformants were grown in YPE (1% ethanol) and plated on synthetic complete medium supplemented with 1% ethanol and containing 5-fluoro-orotic acid (0.1%) at 30° C. to select for isolates that lost the URA3 marker. The integration of P[PDC1]-adh_HL(y)-ADH1t was confirmed by colony PCR with internal primer HY31 (SEQ ID NO: 286) and external primer oBP731 (SEQ ID NO: 100) and PCR with external primers oBP730 (SEQ ID NO: 99) and oBP731 (SEQ ID NO: 100) using genomic DNA prepared with a YeaStar Genomic DNA kit (Zymo Research). A correct isolate of the following genotype was designated PNY1528: CEN.PK 113-7D MATa ura3Δ::loxP his3Δ pdc6Δ pdc1Δ::P[PDC1]-DHAD|ilvD_Sm-PDC1t-P [FBA1]-ALS|alsS_Bs-CYC1t pdc5Δ::P[PDC5]-ADH|sadB_Ax-PDC5t gpd2Δ::loxP fra2Δ::P[PDC1]-ADH|adh_H1-ADH1t adh1Δ::UAS(PGK1)P[FBA1]-kivD_L1(y)-ADH1t yprcΔ15Δ::P[PDC5]-ADH|adh_H1-ADH1t.

### Construction of PNY1530

PNY1530 was constructed by transforming PNY1528 with plasmid pYZ107F-OLE1p (SEQ ID NO: 166) using a Frozen-EZ Yeast Transformation II kit (Zymo Research; Orange, Calif.). Plasmid pYZ107F-OLE1p (SEQ ID NO: 166) was constructed to contain a chimeric gene having the coding region of the ilvD gene from Streptococcus mutans (nt position 5356-3644) expressed from the Saccharomyces cerevisiae OLE1 promoter (nt 5961-5366) and followed by the FBA1 terminator (nt 3611-3299) for expression of DHAD, and a chimeric gene having the coding region of the ilvC gene from Lactococcus lactis (nt 1628-2650) expressed from the Saccharomyces cerevisiae ILV5 promoter (nt 434-1614) and followed by the ILV5 terminator (nt 2664-3286) for expression of KARI.

### Construction of PNY2068

Saccharomyces cerevisiae strain PNY0827 was used as the host cell for further genetic manipulation. PNY0827 refers to a strain derived from Saccharomyces cerevisiae which has been deposited at the ATCC under the Budapest Treaty on Sep. 22, 2011 at the American Type Culture Collection, Patent Depository 10801 University Boulevard, Manassas, Va. 20110-2209 and has the patent deposit designation PTA-12105.

A. Deletion of URA3 and Sporulation into Haploids

In order to delete the endogenous URA3 coding region, a deletion cassette was PCR-amplified from pLA54 (SEQ ID NO: 167) which contains a P_{TEF1}-kanMX4-TEF1t cassette flanked by loxP sites to allow homologous recombination in vivo and subsequent removal of the KANMX4 marker. PCR was done by using Phusion High Fidelity PCR Master Mix (New England BioLabs; Ipswich, Mass.) and primers BK505 (SEQ ID NO: 101) and BK506 (SEQ ID NO: 102). The URA3 portion of each primer was derived from the 5′ region 180 bp upstream of the URA3 ATG and 3′ region 78 bp downstream of the coding region such that integration of the kanMX4 cassette results in replacement of the URA3 coding region. The PCR product was transformed into PNY0827 using standard genetic techniques (Methods in Yeast Genetics, 2005, Cold Spring Harbor Laboratory Press, Cold Spring Harbor, N.Y., pp. 201-202) and transformants were selected on YEP medium supplemented 2% glucose and 100 μg/ml Geneticin at 30° C. Transformants were screened by colony PCR with primers LA468 (SEQ ID NO: 103) and LA492 (SEQ ID NO: 104) to verify presence of the integration cassette. A heterozygous diploid was obtained: NYLA98, which has the genotype MATa/α URA3/ura3::loxP-kanMX4-loxP. To obtain haploids, NYLA98 was sporulated using standard methods (Appl. Environ Microbiol. (1995) 61:630-638). Tetrads were dissected using a micromanipulator and grown on rich YPE medium supplemented with 2% glucose. Tetrads containing four viable spores were patched onto synthetic complete medium lacking uracil supplemented with 2% glucose, and the mating type was verified by multiplex colony PCR using primers AK109-1 (SEQ ID NO: 105), AK109-2 (SEQ ID NO: 106), and AK109-3 (SEQ ID NO: 107). From this were identified haploid strains called NYLA103, which has the genotype: MATα ura3Δ::loxP-kanMX4-loxP, and NYLA106, which has the genotype: MATa ura3Δ::loxP-kanMX4-loxP.

### B. Deletion of His3

To delete the endogenous HIS3 coding region, a scarless deletion cassette was used. The four fragments for the PCR cassette for the scarless HIS3 deletion were amplified using Phusion High Fidelity PCR Master Mix (New England BioLabs; Ipswich, Mass.) and CEN.PK 113-7D genomic DNA as template, prepared with a Gentra Puregene Yeast/Bact kit (Qiagen; Valencia, Calif.). HIS3 Fragment A was amplified with primer oBP452 (SEQ ID NO: 108) and primer oBP453 (SEQ ID NO: 109), containing a 5′ tail with homology to the 5′ end of HIS3 Fragment B. HIS3 Fragment B was amplified with primer oBP454 (SEQ ID NO: 110), containing a 5′ tail with homology to the 3′ end of HIS3 Fragment A, and primer oBP455 (SEQ ID NO: 111) containing a 5′ tail with homology to the 5′ end of HIS3 Fragment U. HIS3 Fragment U was amplified with primer oBP456 (SEQ ID NO: 112), containing a 5′ tail with homology to the 3′ end of HIS3 Fragment B, and primer oBP457 (SEQ ID NO: 113), containing a 5′ tail with homology to the 5′ end of HIS3 Fragment C. HIS3 Fragment C was amplified with primer oBP458 (SEQ ID NO: 114), containing a 5′ tail with homology to the 3′ end of HIS3 Fragment U, and primer oBP459 (SEQ ID NO: 115). PCR products were purified with a PCR Purification kit (Qiagen). HIS3 Fragment AB was created by overlapping PCR by mixing HIS3 Fragment A and HIS3 Fragment B and amplifying with primers oBP452 (SEQ ID NO: 108) and oBP455 (SEQ ID NO: 111). HIS3 Fragment UC was created by overlapping PCR by mixing HIS3 Fragment U and HIS3 Fragment C and amplifying with primers oBP456 (SEQ ID NO: 112) and oBP459 (SEQ ID NO: 115). The resulting PCR products were purified on an agarose gel followed by a Gel Extraction kit (Qiagen). The HIS3 ABUC cassette was created by overlapping PCR by mixing HIS3 Fragment AB and HIS3 Fragment UC and amplifying with primers oBP452 (SEQ ID NO: 108) and oBP459 (SEQ ID NO: 115). The PCR product was purified with a PCR Purification kit (Qiagen). Competent cells of NYLA106 were transformed with the HIS3 ABUC PCR cassette and were plated on synthetic complete medium lacking uracil supplemented with 2% glucose at 30° C. Transformants were screened to verify correct integration by replica plating onto synthetic complete medium lacking histidine and supplemented with 2% glucose at 30° C. Genomic DNA preps were made to verify the integration by PCR using primers oBP460 (SEQ ID NO: 116) and LA135 (SEQ ID NO: 117) for the 5′ end and primers oBP461 (SEQ ID NO: 118) and LA92 (SEQ ID NO: 119) for the 3′ end. The URA3 marker was recycled by plating on synthetic complete medium supplemented with 2% glucose and 5-FOA at 30° C. following standard protocols. Marker removal was confirmed by patching colonies from the 5-FOA plates onto SD-URA medium to verify the absence of growth. The resulting identified strain, called PNY2003 has the genotype: MATa ura3Δ::loxP-kanMX4-loxP his3Δ.

### C. Deletion of PDC1

To delete the endogenous PDC1 coding region, a deletion cassette was PCR-amplified from pLA59 (SEQ ID NO: 168), which contains a URA3 marker flanked by degenerate loxP sites to allow homologous recombination in vivo and subsequent removal of the URA3 marker. PCR was done by using Phusion High Fidelity PCR Master Mix (New England BioLabs; Ipswich, Mass.) and primers LA678 (SEQ ID NO: 120) and LA679 (SEQ ID NO: 121). The PDC1 portion of each primer was derived from the 5′ region 50 bp downstream of the PDC1 start codon and 3′ region 50 bp upstream of the stop codon such that integration of the URA3 cassette results in replacement of the PDC1 coding region but leaves the first 50 bp and the last 50 bp of the coding region. The PCR product was transformed into PNY2003 using standard genetic techniques and transformants were selected on synthetic complete medium lacking uracil and supplemented with 2% glucose at 30° C. Transformants were screened to verify correct integration by colony PCR using primers LA337 (SEQ ID NO: 122), external to the 5′ coding region and LA135 (SEQ ID NO: 117), an internal primer to URA3. Positive transformants were then screened by colony PCR using primers LA692 (SEQ ID NO: 123) and LA693 (SEQ ID NO: 124), internal to the PDC1 coding region. The URA3 marker was recycled by transforming with pLA34 (SEQ ID NO: 169) containing the CRE recombinase under the GAL1 promoter and plated on synthetic complete medium lacking histidine and supplemented with 2% glucose at 30° C. Transformants were plated on rich medium supplemented with 0.5% galactose to induce the recombinase. Marker removal was confirmed by patching colonies to synthetic complete medium lacking uracil and supplemented with 2% glucose to verify absence of growth. The resulting identified strain, called PNY2008 has the genotype: MATa ura3Δ::loxP-kanMX4-loxP his3Δ pdc1Δ::loxP71/66.

### D. Deletion of PDC5

To delete the endogenous PDC5 coding region, a deletion cassette was PCR-amplified from pLA59 (SEQ ID NO: 168), which contains a URA3 marker flanked by degenerate loxP sites to allow homologous recombination in vivo and subsequent removal of the URA3 marker. PCR was done by using Phusion High Fidelity PCR Master Mix (New England BioLabs; Ipswich, Mass.) and primers LA722 (SEQ ID NO: 125) and LA733 (SEQ ID NO: 126). The PDC5 portion of each primer was derived from the 5′ region 50 bp upstream of the PDC5 start codon and 3′ region 50 bp downstream of the stop codon such that integration of the URA3 cassette results in replacement of the entire PDC5 coding region. The PCR product was transformed into PNY2008 using standard genetic techniques and transformants were selected on synthetic complete medium lacking uracil and supplemented with 1% ethanol at 30° C. Transformants were screened to verify correct integration by colony PCR using primers LA453 (SEQ ID NO: 127), external to the 5′ coding region and LA135 (SEQ ID NO: 117), an internal primer to URA3. Positive transformants were then screened by colony PCR using primers LA694 (SEQ ID NO: 128) and LA695 (SEQ ID NO: 129), internal to the PDC5 coding region. The URA3 marker was recycled by transforming with pLA34 (SEQ ID NO: 169) containing the CRE recombinase under the GAL1 promoter and plated on synthetic complete medium lacking histidine and supplemented with 1% ethanol at 30° C. Transformants were plated on rich YEP medium supplemented with 1% ethanol and 0.5% galactose to induce the recombinase. Marker removal was confirmed by patching colonies to synthetic complete medium lacking uracil and supplemented with 1% ethanol to verify absence of growth. The resulting identified strain, called PNY2009 has the genotype: MATα ura3Δ::loxP-kanMX4-loxP his3 pdc1Δ::loxP71/66 pdc5Δ::loxP71/66.

### E. Deletion of FRA2

The FRA2 deletion was designed to delete 250 nucleotides from the 3′ end of the coding sequence, leaving the first 113 nucleotides of the FRA2 coding sequence intact. An in-frame stop codon was present 7 nucleotides downstream of the deletion. The four fragments for the PCR cassette for the scarless FRA2 deletion were amplified using Phusion High Fidelity PCR Master Mix (New England BioLabs; Ipswich, Mass.) and CEN.PK 113-7D genomic DNA as template, prepared with a Gentra Puregene Yeast/Bact kit (Qiagen; Valencia, Calif.). FRA2 Fragment A was amplified with primer oBP594 (SEQ ID NO: 130) and primer oBP595 (SEQ ID NO: 131), containing a 5′ tail with homology to the 5′ end of FRA2 Fragment B. FRA2 Fragment B was amplified with primer oBP596 (SEQ ID NO: 132), containing a 5″ tail with homology to the 3′ end of FRA2 Fragment A, and primer oBP597 (SEQ ID NO: 133), containing a 5′ tail with homology to the 5′ end of FRA2 Fragment U. FRA2 Fragment U was amplified with primer oBP598 (SEQ ID NO: 134), containing a 5′ tail with homology to the 3′ end of FRA2 Fragment B, and primer oBP599 (SEQ ID NO: 135), containing a 5′ tail with homology to the 5′ end of FRA2 Fragment C. FRA2 Fragment C was amplified with primer oBP600 (SEQ ID NO: 136), containing a 5′ tail with homology to the 3′ end of FRA2 Fragment U, and primer oBP601 (SEQ ID NO: 137). PCR products were purified with a PCR Purification kit (Qiagen). FRA2 Fragment AB was created by overlapping PCR by mixing FRA2 Fragment A and FRA2 Fragment B and amplifying with primers oBP594 (SEQ ID NO: 130) and oBP597 (SEQ ID NO: 133). FRA2 Fragment UC was created by overlapping PCR by mixing FRA2 Fragment U and FRA2 Fragment C and amplifying with primers oBP598 (SEQ ID NO: 134) and oBP601 (SEQ ID NO: 137). The resulting PCR products were purified on an agarose gel followed by a Gel Extraction kit (Qiagen). The FRA2 ABUC cassette was created by overlapping PCR by mixing FRA2 Fragment AB and FRA2 Fragment UC and amplifying with primers oBP594 (SEQ ID NO: 130) and oBP601 (SEQ ID NO: 137). The PCR product was purified with a PCR Purification kit (Qiagen).

To delete the endogenous FRA2 coding region, the scarless deletion cassette obtained above was transformed into PNY2009 using standard techniques and plated on synthetic complete medium lacking uracil and supplemented with 1% ethanol. Genomic DNA preps were made to verify the integration by PCR using primers oBP602 (SEQ ID NO: 138) and LA135 (SEQ ID NO: 117) for the 5′ end, and primers oBP602 (SEQ ID NO: 138) and oBP603 (SEQ ID NO: 139) to amplify the whole locus. The URA3 marker was recycled by plating on synthetic complete medium supplemented with 1% ethanol and 5-FOA (5-Fluoroorotic Acid) at 30° C. following standard protocols. Marker removal was confirmed by patching colonies from the 5-FOA plates onto synthetic complete medium lacking uracil and supplemented with 1% ethanol to verify the absence of growth. The resulting identified strain, PNY2037, has the genotype: MATa ura3Δ::loxP-kanMX4-loxP his3 pdc1Δ::loxP71/66 pdc5Δ::loxP71/66 fra2Δ.

### F. Addition of 2 Micron Plasmid

The loxP71-URA3-loxP66 marker was PCR-amplified using Phusion DNA polymerase (New England BioLabs; Ipswich, Mass.) from pLA59 (SEQ ID NO: 168), and transformed along with the LA811x817 (SEQ ID NOs: 170, 171) and LA812x818 (SEQ ID NOs: 172, 173) 2-micron plasmid fragments into strain PNY2037 on SE-URA plates at 30° C. The resulting strain PNY2037 2μ::loxP71-URA3-loxP66 was transformed with pLA34 (also called pRS423::cre) (SEQ ID NO: 169) and selected on SE-HIS-URA plates at 30° C. Transformants were patched onto YP-1% galactose plates and allowed to grow for 48 hrs at 30° C. to induce Cre recombinase expression. Individual colonies were then patched onto SE-URA, SE-HIS, and YPE plates to confirm URA3 marker removal. The resulting identified strain, PNY2050, has the genotype: MATa ura3Δ::loxP-kanMX4-loxP, his3 pdc1Δ::loxP71/66 pdc5Δ::loxP71/66 fra2Δ 2-micron.

### G. Deletion of GPD2

To delete the endogenous GPD2 coding region, a deletion cassette was PCR-amplified from pLA59 (SEQ ID NO: 168), which contains a URA3 marker flanked by degenerate loxP sites to allow homologous recombination in vivo and subsequent removal of the URA3 marker. PCR was done by using Phusion High Fidelity PCR Master Mix (New England BioLabs; Ipswich, Mass.) and primers LA512 (SEQ ID NO: 140) and LA513 (SEQ ID NO: 141). The GPD2 portion of each primer was derived from the 5′ region 50 bp upstream of the GPD2 start codon and 3′ region 50 bp downstream of the stop codon such that integration of the URA3 cassette results in replacement of the entire GPD2 coding region. The PCR product was transformed into PNY2050 using standard genetic techniques and transformants were selected on synthetic complete medium lacking uracil and supplemented with 1% ethanol at 30° C. Transformants were screened to verify correct integration by colony PCR using primers LA516 (SEQ ID NO: 142), external to the 5′ coding region and LA135 (SEQ ID NO: 117), internal to URA3. Positive transformants were then screened by colony PCR using primers LA514 (SEQ ID NO: 143) and LA515 (SEQ ID NO: 144), internal to the GPD2 coding region. The URA3 marker was recycled by transforming with pLA34 (SEQ ID NO: 169) containing the CRE recombinase under the GAL1 promoter and plated on synthetic complete medium lacking histidine and supplemented with 1% ethanol at 30° C. Transformants were plated on rich medium supplemented with 1% ethanol and 0.5% galactose to induce the recombinase. Marker removal was confirmed by patching colonies to synthetic complete medium lacking uracil and supplemented with 1% ethanol to verify absence of growth. The resulting identified strain, PNY2056, has the genotype: MATa ura3Δ::loxP-kanMX4-loxP his3Δ pdc1Δ::loxP71/66 pdc5Δ::loxP71/66fra2Δ 2-micron gpd2A.

### H. Deletion of YMR226 and Integration of AlsS

To delete the endogenous YMR226C coding region, an integration cassette was PCR-amplified from pLA71 (SEQ ID NO: 174), which contains the gene acetolactate synthase from the species Bacillus subtilis with a FBA1 promoter and a CYC1 terminator, and a URA3 marker flanked by degenerate loxP sites to allow homologous recombination in vivo and subsequent removal of the URA3 marker. PCR was done by using KAPA HiFi from Kapa Biosystems, Woburn, Mass. and primers LA829 (SEQ ID NO: 145) and LA834 (SEQ ID NO: 146). The YMR226C portion of each primer was derived from the first 60 bp of the coding sequence and 65 bp that are 409 bp upstream of the stop codon. The PCR product was transformed into PNY2056 using standard genetic techniques and transformants were selected on synthetic complete medium lacking uracil and supplemented with 1% ethanol at 30° C. Transformants were screened to verify correct integration by colony PCR using primers N1257 (SEQ ID NO: 147), external to the 5′ coding region and LA740 (SEQ ID NO: 148), internal to the FBA1 promoter. Positive transformants were then screened by colony PCR using primers N1257 (SEQ ID NO: 147) and LA830 (SEQ ID NO: 149), internal to the YMR226C coding region, and primers LA830 (SEQ ID NO: 149), external to the 3′ coding region, and LA92 (SEQ ID NO: 119), internal to the URA3 marker. The URA3 marker was recycled by transforming with pLA34 (SEQ ID NO: 169) containing the CRE recombinase under the GAL1 promoter and plated on synthetic complete medium lacking histidine and supplemented with 1% ethanol at 30° C. Transformants were plated on rich medium supplemented with 1% ethanol and 0.5% galactose to induce the recombinase. Marker removal was confirmed by patching colonies to synthetic complete medium lacking uracil and supplemented with 1% ethanol to verify absence of growth. The resulting identified strain, PNY2061, has the genotype: MATa ura3Δ::loxP-kanMX4-loxP his3Δ pdc1Δ::loxP71/66 pdc5Δ::loxP71/66fra2Δ 2-micron gpd2Δ ymr226cΔ::P_{FBA1}-alsS_Bs-CYC1t-loxP71/66.

### I. Deletion of ALD6 and Integration of KivD

To delete the endogenous ALD6 coding region, an integration cassette was PCR-amplified from pLA78 (SEQ ID NO: 175), which contains the kivD gene from the species Listeria grayi with a hybrid FBA1 promoter and a TDH3 terminator, and a URA3 marker flanked by degenerate loxP sites to allow homologous recombination in vivo and subsequent removal of the URA3 marker. PCR was done by using KAPA HiFi from Kapa Biosystems, Woburn, Mass. and primers LA850 (SEQ ID NO: 150) and LA851 (SEQ ID NO: 151). The ALD6 portion of each primer was derived from the first 65 bp of the coding sequence and the last 63 bp of the coding region. The PCR product was transformed into PNY2061 using standard genetic techniques and transformants were selected on synthetic complete medium lacking uracil and supplemented with 1% ethanol at 30° C. Transformants were screened to verify correct integration by colony PCR using primers N1262 (SEQ ID NO: 152), external to the 5′ coding region and LA740 (SEQ ID NO: 148), internal to the FBA1 promoter. Positive transformants were then screened by colony PCR using primers N1263 (SEQ ID NO: 153), external to the 3′ coding region, and LA92 (SEQ ID NO: 176), internal to the URA3 marker. The URA3 marker was recycled by transforming with pLA34 (SEQ ID NO: 169) containing the CRE recombinase under the GAL1 promoter and plated on synthetic complete medium lacking histidine and supplemented with 1% ethanol at 30° C. Transformants were plated on rich medium supplemented with 1% ethanol and 0.5% galactose to induce the recombinase. Marker removal was confirmed by patching colonies to synthetic complete medium lacking uracil and supplemented with 1% ethanol to verify absence of growth. The resulting identified strain, PNY2065, has the genotype: MATa ura3Δ::loxP-kanMX4-loxP his3A pdc1Δ::loxP71/66 pdc5Δ::loxP71/66fra2Δ 2-micron gpd2dymr226cΔ::P_{FBA1}-alsS_Bs-CYC1t-loxP71/66 ald6Δ::(UAS)PGK1-P_{FBA1}-kivD_Lg-TDH3t-loxP71.

### J. Deletion of ADH1 and Integration of ADH

ADH1 is the endogenous alcohol dehydrogenase present in Saccharomyces cerevisiae. As described below, the endogenous ADH1 was replaced with alcohol dehydrogenase (ADH) from Beijerinckii indica.

To delete the endogenous ADH1 coding region, an integration cassette was PCR-amplified from pLA65 (SEQ ID NO: 177), which contains the alcohol dehydrogenase from the species Beijerinckii indica with an ILV5 promoter and a ADH1 terminator, and a URA3 marker flanked by degenerate loxP sites to allow homologous recombination in vivo and subsequent removal of the URA3 marker. PCR was done by using KAPA HiFi from Kapa Biosystems, Woburn, Mass. and primers LA855 (SEQ ID NO: 154) and LA856 (SEQ ID NO: 155). The ADH1 portion of each primer was derived from the 5′ region 50 bp upstream of the ADH1 start codon and the last 50 bp of the coding region. The PCR product was transformed into PNY2065 using standard genetic techniques and transformants were selected on synthetic complete medium lacking uracil and supplemented with 1% ethanol at 30° C. Transformants were screened to verify correct integration by colony PCR using primers LA414 (SEQ ID NO: 156), external to the 5′ coding region and LA749 (SEQ ID NO: 157), internal to the ILV5 promoter. Positive transformants were then screened by colony PCR using primers LA413 (SEQ ID NO: 158), external to the 3′ coding region, and LA92 (SEQ ID NO: 119), internal to the URA3 marker. The URA3 marker was recycled by transforming with pLA34 (SEQ ID NO: 169) containing the CRE recombinase under the GAL1 promoter and plated on synthetic complete medium lacking histidine and supplemented with 1% ethanol at 30° C. Transformants were plated on rich medium supplemented with 1% ethanol and 0.5% galactose to induce the recombinase. Marker removal was confirmed by patching colonies to synthetic complete medium lacking uracil and supplemented with 1% ethanol to verify absence of growth. The resulting identified strain, called PNY2066 has the genotype: MATa ura3Δ::loxP-kanMX4-loxP his3Δ pdc1Δ::loxP71/66 pdc5Δ::loxP71/66fra2Δ 2-micron gpd2Δ ymr226cΔ::P_{FBA1}-alsS_Bs-CYC1t-loxP71/66 ald6Δ:: (UAS)PGK1-P_{FBA1}-kivD_Lg-TDH3t-loxP71/66 adh1Δ::P_{ILV5}-ADH_Bi(y)-ADH1t-loxP71/66.

K. Integration of ADH into pdc1Δ Locus

To integrate an additional copy of ADH at the pdc1Δ region, an integration cassette was PCR-amplified from pLA65 (SEQ ID NO: 177), which contains the alcohol dehydrogenase from the species Beijerinckii indica with an ADH1 terminator, and a URA3 marker flanked by degenerate loxP sites to allow homologous recombination in vivo and subsequent removal of the URA3 marker. PCR was done by using KAPA HiFi from Kapa Biosystems, Woburn, Mass. and primers LA860 (SEQ ID NO: 159) and LA679 (SEQ ID NO: 160). The PDC1 portion of each primer was derived from the 5′ region 60 bp upstream of the PDC1 start codon and 50 bp that are 103 bp upstream of the stop codon. The endogenous PDC1 promoter was used. The PCR product was transformed into PNY2066 using standard genetic techniques and transformants were selected on synthetic complete medium lacking uracil and supplemented with 1% ethanol at 30° C. Transformants were screened to verify correct integration by colony PCR using primers LA337 (SEQ ID NO: 161), external to the 5′ coding region and N1093 (SEQ ID NO: 162), internal to the BiADH gene. Positive transformants were then screened by colony PCR using primers LA681 (SEQ ID NO: 163), external to the 3′ coding region, and LA92 (SEQ ID NO: 119), internal to the URA3 marker. The URA3 marker was recycled by transforming with pLA34 (SEQ ID NO: 169) containing the CRE recombinase under the GAL1 promoter and plated on synthetic complete medium lacking histidine and supplemented with 1% ethanol at 30° C. Transformants were plated on rich medium supplemented with 1% ethanol and 0.5% galactose to induce the recombinase. Marker removal was confirmed by patching colonies to synthetic complete medium lacking uracil and supplemented with 1% ethanol to verify absence of growth. The resulting identified strain, called PNY2068 has the genotype: MATa ura3Δ::loxP-kanMX4-loxP his3Δ pdc1Δ::loxP71/66 pdc5Δ::loxP71/66fra2Δ 2-micron gpd2Δymr226cΔ::P_{FBA1}-alsS_Bs-CYC1t-loxP71/66 ald6Δ::(UAS)PGK1-P_{FBA1}-kivD_Lg-TDH3t-loxP71/66 adh1Δ::P_{ILV5}-ADH_Bi(y)-ADH1t-loxP71/66pdc1Δ::P_{PDC1}-ADH_Bi(y)-ADH1t-loxP71/66.

### Construction of PNY2071

Plasmids for expression of a variant of Anaerostipes caccae KARI (pLH702, SEQ ID NO: 178) and DHAD (pYZ067DkivDDhADH, SEQ ID NO: 179) were introduced into PNY2068 using standard techniques, resulting in strain PNY2071.

### Construction of PNY1716

The yeast strain PNY860 (ATCC Patent Deposit Designation PTA-12007, deposited on Jul. 21, 2011) was tested for sporulation competence (Codon, et al., Appl. Environ. Microbiol. 61:630-638, 1995) by growth overnight at 30° C. in 2 mL pre-sporulation medium (0.8% yeast extract, 0.3% peptone, 10% glucose) in a roller drum, followed by 1:10 dilution into fresh pre-sporulation medium and further growth for 4 hr. Cells were recovered by centrifugation and resuspended in 2 mL sporulation medium (0.5% potassium acetate) and incubated for 4 days in a roller drum at 30° C. Microscopic examination revealed that sporulation had occurred. Approximately 30% of the cells were in the form of asci, and about half of the asci contained four spores. The sporulation culture (100 μL) was recovered by centrifugation and resuspended in Zymolyase® (50 μg/mL in 1 M sorbitol), and incubated for 20 min at room temperature. An aliquot (5 μL) was transferred to a Petri plate, and 18 tetrads were dissected using a Singer MSM dissection microscope (Singer Instrument Co. Ltd., Somerset UK) according to the manufacturer's instructions. The plate was incubated 3 days at 30° C. and the spore viability was scored.

To identify mating types, four spore colonies from two tetrads were analyzed by colony PCR (see, e.g., Huxley, et al., Trends Genet. 6:236, 1990) using Phusion® High Fidelity PCR Master Mix (New England BioLabs Inc.; Ipswich, Mass.) with three oligonucleotide primers, AK09-1_MAT (SEQ ID NO: 183), AK09-2_HML (SEQ ID NO: 184), and AK09-03_HMR (SEQ ID NO: 185).

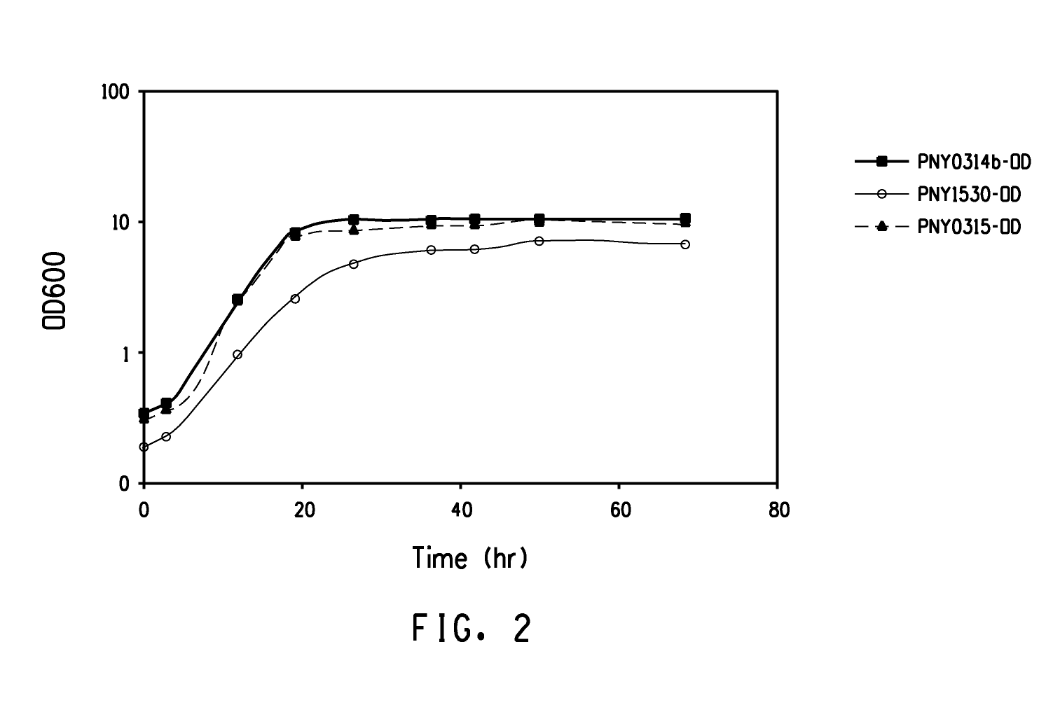

Cells from colonies were lysed by suspension in 0.02 M NaOH and heating to 99° C. for 10 min. A portion of this lysate was then used as the template in a PCR reaction using Taq polymerase (Promega, Madison Wis.) as recommended by the manufacturer. PCR products were analyzed by agarose gel electrophoresis. Strains of mating type α are expected to generate a 404 bp product, strains of mating type a are expected to produce a 544 bp product, and diploids should produce both bands. FIG. 2 shows that the parental strain, PNY860, produces two bands, and the spore progeny produce only one prominent band, of ˜400 bp or ˜550 bp (although some produced faint bands of the other size). These results suggest that PNY860 is a diploid and is largely heterothallic (although a low level of mating type switching may have occurred).

Based on the PCR fragment sizes, the mating types can be inferred to be as follows in Table 7:

TABLE 7

| Yeast Strain | Mating Type |
|---|---|
| PNY860 | Diploid |
| PNY860-1A | a |
| PNY860-1B | α |
| PNY860-1C | a |
| PNY860-1D | α |
| PNY860-2A | a |
| PNY860-2B | α |
| PNY860-2C | a |
| PNY860-2D | α |

To confirm these assignments, spores from tetrad 1 (PNY860-1) were crossed, and mating was scored by looking for zygote formation by microscopy, with the following results in Table 8:

TABLE 8

| Cross | Expected | Observed |
|---|---|---|
| A × B | Mate | Mate |
| C × D | Mate | Mate |
| A × C | No mate | No mate |
| C × D | No mate | No mate |

The yeast strains were designated as follows: PNY860-1A was designated as PNY891, PNY860-1B was designated as PNY0892, PNY860-1C was designated as PNY893, and PNY860-1D was designated as PNY0894.

The haploid strains (PNY891 MATa and PNY0894 MATα) were chosen as a host for isobutanol production. Gene deletion and integration were performed in the haploid strains to create a strain background suitable for isobutanol production. Chromosomal gene deletion was performed by homologous recombination with a PCR cassette containing homology upstream and downstream of the target gene, and either a G-418 resistance marker or URA3 gene for selection of transformants. For gene integration, the gene to be integrated was included in the PCR cassette. The selective marker recycle was achieved using either the Cre-lox system or a scarless deletion method (Akada, et al., Yeast 23: 399, 2006).

First, gene deletion (URA3, HISS, PDC6, and PDC1) and integration (ilvD into the PDC1 site) were performed in the PNY891 MATa to generate PNY1703 (MATa ura3Δ::loxP his3Δ::loxP pdc6Δ pdc1Δ::ilvD). Second, PNY1703 was mated with PNY0894 MATα to make a diploid. The resulting diploid was sporulated and then tetrad-dissected, and spore segregants were screened for growth phenotype on glucose and ethanol media, and genotype carrying ura3Δ::loxP his3Δ::loxP pdc6Δ pdc1Δ::ilvD. Two mating type haploids, PNY1713 (MATα ura3Δ::loxP his3Δ::loxP pdc6Δ pdc1Δ::ilvD) and PNY1714 (MATa ura3Δ::loxP his3Δ::loxP pdc6Δ pdc1Δ::ilvD) were isolated. Third, gene deletion (PDC5, FRA2, GPD2, BDH1, and YMR226c) and integration (kivD, ilvD, alsS, and ilvD-adh into the PDC5, FRA2, GPD2, and BDH1 sites, respectively) were performed in the PNY1714 strain background to construct PNY1758 (MATa ura3Δ::loxP his3Δ::loxP pdc6Δ pdc1Δ::ilvD pdc5Δ::kivD(y)fra2Δ::UAS(PGK1)-FBA1p-dvD(y)gpd2Δ::loxP71/66-FBA1p-alsS bdh1Δ::UAS(PGK1)-ENO2p-dvD-ILV5p-adh ymr226cΔ). Fourth, PNY1758 was transformed with two plasmids, pWZ009 (SEQ ID NO: 276) containing K9D3.KARI gene and pWZ001 (SEQ ID NO: 277) containing ilvD gene, to construct the isobutanologen, PNY1775 (MATa ura3Δ::loxP his3Δ::loxP pdc6Δ pdc1Δ::ilvD pdc5Δ::kivD(y)fra2Δ::UAS(PGK1)-FBA1p-ilvD(y)gpd2Δ::loxP71/66-FBA1p-alsS bdh1Δ::UAS(PGK1)-ENO2p-ilvD-ILV5p-adh ymr226cΔ/pWZ009, pWZ001).

### A. URA3 Deletion

To delete the endogenous URA3 coding region, a deletion cassette was PCR amplified from pLA54 (SEQ ID NO: 167) which contains a TEF1p-kanMX-TEF1t cassette flanked by loxP sites to allow homologous recombination in vivo and subsequent removal of the KanMX marker. PCR was performed using Phusion® DNA polymerase (New England BioLabs Inc., Ipswich, Mass.) and primers BK505 (SEQ ID NO: 101) and BK506 (SEQ ID NO: 102). The URA3 portion of each primer was derived from the 5′ region 180 bp upstream of the URA3 ATG and 3′ region 78 bp downstream of the coding region such that integration of the KanMX cassette results in replacement of the URA3 coding region. The PCR product was transformed into PNY891, a haploid strain, using standard genetic techniques (Methods in Yeast Genetics, 2005, Cold Spring Harbor Laboratory Press, Cold Spring Harbor, N.Y., pp. 201-202) and transformants were selected on rich media supplemented with 2% glucose and G-418 (Geneticin®, 100 μg/mL) at 30° C. Transformants were patched onto rich media supplemented with 2% glucose and replica plated onto synthetic complete media lacking uracil and supplemented with 2% glucose to identify uracil auxotrophs. These patches were screened by colony PCR with primers LA468 (SEQ ID NO: 103) and LA492 (SEQ ID NO: 104) to verify presence of the integration cassette. A URA3 mutant was obtained; NYLA96 (MATa ura3Δ::loxP-kanMX-loxP).

### B. HIS3 Deletion

To delete the endogenous HIS3 coding region, a deletion cassette was PCR amplified from pLA33 (SEQ ID NO: 278) which contains a URA3p-URA3-URA3t cassette flanked by loxP sites to allow homologous recombination in vivo and subsequent removal of the URA3 marker. PCR was performed using Phusion® DNA polymerase (New England BioLabs Inc., Ipswich, Mass.) and primers 315 (SEQ ID NO: 186) and 316 (SEQ ID NO: 187). The HIS3 portion of each primer was derived from the 5′ region 50 bp upstream of the HIS3 ATG and 3′ region 50 bp downstream of the coding region such that integration of the URA3 cassette results in replacement of the HIS3 coding region. The PCR product was transformed into NYLA96 using standard genetic techniques (Methods in Yeast Genetics, 2005, Cold Spring Harbor Laboratory Press, Cold Spring Harbor, N.Y., pp. 201-202) with selection on synthetic complete media lacking uracil supplemented with 2% glucose at 30° C. Transformants were screened by colony PCR with primers 92 (SEQ ID NO: 188) and 346 (SEQ ID NO: 189) to verify presence of the integration cassette. The URA3 marker was recycled by transforming with pLA34 (SEQ ID NO: 169) and plated on synthetic complete media lacking histidine and supplemented with 2% glucose at 30° C. Transformants were plated on yeast extract+peptone (YP) agar plate supplemented with 0.5% galactose to induce expression of Cre recombinase. Marker removal was confirmed by patching colonies to synthetic complete media lacking uracil and supplemented with 2% glucose to verify absence of growth. Also, marker removal of the KanMX cassette, used to delete URA3, was confirmed by patching colonies to rich media supplemented with 2% glucose and G-418 (Geneticin®, 100 μg/mL) at 30° C. to verify absence of growth. The resulting URA3 and HIS3 deletion strain was named NYLA107 (MATa ura3Δ::loxP his3Δ::loxP).

### C. PDC6 Deletion

Saccharomyces cerevisiae has three PDC genes (PDC1, PDC5, PDC6), encoding three different isozymes of pyruvate decarboxylase. Pyruvate decarboxylase catalyzes the first step in ethanol fermentation, producing acetaldehyde from the pyruvate generated in glycolysis.

The PDC6 coding sequence was deleted by homologous recombination with a PCR cassette (A-B-U-C) containing homology upstream (fragment A) and downstream (fragment B) of the PDC6 coding region, a URA3 gene along with the promoter (250 bp upstream of the URA3 gene) and terminator (150 bp downstream of the URA3 gene) (fragment U) for selection of transformants, and the 3′ region of the PDC6 coding region (fragment C), according to a scarless deletion method (Akada, et al., Yeast 23: 399, 2006). The four fragments (A, B, U, C) for the PCR cassette for the scarless PDC6 deletion were amplified from PNY891 genomic DNA as template using Phusion® High Fidelity PCR Master Mix (New England BioLabs Inc.; Ipswich, Mass.). PNY891 genomic DNA was prepared with a Gentra® Puregene® Yeast/Bact. kit (Qiagen, Valencia, Calif.). PDC6 Fragment A was amplified with primer oBP440 (SEQ ID NO: 190) and primer oBP441 (SEQ ID NO: 191), containing a 3′ tail with homology to the 5′ end of PDC6 Fragment B. PDC6 Fragment B was amplified with primer oBP442 (SEQ ID NO: 192), containing a 5′ tail with homology to the 3′ end of PDC6 Fragment A, and primer oBP443 (SEQ ID NO: 193), containing a 5′ tail with homology to the 5′ end of PDC6 Fragment U. PDC6 Fragment U was amplified with primer oBP444 (SEQ ID NO: 194), containing a 5′ tail with homology to the 3′ end of PDC6 Fragment B, and primer oBP445 (SEQ ID NO: 195), containing a 5′ tail with homology to the 5′ end of PDC6 Fragment C. PDC6 Fragment C was amplified with primer oBP446 (SEQ ID NO: 196), containing a 5′ tail with homology to the 3′ end of PDC6 Fragment U, and primer oBP447 (SEQ ID NO: 197). PCR products were purified with a PCR purification kit (Qiagen, Valencia, Calif.). PDC6 Fragment A-B was created by overlapping PCR by mixing PDC6 Fragment A and PDC6 Fragment B and amplifying with primers oBP440 (SEQ ID NO: 190) and oBP443 (SEQ ID NO: 193). PDC6 Fragment U-C was created by overlapping PCR by mixing PDC6 Fragment U and PDC6 Fragment C and amplifying with primers oBP444 (SEQ ID NO: 194) and oBP447 (SEQ ID NO: 197). The resulting PCR products were gel-purified on an agarose gel followed by a gel extraction kit (Qiagen, Valencia, Calif.). The PDC6 A-B-U-C cassette was created by overlapping PCR by mixing PDC6 Fragment A-B and PDC6 Fragment U-C and amplifying with primers oBP440 (SEQ ID NO: 190) and oBP447 (SEQ ID NO: 197). The PCR product was purified with a PCR purification kit (Qiagen, Valencia, Calif.).

Competent cells of NYLA107 were made and transformed with the PDC6 A-B-U-C PCR cassette using a Frozen-EZ Yeast Transformation II™ kit (Zymo Research Corporation, Irvine, Calif.). Transformation mixtures were plated on synthetic complete media lacking uracil supplemented with 2% glucose at 30° C. Transformants with a pdc6 knockout were screened for by PCR with primers oBP448 (SEQ ID NO: 198) and oBP449 (SEQ ID NO: 199) using genomic DNA prepared with a Gentra® Puregene® Yeast/Bact. kit (Qiagen, Valencia, Calif.). To remove the URA3 marker from the chromosome, a correct transformant was grown overnight in YPD and plated on synthetic complete medium containing 5-fluoroorotic acid (0.1%) at 30° C. to select for isolates that lost the URA3 marker. The deletion and marker removal were confirmed by PCR and sequencing with primers oBP448 (SEQ ID NO: 198) and oBP449 (SEQ ID NO: 199) using genomic DNA prepared with a Gentra® Puregene® Yeast/Bact. kit (Qiagen, Valencia, Calif.). The absence of the PDC6 gene from the isolate was demonstrated by a negative PCR result using primers specific for the coding sequence of PDC6, oBP554 (SEQ ID NO: 200) and oBP555 (SEQ ID NO: 201). The correct isolate was selected as strain PNY1702 (MATa ura3Δ::loxP his3Δ::loxP pdc6Δ).

D. PDC1 Deletion and ilvD Integration

The PDC1 coding region was deleted and replaced with the ilvD coding region from Streptococcus mutans ATCC No. 700610 bp homologous recombination with a PCR cassette (A-ilvD-B-U-C) containing homology upstream (fragment A) and downstream (fragment B) of the PDC1 coding region, the ilvD coding region (fragment ilvD), a URA3 gene along with the promoter and terminator (fragment U) for selection of transformants, and the 3′ region of the PDC1 coding region (fragment C). The A fragment followed by the ilvD coding region from Streptococcus mutans for the PCR cassette for the PDC1 deletion-ilvD integration was amplified using Phusion® High Fidelity PCR Master Mix (New England BioLabs Inc.; Ipswich, Mass.) and NYLA83 (described in U.S. Patent Application Publication No. 2011/0312043, which is incorporated herein by reference) genomic DNA as template, prepared with a Gentra® Puregene® Yeast/Bact. kit (Qiagen, Valencia, Calif.). PDC1 Fragment A-ilvD was amplified with primer oBP513 (SEQ ID NO: 202) and primer oBP515 (SEQ ID NO: 203), containing a 5′ tail with homology to the 5′ end of PDC1 Fragment B. The B, U, and C fragments for the PCR cassette for the PDC1 deletion-ilvD integration were amplified using Phusion® High Fidelity PCR Master Mix (New England BioLabs Inc.; Ipswich, Mass.) and PNY891 genomic DNA as template, prepared with a Gentra® Puregene® Yeast/Bact. kit (Qiagen, Valencia, Calif.). PDC1 Fragment B was amplified with primer oBP516 (SEQ ID NO: 204), containing a 5′ tail with homology to the 3′ end of PDC1 Fragment A-ilvD, and primer oBP517 (SEQ ID NO: 205), containing a 5′ tail with homology to the 5′ end of PDC1 Fragment U. PDC1 Fragment U was amplified with primer oBP518 (SEQ ID NO: 206), containing a 5′ tail with homology to the 3′ end of PDC1 Fragment B and primer oBP519 (SEQ ID NO: 207), containing a 5′ tail with homology to the 5′ end of PDC1 Fragment C. The PDC1 Fragment C was amplified with primer oBP520 (SEQ ID NO: 208), containing a 5′ tail with homology to the 3′ end of PDC1 Fragment U, and primer oBP521 (SEQ ID NO: 209). PCR products were purified with a PCR purification kit (Qiagen, Valencia, Calif.). PDC1 Fragment A-ilvD-B was created by overlapping PCR by mixing PDC1 Fragment A-ilvD and PDC1 Fragment B and amplifying with primers oBP513 and oBP517. PDC1 Fragment U-C was created by overlapping PCR by mixing PDC1 Fragment U and PDC1 Fragment C and amplifying with primers oBP518 (SEQ ID NO: 206) and oBP521 (SEQ ID NO: 209). The resulting PCR products were gel-purified on an agarose gel followed by a gel extraction kit (Qiagen, Valencia, Calif.). The PDC1 A-ilvD-BU-C cassette was created by overlapping PCR by mixing PDC1 Fragment A-ilvD-B and PDC1 Fragment U-C and amplifying with primers oBP513 (SEQ ID NO: 202) and oBP521 (SEQ ID NO: 209). The PCR product was purified with a PCR purification kit (Qiagen, Valencia, Calif.).

Competent cells of PNY1702 were made and transformed with the PDC1 A-ilvDB-U-C PCR cassette using a Frozen-EZ Yeast Transformation II™ kit (Zymo Research Corporation, Irvine, Calif.). Transformation mixtures were plated on synthetic complete media lacking uracil supplemented with 2% glucose at 30 C. Transformants with a pdc1 knockout ilvD integration were screened for by PCR with primers oBP511 (SEQ ID NO: 287) and oBP512 (SEQ ID NO: 213) using genomic DNA prepared with a Gentra® Puregene® Yeast/Bact. kit (Qiagen, Valencia, Calif.). The absence of the PDC1 gene from the isolate was demonstrated by a negative PCR result using primers specific for the coding sequence of PDC1, oBP550 (SEQ ID NO: 210) and oBP551 (SEQ ID NO: 211). To remove the URA3 marker from the chromosome, a correct transformant was grown overnight in YPD and plated on synthetic complete medium containing 5-fluoro-orotic acid (0.1%) at 30° C. to select for isolates that lost the URA3 marker. The deletion of PDC1, integration of ilvD, and marker removal were confirmed by PCR with primers ilvDSm(1354F) (SEQ ID NO: 212) and oBP512 (SEQ ID NO: 213) and sequencing with primers ilvDSm(788R) (SEQ ID NO: 214) and ilvDSm(1354F) (SEQ ID NO: 212) using genomic DNA prepared with a Gentra® Puregene® Yeast/Bact. kit (Qiagen, Valencia, Calif.). The correct isolate was selected as strain PNY1703 (MATa ura3Δ::loxP pdc6Δ pdc1Δ::ilvD).

### E. Isolation of PNY1713 and PNY1714

Diploid (MATa/α) cells were created by crossing PNY1703 MATa and PNY0894 MATα on YPD at 30° C. overnight. Potential diploids were streaked onto an YPD plate and incubated at 30° C. for 4 days to isolate single colonies. To identify diploid, colony PCR (Huxley, et al., Trends Genet. 6:236, 1990) was carried out using Phusion® High Fidelity PCR Master Mix (New England BioLabs Inc.; Ipswich, Mass.) with three oligonucleotide primers, MAT1 (SEQ ID NO: 215) corresponding to a sequence at the right of and directed toward the MAT locus, MAT2 (SEQ ID NO: 216) corresponding to a sequence within the α-specific region located at MATα and HMLα, and MAT3 (SEQ ID NO: 217) corresponding to a sequence within the a-specific region located at MATα and HMRa. Diploid colonies were determined by yielding two PCR products, MATα-specific 404 bp and MATa-specific 544 bp. The resulting diploids were grown in pre-sporulation medium and then inoculated into sporulation medium (Codón, et al., Appl. Environ. Microbiol. 61:630, 1995). After 3 days, the sporulation efficiency was checked by microscope. Spores were digested with 0.05 mg/mL Zymolyase® (Zymo Research Corporation, Irvine, Calif.; using the procedure from Methods in Yeast Genetics, 2000, Cold Spring Harbor Laboratory Press, Cold Spring Harbor, N.Y.). Eight (8) plates of tetrads were dissected (18 tetrads per plate, totaling 144 tetrads, 576 spores) on YPD plates and placed at 30oC for 4 days. To screen the spore progeny for genotype ura34 and his34 and growth phenotype on ethanol and glucose media, the spores on YPD plates were sequentially replica plated to 1) the synthetic complete (SC) media lacking uracil (ura) supplemented with 2% glucose, 2) SC lacking histidine (his) supplemented with 2% glucose, and then 3) SC supplemented with 0.5% ethanol media using a yeast replica plating apparatus (Corastyles, Hendersonville, N.C.). Spores that failed to grow on SC-ura and SC-his plates, but grew on SC+0.5% ethanol and YPD plates were selected and PCR-analyzed to determine their mating-type (Huxley, et al., Trends Genet. 6:236, 1990). To determine if the spores contain pdc1Δ::ilvD, the selected spores were checked by colony PCR using primers oBP512 (SEQ ID NO: 213) and ilvDSm(1354F) (SEQ ID NO: 212). Spores containing pdc1Δ::ilvD produce an expected PCR product of 962 bp, but those without the deletion produce no PCR product. The positive spores were then PCR checked for the deletion of PDC6 using primers BP448 (SEQ ID NO: 218) and BP449 (SEQ ID NO: 219). The expected PCR sizes of the fragments were 1.3 kb for cells containing the pdc6Δ and 2.9 kb for cells containing the wild-type PDC6 gene. The correct isolates were selected for both mating types, and designated as PNY1713 (MATα ura3Δ::loxP his3Δ::loxP pdc6Δ pdc1Δ::ilvD) and PNY1714 (MATa ura3Δ::loxP pdc64 pdc1Δ::ilvD).

F. PDC5 Deletion and kivD(y) Integration

The PDC5 coding region was deleted and replaced with the kivD coding region from Lactococcus lactis by homologous recombination with a PCR cassette (A-kivD(y)-BU-C) containing homology upstream (fragment A) and downstream (fragment B) of the PDC5 coding region, the kivD(y) coding region (fragment kivD(y)), codon optimized for expression in Saccharomyces cerevisiae, a URA3 gene along with the promoter and terminator (fragment U) for selection of transformants, and the 3′ region of the PDC5 coding region (fragment C).

PDC5 Fragment A was amplified from PNY891 genomic DNA as template using Phusion® High Fidelity PCR Master Mix (New England BioLabs Inc.; Ipswich, Mass.) with primer T-A(PDC5) (SEQ ID NO: 220) and primer B-A(kivD) (SEQ ID NO: 221), containing a 3′ tail with homology to the 5′ end of kivD(y). The coding sequence of kivD(y) was amplified from pLH468 (SEQ ID NO: 285) as template with primer T-kivD(A) (SEQ ID NO: 222), containing a 5′ tail with homology to the 3′ end of PDC5 Fragment A, and primer BkivD(B) (SEQ ID NO: 223), containing a 3′ tail with homology to the 5′ end of PDC5 Fragment B. PDC5 Fragment A-kivD(y) was created by overlapping PCR by mixing PDC5 Fragment A and kivD(y) and amplifying with primers T-A(PDC5) and B-A(kivD). PDC5 Fragment B was cloned into pUC19-URA3MCS to create the B-U portion of the PDC5 AkivD(y)-B-U-C PCR cassette. The resulting plasmid was designated as pUC19-URA3-sadBPDC5fragmentB (SEQ ID NO: 279). A plasmid pUC19-URA3-sadB-PDC5fragmentB was used as a template for amplification of PDC5 Fragment B-Fragment U using primers TB(kivD) (SEQ ID NO: 224), containing a 5′ tail with homology to the 3′ end of kivD(y) Fragment, and oBP546 (SEQ ID NO: 225), containing a 3′ tail with homology to the 5′ end of PDC5 Fragment C. PDC5 Fragment C was amplified with primer oBP547 (SEQ ID NO: 226), containing a 5′ tail with homology to the 3′ end of PDC5 Fragment B-Fragment U, and primer oBP539 (SEQ ID NO: 227). PCR products were purified with a PCR purification kit (Qiagen, Valencia, Calif.). PDC5 Fragment B-Fragment U-Fragment C was created by overlapping PCR by mixing PDC5 Fragment B-Fragment U and PDC5 Fragment C and amplifying with primers T-B(kivD) (SEQ ID NO: 224) and oBP539 (SEQ ID NO: 227). The resulting PCR product was purified on an agarose gel followed by a gel extraction kit (Qiagen, Valencia, Calif.). The PDC5 A-kivD(y)-B-U-C cassette was created by overlapping PCR by mixing PDC5 Fragment A-kivD(y) Fragment and PDC5 Fragment B-Fragment UPDC5 Fragment C and amplifying with primers T-A(PDC5) (SEQ ID NO: 220) and oBP539 (SEQ ID NO: 227). The PCR product was purified with a PCR purification kit (Qiagen, Valencia, Calif.).

Competent cells of PNY1714 were made and transformed with the PDC5 AkivD(y)-B-U-C PCR cassette using a Frozen-EZ Yeast Transformation II™ kit (Zymo Research Corporation, Irvine, Calif.). Transformation mixtures were plated on synthetic complete media lacking uracil supplemented with 0.5% ethanol (no glucose) at 30° C. Transformants with a pdc5 knockout kivD integration were screened for by PCR with primers oBP540 (SEQ ID NO: 228) and kivD(652R) (SEQ ID NO: 229) using genomic DNA prepared with a Gentra® Puregene® Yeast/Bact. kit (Qiagen, Valencia, Calif.). The absence of the PDC5 gene from the isolate was demonstrated by a negative PCR result using primers specific for the coding sequence of PDC5, oBP552 (SEQ ID NO: 230) and oBP553 (SEQ ID NO: 231). To remove the URA3 marker from the chromosome, each correct transformant of both MATα and MATa strains was grown overnight in YPE (0.5% ethanol) and plated on synthetic complete medium supplemented with ethanol (no glucose) and containing 5-fluoro-orotic acid (0.1%) at 30° C. to select for isolates that lost the URA3 marker. The deletion of PDC5, integration of kivD(y), and marker removal were confirmed by PCR with primers oBP540 and oBP541 using genomic DNA prepared with a Gentra® Puregene® Yeast/Bact. kit (Qiagen, Valencia, Calif.). The correct integration of the kivD(y) coding region was confirmed by DNA sequence with primers, kivD(652R) (SEQ ID NO: 229), kivD(602F) (SEQ ID NO: 232), and kivD(1250F) (SEQ ID NO: 233). The correct isolates were designated as strain PNY1716 (MATa ura3Δ::loxP his3Δ::loxP pdc6Δ pdc1Δ::ilvD pdc5Δ::kivD(y)).

### Construction of PNY0684

PNY0684 was constructed by (1) the integration of a cassette USA.ENO2p. BiADH at the pdc6Δ deletion region, (2) HIS3 restoration, (3) deletion of the YMR226C coding region and replacement with a cassette PDC5p.alsS, and (4) replacement of kivD(y) with kivD.Lg.y at the pdc6Δ deletion region in PNY1716 (MATα ura3Δ::loxP pdc64 pdc1Δ::ilvD pdc5Δ::kivD(y)), and (5) transformation with pNZ001.

A. USA.ENO2p.Bi.ADH Integration at the pdc6A Deletion Region:

Integration of UAS.ENO2p.Bi.ADH at the pdc6Δ deletion region was made in PNY1716 bp homologous recombination. The integration cassette A-USA.ENO2p.Bi.ADH-B-U-C contains the homology upstream (fragment A) and downstream (fragment B) of the PDC6 terminator region, hybrid promoter UAS(PGK1)-ENO2p, ADH coding region from Beijerinckia indica, ADHt terminator, and a URA3 gene along with the promoter and terminator (fragment U) for selection of transformants, and the terminator region of the PDC6 coding region (fragment C).

The fragment A (500 bp) was PCR-amplified from the genomic DNA of PNY0891 using Phusion® DNA polymerase (New England BioLabs Inc., Ipswich, Mass.) with primers JZ067 (SEQ ID NO: 234) and JZ088 (SEQ ID NO: 235). The USA.ENO2p.Bi.ADH cassette (2,147 bp) was PCR-amplified from a plasmid pWS360(USA.ENO2p) (SEQ ID NO: 280) with primers JZ087 (SEQ ID NO: 236) and JZ068 (SEQ ID NO: 237). The fragment B (500 bp) was PCR-amplified from the genomic DNA of PNY0891 with primers JZ069 (SEQ ID NO: 238) and JZ070 (SEQ ID NO: 239). The fragment U (1,232 bp) was PCR-amplified from the genomic DNA of PNY0891 with primers JZ071 (SEQ ID NO: 240) and JZ072 (SEQ ID NO: 241). The fragment C (500 bp) was PCR-amplified from the genomic DNA of PNY0891 with primers JZ073 (SEQ ID NO: 242) and JZ074 (SEQ ID NO: 243). The resulting PCR product was purified on an agarose gel followed by a gel extraction kit (Qiagen, Valencia, Calif.). The B-U-C cassette was created by overlapping PCR by mixing the fragment B, fragment U, and fragment C and amplifying with primers JZ069 and JZ074. The PCR product was purified with a PCR purification kit (Qiagen, Valencia, Calif.). PCR cassette A-USA.ENO2p.Bi.ADH-B-U-C was created by overlapping PCR by mixing the fragment A, USA.ENO2p.Bi.ADH cassette, and B-U-C cassette and amplifying with primers JZ067 and JZ074. The resulting PCR product was purified on an agarose gel followed by a gel extraction kit (Qiagen, Valencia, Calif.).

Competent cells of PNY1716 were made and transformed with the PCR A-USA.ENO2p.Bi.ADH-B-U-C using a Frozen-EZ Yeast Transformation II™ kit (Zymo Research Corporation, Irvine, Calif.). Transformation mixtures were plated on synthetic complete media lacking uracil supplemented with 0.5% ethanol (no glucose) at 30° C. Transformants with a USA.ENO2p.Bi.ADH-B-U integration were screened for by PCR with primers JZ061 (SEQ ID NO: 244) and JZ060 (SEQ ID NO: 245) using genomic DNA prepared with a Gentra® Puregene® Yeast/Bact. kit (Qiagen, Valencia, Calif.). To remove the URA3 marker from the chromosome, correct transformants were grown overnight in YPE (0.5% ethanol) and plated on synthetic complete medium supplemented with ethanol (no glucose) and containing 5-fluoro-orotic acid (0.1%) at 30° C. to select for isolates that lost the URA3 marker. The integration of USA.ENO2p.Bi.ADH and URA3 marker removal was confirmed by PCR with primers JZ061, and JZ062 (SEQ ID NO: 246) using genomic DNA prepared with a Gentra® Puregene® Yeast/Bact. kit (Qiagen, Valencia, Calif.). The integration of USA.ENO2p.Bi.ADH also was confirmed by DNA sequencing with primers JZ087, JZ060, and 643R (SEQ ID NO: 247) using genomic DNA prepared with a Gentra® Puregene® Yeast/Bact. kit (Qiagen, Valencia, Calif.). The correct isolates were designated as strains PNY1762 (MATa ura3Δ::loxP his3Δ::loxP pdc6Δ::UAS.ENO2p.Bi.ADH pdc1Δ::ilvD pdc5Δ::kivD(y)).

### B. HIS3+ Restoration

The deleted HIS3 coding sequence was restored in strain PNY1762 bp homologous recombination with a PCR cassette containing the HIS3 coding region and upstream and downstream homologies.

The HIS3 coding PCR cassette containing the HIS3 coding region and upstream and downstream flanking regions was amplified from PNY891 genomic DNA as template with primer T-HIS3(up300) (SEQ ID NO: 248) and primer B-HIS3(down273) (SEQ ID NO: 249). The resulting PCR products were gel-purified on an agarose gel followed by a gel extraction kit (Qiagen, Valencia, Calif.). Competent cells of PNY 1773 were made and transformed with the HIS3+PCR cassette using a Frozen-EZ Yeast Transformation II™ kit (Zymo Research Corporation, Irvine, Calif.). Transformation mixtures were plated on synthetic complete media lacking histidine supplemented with 0.5% ethanol (no glucose) at 30° C. Transformants with a HIS3+integration were screened for growth on synthetic complete media lacking histidine supplemented with 0.5% ethanol (no glucose), and confirmed by colony PCR with primer sets T-HIS3(up300) and primer B-HIS3(down273). The correct isolates were designated as JZ061 (MATa ura3Δ::loxP pdc6Δ::UAS.ENO2p.Bi.ADH pdc1Δ::ilvD pdc5Δ::kivD(y)).

C. Deletion of the YMR226C Coding Region and Replacement with PDC5p.alsS

The YMR226C coding region was deleted and replaced with the PDC5p promoter and alsS coding region in JZ061 strain by homologous recombination with a PCR cassette A-PDC5p.alsS-B-U-C containing the homology upstream (fragment A) and downstream (fragment B) of the YMR226C coding region, promoter PDC5p from Saccharomyces cerevisiae, alsS coding region coding region from Bacillus subtilis subsp. subtilis str. 168 (NC_000964), and a URA3 gene along with the promoter and terminator (fragment U) for selection of transformants, and the 3′-region of the YMR226C coding region (fragment C).

The fragment A (531 bp) was PCR-amplified from the genomic DNA of PNY0891 using Phusion® DNA polymerase (New England BioLabs Inc., Ipswich, Mass.) with primers JZ151 (SEQ ID NO: 250) and JZ317 (SEQ ID NO: 251). The PDC5p.alsS cassette (2,583 bp) was PCR-amplified from pYZ152 (SEQ ID NO: 281) with primers JZ316 (SEQ ID NO: 252) and JZ313 (SEQ ID NO: 253). The fragment B (562 bp) was PCR-amplified from the genomic DNA of PNY0891 with primers JZ312 (SEQ ID NO: 254) and JZ157 (SEQ ID NO: 255). The fragment U (1,260 bp) was PCR-amplified from the genomic DNA of PNY0891 with primers JZ156 (SEQ ID NO: 256) and JZ159 (SEQ ID NO: 257). The fragment C (528 bp) was PCR-amplified from the genomic DNA of PNY0891 with primers JZ158 (SEQ ID NO: 258) and JZ160 (SEQ ID NO: 259). The resulting PCR product was purified on an agarose gel followed by a gel extraction kit (Qiagen, Valencia, Calif.). The B-U-C cassette was created by overlapping PCR by mixing the fragment B, fragment U, and fragment C and amplifying with primers JZ312 and JZ160. The PCR product was purified with a PCR purification kit (Qiagen, Valencia, Calif.). PCR cassette A-PDC5p.alsS-B-U-C(5,228 bp) was created by overlapping PCR by mixing the fragment A, PDC5p.alsS cassette, and B-U-C cassette and amplifying with primers JZ151 and JZ160. The resulting PCR product was purified on an agarose gel followed by a gel extraction kit (Qiagen, Valencia, Calif.).

Competent cells of JZ061 were made and transformed with the PCR cassette A-PDC5p.alsS-B-U-C using a Frozen-EZ Yeast Transformation II™ kit (Zymo Research Corporation, Irvine, Calif.). Transformation mixtures were plated on synthetic complete media lacking uracil supplemented with 0.5% ethanol (no glucose) at 30° C. Transformants with a YMR226C knockout and PDC5p.alsS-B-U-C integration were screened for by PCR with one set of primers URA3F (SEQ ID NO: 260) and JZ161 (SEQ ID NO: 261), and another set of primers URA3R (SEQ ID NO: 262) and JZ320 (SEQ ID NO: 263) using genomic DNA prepared with a Gentra® Puregene® Yeast/Bact. kit (Qiagen, Valencia, Calif.). To remove the URA3 marker from the chromosome, correct transformants were grown overnight in YPE (0.5% ethanol) and plated on synthetic complete medium supplemented with ethanol (no glucose) and containing 5-fluoro-orotic acid (0.1%) at 30° C. to select for isolates that lost the URA3 marker. The integration of PDC5p.alsS and URA3 marker removal was confirmed by PCR with primers JZ150 (SEQ ID NO: 264), and JZ161 using genomic DNA prepared with a Gentra® Puregene® Yeast/Bact. kit (Qiagen, Valencia, Calif.). The integration of PDC5p.alsS also was confirmed by DNA sequencing with primers JZ320, JZ319 (SEQ ID NO: 265), and JZ161 using genomic DNA prepared with a Gentra® Puregene® Yeast/Bact. kit (Qiagen, Valencia, Calif.). The correct isolates were designated as strains JZ063 (MATa ura3Δ::loxP pdc6Δ::UAS.ENO2p.Bi.ADH pdc1Δ::ilvD pdc5Δ::kivD(y) ymr226cΔ::PDC5p. alsS).

D. Replacement of pdc5Δ::kivD(y) with pdc5Δ::kivD.Lg.y

The Lactococuss lactis kivD(y) coding region integrated at the pdc5Δ deletion region in JZ063 was replaced with Listeria grayi kivD gene that was codon-optimized for Saccharomyces cerevisiae (kivD.Lg.y) by homologous recombination.

The kivD.Lg.y integration cassette A-KivD.Lg.y-B-U-C contains the homology upstream (fragment A) and downstream (fragment B) of the PDC5 coding region, kivD.Lg.y coding region from Listeria grayi, URA3 gene along with the promoter and terminator (fragment U) for selection of transformants, and the 3′ region of the kivD.Li.y coding region (fragment C). The fragment A was amplified from PNY0891 genomic DNA as template with primer T-A(PDC5) (SEQ ID NO: 220), and B-A(kivDLg) (SEQ ID NO: 266), containing a 5′ tail with homology to the 5′ end of kivD.Li.y. The kivD.Li.y coding region was amplified from pBP1719 (pUC19-ura3MCS-U(PGK1)Pfbai-kivD Lg(y)-ADH1 BAC-kivD.LI fragment C (SEQ ID NO: 288) with primer T-kivDLg(A) (SEQ ID NO: 267), containing a 5′ tail with homology to the 3′ end of the fragment A, and B-kivDLg(B) (SEQ ID NO: 268), containing a 5′ tail with homology to the 5′ end of the fragment B. The fragment B-U was amplified from pBP904 (pUC19-URA3-sadB-PDC5fragmentB) (SEQ ID NO: 279) with primer T-B(kivDLg) (SEQ ID NO: 269), containing a 5′ tail with homology to the 3′ end of kivD.Li.y, and oBP546(new) (SEQ ID NO: 270), containing a 5′ tail with homology to the 5′ end of the fragment C. The fragment C was amplified with primer oBP547(new) (SEQ ID NO: 271), containing a 5′ tail with homology to the 3′ end of the fragment U, and primer oBP539(new) (SEQ ID NO: 272). PCR products were purified with a PCR purification kit (Qiagen, Valencia, Calif.). The fragment A-KivD.Lg.y was created by overlapping PCR by mixing the fragment A and fragment KivD.Lg.y and amplifying with primers T-A(PDC5) and B-kivDLg(B). The fragment B-U-C was created by overlapping PCR by mixing the fragment B-U and fragment C and amplifying with primers T-B(kivDLg) and oBP539(new). The resulting PCR products were gel-purified on an agarose gel followed by a gel extraction kit (Qiagen, Valencia, Calif.). The A-KivD.Lg.y-B-U-C cassette was created by overlapping PCR by mixing the fragment A-KivD.Lg.y and fragment B-U-C and amplifying with primers T-A(PDC5) and oBP539(new). The PCR product was purified with a PCR purification kit (Qiagen, Valencia, Calif.).

Competent cells of JZ063 were made and transformed with the PCR cassette A-KivD.Lg.y-B-U-C using a Frozen-EZ Yeast Transformation II™ kit (Zymo Research Corporation, Irvine, Calif.). Transformation mixtures were plated on synthetic complete media lacking uracil supplemented with 0.5% ethanol (no glucose) at 30° C. Transformants with a A-KivD.Lg.y-B-U-C integration were screened for by PCR with primer sets oBP540/kivDLg(569R) and kivDLg(530F)/oBP541 using genomic DNA prepared with a Gentra® Puregene® Yeast/Bact. kit (Qiagen, Valencia, Calif.). To remove the URA3 marker from the chromosome, correct transformants were grown overnight in YPE (0.5% ethanol) and plated on synthetic complete medium supplemented with ethanol (no glucose) and containing 5-fluoro-orotic acid (0.1%) at 30° C. to select for isolates that lost the URA3 marker. The replacement of kivD(y) with kivD.Lg.y, and URA3 marker removal were confirmed by DNA sequencing with primers kivDLg(569R) (SEQ ID NO: 273), kivDLg(530F) (SEQ ID NO: 274), and kivDLg(1162F) (SEQ ID NO: 275) using genomic DNA prepared with a Gentra® Puregene® Yeast/Bact. kit (Qiagen, Valencia, Calif.). The correct isolates were designated as JZ065 (MATa ura3Δ::loxP pdc6Δ::UAS.ENO2p.Bi.ADH pdc1Δ::ilvD pdc5Δ::kivD.Lg.y ymr226cΔ::PDC5p. alsS).

E. Transformation with pNZ001

JZ065 were transformed with a plasmid pNZ001 (SEQ ID NO: 284) carrying K9D3.KARI gene from Anaerostipes caccae DSM 14662 and ilvD gene from Streptococcus mutans ATCC No. 700610. Competent cells of JZ065 were made and transformed with a plasmid pNZ001 using a Frozen-EZ Yeast Transformation II™ kit (Zymo Research Corporation, Irvine, Calif.). Transformed cells were plated on synthetic complete media lacking uracil supplemented with 0.5% ethanol (no glucose) at 30° C. Resulting transformant was designated the isobutanologen strain PNY0684 (MATa ura3Δ::loxP pdc6Δ::UAS.ENO2p. Bi.ADH pdc1Δ::ilvD pdc5Δ::kivD.Lg.y ymr226cΔ::PDC5p. alsS/pNZ001).

### Example 1

### Selection for Isobutanol Tolerance

Cultures of PNY1530 were subjected to five rounds of selection in increasing concentrations of isobutanol. The first round of isobutanol selection was initiated by growing PNY1530 to OD₆₀₀=1.8 in 100 ml of SEU culture medium (yeast nitrogen base supplemented with Sigma yeast synthetic dropout medium without uracil (Sigma Y1501) and with 0.2% ethanol). The cells were centrifuged, resuspended in 100 ml of fresh culture medium and grown for several hours to approximately 3 OD₆₀₀ units. The culture was centrifuged and resuspended at OD₆₀₀=100 (approximately 5×10⁸ cfu/ml) in 3 ml of culture medium without ethanol. A small sample was removed from the cell suspension for a viable cell count, and the remaining cell suspension was divided into three cultures containing 1.5% isobutanol, 1.7% isobutanol or 2.0% isobutanol. Each culture was incubated at 30° C. on a roller drum for 24 hours. The cultures were then centrifuged, and the cell pellets were each resuspended in 1 ml of culture medium without isobutanol or ethanol. Small samples were removed from the cultures for viable cell counts, and the remaining portion of each cell suspension (approximately 975 μl) was inoculated into 10 ml of SEU culture medium. The cultures were incubated at 30° C. with shaking. In general, each subsequent round of isobutanol selection was initiated with cells that had survived the highest level of isobutanol selection in the previous round of exposure.

Increased numbers of survivors were observed following each exposure to isobutanol (Table 9). For example, only 1.8% of the cells survived 24 hour exposure to 2.0% isobutanol during Selection I whereas 100% of the population survived 24 hour exposure to 2.0% isobutanol during Selection IV. Similarly, no survivors were detected following exposure to 2.7% isobutanol during Selection II whereas 0.004% of the evolved population survived exposure to 2.7% isobutanol during Selection V. Hence, repeated isobutanol selection followed by growth of survivors resulted in an evolved cell population that was better able to survive exposure to isobutanol.

TABLE 9
Evolving isobutanol tolerance in the isobutanologen PNY1530

| | Percent Survival¹ | | | | |
|---|---|---|---|---|---|
| Concentration² | Selection I | Selection II | Selection III | Selection IV | Selection V |
| 1.5% Isobutanol | 73 | | | | |
| 1.7% Isobutanol | 53 | | | | |
| 2.0% Isobutanol | 1.8 → | 12 → | 21 | 100 | |
| 2.2% Isobutanol | | | 0.8 | 45 | |
| 2.5% Isobutanol | | 0.0003 | 0.0006 → | 14 → | 4.5 |
| 2.7% Isobutanol | | ND³ | | | 0.004 |
| 3.0% Isobutanol | | | | | ND |

¹The arrow (→) indicates survivors that were used to initiate the next round of isobutanol selection.
²Calculated concentrations
³Not detected.

### Example 2

### Selection for Growth in the Presence of Isobutanol by Serial Passage

A population of cells that had survived 24 hour exposure to 2.5% isobutanol during Selection IV (see Table 9) was diluted into SEGU culture medium (SEU with 0.2% glucose) to OD₆₀₀=0.8. The diluted cell suspension was divided into 1.5 ml cultures, dispensed into 2 ml sterile screw cap tubes and supplemented with various concentrations of isobutanol. The cultures were incubated at 30° C. on a roller drum. After 24 hours, the cultures were diluted 1:2 with the SEGU culture medium comprising the same amount of isobutanol as the previous culture. After an additional 24 hours, 0.5% isobutanol was found to be the highest concentration that permitted growth. The 0.5% culture was serially sub-cultured 10 times by diluting the culture to approximately OD₆₀₀=0.5 in SEGU culture medium containing 0.5% isobutanol and incubating the diluted culture at 30° C. for 24 to 48 hours before diluting the culture again.

After the last sub-culture, the 0.5% culture was plated and colonies were inoculated into SEGU in microtiter plates. The Bioscreen C growth curve machine was used to identify variants with better growth characteristics than strain PNY1530. The growth rates of 188 isolates in SEGU culture medium without added isobutanol were compared to each other and to PNY1530, and 30 isolates were chosen for further testing in the BioScreen by culturing the isolates in SEGU with 0%, 1% or 2% isobutanol. Growth of the 30 isolates for 24 hours was analyzed by determining the difference between initial OD₆₀₀ and final OD₆₀₀ (AOD) for each isolate. Isolate 20 and isolate 21 had the highest levels of growth in both 1% and 2% isobutanol (Table 10). In addition, isolate 22 had higher growth in 2% isobutanol than all of the other isolates except 20 and 21. Isolates 20, 21 and 22 were chosen for additional characterization. However, isolate 20 failed to grow well in subsequent flask experiments. Therefore, further experimentation proceeded with isolate 21 (PNY0314) and isolate 22 (PNY0315).

TABLE 10
BioScreen C growth of evolved PNY1530
isolates in 0%, 1%, or 2% isobutanol

| | ΔOD¹ | | |
|---|---|---|---|
| | 0% | 1% | 2% |
| Isolate | Isobutanol | Isobutanol | Isobutanol |
| 1 | 0.401 | 0.142 | 0.057 |
| 2 | 0.354 | 0.137 | 0.079 |
| 3 | 0.394 | 0.12 | 0.035 |
| 4 | 0.329 | 0.143 | 0.093 |
| 5 | 0.383 | 0.125 | 0.087 |
| 6 | 0.328 | 0.151 | 0.097 |
| 7 | 0.357 | 0.12 | 0.085 |
| 8 | 0.382 | 0.125 | 0.09 |
| 9 | 0.390 | 0.171 | 0.063 |
| 10 | 0.325 | 0.157 | 0.094 |
| 11 | 0.340 | 0.138 | 0.033 |
| 12 | 0.313 | 0.121 | 0.057 |
| 13 | 0.274 | 0.12 | 0.008 |
| 14 | 0.282 | 0.12 | 0.014 |
| 15 | 0.183 | 0.113 | 0.018 |
| 16 | 0.261 | 0.124 | 0.067 |
| 17 | 0.270 | 0.122 | 0.093 |
| 18 | 0.260 | 0.157 | 0.089 |
| 19 | 0.246 | 0.135 | 0.051 |
| 20 | 0.236 | 0.147 | 0.149 |
| 21 | 0.274 | 0.126 | 0.131 |
| 22 | 0.215 | 0.079 | 0.114 |
| 23 | 0.178 | 0.089 | 0.03 |
| 24 | 0.174 | 0.06 | 0.047 |
| 25 | 0.186 | 0.089 | 0.058 |
| 26 | 0.187 | 0.089 | 0.047 |
| 27 | 0.143 | 0.081 | 0.065 |
| 28 | 0.192 | 0.071 | 0.021 |
| 29 | 0.198 | 0.114 | 0.008 |
| 30 | 0.184 | 0.106 | 0.047 |
| PNY1530 | 0.069 | 0.088 | 0.034 |

¹ΔOD = (initial OD₆₀₀ − final OD₆₀₀)

### Example 3

### Glucose Utilization by PNY1530, PNY0314, and PNY0315 in Culture Medium with 1% Isobutanol

The abilities of PNY0314, PNY0315 and PNY1530 to metabolize glucose in the presence of 1% isobutanol were compared in a shake flask experiment.

Each strain was grown overnight in 200 ml of SEGU at 30° C. with shaking in non-vented 500 ml culture flasks, centrifuged, and then resuspended to OD₆₀₀=5.9−6.0 in SEU with 20 g/L glucose. Samples (500 μl) were withdrawn from the cultures at 2 hour intervals for glucose analysis. The samples were mixed with 500 μl of 10% TCA, centrifuged and analyzed using an YSI 2700 Select analyzer with probe assembly Part #110923.

During the first 7 to 8 hours of the experiment, PNY0314 and PNY0315 utilized glucose at rates (0.71 and 0.80 g/gdcw/h respectively) that were comparable to or slightly higher than PNY1530 (0.68 g/gdcw/h) in the absence of isobutanol (data not shown). During the same time, PNY0314 and PNY0315 metabolized glucose in cultures supplemented with 1% isobutanol at rates that were approximately 30% higher than PNY1530 (Table 11).

TABLE 11
Glucose Utilization by PNY1530, PNY0314 and
PNY0315 in cultures containing 1% Isobutanol.

| | Glucose Remaining¹ (g/L) | | |
|---|---|---|---|
| Time (hr) | PNY1530 | PNY0314 | PNY0315 |
| 0.0 | 20.47 | 20.47 | 20.47 |
| 1.0 | 20.47 | 20.26 | 20.06 |
| 3.0 | 18.82 | 18.64 | 18.29 |
| 5.0 | 17.60 | 16.80 | 16.67 |
| 7.0 | 17.27 | 15.86 | 15.64 |
| 24.0 | 15.53 | 12.76 | 13.29 |
| Glucose | 0.242 | 0.313 | 0.322 |
| Utilization Rate² | | | |
| (g/gdcw/h) | | | |

¹Average of two cultures for each strain
²Rates calculated for time 1 to 7 hours.

### Example 4

### Fermentation with PNY1530, PNY0314 and PNY0315

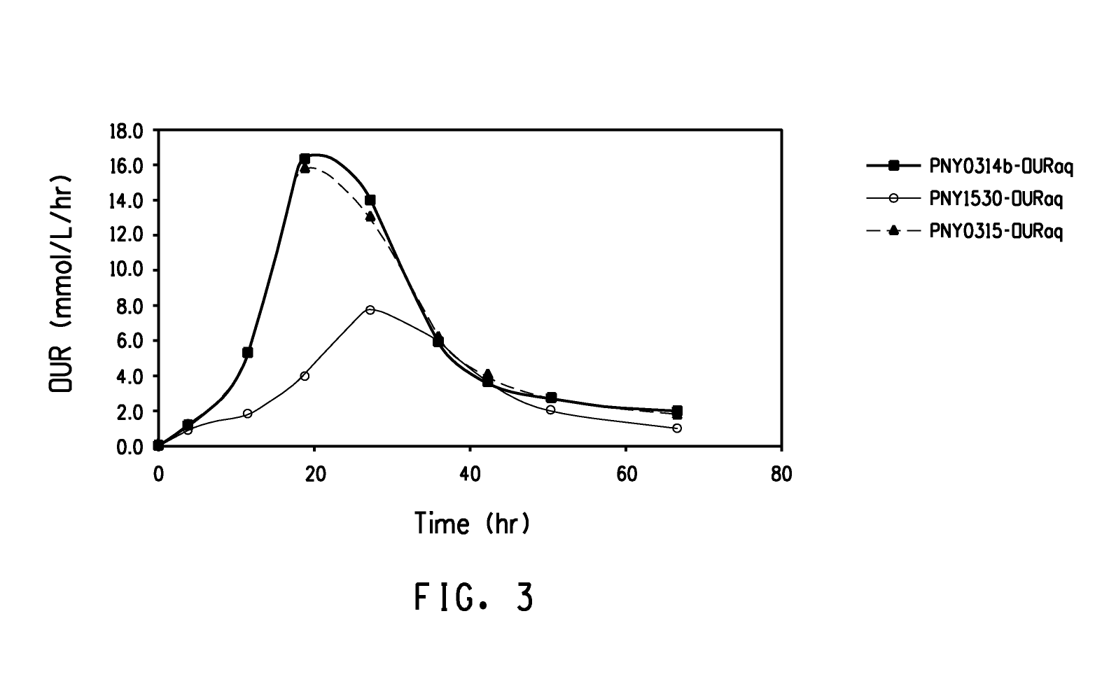

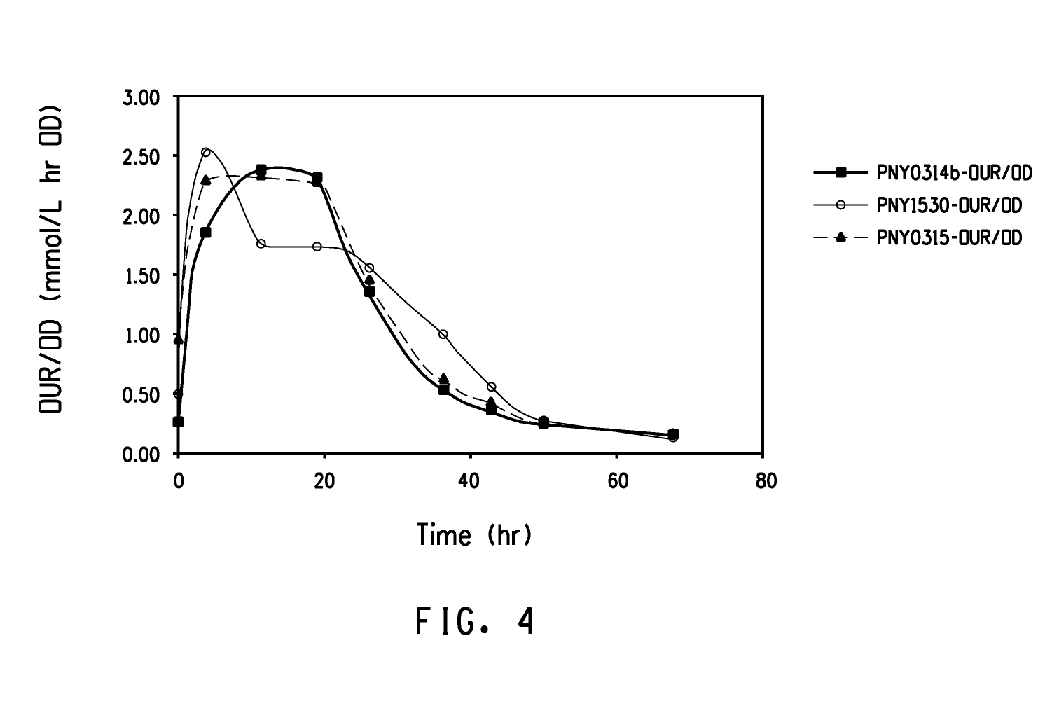

The growth characteristics of PNY1530, PNY0314 and PNY0315 were examined in a batch fermentation process with synthetic medium containing glucose. PNY0314 and PNY0315 grew at higher rates during the logarithmic growth phase and produced more biomass by the onset of stationary phase than PNY1530 (FIG. 2). PNY0314 and PNY0315 also had higher O₂ uptake rates compared to PNY1530 (FIG. 3). However, the specific O₂ uptake rates of PNY0314 and PNY0315 were higher than PNY1530 only for a relatively short period from about the 10 hour sample to the 20 hour sample (FIG. 4), with the specific O₂ uptake rates of PNY0315 and PNY0315 generally being lower than PNY1530 after the 20 hour sample.

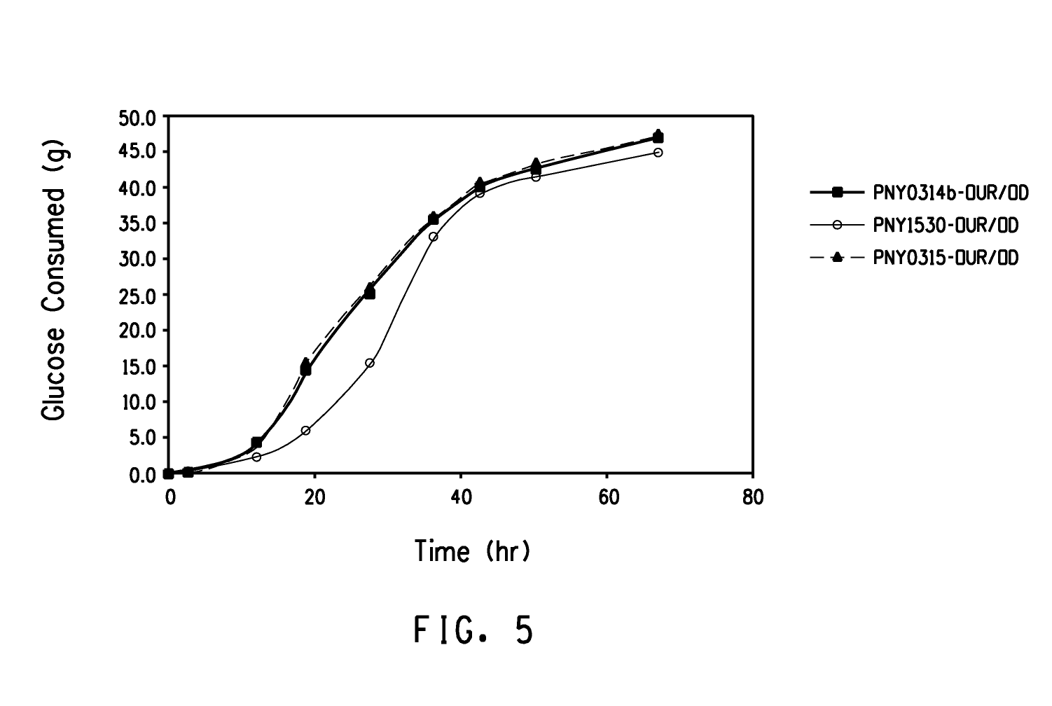

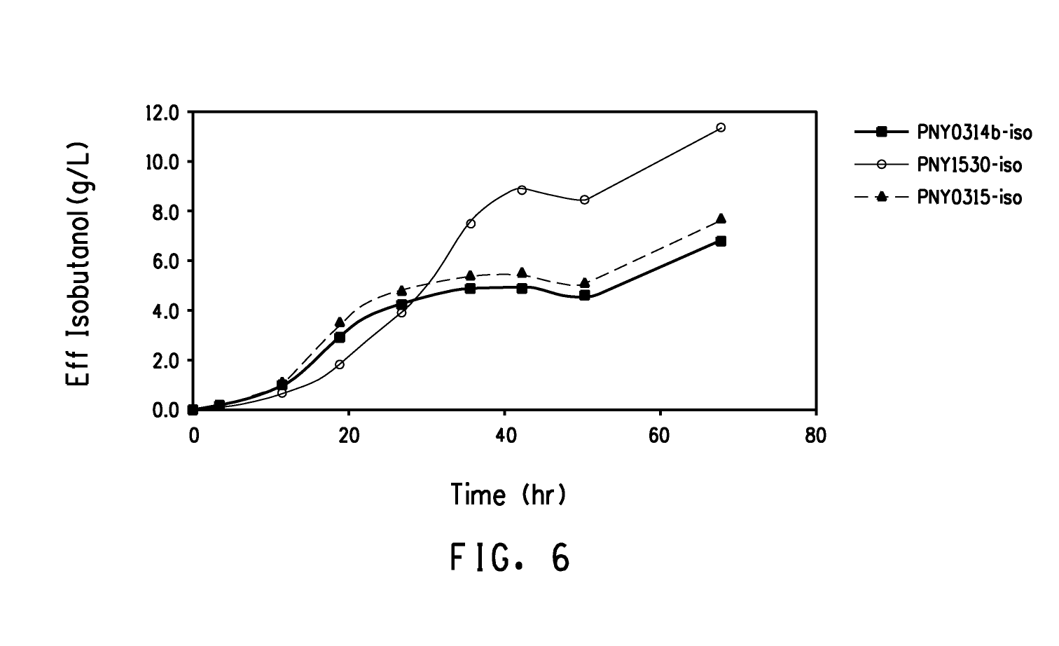

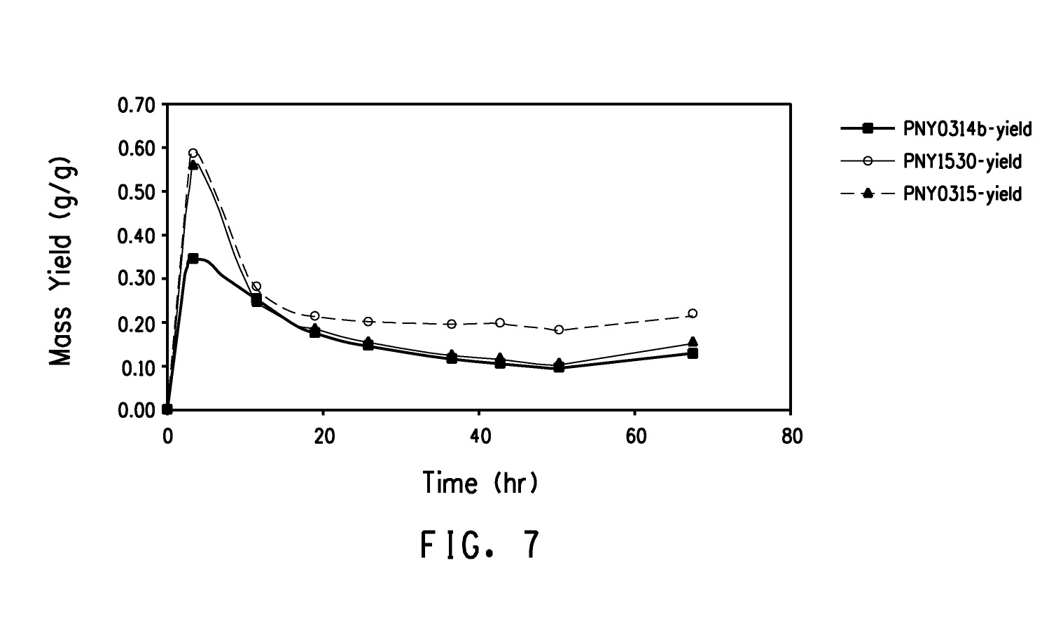

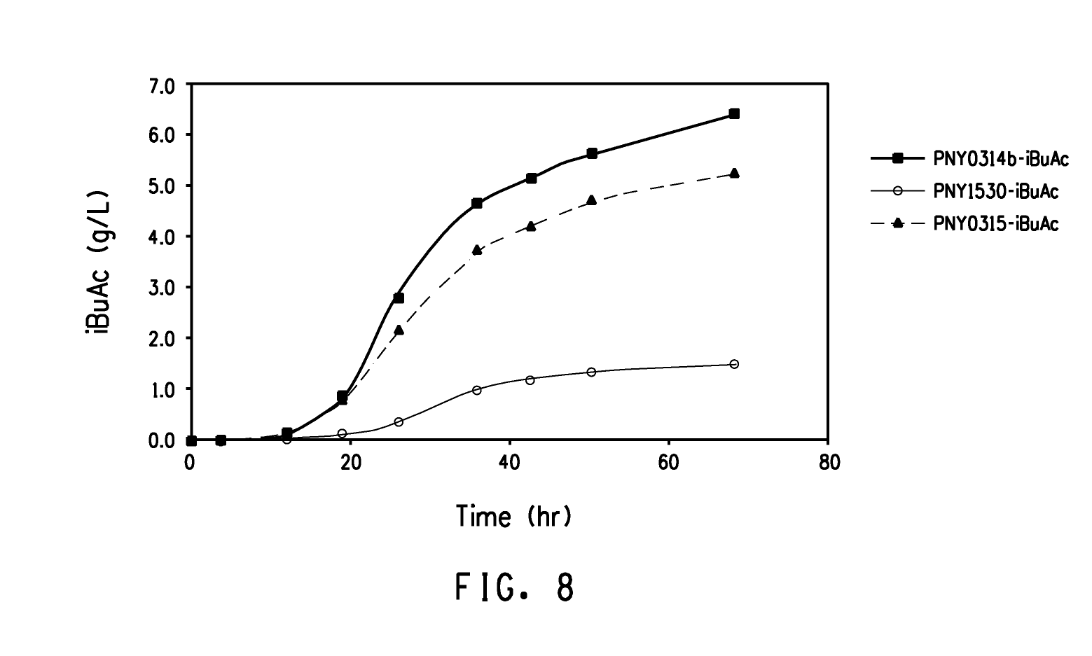

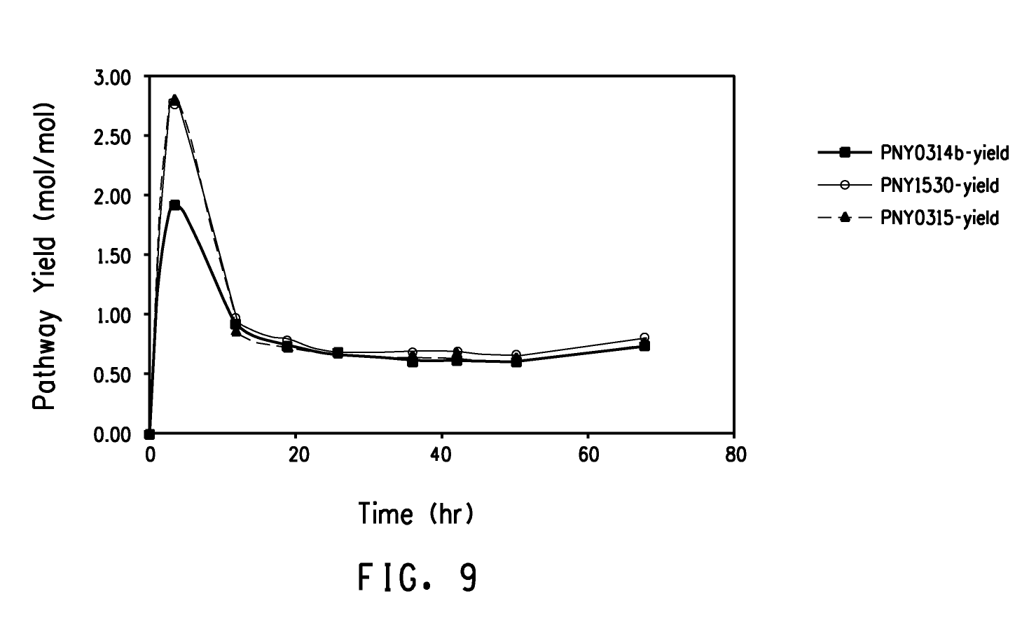

Although PNY0314 and PNY0315 consumed more glucose than PNY1530 throughout the experiment (FIG. 5), the two variants produced less isobutanol than the control strain (FIG. 6). As a result, PNY0314 and PNY0315 had lower mass yields for isobutanol than PNY1530 (FIG. 7). However, PNY0314 and PNY0315 produced more isobutyric acid than PNY1530 (FIG. 8). The increased levels of isobutyric acid accounted for the lower isobutanol titers (FIG. 6) and yields (FIG. 7) displayed by PNY0314 and PNY0315. However, the pathway yields for all three strains were essentially the same (FIG. 9), indicating that the same amounts of glucose-derived carbon entered the isobutanol pathway in all three strains. In addition, PNY1530 produced more glycerol than PNY0314 and PNY0315 (FIG. 10), indicating that PNY0314 and PNY0315 were likely under less physiological stress than PNY1530.

Taken together, the results of the fermentation experiment indicated that PNY0314 and PNY0315 directed more carbon to biomass production than PNY1530 but did so without diverting carbon from the isobutanol pathway.

### Example 5

### Isolation and Characterization of PNY0342

Strain PNY0342 was isolated from a population of cells that had been evolved in a chemostat in growth medium supplemented with glucose and isobutanol to select for cells that were better able to grow and utilize glucose in the presence of isobutanol.

Isobutanologen PNY2242 (MATa ura3Δ::loxP his3A pdc6Δ pdc1Δ::P[PDC1]-DHAD|ilvD_Sm-PDC1t-P[FBA1]-ALS|alsS_Bs-CYC1t pdc5Δ::P[PDC5]-ADH|sadB_Ax-PDC5t gpd2Δ::loxP fra2Δ::P[PDC1]-ADH|adh_H1-ADH1t adh1Δ::UAS(PGK1)P[FBA1]-kivD_L1(y)-ADH1t yprcΔ15Δ::P[PDC5]-ADH|adh_H1-ADH1t ymr226cΔ ald6Δ::loxP; pLH702, pYZ067DkivDDhADH), disclosed in U.S. Patent Appl. No. 2013/0071891, which is herein incorporated by reference, was inoculated into an Appilikon Fermentor (Appilikon Inc., Clinton, N.J.) that was operated as a chemostat. The bioreactor system was composed of a 1-L dished bottom reactor, Controller ADI 1032 P100, and stirrer unit with marine and turbine impellers. Bio Controller ADI 1030 Z510300020 with appropriate sensors monitored pH, dissolved oxygen, and temperature. A Cole Parmer pump and pump head were used for addition of NaOH to maintain pH 4.1. The temperature was maintained at 30° C. by using a circulating water bath. Medium volume in the chemostat vessel was 1000 mL. The chemostat was not sparged with gas, and a low stirrer speed of 50 rpm was used to prevent settling of the cells. Cell density in the bioreactor was monitored by measuring the optical density at 600 nm (OD₆₀₀).

The chemostat was inoculated with an overnight culture of PNY2242, and after 24 hours of batch mode operation, the chemostat was operated in continuous feed mode. The initial flow rate of 0.5 mL/minute (dilution rate=0.03 h⁻¹) was increased to 0.7 mL/min (dilution rate=0.042 h⁻¹) on Day 39. These flow rates correspond to doubling times of 23.1 h and 16.5 h, respectively. The isobutanol concentration and the glucose concentration in the influent medium (6.7 g yeast nitrogen Base without amino acids, Yeast drop out Y2001 1.4 g/L, Leucine 380 mg/L, Tryptophan 76 mg/L, Thiamine 20 mg/L, 1 ml of ergastrol stock (2 g ergastrol+100 ml Ethanol+100 ml Tween 80), 2% ethanol, 0.5% glucose) are closely related in that increasing either the influent isobutanol concentration or the influent glucose concentration resulted in an increase of the isobutanol concentration in the chemostat vessel. Hence, the isobutanol concentration in the chemostat increased to 0.13% by Day 6 before addition of isobutanol to the chemostat through the influent medium. The amount of isobutanol entering the bioreactor through the feed was gradually increased to approximately 0.7% (w/v), and the influent glucose concentration was increased in 2 steps from the initial concentration of 0.5% to a concentration of 1%. As a result, the isobutanol concentration gradually increased to a peak value of 0.88% by Day 75.

Cells from a sample collected from the chemostat on day 95 were plated onto SEG agar, and typical colonies were chosen randomly for analysis. Isolates were grown and compared to PNY2242 for utilization of glucose in the presence and absence of isobutanol (Table 12). Glucose utilization was measured in cultures that were concentrated to 8 OD units. Strain PNY0342 was identified as an isolate that had glucose utilization rates for the first six hours of the experiment that were essentially the same as the PNY2242 control in the absence of added isobutanol but significantly higher than PNY2242 in the presence of 1.5% isobutanol.

TABLE 12
Glucose Utilization by PNY2242 and PNY0342

Glucose Utilization Rate

| | 0% Isobutanol | 1.5% Isobutanol |
|---|---|---|
| Strain | 6 Hour Rate (g/gdcw/h) | 6 Hour Rate (g/gdcw/h) |
| PNY2242 | 1.01 | 0.12 |
| PNY0342 | 1.04 | 0.19 |

### Example 6

### Isolation and Characterization of PNY0347 and PNY0348

Strains PNY0347 and PNY0348 were isolated from a population of cells that had been evolved in a chemostat in growth medium supplemented with glucose and isobutanol to select for cells that were better able to grow and utilize glucose in the presence of isobutanol.

Isobutanologen PNY2071 was inoculated into an Appilikon Fermentor (Appilikon Inc., Clinton, N.J.) that was operated as a chemostat. The bioreactor system was composed of a 1-L dished bottom reactor, Controller ADI 1032 P100, and stirrer unit with marine and turbine impellers. Bio Controller ADI 1030 Z510300020 with appropriate sensors monitored pH, dissolved oxygen, and temperature. A Cole Parmer pump and pump head were used for addition of NaOH to maintain pH 4.1. The temperature was maintained at 30° C. by using a circulating water bath. Medium volume in the chemostat vessel was 1000 mL. The chemostat was not sparged with gas, and a low stirrer speed of 50 rpm was used to prevent settling of the cells. Cell density in the bioreactor was monitored by measuring the optical density at 600 nm (OD₆₀₀).

A chemostat was inoculated with an overnight culture of PNY2071, and after 24 hours of batch mode operation, the chemostat was operated in continuous feed mode with a flow rate of 0.7 ml/min (dilution rate=0.042 h⁻¹). The amount of isobutanol entering the bioreactor through the feed was gradually increased to approximately 0.8% (w/v), and the influent glucose concentration was increased in 3 steps from the initial concentration of 0.5% to a concentration of 1%. As a result, the isobutanol concentration gradually increased to a peak value of 1% by Day 48.

Cells from a sample collected from the chemostat on day 48 were plated onto SEG agar, and typical colonies were chosen randomly for analysis. Isolates were grown and compared to PNY2071 for utilization of glucose in the presence and absence of isobutanol (Table 13). Glucose utilization was measured in cultures that were concentrated to 6.9 OD units. Strains PNY0347 and PNY0348 were identified as isolates that had glucose utilization rates for the first six hours of the experiment that were essentially the same as the PNY2071 control in the absence of added isobutanol but significantly higher than PNY2071 in the presence of 1.5% isobutanol.

TABLE 13
Glucose Utilization by PNY2242 and PNY0342

Glucose Utilization Rate

| | 0% Isobutanol | 1.5% Isobutanol |
|---|---|---|
| Strain | 6 Hour Rate (g/gdcw/h) | 6 Hour Rate (g/gdcw/h) |
| PNY2071 | 1.05 | 0.41 |
| PNY0347 | 0.94 | 0.54 |
| PNY0348 | 1.5 | 0.57 |

### Example 7

### Isolation and Characterization of PNY0684E1 and PNY0684E5

Strains PNY0684E1 and PNY0684E5 were isolated from a population of cells that had been evolved in medium with increasing concentrations of sucrose.

Strain PNY0684 was inoculated into 20 ml of CIG medium (6.7 g/L Yeast Nitrogen Base, 1 ml/L Delft vitamins, 100 mM MES, pH 6.0, 5 g/L yeast extract, 5 g/L ethanol) in a 125 ml vented flask. The initial sucrose concentration was 2 g/L for the first two days and was then gradually increased as time progressed: 4 g/L for 4 days, 6 g/L for 4 days, 10 g/L for 3 days, 20 g/L for 7 days, 25 g/L for 14 days and 30 g/L for 14 days. The culture was incubated at 30° C. with shaking at 120 rpm. The culture was diluted 1:10 with fresh culture medium approximately every 24 hours. PNY0684E1 was isolated from the culture on day 30, after 106 generations, and PNY0684E5 was isolated from the culture on day 50, after 187 generations. PNY0684 had an aerobic growth rate of 0.032 μ(h⁻¹) and PNY0684E1 and PNY0684E5 had growth rates of 0.122 μ(h⁻¹) and 0.128 μ(h⁻¹) respectively in CIG medium. In addition, at the end of 24 h PNY0684 reached a final OD₆₀₀ of 1.0 and PNY0684E1 and PNY0684E5 reached a final OD₆₀₀ of 10.2 and 12.2 respectively. In 24 h, PNY0684 had utilized 11.84 g/L of Glucose equivalent and PNY0684E1 had utilized 33.62 g/L (glucose equivalent) and PNY0684E5 had utilized 39.94 g/L glucose equivalent)

### Example 8

### Identification of Mutations in PNY0314 and PNY0315

A Puregene Yeast/Bact. Kit (Catalog #158567, Qiagen, Valencia, Calif.) was used to extract genomic DNA from cells grown in 100 ml of SEGU culture medium with shaking at 30° C. for 20 hours. The genomic DNA was used for sequencing using an Illumina HiSeq2000 sequencer (Illumina, San Diego, Calif.) according to standard procedures.

The PNY1530, PNY0314 and the PNY0315 genomic sequences were each assembled by alignment with the CEN.PK113-7D genomic sequence as the reference (BMC Genomics (2010) 11:723). Differences between the reference sequence and each isobutanologen sequence were compiled into spreadsheet lists that were sorted according to chromosome number and base pair position relative to the reference strain. The three lists were then aligned, and mutations were identified that were present in the evolved strains but absent from PNY1530.

The analysis considered ORFs that had been altered by base pair changes in both PNY0314 and PNY0315 (Table 14). Although five of the seven identified ORFs have at least one base pair change at the same position (NUM1, PAU10, YGR109W-B, HSP32 and ATG13), four ORFs have one or more mutations that do not match (FLO9, PAU10, CYR1 and HSP32). Base pair changes represented by higher levels of coverage (i.e., higher sums of the nA;nC;nG;nT numbers in Table 14) can be viewed with higher degrees of confidence. In any event, this observation may indicate that either the non-matching mutations represent problems with sequencing, or certain genes accumulated independent mutations after the PNY0314 and PNY0315 lines diverged. It is most likely that mutations which are identical in both strains (e.g., the T to C change at position 758822 on chromosome 4 in NUM1) occurred before PNY0314 and PNY0315 diverged, and the non-matching mutations (e.g., the mutations in FLO9 on chromosome 1 at position 26035 in PNY0315 and at position 26172 in PNY0314) occurred after the two strains diverged.

FLO9 and CYR1 are the two ORFs that have only independent mutations in both PNY0314 and PNY0315. No matching mutations are present in these ORFs. The presence of independent mutations in CYR1 and FLO9 in both PNY0314 and PNY0315 suggests that these genes may be particularly important to the evolved phenotypes of PNY0314 and PNY0315.

FLO9 encodes a lectin-like protein that is involved in flocculation (Journal of Applied Microbiology (2011) 110:1-18). Null mutations in FLO9 result in reduced filamentous and invasive growth (Genetics (1996) 144:967-978). Exposure to fusel alcohols such as isobutanol results in invasive and filamentous growth (Folia Microbiologica (2008) 53:3-14). Since invasive/filamentous growth may be an adaptation to solid media, mutations in FLO9 may enable cells to grow better in suspension in liquid media.

TABLE 14
Mutations detected by sequencing of PNY0314 and PNY0315

| Strain | Mutation | Chromosome | Ref | nA; nC; nG; nT | Call | Gene | Function |
|---|---|---|---|---|---|---|---|
| PNY0315 | 26035 | 1 | G | 3; 0; 1; 0 | A | FLO9 | Lectin-like protein with |
| | | | | | | | similarity to Flo1p |
| PNY0314 | 26172 | 1 | T | 0; 15; 0; 4 | C | | |
| PNY0314 | 27110 | 1 | A | 1; 0; 4; 0 | G | | |
| PNY0314 | 758822 | 4 | C | 0; 7; 0; 24 | T | NUM1 | Protein required for |
| | | | | | | | nuclear migration |
| PNY0315 | 758822 | 4 | C | 0; 17; 0; 55 | T | | |
| PNY0314 | 1523311 | 4 | A | 0; 0; 5; 0 | G | PAU10 | Protein of unknown |
| | | | | | | | function |
| PNY0315 | 1523311 | 4 | A | 3; 0; 20; 0 | G | | |
| PNY0314 | 1523329 | 4 | C | 0; 1; 0; 4 | T | | |
| PNY0314 | 1523341 | 4 | G | 5; 0; 1; 0 | A | | |
| PNY0315 | 1523341 | 4 | G | 18; 0; 4; 0 | A | | |
| PNY0315 | 1523401 | 4 | C | 0; 2; 0; 9 | T | | |
| PNY0314 | 711742 | 7 | C | 0; 4; 0; 18 | T | YGR109W-B | Retrotransposon TYA |
| | | | | | | | Gag and TYB Pol genes |
| PNY0315 | 711742 | 7 | C | 0; 16; 0; 51 | T | | |
| PNY0315 | 430591 | 10 | C | 0; 0; 0; 69 | T | CYR1 | Adenylate cyclase, |
| | | | | | | | required for cAMP |
| | | | | | | | production and cAMP- |
| | | | | | | | dependent protein kinase |
| | | | | | | | signaling |
| PNY0314 | 430767 | 10 | C | 29; 0; 0; 0 | A | | |
| PNY0314 | 12429 | 16 | C | 0; 2; 0; 8 | T | HSP32 | Heat-Shock Protein |
| PNY0315 | 12429 | 16 | C | 0; 2; 0; 8 | T | | |
| PNY0315 | 12519 | | A | 1; 5; 0; 0 | C | | |
| PNY0314 | 908163 | 16 | C | 3; 0; 0; 0 | A | ATG13 | Regulatory subunit of |
| | | | | | | | the Atg1p signaling |
| | | | | | | | complex |
| PNY0315 | 908163 | 16 | C | 5; 0; 0; 0 | A | | |

### Example 9

### Identification of Mutations in PNY0342, PNY0347, PNY0348, PNY0684E1 and PNY0684E5

Samples of genomic DNA from PNY2242, PNY0342, PNY2071, PNY0347, PNY0348, PNY0684, PNY0684E1 and PNY0684E5 were extracted from cells (Puregene Yeast/Bact. Kit (Catalog #158567, Qiagen, Valencia, Calif.)) and used for sequencing using an Illumina HiSeq2000 sequencer (Illumina, San Diego, Calif.) according to standard procedures. The genomic sequences were assembled by alignment with the CEN.PK113-7D genomic sequence as the reference (BMC Genomics (2010) 11:723). Differences between the reference sequence and each isobutanologen sequence were compiled into Excel spread sheet lists that were sorted according to chromosome number and base pair position relative to the appropriate reference strain. The lists were then aligned, and mutations were identified that were present in the evolved strains but absent from the corresponding parent strains. The analysis identified mutations in FLO1, FLO5, and FLO9 were present in one or more of the evolved strains (Table 15).

TABLE 15
Mutations detected by sequencing of PNY0342, PNY0347, PNY0348, PNY0684E1
and PNY0684E5

| | | Gene | | | | | | |
|---|---|---|---|---|---|---|---|---|
| | | (ORF) Name | Base | | Position | Reference | | Variant |
| | | (Common/ | Position | Reference | of Amino | Amino | Variant | Amino |
| Strain | Chromosome | Systematic) | in ORF | Base | Acid | Acid | Base | Acid |
| PNY0314 | chr1 | FLO9/YAL063C | 860 | T | 287 | F | C | S |
| PNY0314 | chr1 | FLO9/YAL063C | 1798 | A | 600 | S | G | G |
| PNY0342 | chr1 | FLO9/YAL063C | 2897 | C | 966 | T | G | A |
| PNY0347 | chr1 | FLO9/YAL063C | 3661 | A | 1221 | T | G | A |
| PNY0684E1 | chr1 | FLO1/YAR050W | 1046 | G | 349 | R | C | P |
| PNY0684E5 | chr1 | FLO1/YAR050W | 1046 | G | 349 | R | C | P |
| PNY0348 | chr1 | FLO1/YAR050W | 4219 | G | 1407 | G | A | S |
| PNY0347 | chr8 | FLO5/YHR211W | 2543 | C | 848 | T | T | I |
| PNY0348 | chr8 | FLO5/YHR211W | 2543 | C | 848 | T | T | I |

### Example 10

### (Prophetic): Construction of an Isobutanologen Expressing FLO Gene Variants

The amino acid mutations identified in the FLO1, FLO5, or FLO9 genes in Example 1-9 are created in the isobutanologen strain PNY1530. The FLO gene mutations in Table 13 are introduced into the chromosome of the isobutanologen strain by homologous recombination with a PCR cassette containing homology upstream and downstream of the target FLO gene mutations and a URA3 gene for selection of transformants. Recycle of the selective marker is achieved using a scarless deletion method (Yeast (2006) 23:399-405). In order to use a URA3 gene as selective marker, the parental strains of PNY1530, which don't have a plasmid carrying KARI, DHAD and URA3 genes, are used.

To introduce the FLO9 F287S mutation (a base change from T to C at 860 base position) in PNY1530, 500 bp downstream of the FLO9 860 base position, nucleotides 861-1360 of SEQ ID NO: 180, is used as the downstream homology region (fragment C) for integration of the cassette. The fragment C is PCR-amplified with an upstream primer containing a NotI restriction site and a downstream primer containing a PacI restriction site and cloned into the corresponding sites in the integration vector pUC19-URA3MCS downstream of URA3 (SEQ ID NO: 164) to generate pUC19-URA3MCS-fragmentC vector. 500 bp upstream of the FLO9 860 base position, nucleotides 360-859 of SEQ ID NO: 181, is used as the upstream homology region (fragment A) for integration of the cassette. The fragment A 500 bp region (nucleotides 360-859), along with the 501 bp (T860C) (nucleotides 860-1360) containing the base change from T to C at 860 base position, and the 500 bp sequence (fragment B), nucleotides 1361-1860 of SEQ ID NO: 182, from immediately downstream of the 501 bp (T860C) region is synthesized (IDT, Coralville, Iowa). The resulting synthesized 3-part DNA product amplified with an upstream primer containing a PmeI restriction site and a downstream primer containing a FseI restriction site is cloned into the corresponding sites upstream of URA3 in the pUC19-URA3MCS-fragmentC vector to construct fragment pUC19-fragmentA-501 bp (T860C)-fragmentB-URA3MCS-fragmentC.

The mutations, FLO9 S600G, FLO9 T966A, FLO9 T1221A, FLO1 R349P, FLO1 G1407S, and FLO5 T848I, also are individually introduced into the chromosome of PNY1530 by the scarless deletion method with a cassette containing the appropriate base change, and upstream and downstream fragments as described above.

The integration cassettes from each integration vector are amplified and used to transform PNY1556 using a Frozen-EZ Yeast Transformation II kit (Zymo Research; Orange, Calif.). Transformation mixtures are plated on synthetic complete media lacking uracil supplemented with 0.5% ethanol at 30° C. Transformants are checked by PCR for integration at the correct locus. Two independent transformants for each cassette are grown in YPE (0.5% ethanol) and plated on synthetic complete medium supplemented with 0.5% ethanol and containing 5-fluoro-orotic acid (0.1%) at 30° C. to select for isolates that lost the URA3 marker. The replacement of the native FLO9, FLO1, and FLO5 gene sequences with the FLO variants, FLO9 F287S, FLO9 S600G, FLO9 T966A, FLO9 T1221A, FLO1 R349P, FLO1 G1407S, and FLO5 T848I in PNY1530 are confirmed by PCR and sequencing.

All seven strains are transformed with plasmid pYZ107F-OLE1p (SEQ ID NO: 166) using a Frozen-EZ Yeast Transformation II kit (Zymo Research; Orange, Calif.), and plated on synthetic complete media lacking uracil supplemented with 0.5% ethanol at 30° C.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A yeast microorganism comprising a pyruvate utilizing biosynthetic pathway, wherein the microorganism further comprises:
a) at least one genetic modification in an endogenous cell wall protein gene;
b) at least one genetic modification in an endogenous pyruvate decarboxylase gene; and
wherein the microorganism has an increase in tolerance to butanol as compared to a microorganism that lacks the at least one genetic modification.

2. The microorganism of claim 1, wherein the pyruvate decarboxylase gene is PDC1, PDC5, PDC6, or combinations thereof.

3. The microorganism of claim 1, wherein the genetic modification in the endogenous cell wall protein gene results in a decrease in flocculation and/or filamentous growth as compared to a microorganism that lacks the at least one genetic modification in an endogenous cell wall protein gene.

4. The microorganism of claim 3, wherein the cell wall protein gene is FLO1, FLO5, FLO9, FLO10, FLO11, or combinations thereof.

5. (canceled)

6. The microorganism of claim 1, comprising at least one genetic modification in an endogenous cell wall protein gene encoding a polypeptide having at least 80% sequence identity to SEQ ID NO: 30, SEQ ID NO: 31, or SEQ ID NO: 32.

7-8. (canceled)

9. The microorganism of claim 1, wherein the genetic modification is in a regulatory sequence of the endogenous cell wall protein gene.

10. The microorganism of claim 1, which further comprises a genetic modification in a gene that regulates the endogenous cell wall protein gene.

11. The microorganism of claim 10, wherein the genetic modification is in FLOG.

12. The microorganism of claim 1, which further comprises a genetic modification in a gene selected from the group consisting of CYR1, NUM1, PAU10, YGR109W-B, HSP32, ATG13, and combinations thereof.

13. The microorganism of claim 1, which further comprises a genetic modification in an endogenous glycerol-3-phosphate dehydrogenase (GPD) genes.

14. (canceled)

15. The microorganism of claim 1, which further comprises a genetic modification in FRA2.

16. The microorganism of claim 1, wherein the pyruvate utilizing biosynthetic pathway is an engineered C3-C6 alcohol production pathway.

17. The microorganism of claim 16, wherein the C3-C6 alcohol is selected from the group consisting of propanol, butanol, pentanol, and hexanol.

18-19. (canceled)

20. The microorganism of claim 16, wherein the engineered pathway comprises the following substrate to product conversions:
a. pyruvate to acetolactate;
b. acetolactate to 2,3-dihydroxyisovalerate;
c. 2,3-dihydroxyisovalerate to α-ketoisovalerate;
d. α-ketoisovalerate to isobutyraldehyde; and
e. isobutyraldehyde to isobutanol; and wherein
i. the substrate to product conversion of step (a) is performed by a recombinantly expressed acetolactate synthase enzyme;
ii. the substrate to product conversion of step (b) is performed by a recombinantly expressed acetohydroxy acid isomeroreductase enzyme;
iii. the substrate to product conversion of step (c) is performed by a recombinantly expressed acetohydroxy acid dehydratase enzyme;
iv. the substrate to product conversion of step (d) is performed by a recombinantly expressed decarboxylase enzyme; and
v. the substrate to product conversion of step (e) is performed by an alcohol dehydrogenase enzyme;
whereby isobutanol is produced from pyruvate via the substrate to product conversions of steps (a)-(e).

21-24. (canceled)

25. The microorganism of claim 1, wherein the microorganism is a member of a genus selected from the group consisting of Saccharomyces, Schizosaccharomyces, Hansenula, Candida, Kluyveromyces, Yarrowia, Issatchenkia, and Pichia.

26-27. (canceled)

28. The microorganism of claim 1, wherein the microorganism has an increased glucose utilization rate in the presence of butanol as compared to a microorganism lacking the at least one genetic modification to an endogenous cell wall protein gene.

29. A method of producing a fermentation product from a pyruvate utilizing biosynthetic pathway comprising:
a. providing the microorganism according to claim 1; and
b. growing the microorganism under conditions whereby the fermentation product is produced from pyruvate.

30. (canceled)

31. The method of claim 29, wherein the fermentation product is a C3-C6 alcohol selected from the group consisting of propanol, butanol, pentanol, and hexanol.

32-34. (canceled)

35. The method of claim 31, further comprising (c) recovering the butanol.

36. (canceled)

37. The method of claim 35 further comprising (d) removing solids from the fermentation medium.

38-66. (canceled)
